SEQUENCE LISTING (226 sequences)

SEQ ID NO: 1 - protein, 228 aa, synthetic construct
  REGION 1..228: note = Human IgG1 Fc domain having the N-terminal sequenceCDKTHTCPPCPAPE
CDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - DNA, 684 bp, synthetic construct
  misc_feature 1..684: note = DNA encoding human IgG1 Fc domain having the N-terminal sequenceCDKTHTCPPCPAPE
  source 1..684: mol_type = other DNA
tgtgacaaaactcacacatgcccaccgtgcccagcacctgaactcctgggggggccctcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 3 - protein, 251 aa, synthetic construct
  REGION 1..251: note = pre-Fc chimeric polypeptide having SHH signal peptide
MLLLARCLLLVLVSSLLVCSGLACDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 4 - DNA, 785 bp, synthetic construct
  misc_feature 1..785: note = DNA encoding pre-Fc chimeric polypeptide having SHH signalpeptide
  source 1..785: mol_type = other DNA
aagcttgaattcccaccatgctgctgctggcgagatgtctgctgctagtcctcgtctcctcgctgctggtatgctcgggactggcgtgtgacaaaactcacacatgcccaccgtgcccagcacctgaactcctgggggggccctcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgactcgagcggccg

SEQ ID NO: 5 - protein, 251 aa, synthetic construct
  REGION 1..251: note = pre-Fc chimeric polypeptide having IFN signal peptide
MALTFALLVALLVLSCKSSCSVGCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - DNA, 785 bp, synthetic construct
  misc_feature 1..785: note = DNA encoding pre-Fc chimeric polypeptide having IFN signalpeptide
  source 1..785: mol_type = other DNA
aagcttgaattcccaccatggccttgacctttgctttactggtggccctcctggtgctcagctgcaagtcaagctgctctgtgggctgtgacaaaactcacacatgcccaccgtgcccagcacctgaactcctgggggggccctcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgactcgagcggccg

SEQ ID NO: 7 - protein, 245 aa, synthetic construct
  REGION 1..245: note = pre-Fc chimeric polypeptide having CETP signal peptide
MLAATVLTLALLGNAHACDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 8 - DNA, 767 bp, synthetic construct
  misc_feature 1..767: note = DNA encoding pre-Fc chimeric polypeptide having CETP signalpeptide
  source 1..767: mol_type = other DNA
aagcttgaattcccaccatgctggctgccacagtcctgaccctggccctgctgggcaatgcccatgcctgtgacaaaactcacacatgcccaccgtgcccagcacctgaactcctgggggggccctcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgactcgagcggccg

SEQ ID NO: 9 - protein, 222 aa, synthetic construct
  REGION 1..222: note = Human IgG1 Fc domain having the N-terminal sequence CPPCPAPE
CPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - DNA, 666 bp, synthetic construct
  misc_feature 1..666: note = DNA encoding human IgG1 Fc domain having the N-terminal sequenceCPPCPAPE
  source 1..666: mol_type = other DNA
tgcccaccgtgcccagcacctgaactcctgggggggccctcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 11 - protein, 245 aa, synthetic construct
  REGION 1..245: note = pre-Fc chimeric polypeptide having SHH signal peptide
MLLLARCLLLVLVSSLLVCSGLACPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 12 - DNA, 767 bp, synthetic construct
  misc_feature 1..767: note = DNA encoding pre-Fc chimeric polypeptide having SHH signalpeptide
  source 1..767: mol_type = other DNA
aagcttgaattcccaccatgctgctgctggcgagatgtctgctgctagtcctcgtctcctcgctgctggtatgctcgggactggcgtgcccaccgtgcccagcacctgaactcctgggggggccctcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgactcgagcggccg

SEQ ID NO: 13 - protein, 245 aa, synthetic construct
  REGION 1..245: note = pre-Fc chimeric polypeptide having IFN signal peptide
MALTFALLVALLVLSCKSSCSVGCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 14 - DNA, 767 bp, synthetic construct
  misc_feature 1..767: note = DNA encoding pre-Fc chimeric polypeptide having IFN signalpeptide
  source 1..767: mol_type = other DNA
aagcttgaattcccaccatggccttgacctttgctttactggtggccctcctggtgctcagctgcaagtcaagctgctctgtgggctgcccaccgtgcccagcacctgaactcctgggggggccctcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgactcgagcggccg

SEQ ID NO: 15 - protein, 239 aa, synthetic construct
  REGION 1..239: note = pre-Fc chimeric polypeptide having CETP signal peptide
MLAATVLTLALLGNAHACPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 16 - DNA, 749 bp, synthetic construct
  misc_feature 1..749: note = DNA encoding pre-Fc chimeric polypeptide having CETP signalpeptide
  source 1..749: mol_type = other DNA
aagcttgaattcccaccatgctggctgccacagtcctgaccctggccctgctgggcaatgcccatgcctgcccaccgtgcccagcacctgaactcctgggggggccctcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgactcgagcggccg

SEQ ID NO: 17 - protein, 219 aa, synthetic construct
  REGION 1..219: note = Human IgG1 Fc domain having the N-terminal sequence CPAPE
CPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 18 - DNA, 657 bp, synthetic construct
  misc_feature 1..657: note = DNA encoding human IgG1 Fc domain having the N-terminal sequenceCPAPE
  source 1..657: mol_type = other DNA
tgcccagcacctgaactcctgggggggccctcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 19 - protein, 242 aa, synthetic construct
  REGION 1..242: note = pre-Fc chimeric polypeptide having SHH signal peptide
MLLLARCLLLVLVSSLLVCSGLACPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 20 - DNA, 758 bp, synthetic construct
  misc_feature 1..758: note = DNA encoding pre-Fc chimeric polypeptide having SHH signalpeptide
  source 1..758: mol_type = other DNA
aagcttgaattcccaccatgctgctgctggcgagatgtctgctgctagtcctcgtctcctcgctgctggtatgctcgggactggcgtgcccagcacctgaactcctgggggggccctcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgactcgagcggccg

SEQ ID NO: 21 - protein, 242 aa, synthetic construct
  REGION 1..242: note = pre-Fc chimeric polypeptide having IFN signal peptide
MALTFALLVALLVLSCKSSCSVGCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 22 - DNA, 758 bp, synthetic construct
  misc_feature 1..758: note = DNA encoding pre-Fc chimeric polypeptide having IFN signalpeptide
  source 1..758: mol_type = other DNA
aagcttgaattcccaccatggccttgacctttgctttactggtggccctcctggtgctcagctgcaagtcaagctgctctgtgggctgcccagcacctgaactcctgggggggccctcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgactcgagcggccg

SEQ ID NO: 23 - protein, 236 aa, synthetic construct
  REGION 1..236: note = pre-Fc chimeric polypeptide having CETP signal peptide
MLAATVLTLALLGNAHACPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 24 - DNA, 740 bp, synthetic construct
  misc_feature 1..740: note = DNA encoding pre-Fc chimeric polypeptide having CETP signalpeptide
  source 1..740: mol_type = other DNA
aagcttgaattcccaccatgctggctgccacagtcctgaccctggccctgctgggcaatgcccatgcctgcccagcacctgaactcctgggggggccctcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgactcgagcggccg

SEQ ID NO: 25 - protein, 225 aa, synthetic construct
  REGION 1..225: note = Human IgG2 Fc domain having the N-terminal sequence CCVECPPCPAPE
CCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 26 - DNA, 675 bp, synthetic construct
  misc_feature 1..675: note = DNA encoding human IgG2 Fc domain having the N-terminal sequenceCCVECPPCPAPE
  source 1..675: mol_type = other DNA
tgttgtgtcgagtgcccaccgtgcccagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcgtggaggtgcataatgccaagacaaagccacgggaggagcagttcaacagcacgttccgtgtggtcagcgtcctcaccgttgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacacctcccatgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 27 - protein, 248 aa, synthetic construct
  REGION 1..248: note = pre-Fc chimeric polypeptide having SHH signal peptide
MLLLARCLLLVLVSSLLVCSGLACCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 28 - DNA, 776 bp, synthetic construct
  misc_feature 1..776: note = DNA encoding pre-Fc chimeric polypeptide having SHH signalpeptide
  source 1..776: mol_type = other DNA
aagcttgaattcccaccatgctgctgctggcgagatgtctgctgctagtcctcgtctcctcgctgctggtatgctcgggactggcgtgttgtgtcgagtgcccaccgtgcccagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcgtggaggtgcataatgccaagacaaagccacgggaggagcagttcaacagcacgttccgtgtggtcagcgtcctcaccgttgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacacctcccatgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgactcgagcggccg

SEQ ID NO: 29 - protein, 248 aa, synthetic construct
  REGION 1..248: note = pre-Fc chimeric polypeptide having IFN signal peptide
MALTFALLVALLVLSCKSSCSVGCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 30 - DNA, 776 bp, synthetic construct
  misc_feature 1..776: note = DNA encoding pre-Fc chimeric polypeptide having IFN signalpeptide
  source 1..776: mol_type = other DNA
aagcttgaattcccaccatggccttgacctttgctttactggtggccctcctggtgctcagctgcaagtcaagctgctctgtgggctgttgtgtcgagtgcccaccgtgcccagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcgtggaggtgcataatgccaagacaaagccacgggaggagcagttcaacagcacgttccgtgtggtcagcgtcctcaccgttgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacacctcccatgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgactcgagcggccg

SEQ ID NO: 31 - protein, 242 aa, synthetic construct
  REGION 1..242: note = pre-Fc chimeric polypeptide having CETP signal peptide
MLAATVLTLALLGNAHACCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 32 - DNA, 758 bp, synthetic construct
  misc_feature 1..758: note = DNA encoding pre-Fc chimeric polypeptide having CETP signalpeptide
  source 1..758: mol_type = other DNA
aagcttgaattcccaccatgctggctgccacagtcctgaccctggccctgctgggcaatgcccatgcctgttgtgtcgagtgcccaccgtgcccagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcgtggaggtgcataatgccaagacaaagccacgggaggagcagttcaacagcacgttccgtgtggtcagcgtcctcaccgttgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacacctcccatgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgactcgagcggccg

SEQ ID NO: 33 - protein, 224 aa, synthetic construct
  REGION 1..224: note = Human IgG2 Fc domain having the N-terminal sequence CVECPPCPAPE
CVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 34 - DNA, 672 bp, synthetic construct
  misc_feature 1..672: note = DNA encoding human IgG2 Fc domain having the N-terminal sequenceCVECPPCPAPE
  source 1..672: mol_type = other DNA
tgtgtcgagtgcccaccgtgcccagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcgtggaggtgcataatgccaagacaaagccacgggaggagcagttcaacagcacgttccgtgtggtcagcgtcctcaccgttgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacacctcccatgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 35 - protein, 247 aa, synthetic construct
  REGION 1..247: note = pre-Fc chimeric polypeptide having SHH signal peptide
MLLLARCLLLVLVSSLLVCSGLACVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 36 - DNA, 773 bp, synthetic construct
  misc_feature 1..773: note = DNA encoding pre-Fc chimeric polypeptide having SHH signalpeptide
  source 1..773: mol_type = other DNA
aagcttgaattcccaccatgctgctgctggcgagatgtctgctgctagtcctcgtctcctcgctgctggtatgctcgggactggcgtgtgtcgagtgcccaccgtgcccagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcgtggaggtgcataatgccaagacaaagccacgggaggagcagttcaacagcacgttccgtgtggtcagcgtcctcaccgttgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacacctcccatgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgactcgagcggccg

SEQ ID NO: 37 - protein, 247 aa, synthetic construct
  REGION 1..247: note = pre-Fc chimeric polypeptide having IFN signal peptide
MALTFALLVALLVLSCKSSCSVGCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 38 - DNA, 773 bp, synthetic construct
  misc_feature 1..773: note = DNA encoding pre-Fc chimeric polypeptide having IFN signalpeptide
  source 1..773: mol_type = other DNA
aagcttgaattcccaccatggccttgacctttgctttactggtggccctcctggtgctcagctgcaagtcaagctgctctgtgggctgtgtcgagtgcccaccgtgcccagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcgtggaggtgcataatgccaagacaaagccacgggaggagcagttcaacagcacgttccgtgtggtcagcgtcctcaccgttgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacacctcccatgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgactcgagcggccg

SEQ ID NO: 39 - protein, 241 aa, synthetic construct
  REGION 1..241: note = pre-Fc chimeric polypeptide having CETP signal peptide
MLAATVLTLALLGNAHACVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 40 - DNA, 755 bp, synthetic construct
  misc_feature 1..755: note = DNA encoding pre-Fc chimeric polypeptide having CETP signalpeptide
  source 1..755: mol_type = other DNA
aagcttgaattcccaccatgctggctgccacagtcctgaccctggccctgctgggcaatgcccatgcctgtgtcgagtgcccaccgtgcccagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcgtggaggtgcataatgccaagacaaagccacgggaggagcagttcaacagcacgttccgtgtggtcagcgtcctcaccgttgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacacctcccatgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgactcgagcggccg

SEQ ID NO: 41 - protein, 221 aa, synthetic construct
  REGION 1..221: note = Human IgG2 Fc domain having the N-terminal sequence CPPCPAPE
CPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 42 - DNA, 663 bp, synthetic construct
  misc_feature 1..663: note = DNA encoding human IgG2 Fc domain having the N-terminal sequenceCPPCPAPE
  source 1..663: mol_type = other DNA
tgcccaccgtgcccagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcgtggaggtgcataatgccaagacaaagccacgggaggagcagttcaacagcacgttccgtgtggtcagcgtcctcaccgttgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacacctcccatgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 43 - protein, 244 aa, synthetic construct
  REGION 1..244: note = pre-Fc chimeric polypeptide having SHH signal peptide
MLLLARCLLLVLVSSLLVCSGLACPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 44 - DNA, 764 bp, synthetic construct
  misc_feature 1..764: note = DNA encoding pre-Fc chimeric polypeptide having SHH signalpeptide
  source 1..764: mol_type = other DNA
aagcttgaattcccaccatgctgctgctggcgagatgtctgctgctagtcctcgtctcctcgctgctggtatgctcgggactggcgtgcccaccgtgcccagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcgtggaggtgcataatgccaagacaaagccacgggaggagcagttcaacagcacgttccgtgtggtcagcgtcctcaccgttgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacacctcccatgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgactcgagcggccg

SEQ ID NO: 45 - protein, 244 aa, synthetic construct
  REGION 1..244: note = pre-Fc chimeric polypeptide having IFN signal peptide
MALTFALLVALLVLSCKSSCSVGCPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 46 - DNA, 764 bp, synthetic construct
  misc_feature 1..764: note = DNA encoding pre-Fc chimeric polypeptide having IFN signalpeptide
  source 1..764: mol_type = other DNA
aagcttgaattcccaccatggccttgacctttgctttactggtggccctcctggtgctcagctgcaagtcaagctgctctgtgggctgcccaccgtgcccagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcgtggaggtgcataatgccaagacaaagccacgggaggagcagttcaacagcacgttccgtgtggtcagcgtcctcaccgttgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacacctcccatgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgactcgagcggccg

SEQ ID NO: 47 - protein, 238 aa, synthetic construct
  REGION 1..238: note = pre-Fc chimeric polypeptide having CETP signal peptide
MLAATVLTLALLGNAHACPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 48 - DNA, 746 bp, synthetic construct
  misc_feature 1..746: note = DNA encoding pre-Fc chimeric polypeptide having CETP signalpeptide
  source 1..746: mol_type = other DNA
aagcttgaattcccaccatgctggctgccacagtcctgaccctggccctgctgggcaatgcccatgcctgcccaccgtgcccagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcgtggaggtgcataatgccaagacaaagccacgggaggagcagttcaacagcacgttccgtgtggtcagcgtcctcaccgttgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacacctcccatgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgactcgagcggccg

SEQ ID NO: 49 - protein, 218 aa, synthetic construct
  REGION 1..218: note = Human IgG2 Fc domain having the N-terminal sequence CPAPE
CPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 50 - DNA, 654 bp, synthetic construct
  misc_feature 1..654: note = DNA encoding human IgG2 Fc domain having the N-terminal sequenceCPAPE
  source 1..654: mol_type = other DNA
tgcccagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcgtggaggtgcataatgccaagacaaagccacgggaggagcagttcaacagcacgttccgtgtggtcagcgtcctcaccgttgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacacctcccatgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 51 - protein, 241 aa, synthetic construct
  REGION 1..241: note = pre-Fc chimeric polypeptide having SHH signal peptide
MLLLARCLLLVLVSSLLVCSGLACPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 52 - DNA, 755 bp, synthetic construct
  misc_feature 1..755: note = DNA encoding pre-Fc chimeric polypeptide having SHH signalpeptide
  source 1..755: mol_type = other DNA
aagcttgaattcccaccatgctgctgctggcgagatgtctgctgctagtcctcgtctcctcgctgctggtatgctcgggactggcgtgcccagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcgtggaggtgcataatgccaagacaaagccacgggaggagcagttcaacagcacgttccgtgtggtcagcgtcctcaccgttgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacacctcccatgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgactcgagcggccg

SEQ ID NO: 53 - protein, 241 aa, synthetic construct
  REGION 1..241: note = pre-Fc chimeric polypeptide having IFN signal peptide
MALTFALLVALLVLSCKSSCSVGCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 54 - DNA, 755 bp, synthetic construct
  misc_feature 1..755: note = DNA encoding pre-Fc chimeric polypeptide having IFN signalpeptide
  source 1..755: mol_type = other DNA
aagcttgaattcccaccatggccttgacctttgctttactggtggccctcctggtgctcagctgcaagtcaagctgctctgtgggctgcccagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcgtggaggtgcataatgccaagacaaagccacgggaggagcagttcaacagcacgttccgtgtggtcagcgtcctcaccgttgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacacctcccatgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgactcgagcggccg

SEQ ID NO: 55 - protein, 235 aa, synthetic construct
  REGION 1..235: note = pre-Fc chimeric polypeptide having CETP signal peptide
MLAATVLTLALLGNAHACPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 56 - DNA, 737 bp, synthetic construct
  misc_feature 1..737: note = DNA encoding pre-Fc chimeric polypeptide having CETP signalpeptide
  source 1..737: mol_type = other DNA
aagcttgaattcccaccatgctggctgccacagtcctgaccctggccctgctgggcaatgcccatgcctgcccagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcgtggaggtgcataatgccaagacaaagccacgggaggagcagttcaacagcacgttccgtgtggtcagcgtcctcaccgttgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacacctcccatgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgactcgagcggccg

SEQ ID NO: 57 - protein, 267 aa, synthetic construct
  REGION 1..267: note = Human IgG3 Fc domain having the N-terminal sequence(CPRCPEPKSDTPPP)3-CPRCPAPE
CPRCPEPKSCDTPPPCPRCPEPKSCDTPPPCPRCPEPKSCDTPPPCPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 58 - DNA, 801 bp, synthetic construct
  misc_feature 1..801: note = DNA encoding human IgG3 Fc domain having the N-terminal sequence(CPRCPEPKSDTPPP)3-CPRCPAPE
  source 1..801: mol_type = other DNA
tgcccacggtgcccagagcccaaatcttgtgacacacctcccccgtgcccacggtgcccagagcccaaatcttgtgacacacctcccccatgcccacggtgcccagagcccaaatcttgtgacacacctcccccgtgcccaaggtgcccagcacctgaactcctgggaggaccgtcagtcttcctcttccccccaaaacccaaggatacccttatgatttcccggacccctgaggtcacgtgcgtggtggtggacgtgagccacgaagaccccgaggtccagttcaagtggtacgtggacggcgtggaggtgcataatgccaagacaaagccgcgggaggagcagtacaacagcacgttccgtgtggtcagcgtcctcaccgtcctgcaccaggactggctgaacggcaaggagtacaagtgcaaggtctccaacaaagccctcccagcccccatcgagaaaaccatctccaaaaccaaaggacagccccgagaaccacaggtgtacaccctgcccccatcccgggaggagatgaccaagaaccaggtcagcctgacctgcctggtcaaaggcttctaccccagcgacatcgccgtggagtgggagagcagcgggcagccggagaacaactacaacaccacgcctcccatgctggactccgacggctccttcttcctctacagcaagctcaccgtggacaagagcaggtggcagcaggggaacatcttctcatgctccgtgatgcatgaggctctgcacaaccgcttcacgcagaagagcctctccctgtctccgggtaaa

SEQ ID NO: 59 - protein, 290 aa, synthetic construct
  REGION 1..290: note = pre-Fc chimeric polypeptidehaving SHH signal peptide
MLLLARCLLLVLVSSLLVCSGLACPRCPEPKSCDTPPPCPRCPEPKSCDTPPPCPRCPEPKSCDTPPPCPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 60 - DNA, 902 bp, synthetic construct
  misc_feature 1..902: note = DNA encoding pre-Fc chimeric polypeptidehaving SHH signalpeptide
  source 1..902: mol_type = other DNA
aagcttgaattcccaccatgctgctgctggcgagatgtctgctgctagtcctcgtctcctcgctgctggtatgctcgggactggcgtgcccacggtgcccagagcccaaatcttgtgacacacctcccccgtgcccacggtgcccagagcccaaatcttgtgacacacctcccccatgcccacggtgcccagagcccaaatcttgtgacacacctcccccgtgcccaaggtgcccagcacctgaactcctgggaggaccgtcagtcttcctcttccccccaaaacccaaggatacccttatgatttcccggacccctgaggtcacgtgcgtggtggtggacgtgagccacgaagaccccgaggtccagttcaagtggtacgtggacggcgtggaggtgcataatgccaagacaaagccgcgggaggagcagtacaacagcacgttccgtgtggtcagcgtcctcaccgtcctgcaccaggactggctgaacggcaaggagtacaagtgcaaggtctccaacaaagccctcccagcccccatcgagaaaaccatctccaaaaccaaaggacagccccgagaaccacaggtgtacaccctgcccccatcccgggaggagatgaccaagaaccaggtcagcctgacctgcctggtcaaaggcttctaccccagcgacatcgccgtggagtgggagagcagcgggcagccggagaacaactacaacaccacgcctcccatgctggactccgacggctccttcttcctctacagcaagctcaccgtggacaagagcaggtggcagcaggggaacatcttctcatgctccgtgatgcatgaggctctgcacaaccgcttcacgcagaagagcctctccctgtctccgggtaaatgactcgagcggccg

SEQ ID NO: 61 - protein, 290 aa, synthetic construct
  REGION 1..290: note = pre-Fc chimeric polypeptidehaving IFN signal peptide
MALTFALLVALLVLSCKSSCSVGCPRCPEPKSCDTPPPCPRCPEPKSCDTPPPCPRCPEPKSCDTPPPCPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 62 - DNA, 902 bp, synthetic construct
  misc_feature 1..902: note = DNA encoding pre-Fc chimeric polypeptide having IFN signalpeptide
  source 1..902: mol_type = other DNA
aagcttgaattcccaccatggccttgacctttgctttactggtggccctcctggtgctcagctgcaagtcaagctgctctgtgggctgcccacggtgcccagagcccaaatcttgtgacacacctcccccgtgcccacggtgcccagagcccaaatcttgtgacacacctcccccatgcccacggtgcccagagcccaaatcttgtgacacacctcccccgtgcccaaggtgcccagcacctgaactcctgggaggaccgtcagtcttcctcttccccccaaaacccaaggatacccttatgatttcccggacccctgaggtcacgtgcgtggtggtggacgtgagccacgaagaccccgaggtccagttcaagtggtacgtggacggcgtggaggtgcataatgccaagacaaagccgcgggaggagcagtacaacagcacgttccgtgtggtcagcgtcctcaccgtcctgcaccaggactggctgaacggcaaggagtacaagtgcaaggtctccaacaaagccctcccagcccccatcgagaaaaccatctccaaaaccaaaggacagccccgagaaccacaggtgtacaccctgcccccatcccgggaggagatgaccaagaaccaggtcagcctgacctgcctggtcaaaggcttctaccccagcgacatcgccgtggagtgggagagcagcgggcagccggagaacaactacaacaccacgcctcccatgctggactccgacggctccttcttcctctacagcaagctcaccgtggacaagagcaggtggcagcaggggaacatcttctcatgctccgtgatgcatgaggctctgcacaaccgcttcacgcagaagagcctctccctgtctccgggtaaatgactcgagcggccg

SEQ ID NO: 63 - protein, 284 aa, synthetic construct
  REGION 1..284: note = pre-Fc chimeric polypeptide having CETP signal peptide
MLAATVLTLALLGNAHACPRCPEPKSCDTPPPCPRCPEPKSCDTPPPCPRCPEPKSCDTPPPCPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 64 - DNA, 884 bp, synthetic construct
  misc_feature 1..884: note = DNA encoding pre-Fc chimeric polypeptide having CETP signalpeptide
  source 1..884: mol_type = other DNA
aagcttgaattcccaccatgctggctgccacagtcctgaccctggccctgctgggcaatgcccatgcctgcccacggtgcccagagcccaaatcttgtgacacacctcccccgtgcccacggtgcccagagcccaaatcttgtgacacacctcccccatgcccacggtgcccagagcccaaatcttgtgacacacctcccccgtgcccaaggtgcccagcacctgaactcctgggaggaccgtcagtcttcctcttccccccaaaacccaaggatacccttatgatttcccggacccctgaggtcacgtgcgtggtggtggacgtgagccacgaagaccccgaggtccagttcaagtggtacgtggacggcgtggaggtgcataatgccaagacaaagccgcgggaggagcagtacaacagcacgttccgtgtggtcagcgtcctcaccgtcctgcaccaggactggctgaacggcaaggagtacaagtgcaaggtctccaacaaagccctcccagcccccatcgagaaaaccatctccaaaaccaaaggacagccccgagaaccacaggtgtacaccctgcccccatcccgggaggagatgaccaagaaccaggtcagcctgacctgcctggtcaaaggcttctaccccagcgacatcgccgtggagtgggagagcagcgggcagccggagaacaactacaacaccacgcctcccatgctggactccgacggctccttcttcctctacagcaagctcaccgtggacaagagcaggtggcagcaggggaacatcttctcatgctccgtgatgcatgaggctctgcacaaccgcttcacgcagaagagcctctccctgtctccgggtaaatgactcgagcggccg

SEQ ID NO: 65 - protein, 222 aa, synthetic construct
  REGION 1..222: note = Human IgG3 Fc domain having the N-terminal sequence CPRCPAPE
CPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 66 - DNA, 681 bp, synthetic construct
  misc_feature 1..681: note = DNAencoding human IgG3 Fc domain having the N-terminal sequenceCPRCPAPE
  source 1..681: mol_type = other DNA
tgcccaaggtgcccagcacctgaactcctgggaggaccgtcagtcttcctcttccccccaaaacccaaggatacccttatgatttcccggacccctgaggtcacgtgcgtggtggtggacgtgagccacgaagaccccgaggtccagttcaagtggtacgtggacggcgtggaggtgcataatgccaagacaaagccgcgggaggagcagtacaacagcacgttccgtgtggtcagcgtcctcaccgtcctgcaccaggactggctgaacggcaaggagtacaagtgcaaggtctccaacaaagccctcccagcccccatcgagaaaaccatctccaaaaccaaaggacagccccgagaaccacaggtgtacaccctgcccccatcccgggaggagatgaccaagaaccaggtcagcctgacctgcctggtcaaaggcttctaccccagcgacatcgccgtggagtgggagagcagcgggcagccggagaacaactacaacaccacgcctcccatgctggactccgacggctccttcttcctctacagcaagctcaccgtggacaagagcaggtggcagcaggggaacatcttctcatgctccgtgatgcatgaggctctgcacaaccgcttcacgcagaagagcctctccctgtctccgggtaaatgactcgagcggccg

SEQ ID NO: 67 - protein, 245 aa, synthetic construct
  REGION 1..245: note = pre-Fc chimeric polypeptide having SHH signal peptide
MLLLARCLLLVLVSSLLVCSGLACPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 68 - DNA, 767 bp, synthetic construct
  misc_feature 1..767: note = DNA encoding pre-Fc chimeric polypeptide having SHH signalpeptide
  source 1..767: mol_type = other DNA
aagcttgaattcccaccatgctgctgctggcgagatgtctgctgctagtcctcgtctcctcgctgctggtatgctcgggactggcgtgcccaaggtgcccagcacctgaactcctgggaggaccgtcagtcttcctcttccccccaaaacccaaggatacccttatgatttcccggacccctgaggtcacgtgcgtggtggtggacgtgagccacgaagaccccgaggtccagttcaagtggtacgtggacggcgtggaggtgcataatgccaagacaaagccgcgggaggagcagtacaacagcacgttccgtgtggtcagcgtcctcaccgtcctgcaccaggactggctgaacggcaaggagtacaagtgcaaggtctccaacaaagccctcccagcccccatcgagaaaaccatctccaaaaccaaaggacagccccgagaaccacaggtgtacaccctgcccccatcccgggaggagatgaccaagaaccaggtcagcctgacctgcctggtcaaaggcttctaccccagcgacatcgccgtggagtgggagagcagcgggcagccggagaacaactacaacaccacgcctcccatgctggactccgacggctccttcttcctctacagcaagctcaccgtggacaagagcaggtggcagcaggggaacatcttctcatgctccgtgatgcatgaggctctgcacaaccgcttcacgcagaagagcctctccctgtctccgggtaaatgactcgagcggccg

SEQ ID NO: 69 - protein, 245 aa, synthetic construct
  REGION 1..245: note = pre-Fc chimeric polypeptide having IFN signal peptide
MALTFALLVALLVLSCKSSCSVGCPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 70 - DNA, 767 bp, synthetic construct
  misc_feature 1..767: note = DNA encoding pre-Fc chimeric polypeptide having IFN signalpeptide
  source 1..767: mol_type = other DNA
aagcttgaattcccaccatggccttgacctttgctttactggtggccctcctggtgctcagctgcaagtcaagctgctctgtgggctgcccaaggtgcccagcacctgaactcctgggaggaccgtcagtcttcctcttccccccaaaacccaaggatacccttatgatttcccggacccctgaggtcacgtgcgtggtggtggacgtgagccacgaagaccccgaggtccagttcaagtggtacgtggacggcgtggaggtgcataatgccaagacaaagccgcgggaggagcagtacaacagcacgttccgtgtggtcagcgtcctcaccgtcctgcaccaggactggctgaacggcaaggagtacaagtgcaaggtctccaacaaagccctcccagcccccatcgagaaaaccatctccaaaaccaaaggacagccccgagaaccacaggtgtacaccctgcccccatcccgggaggagatgaccaagaaccaggtcagcctgacctgcctggtcaaaggcttctaccccagcgacatcgccgtggagtgggagagcagcgggcagccggagaacaactacaacaccacgcctcccatgctggactccgacggctccttcttcctctacagcaagctcaccgtggacaagagcaggtggcagcaggggaacatcttctcatgctccgtgatgcatgaggctctgcacaaccgcttcacgcagaagagcctctccctgtctccgggtaaatgactcgagcggccg

SEQ ID NO: 71 - protein, 239 aa, synthetic construct
  REGION 1..239: note = pre-Fc chimeric polypeptide having CETP signal peptide
MLAATVLTLALLGNAHACPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 72 - DNA, 749 bp, synthetic construct
  misc_feature 1..749: note = DNA encoding pre-Fc chimeric polypeptide having CETP signalpeptide
  source 1..749: mol_type = other DNA
aagcttgaattcccaccatgctggctgccacagtcctgaccctggccctgctgggcaatgcccatgcctgcccaaggtgcccagcacctgaactcctgggaggaccgtcagtcttcctcttccccccaaaacccaaggatacccttatgatttcccggacccctgaggtcacgtgcgtggtggtggacgtgagccacgaagaccccgaggtccagttcaagtggtacgtggacggcgtggaggtgcataatgccaagacaaagccgcgggaggagcagtacaacagcacgttccgtgtggtcagcgtcctcaccgtcctgcaccaggactggctgaacggcaaggagtacaagtgcaaggtctccaacaaagccctcccagcccccatcgagaaaaccatctccaaaaccaaaggacagccccgagaaccacaggtgtacaccctgcccccatcccgggaggagatgaccaagaaccaggtcagcctgacctgcctggtcaaaggcttctaccccagcgacatcgccgtggagtgggagagcagcgggcagccggagaacaactacaacaccacgcctcccatgctggactccgacggctccttcttcctctacagcaagctcaccgtggacaagagcaggtggcagcaggggaacatcttctcatgctccgtgatgcatgaggctctgcacaaccgcttcacgcagaagagcctctccctgtctccgggtaaatgactcgagcggccg

SEQ ID NO: 73 - protein, 219 aa, synthetic construct
  REGION 1..219: note = Human IgG3 Fc domain having the N-terminal sequence CPAPE
CPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 74 - DNA, 672 bp, synthetic construct
  misc_feature 1..672: note = DNA encoding human IgG3 Fc domain having the N-terminal sequenceCPAPE
  source 1..672: mol_type = other DNA
tgcccagcacctgaactcctgggaggaccgtcagtcttcctcttccccccaaaacccaaggatacccttatgatttcccggacccctgaggtcacgtgcgtggtggtggacgtgagccacgaagaccccgaggtccagttcaagtggtacgtggacggcgtggaggtgcataatgccaagacaaagccgcgggaggagcagtacaacagcacgttccgtgtggtcagcgtcctcaccgtcctgcaccaggactggctgaacggcaaggagtacaagtgcaaggtctccaacaaagccctcccagcccccatcgagaaaaccatctccaaaaccaaaggacagccccgagaaccacaggtgtacaccctgcccccatcccgggaggagatgaccaagaaccaggtcagcctgacctgcctggtcaaaggcttctaccccagcgacatcgccgtggagtgggagagcagcgggcagccggagaacaactacaacaccacgcctcccatgctggactccgacggctccttcttcctctacagcaagctcaccgtggacaagagcaggtggcagcaggggaacatcttctcatgctccgtgatgcatgaggctctgcacaaccgcttcacgcagaagagcctctccctgtctccgggtaaatgactcgagcggccg

SEQ ID NO: 75 - protein, 242 aa, synthetic construct
  REGION 1..242: note = pre-Fc chimeric polypeptide having SHH signal peptide
MLLLARCLLLVLVSSLLVCSGLACPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 76 - DNA, 758 bp, synthetic construct
  misc_feature 1..758: note = DNA encoding pre-Fc chimeric polypeptide having SHH signalpeptide
  source 1..758: mol_type = other DNA
aagcttgaattcccaccatgctgctgctggcgagatgtctgctgctagtcctcgtctcctcgctgctggtatgctcgggactggcgtgcccagcacctgaactcctgggaggaccgtcagtcttcctcttccccccaaaacccaaggatacccttatgatttcccggacccctgaggtcacgtgcgtggtggtggacgtgagccacgaagaccccgaggtccagttcaagtggtacgtggacggcgtggaggtgcataatgccaagacaaagccgcgggaggagcagtacaacagcacgttccgtgtggtcagcgtcctcaccgtcctgcaccaggactggctgaacggcaaggagtacaagtgcaaggtctccaacaaagccctcccagcccccatcgagaaaaccatctccaaaaccaaaggacagccccgagaaccacaggtgtacaccctgcccccatcccgggaggagatgaccaagaaccaggtcagcctgacctgcctggtcaaaggcttctaccccagcgacatcgccgtggagtgggagagcagcgggcagccggagaacaactacaacaccacgcctcccatgctggactccgacggctccttcttcctctacagcaagctcaccgtggacaagagcaggtggcagcaggggaacatcttctcatgctccgtgatgcatgaggctctgcacaaccgcttcacgcagaagagcctctccctgtctccgggtaaatgactcgagcggccg

SEQ ID NO: 77 - protein, 242 aa, synthetic construct
  REGION 1..242: note = pre-Fc chimeric polypeptide having IFN signal peptide
MALTFALLVALLVLSCKSSCSVGCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 78 - DNA, 758 bp, synthetic construct
  misc_feature 1..758: note = DNA encoding pre-Fc chimeric polypeptide having IFN signalpeptide
  source 1..758: mol_type = other DNA
aagcttgaattcccaccatggccttgacctttgctttactggtggccctcctggtgctcagctgcaagtcaagctgctctgtgggctgcccagcacctgaactcctgggaggaccgtcagtcttcctcttccccccaaaacccaaggatacccttatgatttcccggacccctgaggtcacgtgcgtggtggtggacgtgagccacgaagaccccgaggtccagttcaagtggtacgtggacggcgtggaggtgcataatgccaagacaaagccgcgggaggagcagtacaacagcacgttccgtgtggtcagcgtcctcaccgtcctgcaccaggactggctgaacggcaaggagtacaagtgcaaggtctccaacaaagccctcccagcccccatcgagaaaaccatctccaaaaccaaaggacagccccgagaaccacaggtgtacaccctgcccccatcccgggaggagatgaccaagaaccaggtcagcctgacctgcctggtcaaaggcttctaccccagcgacatcgccgtggagtgggagagcagcgggcagccggagaacaactacaacaccacgcctcccatgctggactccgacggctccttcttcctctacagcaagctcaccgtggacaagagcaggtggcagcaggggaacatcttctcatgctccgtgatgcatgaggctctgcacaaccgcttcacgcagaagagcctctccctgtctccgggtaaatgactcgagcggccg

SEQ ID NO: 79 - protein, 235 aa, synthetic construct
  REGION 1..235: note = pre-Fc chimeric polypeptide having CETP signal peptide
MLAATVLTLALLGNAHACPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPG

SEQ ID NO: 80 - DNA, 740 bp, synthetic construct
  misc_feature 1..740: note = DNA encoding pre-Fc chimeric polypeptide having CETP signalpeptide
  source 1..740: mol_type = other DNA
aagcttgaattcccaccatgctggctgccacagtcctgaccctggccctgctgggcaatgcccatgcctgcccagcacctgaactcctgggaggaccgtcagtcttcctcttccccccaaaacccaaggatacccttatgatttcccggacccctgaggtcacgtgcgtggtggtggacgtgagccacgaagaccccgaggtccagttcaagtggtacgtggacggcgtggaggtgcataatgccaagacaaagccgcgggaggagcagtacaacagcacgttccgtgtggtcagcgtcctcaccgtcctgcaccaggactggctgaacggcaaggagtacaagtgcaaggtctccaacaaagccctcccagcccccatcgagaaaaccatctccaaaaccaaaggacagccccgagaaccacaggtgtacaccctgcccccatcccgggaggagatgaccaagaaccaggtcagcctgacctgcctggtcaaaggcttctaccccagcgacatcgccgtggagtgggagagcagcgggcagccggagaacaactacaacaccacgcctcccatgctggactccgacggctccttcttcctctacagcaagctcaccgtggacaagagcaggtggcagcaggggaacatcttctcatgctccgtgatgcatgaggctctgcacaaccgcttcacgcagaagagcctctccctgtctccgggtaaatgactcgagcggccg

SEQ ID NO: 81 - protein, 222 aa, synthetic construct
  REGION 1..222: note = Human IgG4 Fc domain having the N-terminal sequence CPSCPAPE
CPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 82 - DNA, 666 bp, synthetic construct
  misc_feature 1..666: note = DNA encoding human IgG4 Fc domain having the N-terminal sequenceCPSCPAPE
  source 1..666: mol_type = other DNA
tgcccatcatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaa

SEQ ID NO: 83 - protein, 245 aa, synthetic construct
  REGION 1..245: note = pre-Fc chimeric polypeptide having a SHH signal peptide
MLLLARCLLLVLVSSLLVCSGLACPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 84 - DNA, 767 bp, synthetic construct
  misc_feature 1..767: note = DNA encoding pre-Fc chimeric polypeptide having a SHH signalpeptide
  source 1..767: mol_type = other DNA
aagcttgaattcccaccatgctgctgctggcgagatgtctgctgctagtcctcgtctcctcgctgctggtatgctcgggactggcgtgcccatcatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaatgactcgagcggccg

SEQ ID NO: 85 - protein, 245 aa, synthetic construct
  REGION 1..245: note = pre-Fc chimeric polypeptide having a IFN signal peptide
MALTFALLVALLVLSCKSSCSVGCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 86 - DNA, 767 bp, synthetic construct
  misc_feature 1..767: note = DNA encoding pre-Fc chimeric polypeptide having a IFN signalpeptide
  source 1..767: mol_type = other DNA
aagcttgaattcccaccatggccttgacctttgctttactggtggccctcctggtgctcagctgcaagtcaagctgctctgtgggctgcccatcatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaatgactcgagcggccg

SEQ ID NO: 87 - protein, 239 aa, synthetic construct
  REGION 1..239: note = pre-Fc chimeric polypeptide having a CETP signal peptide
MLAATVLTLALLGNAHACPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 88 - DNA, 749 bp, synthetic construct
  misc_feature 1..749: note = DNA encoding pre-Fc chimeric polypeptide having a CETP signalpeptide
  source 1..749: mol_type = other DNA
aagcttgaattcccaccatgctggctgccacagtcctgaccctggccctgctgggcaatgcccatgcctgcccatcatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaatgactcgagcggccg

SEQ ID NO: 89 - protein, 219 aa, synthetic construct
  REGION 1..219: note = Human IgG4 Fc domain having the N-terminal sequence CPAPE
CPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 90 - DNA, 657 bp, synthetic construct
  misc_feature 1..657: note = DNA encoding human IgG4 Fc domain having the N-terminal sequenceCPAPE
  source 1..657: mol_type = other DNA
tgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaa

SEQ ID NO: 91 - protein, 242 aa, synthetic construct
  REGION 1..242: note = pre-Fc chimeric polypeptide having a SHH signal peptide
MLLLARCLLLVLVSSLLVCSGLACPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 92 - DNA, 758 bp, synthetic construct
  misc_feature 1..758: note = DNA encoding pre-Fc chimeric polypeptide having a SHH signalpeptide
  source 1..758: mol_type = other DNA
aagcttgaattcccaccatgctgctgctggcgagatgtctgctgctagtcctcgtctcctcgctgctggtatgctcgggactggcgtgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaatgactcgagcggccg

SEQ ID NO: 93 - protein, 242 aa, synthetic construct
  REGION 1..242: note = pre-Fc chimeric polypeptide having a IFN signal peptide
MALTFALLVALLVLSCKSSCSVGCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 94 - DNA, 758 bp, synthetic construct
  misc_feature 1..758: note = DNA encoding pre-Fc chimeric polypeptide having a IFN signalpeptide
  source 1..758: mol_type = other DNA
aagcttgaattcccaccatggccttgacctttgctttactggtggccctcctggtgctcagctgcaagtcaagctgctctgtgggctgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaatgactcgagcggccg

SEQ ID NO: 95 - protein, 236 aa, synthetic construct
  REGION 1..236: note = pre-Fc chimeric polypeptide having a CETP signal peptide
MLAATVLTLALLGNAHACPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 96 - DNA, 740 bp, synthetic construct
  misc_feature 1..740: note = DNA encoding pre-Fc chimeric polypeptide having a CETP signalpeptide
  source 1..740: mol_type = other DNA
aagcttgaattcccaccatgctggctgccacagtcctgaccctggccctgctgggcaatgcccatgcctgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaatgactcgagcggccg

SEQ ID NO: 97 - DNA, 5428 bp, synthetic construct
  misc_feature 1..5428: note = pCDNA3.1(+)
  source 1..5428: mol_type = other DNA
gacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttggtaccgagctcggatccactagtccagtgtggtggaattctgcagatatccagcacagtggcggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 98 - DNA, 4195 bp, synthetic construct
  misc_feature 1..4195: note = pSA
  source 1..4195: mol_type = other DNA
ctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcaagcggccgcaacccgggaaaagcttggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccgggaacggtgcattggaacgcggattccccgtgccaagagtgacgtaagtaccgcctatagagtctataggcccacccccttggcttcttatgcatgctcccctgctccgacccgggctcctcgcccgcccggacccacaggccaccctcaaccgtcctggccccggacccaaaccccacccctcactctgcttctccccgcaggagaattcaatcgcgaaagggcccaaagatctgccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggagctagcatcccgcccctaactccgccctgttccgcccattctccgccccatggctgactaattttttttatttatgcagaggccgaggccgcctcggcctctgagctattccagaagtagtgaggaggcttttttggaggcctaggcttttgcgtcgagaagcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 99 - DNA, 824 bp, synthetic construct
  misc_feature 1..824: note = Artificial Construct
  source 1..824: mol_type = other DNA
gttaacgaattcccaccatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaagatctgttaac

SEQ ID NO: 100 - DNA, 6739 bp, synthetic construct
  misc_feature 1..6739: note = pCDNA3-TNR1B-Mth
  source 1..6739: mol_type = other DNA
gacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttgaattcccaccatggcgcccgtcgccgtctgggccgcgctggccgtcggactggagctctgggctgcggcgcacgccttgcccgcccaggtggcatttacaccctacgccccggagcccgggagcacatgccggctcagagaatactatgaccagacagctcagatgtgctgcagcaaatgctcgccgggccaacatgcaaaagtcttctgtaccaagacctcggacaccgtgtgtgactcctgtgaggacagcacatacacccagctctggaactgggttcccgagtgcttgagctgtggctcccgctgtagctctgaccaggtggaaactcaagcctgcactcgggaacagaaccgcatctgcacctgcaggcccggctggtactgcgcgctgagcaagcaggaggggtgccggctgtgcgcgccgctgcgcaagtgccgcccgggcttcggcgtggccagaccaggaactgaaacatcagacgtggtgtgcaagccctgtgccccggggacgttctccaacacgacttcatccacggatatttgcaggccccaccagatctgtaacgtggtggccatccctgggaatgcaagcatggatgcagtctgcacgtccacgtcccccacccggagtatggccccaggggcagtacacttaccccagccagtgtccacacgatcccaacacacgcagccaactccagaacccagcactgctccaagcacctccttcctgctcccaatggggcccagccccccagctgaagggagcactggcgacgggtgcgtatccggtgacaccattgtaatgactagtggcgggccccgcactgtggctgaactggagggcaaaccgttcaccgcactgattcgcggctctggctacccatgcccctcaggtttcttccgcacctgtgaacgtgacgtatatgatctgcgtacacgtgagggtcattgcttacgtttgacccatgatcaccgtgttctggtgatggatggtggcctggaatggcgtgccgcgggtgaactggaacgcggcgaccgcctggtgatggatgatgcagctggcgagtttccggcactggcaaccttccgtggcctgcgtggcgctggccgccaggatgtttatgacgctactgtttacggtgctagcgcattcactgctaatggcttcattgtacacgcatgtggcgagcagcccgggaccggtctgaactcaggcctcacgacaaatcctggtgtatccgcttggcaggtcaacacagcttatactgcgggacaattggtcacatataacggcaagacgtataaatgtttgcagccccacacctccttggcaggatgggaaccatccaacgttcctgccttgtggcagcttcaatgactcgagcggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 101 - protein, 457 aa, synthetic construct
  REGION 1..457: note = pre-TNR1B-intein chimeric polypeptide
MAPVAVWAALAVGLELWAAAHALPAQVAFTPYAPEPGSTCRLREYYDQTAQMCCSKCSPGQHAKVFCTKTSDTVCDSCEDSTYTQLWNWVPECLSCGSRCSSDQVETQACTREQNRICTCRPGWYCALSKQEGCRLCAPLRKCRPGFGVARPGTETSDVVCKPCAPGTFSNTTSSTDICRPHQICNVVAIPGNASMDAVCTSTSPTRSMAPGAVHLPQPVSTRSQHTQPTPEPSTAPSTSFLLPMGPSPPAEGSTGDGCVSGDTIVMTSGGPRTVAELEGKPFTALIRGSGYPCPSGFFRTCERDVYDLRTREGHCLRLTHDHRVLVMDGGLEWRAAGELERGDRLVMDDAAGEFPALATFRGLRGAGRQDVYDATVYGASAFTANGFIVHACGEQPGTGLNSGLTTNPGVSAWQVNTAYTAGQLVTYNGKTYKCLQPHTSLAGWEPSNVPALWQLQ

SEQ ID NO: 102 - protein, 435 aa, synthetic construct
  REGION 1..435: note = mature TNR1B-intein fusion protein
LPAQVAFTPYAPEPGSTCRLREYYDQTAQMCCSKCSPGQHAKVFCTKTSDTVCDSCEDSTYTQLWNWVPECLSCGSRCSSDQVETQACTREQNRICTCRPGWYCALSKQEGCRLCAPLRKCRPGFGVARPGTETSDVVCKPCAPGTFSNTTSSTDICRPHQICNVVAIPGNASMDAVCTSTSPTRSMAPGAVHLPQPVSTRSQHTQPTPEPSTAPSTSFLLPMGPSPPAEGSTGDGCVSGDTIVMTSGGPRTVAELEGKPFTALIRGSGYPCPSGFFRTCERDVYDLRTREGHCLRLTHDHRVLVMDGGLEWRAAGELERGDRLVMDDAAGEFPALATFRGLRGAGRQDVYDATVYGASAFTANGFIVHACGEQPGTGLNSGLTTNPGVSAWQVNTAYTAGQLVTYNGKTYKCLQPHTSLAGWEPSNVPALWQLQ

SEQ ID NO: 103 - DNA, 6103 bp, synthetic construct
  misc_feature 1..6103: note = pCDNA3-SHH-IgG1-Fc11
  source 1..6103: mol_type = other DNA
gacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttgaattcccaccatgctgctgctggcgagatgtctgctgctagtcctcgtctcctcgctgctggtatgctcgggactggcgtgcccaccgtgcccagcacctgaactcctgggggggccctcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgactcgagcggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 104 - protein, 245 aa, synthetic construct; identical to SEQ ID NO: 11
  REGION 1..245: note = pre-Fc6 polypeptide

SEQ ID NO: 105 - protein, 222 aa, synthetic construct; identical to SEQ ID NO: 9
  REGION 1..222: note = mature Fc6 protein

SEQ ID NO: 106 - protein, 237 aa, synthetic construct
  REGION 1..237: note = cleaved alkyne-modified TNR1B
  SITE 237: note = Xaa = cysteine-alkyne
LPAQVAFTPYAPEPGSTCRLREYYDQTAQMCCSKCSPGQHAKVFCTKTSDTVCDSCEDSTYTQLWNWVPECLSCGSRCSSDQVETQACTREQNRICTCRPGWYCALSKQEGCRLCAPLRKCRPGFGVARPGTETSDVVCKPCAPGTFSNTTSSTDICRPHQICNVVAIPGNASMDAVCTSTSPTRSMAPGAVHLPQPVSTRSQHTQPTPEPSTAPSTSFLLPMGPSPPAEGSTGDGX

SEQ ID NO: 107 - protein, 237 aa, synthetic construct; identical to SEQ ID NO: 106
  REGION 1..237: note = azide-modified TNR1B protein
  SITE 237: note = Xaa = cysteine-azide

SEQ ID NO: 108 - protein, 237 aa, synthetic construct
  REGION 1..237: note = cysteine-modified TNR1B
LPAQVAFTPYAPEPGSTCRLREYYDQTAQMCCSKCSPGQHAKVFCTKTSDTVCDSCEDSTYTQLWNWVPECLSCGSRCSSDQVETQACTREQNRICTCRPGWYCALSKQEGCRLCAPLRKCRPGFGVARPGTETSDVVCKPCAPGTFSNTTSSTDICRPHQICNVVAIPGNASMDAVCTSTSPTRSMAPGAVHLPQPVSTRSQHTQPTPEPSTAPSTSFLLPMGPSPPAEGSTGDGC

SEQ ID NO: 109 - protein, 236 aa, synthetic construct
  REGION 1..236: note = thioester-modified TNR1B
  SITE 236: note = Xaa = glycine-thioester
LPAQVAFTPYAPEPGSTCRLREYYDQTAQMCCSKCSPGQHAKVFCTKTSDTVCDSCEDSTYTQLWNWVPECLSCGSRCSSDQVETQACTREQNRICTCRPGWYCALSKQEGCRLCAPLRKCRPGFGVARPGTETSDVVCKPCAPGTFSNTTSSTDICRPHQICNVVAIPGNASMDAVCTSTSPTRSMAPGAVHLPQPVSTRSQHTQPTPEPSTAPSTSFLLPMGPSPPAEGSTGDX

SEQ ID NO: 110 - protein, 227 aa, synthetic construct
  REGION 1..227: note = Az-DKTHT-Fc6 protein
  SITE 1: note = Xaa = azide-aspartic acid
XKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 111 - protein, 227 aa, synthetic construct; identical to SEQ ID NO: 110
  REGION 1..227: note = Az-PEG4-DKTHT-Fc6
  SITE 1: note = Xaa = azide-PEG4-aspartic acid

SEQ ID NO: 112 - protein, 465 aa, synthetic construct
  REGION 1..465: note = Polypeptide with internal non-peptidyl moiety.TNR1B-alkyne-azide-Fc6
  SITE 238: note = Xaa = non-peptidyl moiety
LPAQVAFTPYAPEPGSTCRLREYYDQTAQMCCSKCSPGQHAKVFCTKTSDTVCDSCEDSTYTQLWNWVPECLSCGSRCSSDQVETQACTREQNRICTCRPGWYCALSKQEGCRLCAPLRKCRPGFGVARPGTETSDVVCKPCAPGTFSNTTSSTDICRPHQICNVVAIPGNASMDAVCTSTSPTRSMAPGAVHLPQPVSTRSQHTQPTPEPSTAPSTSFLLPMGPSPPAEGSTGDGCXDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 113 - protein, 465 aa, synthetic construct; identical to SEQ ID NO: 112
  REGION 1..465: note = Polypeptide with internal non-peptidyl moiety.TNR1B-alkyne-azide-PEG4-Fc6
  SITE 238: note = Xaa = non-peptidyl moiety containing PEG4 linker

SEQ ID NO: 114 - protein, 467 aa, synthetic construct
  REGION 1..467: note = Etanercept
LPAQVAFTPYAPEPGSTCRLREYYDQTAQMCCSKCSPGQHAKVFCTKTSDTVCDSCEDSTYTQLWNWVPECLSCGSRCSSDQVETQACTREQNRICTCRPGWYCALSKQEGCRLCAPLRKCRPGFGVARPGTETSDVVCKPCAPGTFSNTTSSTDICRPHQICNVVAIPGNASMDAVCTSTSPTRSMAPGAVHLPQPVSTRSQHTQPTPEPSTAPSTSFLLPMGPSPPAEGSTGDEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 115 - DNA, 4175 bp, synthetic construct
  misc_feature 1..4175: note = pFUSE2ss-DE27-V -CLIg-hk
  source 1..4175: mol_type = other DNA
ggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacctgagatcaacatgtacaggatgcaactcctgtcttgcattgcactaagtcttgcacttgtcacgaattcagacatccagatgacccagtctccatcctccctgtctgcatctgtaggggacagagtcaccatcacttgtcgggcaagtcagggcatcagaaattacttagcctggtatcagcaaaaaccagggaaagcccctaagctcctgatctatgctgcatccactttgcaatcaggggtcccatctcggttcagtggcagtggatctgggacagatttcactctcaccatcagcagcctacagcctgaagatgttgcaacttattactgtcaaaggtataaccgtgcaccgtatacttttggccaggggaccaaggtggaaatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttagagggagctagctcgacatgataagatacattgatgagtttggacaaaccacaactagaatgcagtgaaaaaaatgctttatttgtgaaatttgtgatgctattgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggaattaattctaaaatacagcatagcaaaactttaacctccaaatcaagcctctacttgaatccttttctgagggatgaataaggcataggcatcaggggctgttgccaatgtgcattagctgtttgcagcctcaccttctttcatggagtttaagatatagtgtattttcccaaggtttgaactagctcttcatttctttatgttttaaatgcactgacctcccacattccctttttagtaaaatattcagaaataatttaaatacatcattgcaatgaaaataaatgttttttattaggcagaatccagatgctcaaggcccttcataatatcccccagtttagtagttggacttagggaacaaaggaacctttaatagaaattggacagcaagaaagcgagcttctagctttagttcctggtgtacttgagggggatgagttcctcaatggtggttttgaccagcttgccattcatctcaatgagcacaaagcagtcaggagcatagtcagagatgagctctctgcacatgccacaggggctgaccaccctgatggatctgtccacctcatcagagtaggggtgcctgacagccacaatggtgtcaaagtccttctgcccgttgctcacagcagacccaatggcaatggcttcagcacagacagtgaccctgccaatgtaggcctcaatgtggacagcagagatgatctccccagtcttggtcctgatggccgccccgacatggtgcttgttgtcctcatagagcatggtgatcttctcagtggcgacctccaccagctccagatcctgctgagagatgttgaaggtcttcatgatggctcctcctgtcaggagaggaaagagaagaaggttagtacaattgctatagtgagttgtattatactatgcttatgattaattgtcaaactagggctgcagggttcatagtgccacttttcctgcactgccccatctcctgcccaccctttcccaggcatagacagtcagtgacttaccaaactcacaggagggagaaggcagaagcttgagacagacccgcgggaccgccgaactgcgaggggacgtggctagggcggcttcttttatggtgcgccggccctcggaggcagggcgctcggggaggcctagcggccaatctgcggtggcaggaggcggggccgaaggccgtgcctgaccaatccggagcacataggagtctcagccccccgccccaaagcaaggggaagtcacgcgcctgtagcgccagcgtgttgtgaaatgggggcttgggggggttggggccctgactagtcaaaacaaactcccattgacgtcaatggggtggagacttggaaatccccgtgagtcaaaccgctatccacgcccattgatgtactgccaaaaccgcatcatcatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccgtaagttatgtaacgcctgcaggttaattaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatggctagttaattaacatttaaatcagcggccgcaataaaatatctttattttcattacatctgtgtgttggttttttgtgtgaatcgtaactaacatacgctctccatcaaaacaaaacgaaacaaaacaaactagcaaaataggctgtccccagtgcaagtgcaggtgccagaacatttctctatcgaa

SEQ ID NO: 116 - DNA, 4776 bp, synthetic construct
  misc_feature 1..4776: note = pPUSEss-DE27-V 1-CHIg-hG1-Mth-1
  source 1..4776: mol_type = other DNA
ggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacctgagatcaccggcgaaggagggccaccatgtacaggatgcaactcctgtcttgcattgcactaagtcttgcacttgtcacgaattcggaggtgcagctggtggagtctgggggaggcttggtacagcccggcaggtccctgagactctcctgtgcggcctctggattcacctttgatgattatgccatgcactgggtccggcaagctccagggaagggcctggaatgggtctcagctatcacttggaatagtggtcacatagactatgcggactctgtggagggccgattcaccatctccagagacaacgccaagaactccctgtatctgcaaatgaacagtctgagagctgaggatacggccgtatattactgtgcgaaagtctcgtaccttagcaccgcgtcctcccttgactattggggccaaggtaccctggtcaccgtctcgagtgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcgtatccggtgacaccattgtaatgactagtggcgggccccgcactgtggctgaactggagggcaaaccgttcaccgcactgattcgcggctctggctacccatgcccctcaggtttcttccgcacctgtgaacgtgacgtatatgatctgcgtacacgtgagggtcattgcttacgtttgacccatgatcaccgtgttctggtgatggatggtggcctggaatggcgtgccgcgggtgaactggaacgcggcgaccgcctggtgatggatgatgcagctggcgagtttccggcactggcaaccttccgtggcctgcgtggcgctggccgccaggatgtttatgacgctactgtttacggtgctagcgcattcactgctaatggcttcattgtacacgcatgtggcgagcagcccgggaccggtctgaactcaggcctcacgacaaatcctggtgtatccgcttggcaggtcaacacagcttatactgcgggacaattggtcacatataacggcaagacgtataaatgtttgcagccccacacctccttggcaggatgggaaccatccaacgttcctgccttgtggcagcttcaatgagtcctagctggccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggaattaattctaaaatacagcatagcaaaactttaacctccaaatcaagcctctacttgaatccttttctgagggatgaataaggcataggcatcaggggctgttgccaatgtgcattagctgtttgcagcctcaccttctttcatggagtttaagatatagtgtattttcccaaggtttgaactagctcttcatttctttatgttttaaatgcactgacctcccacattccctttttagtaaaatattcagaaataatttaaatacatcattgcaatgaaaataaatgttttttattaggcagaatccagatgctcaaggcccttcataatatcccccagtttagtagttggacttagggaacaaaggaacctttaatagaaattggacagcaagaaagcgagcttctagcttatcctcagtcctgctcctctgccacaaagtgcacgcagttgccggccgggtcgcgcagggcgaactcccgcccccacggctgctcgccgatctcggtcatggccggcccggaggcgtcccggaagttcgtggacacgacctccgaccactcggcgtacagctcgtccaggccgcgcacccacacccaggccagggtgttgtccggcaccacctggtcctggaccgcgctgatgaacagggtcacgtcgtcccggaccacaccggcgaagtcgtcctccacgaagtcccgggagaacccgagccggtcggtccagaactcgaccgctccggcgacgtcgcgcgcggtgagcaccggaacggcactggtcaacttggccatgatggctcctcctgtcaggagaggaaagagaagaaggttagtacaattgctatagtgagttgtattatactatgcagatatactatgccaatgattaattgtcaaactagggctgcagggttcatagtgccacttttcctgcactgccccatctcctgcccaccctttcccaggcatagacagtcagtgacttaccaaactcacaggagggagaaggcagaagcttgagacagacccgcgggaccgccgaactgcgaggggacgtggctagggcggcttcttttatggtgcgccggccctcggaggcagggcgctcggggaggcctagcggccaatctgcggtggcaggaggcggggccgaaggccgtgcctgaccaatccggagcacataggagtctcagccccccgccccaaagcaaggggaagtcacgcgcctgtagcgccagcgtgttgtgaaatgggggcttgggggggttggggccctgactagtcaaaacaaactcccattgacgtcaatggggtggagacttggaaatccccgtgagtcaaaccgctatccacgcccattgatgtactgccaaaaccgcatcatcatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccgtaagttatgtaacgcctgcaggttaattaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatggctagttaattaacatttaaatcagcggccgcaataaaatatctttattttcattacatctgtgtgttggttttttgtgtgaatcgtaactaacatacgctctccatcaaaacaaaacgaaacaaaacaaactagcaaaataggctgtccccagtgcaagtgcaggtgccagaacatttctctatcgaa

SEQ ID NO: 117 - DNA, 4779 bp, synthetic construct
  misc_feature 1..4779: note = pFUSEss-DE27-V 1-CHIg-hG1-Mth-2
  source 1..4779: mol_type = other DNA
ggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacctgagatcaccggcgaaggagggccaccatgtacaggatgcaactcctgtcttgcattgcactaagtcttgcacttgtcacgaattcggaggtgcagctggtggagtctgggggaggcttggtacagcccggcaggtccctgagactctcctgtgcggcctctggattcacctttgatgattatgccatgcactgggtccggcaagctccagggaagggcctggaatgggtctcagctatcacttggaatagtggtcacatagactatgcggactctgtggagggccgattcaccatctccagagacaacgccaagaactccctgtatctgcaaatgaacagtctgagagctgaggatacggccgtatattactgtgcgaaagtctcgtaccttagcaccgcgtcctcccttgactattggggccaaggtaccctggtcaccgtctcgagtgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacagggtgcgtatccggtgacaccattgtaatgactagtggcgggccccgcactgtggctgaactggagggcaaaccgttcaccgcactgattcgcggctctggctacccatgcccctcaggtttcttccgcacctgtgaacgtgacgtatatgatctgcgtacacgtgagggtcattgcttacgtttgacccatgatcaccgtgttctggtgatggatggtggcctggaatggcgtgccgcgggtgaactggaacgcggcgaccgcctggtgatggatgatgcagctggcgagtttccggcactggcaaccttccgtggcctgcgtggcgctggccgccaggatgtttatgacgctactgtttacggtgctagcgcattcactgctaatggcttcattgtacacgcatgtggcgagcagcccgggaccggtctgaactcaggcctcacgacaaatcctggtgtatccgcttggcaggtcaacacagcttatactgcgggacaattggtcacatataacggcaagacgtataaatgtttgcagccccacacctccttggcaggatgggaaccatccaacgttcctgccttgtggcagcttcaatgagtcctagctggccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggaattaattctaaaatacagcatagcaaaactttaacctccaaatcaagcctctacttgaatccttttctgagggatgaataaggcataggcatcaggggctgttgccaatgtgcattagctgtttgcagcctcaccttctttcatggagtttaagatatagtgtattttcccaaggtttgaactagctcttcatttctttatgttttaaatgcactgacctcccacattccctttttagtaaaatattcagaaataatttaaatacatcattgcaatgaaaataaatgttttttattaggcagaatccagatgctcaaggcccttcataatatcccccagtttagtagttggacttagggaacaaaggaacctttaatagaaattggacagcaagaaagcgagcttctagcttatcctcagtcctgctcctctgccacaaagtgcacgcagttgccggccgggtcgcgcagggcgaactcccgcccccacggctgctcgccgatctcggtcatggccggcccggaggcgtcccggaagttcgtggacacgacctccgaccactcggcgtacagctcgtccaggccgcgcacccacacccaggccagggtgttgtccggcaccacctggtcctggaccgcgctgatgaacagggtcacgtcgtcccggaccacaccggcgaagtcgtcctccacgaagtcccgggagaacccgagccggtcggtccagaactcgaccgctccggcgacgtcgcgcgcggtgagcaccggaacggcactggtcaacttggccatgatggctcctcctgtcaggagaggaaagagaagaaggttagtacaattgctatagtgagttgtattatactatgcagatatactatgccaatgattaattgtcaaactagggctgcagggttcatagtgccacttttcctgcactgccccatctcctgcccaccctttcccaggcatagacagtcagtgacttaccaaactcacaggagggagaaggcagaagcttgagacagacccgcgggaccgccgaactgcgaggggacgtggctagggcggcttcttttatggtgcgccggccctcggaggcagggcgctcggggaggcctagcggccaatctgcggtggcaggaggcggggccgaaggccgtgcctgaccaatccggagcacataggagtctcagccccccgccccaaagcaaggggaagtcacgcgcctgtagcgccagcgtgttgtgaaatgggggcttgggggggttggggccctgactagtcaaaacaaactcccattgacgtcaatggggtggagacttggaaatccccgtgagtcaaaccgctatccacgcccattgatgtactgccaaaaccgcatcatcatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccgtaagttatgtaacgcctgcaggttaattaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatggctagttaattaacatttaaatcagcggccgcaataaaatatctttattttcattacatctgtgtgttggttttttgtgtgaatcgtaactaacatacgctctccatcaaaacaaaacgaaacaaaacaaactagcaaaataggctgtccccagtgcaagtgcaggtgccagaacatttctctatcgaa

SEQ ID NO: 118 - protein, 234 aa, synthetic construct
  REGION 1..234: note = pre-kappa light chain of adalimumab
MYRMQLLSCIALSLALVTNSDIQMTQSPSSLSASVGDRVTITCRASQGIRNYLAWYQQKPGKAPKLLIYAASTLQSGVPSRFSGSGSGTDFTLTISSLQPEDVATYYCQRYNRAPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 119 - protein, 214 aa, synthetic construct
  REGION 1..214: note = mature kappa light chain of adalimumab
DIQMTQSPSSLSASVGDRVTITCRASQGIRNYLAWYQQKPGKAPKLLIYAASTLQSGVPSRFSGSGSGTDFTLTISSLQPEDVATYYCQRYNRAPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 120 - protein, 448 aa, synthetic construct
  REGION 1..448: note = pre-heavy chain-intein chimeric polypeptide
MYRMQLLSCIALSLALVTNSEVQLVESGGGLVQPGRSLRLSCAASGFTFDDYAMHWVRQAPGKGLEWVSAITWNSGHIDYADSVEGRFTISRDNAKNSLYLQMNSLRAEDTAVYYCAKVSYLSTASSLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCVSGDTIVMTSGGPRTVAELEGKPFTALIRGSGYPCPSGFFRTCERDVYDLRTREGHCLRLTHDHRVLVMDGGLEWRAAGELERGDRLVMDDAAGEFPALATFRGLRGAGRQDVYDATVYGASAFTANGFIVHACGEQPGTGLNSGLTTNPGVSAWQVNTAYTAGQLVTYNGKTYKCLQPHTSLAGWEPSNVPALWQLQ

SEQ ID NO: 121 - protein, 428 aa, synthetic construct
  REGION 1..428: note = mature heavy chain-intein fusion protein
EVQLVESGGGLVQPGRSLRLSCAASGFTFDDYAMHWVRQAPGKGLEWVSAITWNSGHIDYADSVEGRFTISRDNAKNSLYLQMNSLRAEDTAVYYCAKVSYLSTASSLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCVSGDTIVMTSGGPRTVAELEGKPFTALIRGSGYPCPSGFFRTCERDVYDLRTREGHCLRLTHDHRVLVMDGGLEWRAAGELERGDRLVMDDAAGEFPALATFRGLRGAGRQDVYDATVYGASAFTANGFIVHACGEQPGTGLNSGLTTNPGVSAWQVNTAYTAGQLVTYNGKTYKCLQPHTSLAGWEPSNVPALWQLQ

SEQ ID NO: 122 - protein, 449 aa, synthetic construct
  REGION 1..449: note = pre-heavy chain-intein chimeric polypeptide
MYRMQLLSCIALSLALVTNSEVQLVESGGGLVQPGRSLRLSCAASGFTFDDYAMHWVRQAPGKGLEWVSAITWNSGHIDYADSVEGRFTISRDNAKNSLYLQMNSLRAEDTAVYYCAKVSYLSTASSLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTGCVSGDTIVMTSGGPRTVAELEGKPFTALIRGSGYPCPSGFFRTCERDVYDLRTREGHCLRLTHDHRVLVMDGGLEWRAAGELERGDRLVMDDAAGEFPALATFRGLRGAGRQDVYDATVYGASAFTANGFIVHACGEQPGTGLNSGLTTNPGVSAWQVNTAYTAGQLVTYNGKTYKCLQPHTSLAGWEPSNVPALWQLQ

SEQ ID NO: 123 - protein, 429 aa, synthetic construct
  REGION 1..429: note = mature heavy chain-intein fusion protein
EVQLVESGGGLVQPGRSLRLSCAASGFTFDDYAMHWVRQAPGKGLEWVSAITWNSGHIDYADSVEGRFTISRDNAKNSLYLQMNSLRAEDTAVYYCAKVSYLSTASSLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTGCVSGDTIVMTSGGPRTVAELEGKPFTALIRGSGYPCPSGFFRTCERDVYDLRTREGHCLRLTHDHRVLVMDGGLEWRAAGELERGDRLVMDDAAGEFPALATFRGLRGAGRQDVYDATVYGASAFTANGFIVHACGEQPGTGLNSGLTTNPGVSAWQVNTAYTAGQLVTYNGKTYKCLQPHTSLAGWEPSNVPALWQLQ

SEQ ID NO: 124 - protein, 230 aa, synthetic construct
  REGION 1..230: note = Part of alkyne-modified adalimumab Fab-1 protein. Modified atC-Terminus.
  SITE 230: note = Xaa = cysteine-alkyne
EVQLVESGGGLVQPGRSLRLSCAASGFTFDDYAMHWVRQAPGKGLEWVSAITWNSGHIDYADSVEGRFTISRDNAKNSLYLQMNSLRAEDTAVYYCAKVSYLSTASSLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTX

SEQ ID NO: 125 - protein, 231 aa, synthetic construct
  REGION 1..231: note = Part of alkyne-modified adalimumab Fab-2 protein. Motified atC-Terminus
  SITE 131: note = Xaa = cysteine-alkyne
  SITE 231: note = misc_feature - Xaa can be any naturally occurring amino acid
EVQLVESGGGLVQPGRSLRLSCAASGFTFDDYAMHWVRQAPGKGLEWVSAITWNSGHIDYADSVEGRFTISRDNAKNSLYLQMNSLRAEDTAVYYCAKVSYLSTASSLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTGX

SEQ ID NO: 126 - protein, 230 aa, synthetic construct; identical to SEQ ID NO: 124
  REGION 1..230: note = Part of azide-modified adalimumab Fab-1 protein. ModifiedC-Terminus
  SITE 230: note = Xaa = cysteine-azide

SEQ ID NO: 127 - protein, 231 aa, synthetic construct; identical to SEQ ID NO: 125
  REGION 1..231: note = Part of azide-modified adalimumab Fab-2 protein. ModifiedC-Terminus
  SITE 231: note = Xaa = cysteine-azide

SEQ ID NO: 128 - protein, 214 aa, Homo sapiens
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 129 - protein, 450 aa, Homo sapiens
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 130 - protein, 215 aa, synthetic construct
  REGION 1..215: note = Trastuzumab light chain having the N-terminal sequence C
CDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 131 - protein, 238 aa, synthetic construct
  REGION 1..238: note = pre-light chimeric polypeptide having SHH signal peptide
MLLLARCLLLVLVSSLLVCSGLACDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 132 - protein, 238 aa, synthetic construct
  REGION 1..238: note = pre-light chimeric polypeptide having IFN signal peptide
MALTFALLVALLVLSCKSSCSVGCDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 133 - protein, 232 aa, synthetic construct
  REGION 1..232: note = pre-light chimeric polypeptide having CETP signal peptide
MLAATVLTLALLGNAHACDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 134 - protein, 216 aa, synthetic construct
  REGION 1..216: note = Trastuzumab light chain having the N-terminal sequence CP
CPDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 135 - protein, 239 aa, synthetic construct
  REGION 1..239: note = pre-light chimeric polypeptide having SHH signal peptide
MLLLARCLLLVLVSSLLVCSGLACPDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 136 - protein, 239 aa, synthetic construct
  REGION 1..239: note = pre-light chimeric polypeptide having IFN signal peptide
MALTFALLVALLVLSCKSSCSVGCPDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 137 - protein, 233 aa, synthetic construct
  REGION 1..233: note = pre-light chimeric polypeptide having CETP signal peptide
MLAATVLTLALLGNAHACPDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 138 - protein, 217 aa, synthetic construct
  REGION 1..217: note = Trastuzumab light chain having the N-terminal sequence CPP
CPPDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 139 - protein, 240 aa, synthetic construct
  REGION 1..240: note = pre-light chimeric polypeptide having SHH signal peptide
MLLLARCLLLVLVSSLLVCSGLACPPDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 140 - protein, 240 aa, synthetic construct
  REGION 1..240: note = pre-light chimeric polypeptide having IFN signal peptide
MALTFALLVALLVLSCKSSCSVGCPPDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 141 - protein, 234 aa, synthetic construct
  REGION 1..234: note = pre-light chimeric polypeptide having CETP signal peptide
MLAATVLTLALLGNAHACPPDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 142 - protein, 217 aa, synthetic construct
  REGION 1..217: note = Trastuzumab light chain having the N-terminal sequence CPR
CPRDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 143 - protein, 240 aa, synthetic construct
  REGION 1..240: note = pre-light chimeric polypeptide having SHH signal peptide
MLLLARCLLLVLVSSLLVCSGLACPRDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 144 - protein, 240 aa, synthetic construct
  REGION 1..240: note = pre-light chimeric polypeptide having IFN signal peptide
MALTFALLVALLVLSCKSSCSVGCPRDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 145 - protein, 234 aa, synthetic construct
  REGION 1..234: note = pre-light chimeric polypeptide having CETP signal peptide
MLAATVLTLALLGNAHACPRDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 146 - protein, 217 aa, synthetic construct
  REGION 1..217: note = Trastuzumab light chain having the N-terminal sequence CPS
CPSDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 147 - protein, 240 aa, synthetic construct
  REGION 1..240: note = pre-light chimeric polypeptide having SHH signal peptide
MLLLARCLLLVLVSSLLVCSGLACPSDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 148 - protein, 240 aa, synthetic construct
  REGION 1..240: note = pre-light chimeric polypeptide having IFN signal peptide
MALTFALLVALLVLSCKSSCSVGCPSDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 149 - protein, 234 aa, synthetic construct
  REGION 1..234: note = pre-light chimeric polypeptide having CETP signal peptide
MLAATVLTLALLGNAHACPSDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 150 - protein, 218 aa, synthetic construct
  REGION 1..218: note = Trastuzumab light chain having the N-terminal sequence CDKT
CDKTDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 151 - protein, 241 aa, synthetic construct
  REGION 1..241: note = pre-light chimeric polypeptide having SHH signal peptide
MLLLARCLLLVLVSSLLVCSGLACDKTDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 152 - protein, 241 aa, synthetic construct
  REGION 1..241: note = pre-light chimeric polypeptide having IFN signal peptide
MALTFALLVALLVLSCKSSCSVGCDKTDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 153 - protein, 235 aa, synthetic construct
  REGION 1..235: note = pre-light chimeric polypeptide having CETP signal peptide
MLAATVLTLALLGNAHACDKTDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 154 - protein, 220 aa, synthetic construct
  REGION 1..220: note = Trastuzumab light chain having the N-terminal sequence CDKTHT
CDKTHTDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 155 - protein, 243 aa, synthetic construct
  REGION 1..243: note = pre-light chimeric polypeptide having SHH signal peptide
MLLLARCLLLVLVSSLLVCSGLACDKTHTDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 156 - protein, 243 aa, synthetic construct
  REGION 1..243: note = pre-light chimeric polypeptide having IFN signal peptide
MALTFALLVALLVLSCKSSCSVGCDKTHTDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 157 - protein, 237 aa, synthetic construct
  REGION 1..237: note = pre-light chimeric polypeptide having CETP signal peptide
MLAATVLTLALLGNAHACDKTHTDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 158 - protein, 217 aa, synthetic construct
  REGION 1..217: note = Trastuzumab light chain having the N-terminal sequence CVE
CVEDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 159 - protein, 240 aa, synthetic construct
  REGION 1..240: note = pre-light chimeric polypeptide having SHH signal peptide
MLLLARCLLLVLVSSLLVCSGLACVEDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 160 - protein, 240 aa, synthetic construct
  REGION 1..240: note = pre-light chimeric polypeptide having IFN signal peptide
MALTFALLVALLVLSCKSSCSVGCVEDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 161 - protein, 234 aa, synthetic construct
  REGION 1..234: note = pre-light chimeric polypeptide having CETP signal peptide
MLAATVLTLALLGNAHACVEDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 162 - protein, 220 aa, synthetic construct
  REGION 1..220: note = Trastuzumab light chain having the N-terminal sequence CDTPPP
CDTPPPDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 163 - protein, 243 aa, synthetic construct
  REGION 1..243: note = pre-light chimeric polypeptide having SHH signal peptide
MLLLARCLLLVLVSSLLVCSGLACDTPPPDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 164 - protein, 243 aa, synthetic construct
  REGION 1..243: note = pre-light chimeric polypeptide having IFN signal peptide
MALTFALLVALLVLSCKSSCSVGCDTPPPDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 165 - protein, 237 aa, synthetic construct
  REGION 1..237: note = pre-light chimeric polypeptide having CETP signal peptide
MLAATVLTLALLGNAHACDTPPPDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 166 - protein, 451 aa, synthetic construct
  REGION 1..451: note = Trastuzumab heavy chain having the N-terminal sequence C
CEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 167 - protein, 474 aa, synthetic construct
  REGION 1..474: note = pre-heavy chain chimeric polypeptide having SHH signal peptide
MLLLARCLLLVLVSSLLVCSGLACEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 168 - protein, 474 aa, synthetic construct
  REGION 1..474: note = pre-heavy chain chimeric polypeptide having IFN signal peptide
MALTFALLVALLVLSCKSSCSVGCEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 169 - protein, 468 aa, synthetic construct
  REGION 1..468: note = pre-heavy chain chimeric polypeptide having CETP signal peptide
MLAATVLTLALLGNAHACEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 170 - protein, 452 aa, synthetic construct
  REGION 1..452: note = Trastuzumab heavy chain having the N-terminal sequence CP
CPEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 171 - protein, 475 aa, synthetic construct
  REGION 1..475: note = pre-heavy chain chimeric polypeptide having SHH signal peptide
MLLLARCLLLVLVSSLLVCSGLACPEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 172 - protein, 475 aa, synthetic construct
  REGION 1..475: note = pre-heavy chain chimeric polypeptide having IFN signal peptide
MALTFALLVALLVLSCKSSCSVGCPEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 173 - protein, 469 aa, synthetic construct
  REGION 1..469: note = pre-heavy chain chimeric polypeptide having CETP signal peptide
MLAATVLTLALLGNAHACPEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 174 - protein, 453 aa, synthetic construct
  REGION 1..453: note = Trastuzumab heavy chain having the N-terminal sequence CPP
CPPEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 175 - protein, 476 aa, synthetic construct
  REGION 1..476: note = pre-heavy chain chimeric polypeptide having SHH signal peptide
MLLLARCLLLVLVSSLLVCSGLACPPEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 176 - protein, 476 aa, synthetic construct
  REGION 1..476: note = pre-heavy chain chimeric polypeptide having IFN signal peptide
MALTFALLVALLVLSCKSSCSVGCPPEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 177 - protein, 470 aa, synthetic construct
  REGION 1..470: note = pre-heavy chain chimeric polypeptide having CETP signal peptide
MLAATVLTLALLGNAHACPPEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 178 - protein, 453 aa, synthetic construct
  REGION 1..453: note = Trastuzumab heavy chain having the N-terminal sequence CPR
CPREVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 179 - protein, 476 aa, synthetic construct
  REGION 1..476: note = pre-heavy chain chimeric polypeptide having SHH signal peptide
MLLLARCLLLVLVSSLLVCSGLACPREVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 180 - protein, 476 aa, synthetic construct
  REGION 1..476: note = pre-heavy chain chimeric polypeptide having IFN signal peptide
MALTFALLVALLVLSCKSSCSVGCPREVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 181 - protein, 470 aa, synthetic construct
  REGION 1..470: note = pre-heavy chain chimeric polypeptide having CETP signal peptide
MLAATVLTLALLGNAHACPREVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 182 - protein, 453 aa, synthetic construct
  REGION 1..453: note = Trastuzumab heavy chain having the N-terminal sequence CPS
CPSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 183 - protein, 476 aa, synthetic construct
  REGION 1..476: note = pre-heavy chain chimeric polypeptide having SHH signal peptide
MLLLARCLLLVLVSSLLVCSGLACPSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 184 - protein, 476 aa, synthetic construct
  REGION 1..476: note = pre-heavy chain chimeric polypeptide having IFN signal peptide
MALTFALLVALLVLSCKSSCSVGCPSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 185 - protein, 470 aa, synthetic construct
  REGION 1..470: note = pre-heavy chain chimeric polypeptide having CETP signal peptide
MLAATVLTLALLGNAHACPSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 186 - protein, 454 aa, synthetic construct
  REGION 1..454: note = Trastuzumab heavy chain having the N-terminal sequence CDKT
CDKTEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 187 - protein, 477 aa, synthetic construct
  REGION 1..477: note = pre-heavy chain chimeric polypeptide having SHH signal peptide
MLLLARCLLLVLVSSLLVCSGLACDKTEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 188 - protein, 477 aa, synthetic construct
  REGION 1..477: note = pre-heavy chain chimeric polypeptide having IFN signal peptide
MALTFALLVALLVLSCKSSCSVGCDKTEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 189 - protein, 471 aa, synthetic construct
  REGION 1..471: note = pre-heavy chain chimeric polypeptide having CETP signal peptide
MLAATVLTLALLGNAHACDKTEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 190 - protein, 456 aa, synthetic construct
  REGION 1..456: note = Trastuzumab heavy chain having the N-terminal sequence CDKTHT
CDKTHTEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 191 - protein, 479 aa, synthetic construct
  REGION 1..479: note = pre-heavy chain chimeric polypeptide having SHH signal peptide
MLLLARCLLLVLVSSLLVCSGLACDKTHTEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 192 - protein, 479 aa, synthetic construct
  REGION 1..479: note = pre-heavy chain chimeric polypeptide having IFN signal peptide
MALTFALLVALLVLSCKSSCSVGCDKTHTEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 193 - protein, 473 aa, synthetic construct
  REGION 1..473: note = pre-heavy chain chimeric polypeptide having CETP signal peptide
MLAATVLTLALLGNAHACDKTHTEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 194 - protein, 453 aa, synthetic construct
  REGION 1..453: note = Trastuzumab heavy chain having the N-terminal sequence CVE
CVEEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 195 - protein, 476 aa, synthetic construct
  REGION 1..476: note = pre-heavy chain chimeric polypeptide having SHH signal peptide
MLLLARCLLLVLVSSLLVCSGLACVEEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 196 - protein, 476 aa, synthetic construct
  REGION 1..476: note = pre-heavy chain chimeric polypeptide having IFN signal peptide
MALTFALLVALLVLSCKSSCSVGCVEEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 197 - protein, 470 aa, synthetic construct
  REGION 1..470: note = pre-heavy chain chimeric polypeptide having CETP signal peptide
MLAATVLTLALLGNAHACVEEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 198 - protein, 456 aa, synthetic construct
  REGION 1..456: note = Trastuzumab heavy chain having the N-terminal sequence CDTPPP
CDTPPPEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 199 - protein, 479 aa, synthetic construct
  REGION 1..479: note = pre-heavy chain chimeric polypeptide having SHH signal peptide
MLLLARCLLLVLVSSLLVCSGLACDTPPPEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 200 - protein, 479 aa, synthetic construct
  REGION 1..479: note = pre-heavy chain chimeric polypeptide having IFN signal peptide
MALTFALLVALLVLSCKSSCSVGCDTPPPEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 201 - protein, 473 aa, synthetic construct
  REGION 1..473: note = pre-heavy chain chimeric polypeptide having CETP signal peptide
MLAATVLTLALLGNAHACDTPPPEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 202 - protein, 31 aa, Homo sapiens
HGEGTFTSDVSSYLEEQAAKEFIAWLVKGRG

SEQ ID NO: 203 - DNA, 22 bp, Homo sapiens
  source 1..22: mol_type = other DNA
agaggtagtaggttgcatagtt

SEQ ID NO: 204 - protein, 237 aa, synthetic construct; identical to SEQ ID NO: 108
  REGION 1..237: note = TNR1B

SEQ ID NO: 205 - protein, 222 aa, synthetic construct; identical to SEQ ID NO: 9
  REGION 1..222: note = Fc6

SEQ ID NO: 206 - protein, 5 aa, synthetic construct
  REGION 1..5: note = N-Terminus Sequence
CPPCP

SEQ ID NO: 207 - protein, 5 aa, synthetic construct
  REGION 1..5: note = N-Terminal Sequence
CPRCP

SEQ ID NO: 208 - protein, 5 aa, synthetic construct
  REGION 1..5: note = N-Terminal Sequence
CPSCP

SEQ ID NO: 209 - protein, 11 aa, synthetic construct
  REGION 1..11: note = N-Terminal Sequence
CDKTHTCPPCP

SEQ ID NO: 210 - protein, 8 aa, synthetic construct
  REGION 1..8: note = N-Terminal Sequence
CVECPPCP

SEQ ID NO: 211 - protein, 9 aa, synthetic construct
  REGION 1..9: note = N-Terminal Sequence
CCVECPPCP

SEQ ID NO: 212 - protein, 11 aa, synthetic construct
  REGION 1..11: note = N-Terminal Sequence
CDTPPPCPRCP

SEQ ID NO: 213 - protein, 15 aa, synthetic construct
  REGION 1..15: note = Sequence of component "B"
EPKSCDKTHTCPPCP

SEQ ID NO: 214 - protein, 12 aa, synthetic construct
  REGION 1..12: note = Sequence of component "B"
ERKCCVECPPCP

SEQ ID NO: 215 - protein, 62 aa, synthetic construct
  REGION 1..62: note = Sequence of component "B"
ELKTPLGDTTHTCPRCPEPKSCDTPPPCPRCPEPKSCDTPPPCPRCPEPKSCDTPPPCPRCP

SEQ ID NO: 216 - protein, 12 aa, synthetic construct
  REGION 1..12: note = Sequence of component "B"
ESKYGPPCPSCP

SEQ ID NO: 217 - protein, 6 aa, synthetic construct
  REGION 1..6: note = Sequence in linker
DKTHTC

SEQ ID NO: 218 - protein, 5 aa, synthetic construct
  REGION 1..5: note = Sequence in adalimumab Fab
CDKTH

SEQ ID NO: 219 - protein, 4 aa, synthetic construct
  REGION 1..4: note = Hinge sequence
DKTH

SEQ ID NO: 220 - protein, 5 aa, synthetic construct
  REGION 1..5: note = Hinge sequence
DKTHT

SEQ ID NO: 221 - protein, 13 aa, synthetic construct
  REGION 1..13: note = N-terminal region of alkyne-modified TNR1B sequence
EYYDQTAQMCCSK

SEQ ID NO: 222 - protein, 15 aa, synthetic construct
  REGION 1..15: note = C-terminal region of alkyne-modified TNR1B sequence
SMAPGAVHLPQPVST

SEQ ID NO: 223 - protein, 7 aa, synthetic construct
  REGION 1..7: note = N-terminal region of azide-modified Fc6 protein sequence
DTLMISR

SEQ ID NO: 224 - protein, 18 aa, synthetic construct
  REGION 1..18: note = C-terminal region of azide-modified Fc6 protein sequence
TTPPPVLDSDGSFFLYSK

SEQ ID NO: 225 - protein, 18 aa, synthetic construct
  REGION 1..18: note = Portion of adalimumab Fab
CDKTHTCPPCPAPELLGG

SEQ ID NO: 226 - protein, 17 aa, synthetic construct
  REGION 1..17: note = Portion of adalimumab Fab
CDKTHTCPPCPAPELLG

(19) United States
(12) Patent Application Publication
(10) Pub. No.: US 2026/0193324 A1
(43) Pub. Date: Jul. 9, 2026

(54) HYBRID IMMUNOGLOBULIN CONTAINING NON-PEPTIDYL LINKAGE

(71) Applicant: Biomolecular Holdings LLC, Burlingame, CA (US)

(72) Inventor: Daniel J. Capon, Hillsborough, CA (US)

(73) Assignee: Biomolecular Holdings LLC, Burlingame, CA (US)

(21) Appl. No.: 19/383,092

(22) Filed: Nov. 7, 2025

Related U.S. Application Data
(63) Continuation of application No. 17/364,270, filed on Jun. 30, 2021, now Pat. No. 12,509,502.
Application No. 17/364,270 is a division of application No. 15/125,774, filed on Mar. 13, 2015, now Pat. No. 11,066,459.
(60) Provisional application No. 61/953,650, filed on Mar. 14, 2014.

(51) Int. Cl. C07K 16/00; A61K 47/68; C07K 16/24; C07K 16/46

(52) U.S. Cl. CPC C07K 16/00; A61K 47/6801; A61K 47/6855; A61K 47/6875; A61K 47/6889; C07K 16/241; C07K 16/46; C07K 2317/21; C07K 2317/52; C07K 2317/55; C07K 2317/76

(57) ABSTRACT

The present invention provides a compound having the structure: wherein A is a biologically active structure of the compound; wherein Z is a protein component of the compound, which protein component comprises one or more polypeptides, wherein at least one of the one or more polypeptides comprises consecutive amino acids which (i) are identical to a stretch of consecutive amino acids present in a chain of an Fc domain of an antibody; (ii) bind to an Fc receptor; and (iii) have at their N-terminus a sequence selected from the group consisting of a cysteine or selenocysteine; wherein the dashed line between B and Z represents a peptidyl linkage; and wherein the solid line between A and B represents a nonpeptidyl linkage, as well as intermediates dimers thereof, and processes of producing the compounds of the invention.

### CROSS-REFERENCE TO RELATED APPLICATIONS

This application is a continuation of U.S. application Ser. No. 17/364,270, filed Jun. 30, 2021, now U.S. Pat. No. 12,509,502, issued Dec. 30, 2025, which is a divisional of U.S. application Ser. No. 15/125,774, filed Sep. 13, 2016, now U.S. Pat. No. 11,066,459, issued Jul. 20, 2021, which is a § 371 national stage of PCT International Application No. PCT/US2015/020458, filed Mar. 13, 2015, claiming the benefit of U.S. Provisional Application No. 61/953,650, filed Mar. 14, 2014, the content of each of which is hereby incorporated by reference into the application.

### REFERENCE TO A SEQUENCE LISTING

This application incorporates-by-reference nucleotide and/or amino acid sequences which are present in the file named “251107_86150-PCT-ZA-US_SequenceListing_AD.xml,” which is 410,777 bytes in size, and which was created Nov. 7, 2025.

Throughout this application, various publications are referenced. The disclosures of all referenced publications in their entireties are hereby incorporated by reference into this application in order to more fully describe the state of the art to which this invention pertains.

### BACKGROUND OF THE INVENTION

Proteins prefer to form compact globular or fibrous structures, minimizing their exposure to solvent. This tendency is inherent both in the polypeptide backbone with its propensity for hydrogen-bonded secondary structure, and in side chain interactions that promote tertiary folding. Thus, previous efforts to introduce “flexibility” into antibodies using peptides have been largely inadequate. For example, it is common to employ combinations of an amino acid that favors solvent interactions (e.g., serine) with one that breaks up helical structure (e.g., glycine). While this approach is useful in making fusion proteins such as single-chain antibody fragments (scFv), the resulting structures are actually quite compact with no evidence of extendibility (for Exhibit A example, see Robert et al, (2009) Engineered antibody intervention strategies for Alzheimer's disease and related dementias by targeting amyloid and toxic oligomers. Protein Eng. Des. Sel. 22, 199-208). Furthermore, such sequences are likely to create additional problems due to their intrinsic immunogenicity and proteolytic susceptibility.

There is a need for new protein compounds, incorporating nonprotein chains, that are both flexible and extendible, as well as processes for producing such compounds.

### SUMMARY OF THE INVENTION

The present invention provides a compound having the structure:

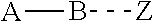

wherein A is a biologically active structure of the compound;
wherein Z is a protein component of the compound, which protein component comprises one or more polypeptides, wherein at least one of the one or more polypeptides comprises consecutive amino acids which (i) are identical to a stretch of consecutive amino acids present in a chain of an F_{c} domain of an antibody; (ii) bind to an F_{c} receptor; and (iii) have at their N-terminus a cysteine or a selenocysteine;
wherein B is a chemical structure linking A and Z;
wherein the dashed line between B and Z represents a peptidyl linkage; and
wherein the solid line between A and B represents a nonpeptidyl linkage.

The present invention also provides a compound having the structure:

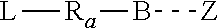

wherein Z is a protein component of the compound, which protein component comprises one or more polypeptides, wherein at least one of the one or more polypeptides comprises consecutive amino acids which (i) are identical to a stretch of consecutive amino acids present in a chain of an F_{c} domain of an antibody; (ii) bind to an F_{c} receptor; and (iii) have at their N-terminus a cysteine or a selenocysteine;
wherein B is a chemical structure linking Rₐ and C;
wherein the dashed line between B and Z represents a peptidyl linkage;
wherein L is selected from the group consisting of: —N₃, an alkyne, a

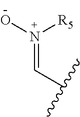

group in which R₅ is an alkyl or aryl group, a

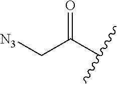 [SMILES: *C(=O)CN=[N+]=[N-]]

group, a tetrazine and a trans-cyclooctene; and
wherein Rₐ is a bond or an organic structure comprising or consisting of a chain of 1, 2, 3, 4, 5, 6, 7, 8, 9, 10 or more moieties, wherein each moiety is independently selected from the group consisting of [PEG(y)]z, polyalkylene glycol, polyoxyalkylated polyol, polyvinyl alcohol, polyvinyl alkyl ether, poly(lactic acid), poly(lactic-glycolic acid), polysaccharide, a branched residue, C₁-C₁₀ alkyl, C₃-C₁₀ cycloalkane, C₂-C₁₀ alkene, C₅-C₁₀ cycloalkene, amine, sulfur, oxygen, succinimide, maleimide, glycerol, triazole, isoxazolidine, C₂-C₅ acyl, C₂-C₅ acylamino, C₂-C₅ acyloxy, succinyl, malonyl, glutaryl, phthalyl, adipoyl, an amino acid, an aryl group, a heteroaryl group, a carbamate, a chemical structure containing a cyclooctane fused to a dihydropyridazine, a chemical structure containing a cyclooctene fused to a triazole, a chemical structure containing a cyclooctene fused to a isoxazolidine, a dibenzocyclooctene, a dibenzoazacyclooctene,

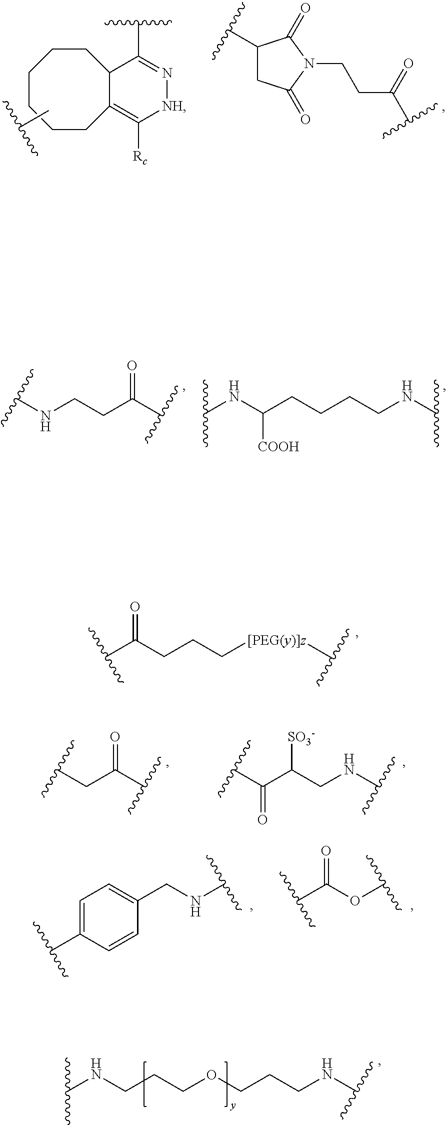

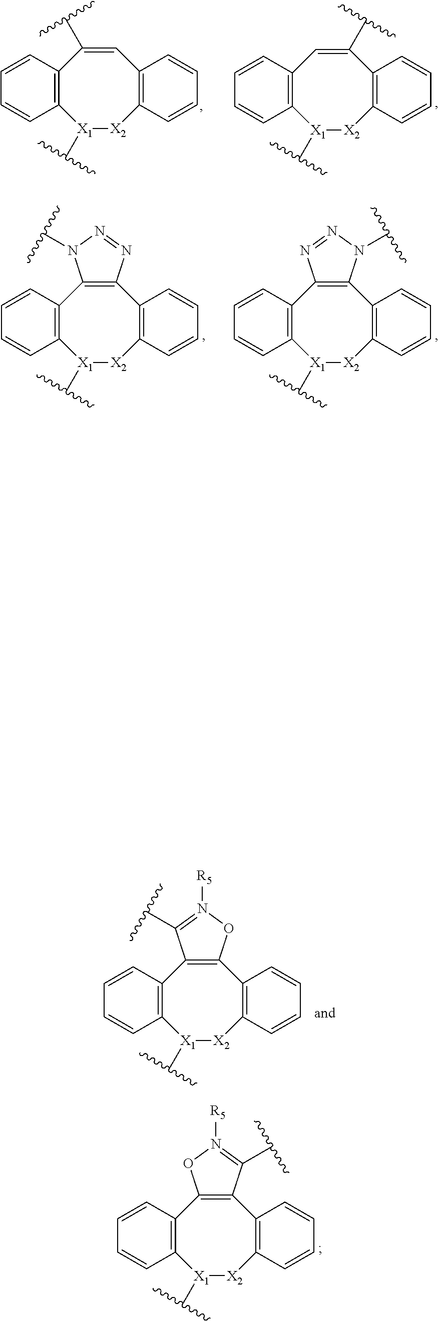

wherein X₁ is CH or N, X₂ is CH₂ or a carbonyl group, and R₅ is an aryl or alkyl group;
wherein [PEG(y)]z is:

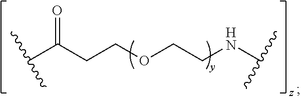

wherein y=1-100 and z=1-10.

The present invention also provides a process for producing a compound having the structure:

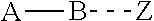

wherein A is a biologically active structure of the compound;
wherein Z is a protein component of the compound, which protein component comprises one or more polypeptides, wherein at least one of the one or more polypeptides comprises consecutive amino acids which (i) are identical to a stretch of consecutive amino acids present in a chain of an F_{c} domain of an antibody; (ii) bind to an F_{c} receptor; and (iii) have at their N-terminus a cysteine or a selenocysteine;
wherein B is a chemical structure linking A and Z;
wherein the dashed line between B and Z represents a peptidyl linkage;
wherein the solid line between A and B represents a nonpeptidyl linkage;
which comprises the following steps:
   a) obtaining an A′ which comprises A or a derivative of A, and a first terminal reactive group;
   b) obtaining a B′ which comprises B or a derivative of B, a second terminal reactive group and a third terminal reactive group, wherein the second terminal reactive group is capable of reacting with the first terminal reactive group to form a non-peptidyl linkage;
   c) obtaining a Z′ which comprises Z or a derivative of Z, and a fourth terminal reactive group, wherein the fourth terminal reactive group is capable of reacting with the third terminal reactive group to form a peptidyl linkage; and
   d) reacting A′, B′ and Z′ in any order to produce the compound.

### BRIEF DESCRIPTION OF THE DRAWINGS

FIG. 1 shows the preparation of alkyne-modified TNR1B by cleavage of a TNR1B-intein fusion protein with cystyl-propargylamide. The intein by-product is removed by chitin chromatography. Azide-modified TNR1B and cycloalkyne-modified TNR1B are similarly prepared using cystyl-3-azidopropylamide, and various cyclooctyne (eg. DIBAC) derivatives of cysteine, respectively.

FIG. 2 shows the cleavage of TNR1B by (1) cysteine, (2) cysteine+mercaptoethane sulfonate (MESNA), (3) cystyl-propargylamide, (4) cystyl-propargylamide+MESNA, and (5) MESNA. All compounds were used at 50 mM concentration.

FIG. 3 shows the preparation of azide-modified Fc6 by ligation (peptidyl) of the Fc6 dimer and azide-DKTHT-thioester (Table 1) DKTHT is SEQ ID NO: 220.

FIG. 4 shows the preparation of azide-modified Fc6 by ligation (peptidyl) of the Fc6 dimer and azide-PEGₙ-DKTHT-thioester (Table 1). Cycloalkyne-modified Fc is similarly prepared by using DIBAC-PEG₁₂-thioester. DKTHT is SEQ ID NO: 220.

FIG. 5 shows SDS-PAGE analysis (reducing conditions) of (1) unmodified Fc6, (2) the Az-DKTHT-Fc6 reaction product of FIG. 3, and (3) the Az-PEG₄-DKTHT-Fc6 reaction product of FIG. 4.

FIG. 6 shows the synthesis of TNR1B-alkyne-azide-Fc6 by ligation (non-peptidyl) of alkyne-modified TNR1B and Az-DKTHT-Fc6. DKTHT is SEQ ID NO: 220.

FIG. 7 shows the synthesis of TNR1B-alkyne-azide-PEGₙ-Fc6 by ligation (non-peptidyl) of alkyne-modified TNR1B and azide-PEGₙ-DKTHT-Fc6. In this example, n=4. DKTHT is SEQ ID NO: 220.

FIG. 8 shows SDS-PAGE analysis (reducing conditions) of (1) alkyne-modified TNR1B alone, (2) alkyne-modified TNR1B+Az-DKTHT-Fc6 in the absence of catalyst, (3) alkyne-modified TNR1B+Az-DKTHT-Fc6+catalyst leading to the product of FIG. 6, and (4) dialyzed alkyne-modified TNR1B+Az-DKTHT-Fc6+catalyst leading to increased formation of the product of FIG. 6 (5) alkyne-modified TNR1B+Az-PEG₄-DKTHT-Fc6 in the absence of catalyst, (6) alkyne-modified TNR1B+Az-PEG₄-DKTHT-Fc6+catalyst leading to the product of FIG. 7, and (7) dialyzed alkyne-modified TNR1B+Az-PEG₄-DKTHT-Fc6+catalyst leading to increased formation of the product of FIG. 7. The arrows correspond to (a) Mr ~75,000, (b) Mr ~65,000, (c) Mr ~43,000, and (d) Mr ~28,000. DKTHT is SEQ ID NO: 220.

FIG. 9 shows SDS-PAGE analysis (reducing conditions) of (1) TNF1B-Fc fusion protein (etanercept) alone, (2) alkyne-modified TNR1B+Az-DKTHT-Fc6+catalyst leading to the product of FIG. 6, (3) TNF1B-Fc fusion protein (etanercept), and (4) alkyne-modified TNR1B+Az-PEG₄-DKTHT-Fc6 leading to the product of FIG. 7. DKTHT is SEQ ID NO: 220. The arrows correspond to (a) Mr ~75,000, (b) Mr ~65,000, (c) Mr ~43,000, and (d) Mr ~28,000.

FIG. 10 shows SDS-PAGE analysis (reducing conditions) of (1) unmodified Fc6+catalyst, (2) alkyne-modified TNR1B+unmodified Fc6+catalyst (3) Az-DKTHT-Fc6+catalyst, (4) alkyne-modified TNR1B+Az-DKTHT-Fc6+catalyst leading to the product of FIG. 6, and (5) alkyne-modified TNR1B alone. The arrows correspond to (a) Mr ~75,000, (b) Mr ~65,000, (c) Mr ~43,000, (d) Mr ~28,000, and (e) Mr ~27,000. DKTHT is SEQ ID NO: 220.

FIG. 11 shows tryptic peptides identified by LC/MS in the TNR1B-alkyne-azide-DKTHT-Fc6 product (Mr ~75,000) of FIG. 10. The underlined peptide sequences are those identified by LC/MS that are derived from the parent TNR1B (upper, SEQ ID NO: 204) and Fc6 (lower, SEQ ID NO: 205) sequences. DKTHT is SEQ ID NO: 220.

FIG. 12 shows SPR analysis of TNF-α binding by the TNR1B-alkyne-azide-DKTHT-Fc6 (left panel) and TNR1B-alkyne-azide-PEG₄-DKTHT-Fc6 (right panel) reaction products of FIG. 9. The kinetic binding data are summarized in Table 2. DKTHT is SEQ ID NO: 220.

FIG. 13 shows the preparation of adalimumab Fab′ in a three-step process: 1) IdeS cleavage to the Fab′2+Fc′ fragments, 2) Protein A chromatography to remove the Fc′ fragment, and 3) mild reduction of the Fab′2 fragment to the Fab′ fragment with 2-mercaptoethylamine (MEA).

FIG. 14 shows SDS-PAGE analysis of (1) adalimumab, (2) adalimumab after IdeS cleavage, (3) adalimumab Fab′2 after Protein A purification, (4) adalimumab Fab′ after MEA treatment of the Protein A purified Fab′2, (5) adalimumab Fab′2 after Protein A purification, and (6) adalimumab Fab′ after MEA treatment of the Protein A purified Fab′2. The samples in lanes 1, 2, 5 and 6 were analysis under reducing conditions; while the samples in lanes 3 and 4 were analyzed under non-reducing conditions. The arrows correspond to the (a) heavy chain, (b) heavy chain Fc′ fragment, (c) heavy chain Fd′ (variable region-containing) fragment, and (d) light chain.

FIG. 15 shows the preparation of cycloalkyne-modified Fab′ by the reaction of adalimumab Fab′ with DIBAC-PEGy-Lys(Mal). In this example, PEGy=PEG₁₂.

FIG. 16 shows SDS-PAGE analysis (non-reducing conditions) of the synthesis and purification of cycloalkyne-modified adalimumab Fab′. Upper panel shows the reaction at (1-6) pH 7.4 and (7-12) pH 7.0. The DIBAC-PEGy-Lys(Mal) to Fab′ ration was (1) 0, (2) 10:1, (3) 5:1, (4) 2.5:1, (5) 1.2:1, (6) 0.6:1, (7) 0, (8) 10, (9) 5, (10) 2.5, (11) 1.2, and (12) 0.6:1. The lower panel shows (1) unreacted Fab′, (2) through (12) Protein L flow-through fractions containing only the cycloalkyne-modified Fab′.

FIG. 17 shows SDS-PAGE analysis (reducing conditions) of (1) Fc6, (2) Az-DKTHT-Fc6, (3) Az-PEG₁₂-DKTHT-Fc6, (4) Az-PEG₂₄-DKTHT-Fc6, and (5) Az-PEG₃₆-DKTHT-Fc6. DKTHT is SEQ ID NO: 220.

FIG. 18 shows size-exclusion chromatography of (a) Az-PEG₃₆-DKTHT-Fc6, (b) Az-PEG₂₄-DKTHT-Fc6, (c) Az-PEG₁₂-DKTHT-Fc6, (d) Az-DKTHT-Fc6, and (e) Fc6. DKTHT is SEQ ID NO: 220.

FIG. 19 shows the synthesis of Fab′-PEGy-alkyne-azide-PEGx-Fc6 by ligation (non-peptidyl) of cycloalkyne-modified adalimumab Fab′ and azide-modified Fc6.

FIG. 20 shows the Fab′-PEGy-alkyne-azide-PEGx-Fc6 product series.

FIG. 21 shows SDS-PAGE analysis of (1) adalimumab whole antibody, (2) adalimumab Fab′, (3) Fab′-PEG₁₂-alkyne, (4) Fab′-PEG₁₂-alkyne+Az-DKTHT-Fc6, (5) Az-DKTHT-Fc6, (6) Fab′-PEG₁₂-alkyne+Az-PEG₁₂-DKTHT-Fc6, (7) Az-PEG₁₂-DKTHT-Fc6, (8) Fab′-PEG₁₂-alkyne+Az-PEG₂₄-DKTHT-Fc6, (9) Az-PEG₂₄-DKTHT-Fc6 alone, (10) Fab′-PEG₁₂-alkyne+Az-PEG₃₆-DKTHT-Fc6, (11) Az-PEG₃₆-DKTHT-Fc6, and (12) Fc6. Samples were run under reducing conditions (upper panel) and non-reducing conditions (lower panel). In the upper panel the arrow shows (a) Fab′-PEGy-alkyne-azide-PEGx-Fc6 heavy chain. In the lower panels the arrows show (a) two-handed Fab′-PEGy-alkyne-azide-PEGx-Fc6 molecules, and (b) one-handed Fab′-PEGy-alkyne-azide-PEGx-Fc6 molecules. DKTHT is SEQ ID NO: 220.

FIG. 22 shows size-exclusion chromatography (SEC) of two-handed reaction products: (a) Fab′-PEG₁₂-alkyne-azide-PEG₃₆-DKTHT-Fc6, (b) Fab′-PEG₁₂-alkyne-azide-PEG₂₄-DKTHT-Fc6, (c) Fab′-PEG₁₂-alkyne-azide-PEG₁₂-DKTHT-Fc6, (d) Fab′-PEG₁₂-alkyne-azide-DKTHT-Fc6, and (e) whole adalimumab. DKTHT is SEQ ID NO: 220.

FIG. 23 shows the inhibition of TNF-α cytotoxity on WEHI cells by reaction products. The upper panel shows the (a) Fc6 control, (b) cycloalkyne-modified Fab′, (c) Fab′-PEG₁₂-alkyne-azide-DKTHT-Fc6, and (d) Fab′-PEG₁₂-alkyne-azide-PEG₁₂-DKTHT-Fc6. The lower panel shows (a) Fc6 control, (b) cycloalkyne-modified Fab′, (c) Fab′-PEG₁₂-alkyne-azide-PEG₂₄-DKTHT-Fc6, and (d) Fab′-PEG₁₂-alkyne-azide-PEG₃₆-DKTHT-Fc6. DKTHT is SEQ ID NO: 220.

FIG. 24 shows the purified N3-Px-Fc proteins by SDS-PAGE under reducing (left) and non-reducing conditions (right): Fc6 control (lanes a), N3-P0-Fc (lanes b), N3-P12-Fc (lanes c), N3-P24-Fc (lanes d), N3-P36-Fc (lanes e), and N3-P48-Fc (lanes f).

FIG. 25 shows shows the structure and synthesis of the cyclooctyne-modified GLP-1 analog (GLP1-P7-DBCO).

FIG. 26 shows the reaction between GLP1-P7-DBCO and the N3-Px-Fc proteins.

FIG. 27 shows the structure of GLP1-triazole-Fc hybrid immunoglobulins.

FIG. 28 shows the purified GLP1-triazole-Fc hybrid immunoglobulins by SDS-PAGE under reducing conditions (left) and non-reducing conditions (right): Fc6 control (lanes a), GLP1-P4-DN-P0-Fc (lanes b), GLP1-P4-DN-P12-Fc (lanes c), GLP1-P4-DN-P24-Fc (lanes d), GLP1-P4-DN-P36-Fc (lanes e), and GLP1-P4-DN-P48-Fc (lanes f).

FIG. 29 directly compares the GLP1-triazole-Fc hybrid immunoglobulins and N3-Px-Fc proteins by SDS-PAGE under reducing conditions: Fc6 control (lane a), N3-P0-Fc (lane b), GLP1-P4-DN-P0-Fc (lane c), N3-P12-Fc (lane d), GLP1-P4-DN-P12-Fc (lane e), N3-P24-Fc (lane f), GLP1-P4-DN-P24-Fc (lane g), N3-P36-Fc (lane h), GLP1-P4-DN-P36-Fc (lane i), N3-P48-Fc (lane j), GLP1-P4-DN-P48-Fc (lane k).

FIG. 30 compares the induction of cAMP synthesis in GLP-1 receptor expressing cells by GLP1-triazole-Fc hybrid immunoglobulins and GLP-1.

FIG. 31 shows the reaction between DIBAC-PEG11-DIBAC and the N3-Px-Fc proteins.

FIG. 32 shows the structure of the cyclooctyne-Px-Fc proteins.

FIG. 33 shows the DIBAC-P11-DN-P0-Fc reaction product by SDS-PAGE under reducing conditions: Fc6 control (lane b), unpurified reaction product (lanes c-e), the purified N3-P0-Fc protein (lane f), and the purified DIBAC-P11-DN-P0-Fc protein (lane g).

FIG. 34 shows the reaction between azide-modified DNA and the cyclooctyne-Px-Fc proteins.

FIG. 35 shows the structure of DNA-triazole-Fc hybrid immunoglobulins.

FIG. 36 shows the DNA-triazole-Fc hybrid immunoglobulins reaction products by SDS-PAGE under reducing conditions: the 5AzD-let7d oligonucleotide concentration (mg/ml) was as follows: markers (lane a), 0 (lane b), 2.5 (lane c), 1.25 (lane d), 0.063 (lane e), 0.031 (lane f), 0.016 (lane g), 0.08 (lane h).

FIG. 37 shows the structure and synthesis of the trastuzumab variant, cys1H-IgG1, and the azide-modified trastuzumab heavy chain (N₃-Px-Hc).

FIG. 38 shows the structure and synthesis of the trastuzumab variant, cys1L-IgG1, the azide-modified trastuzumab light chain (N₃-Px-Lc).

FIG. 39 shows the structure and synthesis of cyclooctyne-modified DM-1 (DM1-P4-DBCO).

FIG. 40 shows the reaction between cyclooctyne-modified DM-1 and the N₃-Px-Hc proteins.

FIG. 41 shows the structure of DM1-P4-triazole-Px-Hc hybrid immunoglobulins.

FIG. 42 shows the reaction between cyclooctyne-modified DM-1 and the N₃-Px-Lc proteins.

FIG. 43 shows the structure of DM1-P4-triazole-Px-Lc hybrid immunoglobulins.

FIG. 44 shows the reaction between Tetrazine-DBCO and the N₃-Px-Fc proteins.

FIG. 45 shows the structure of tetrazine-modified Fc proteins (Tet-Px-Fc).

FIG. 46 shows the purified Tet-Px-Fc proteins by SDS-PAGE under reducing (left) and non-reducing conditions (right): Fc6 control (lanes a), Tet-P0-Fc (lanes b), Tet-P12-Fc (lanes c), Tet-P24-Fc (lanes d), Tet-P36-Fc (lanes e), and Tet-P48-Fc (lanes f).

FIG. 47 shows the reaction between TCO-PEG12-DBCO and the N₃-Px-Fc proteins.

FIG. 48 shows the structure of transcyclooctene-modified Fc proteins (Tco-Px-Fc).

FIG. 49 shows the Tco-P12-Px-Fc proteins by SDS-PAGE under reducing conditions: the Tco-P12-DBCO linker concentration (mg/ml) was as follows: 32 (lane a), 16 (lane b), 8 (lane c), 4 (lane d), 2 (lane e), 1 (lane f), 0.5 (lane g), 0.25 (lane h), 0.125 (lane i), and 0 (lane j).

FIG. 50 shows the reaction products between NH2-PEG23-N3 and DBCO-TT-P12-P36-Fc protein by SDS-PAGE under reducing conditions: the NH2-PEG23-N3 linker concentration (mg/ml) was as follows: 0.12 (lane a), 0.06 (lane b), 0.03 (lane c), 0.015 (lane d), 0.0075 (lane e), 0.0038 (lane f), 0.002 (lane g), 0.001 (lane h), 0 (lane i).

FIG. 51 shows the structure and synthesis of the transcyclooctene-modified GLP-1 analog (GLP1-P6-Tco).

FIG. 52 shows the reaction between GLP1-P6-Tco peptide and the Tet-Px-Fc proteins.

FIG. 53 shows the structure of the GLP1-dihydropyridizine-Fc hybrid immunoglobulins.

FIG. 54 shows the structure and synthesis of the tetrazine-modified GLP-1 analog (GLP1-P6-Tet).

FIG. 55 shows the reaction between GLP1-P6-Tet peptide and the Tco-Px-Fc proteins.

FIG. 56 shows the structure of the GLP1-P6-TT-Px-Fc hybrid immunoglobulins.

FIG. 57 shows the purified GLP1-dihydropyridizine-Fc hybrid immunoglobulins by SDS-PAGE under reducing conditions (left) and non-reducing conditions (right): Fc6 control (lanes a), GLP1-P6-TT-P0-Fc (lanes b), GLP1-P6-TT-P12-Fc (lanes c), GLP1-P6-TT-P24-Fc (lanes d), GLP1-P6-TT-P36-Fc (lanes e), and GLP1-P6-TT-P48-Fc (lanes f).

FIG. 58 directly compares the N3-Px-Fc (I) proteins, the Tet-Px-Fc (II) proteins, and the GLP1-dihydropyridizine-Fc (III) hybrid immunoglobulins by SDS-PAGE under reducing conditions: Fc6 control (lane a), N3-P0-Fc (lane b), Tet-P0-Fc (lane c), GLP1-P6-TT-P0-Fc (lane d), N3-P12-Fc (lane e), Tet-P12-Fc (lane f), GLP1-P6-TT-P12-Fc (lane g), N3-P24-Fc (lane h), Tet-P24-Fc (lane i), GLP1-P6-TT-P24-Fc (lane j), N3-P36-Fc (lane k), Tet-P36-Fc (lane l), GLP1-P6-TT-P36-Fc (lane m), N3-P48-Fc (lane n), Tet-P48-Fc (lane o), GLP1-P6-TT-P48-Fc (lane p).

FIG. 59 shows a time course for the reaction of GLP1-P7-DBCO with N3-P36-Fc and a time course for the reaction of GLP1-P6-Tco with Tet-P36-Fc.

FIG. 60 compares the induction of cAMP synthesis in GLP-1 receptor expressing cells by GLP1-dihydropyridizine-Fc hybrid immunoglobulins and GLP-1.

FIG. 61 shows the structure and synthesis of the transcyclooctene-modified adalimumab Fab (Fab-P3-Tco).

FIG. 62 shows the reaction between Fab-P3-Tco protein and the Tet-Px-Fc proteins.

FIG. 63 shows the structure of the Fab-dihydropyridizine-Fc hybrid immunoglobulins.

FIG. 64 shows the Fab-dihydropyridizine-Fc hybrid immunoglobulins by SDS-PAGE under reducing conditions: markers (lanes a), adalimumab (lane b), Fab-P3-TT-P0-Fc (lane c), Fab-P3-TT-P12-Fc (lane d), Fab-P3-TT-P24-Fc (lanes e), Fab-P3-TT-P36-Fc (lanes f), Fab-P3-TT-P48-Fc (lane g), Fab-P3-Tco (lane h), Tet-P0-Fc (lane i), Tet-P12-Fc (lane j), Tet-P24-Fc (lane k), Tet-P36-Fc (lane l)m Tet-P48-Fc (lane m).

FIG. 65 shows the structure and synthesis of azide-modified and transcyclooctene-modified olanzapine (Ola-P12-Tco).

FIG. 66 shows the reaction between Ola-P12-Tco and the Tet-Px-Fc proteins.

FIG. 67 shows the structure of olanzapine-dihydropyridizine-Fc hybrid immunoglobulins.

### DETAILED DESCRIPTION OF THE INVENTION

The present invention provides a compound having the structure:

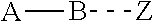

wherein A is a biologically active structure of the compound;
wherein Z is a protein component of the compound, which protein component comprises one or more polypeptides, wherein at least one of the one or more polypeptides comprises consecutive amino acids which (i) are identical to a stretch of consecutive amino acids present in a chain of an F_{c} domain of an antibody; (ii) bind to an F_{c} receptor; and (iii) have at their N-terminus a cysteine or a selenocysteine;
wherein B is a chemical structure linking A and Z;
wherein the dashed line between B and Z represents a peptidyl linkage; and
wherein the solid line between A and B represents a nonpeptidyl linkage.

In some embodiments, the cysteine or selenocysteine naturally occurs in the stretch of consecutive amino acids. In some embodiments, the cysteine or selenocysteine does not naturally occur in the stretch of consecutive amino acids.

In some embodiments, the consecutive amino acids have at their N-terminus a sequence selected from the group consisting of a cysteine, selenocysteine, CP, CPXCP (where X=P, R, or S) (SEQ ID NOs: 206-208), CDKTHTCPPCP (SEQ ID NO: 209), CVECPPCP (SEQ ID NO: 210), CCVECPPCP (SEQ ID NO: 211) and CDTPPPCPRCP (SEQ ID NO: 212).

In some embodiments, the F_{c} domain of an antibody is a naturally occurring F_{c} domain of an antibody.

In some embodiments, the F_{c} domain of an antibody is a variant F_{c} domain of an antibody.

In some embodiments, the variant F_{c} domain of an antibody is a mutated F_{c} domain of an antibody.

In some embodiments, the mutated F_{c} domain is a substitution mutant.

In some embodiments, the substitution mutant has an amino acid substitution at the N-terminus, the C-terminus, or at a position of the F_{c} domain other than the N-terminus or the C-terminus.

In some embodiments, the substitution mutant has 1, 2, 3, 4, 5, 6, 7, 8, 9, 10, 10-15, 15-20, 10-20, or 20-50 amino acid substitutions in the stretch of consecutive amino acids thereof.

In some embodiments, the substitutions are conservative amino acid substitutions.

In some embodiments, the mutated mutated F_{c} domain is an amino acid addition mutant.

In some embodiments, the amino acid addition mutant has 1, 2, 3, 4, 5, 6, 7, 8, 9, 10, 10-15, 15-20, 10-20, or 20-50 added amino acids in the stretch of consecutive amino acids thereof.

In some embodiments, the mutated F_{c} domain is an amino acid deletion mutant.

In some embodiments, the amino acid deletion mutant has 1, 2, 3, 4, 5, 6, 7, 8, 9, 10, 10-15, 15-20, 10-20, or 20-50 deleted amino acids in the stretch of consecutive amino acids thereof.

In some embodiments, the consecutive amino acids are identical to a stretch of at least 0, 20, 30, 40, 50, 60, 70, 80, 90, 100, 110, 120, 130, 140, 150, 160, 170, 180, or 190 consecutive amino acids present in the chain of the F_{c} domain of the antibody.

In some embodiments, the consecutive amino acids are identical to the stretch of amino acids in the hinge region, the CH2 region or the CH3 region of the Fc domain, or a portion thereof.

In some embodiments, the solid line between A and B represents a nonpeptidyl linkage comprising the structure:

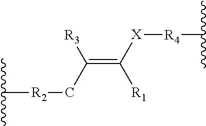

wherein

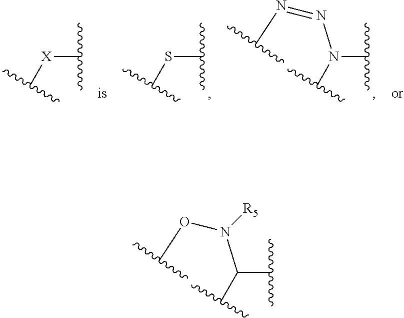

in which R₅ is an alkyl or aryl group
   wherein R₁ is H or is part of an additional structure that is a cyclic structure, wherein the additional cyclic structure comprises R₁ or a portion of R₁, and may also comprise R₂ or a portion of R₂, and the carbon between R₂ and the alkene double bond;
   with the proviso that if

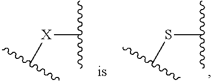

R₃ is a H; if

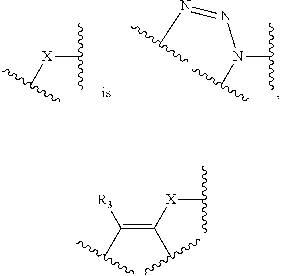

is a triazole ring that comprises

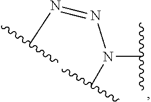 [SMILES: */N=N\N(*)*]

and if

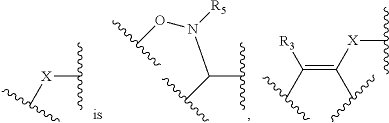

is a N-alkyl or aryl substituted isoxazoline ring that comprises

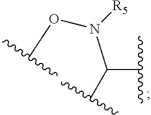

and
wherein R₂ represents an organic structure which connects to one of A or B and R₄ represents an organic structure which connects to the other of A or B.

In some embodiments, the solid line between A and B represents a nonpeptidyl linkage comprising the structure:

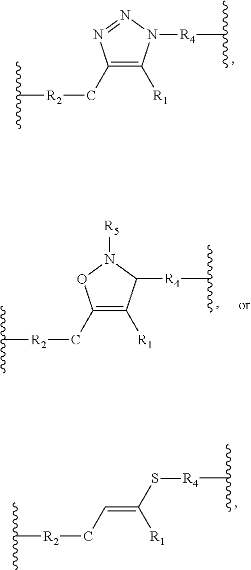

wherein R₁ is H or is part of an additional structure that is a cyclic structure, wherein the additional cyclic structure comprises R₁ or a portion of R₁, and may also comprise R₂ or a portion of R₂, and the carbon between R₂ and the alkene double bond.

In some embodiments, the solid line between A and B represents a nonpeptidyl linkage comprising the structure:

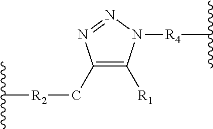

wherein R₁ is H or is part of an additional structure that is a cyclic structure, wherein the additional cyclic structure comprises R₁ or a portion of R₁, and may also comprise R₂ or a portion of R₂, and the carbon between R₂ and the alkene double bond.

In some embodiments, the solid line between A and B represents a nonpeptidyl linkage comprising the structure:

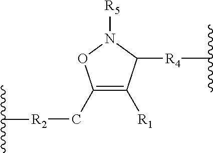

wherein R₁ is part of an additional structure that is a cyclic structure, wherein the additional cyclic structure comprises R₁ or a portion of R₁, and may also comprise R₂ or a portion of R₂, and the carbon between R₂ and the alkene double bond.

In some embodiments, the solid line between A and B represents a nonpeptidyl linkage comprising the structure:

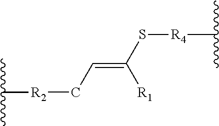

wherein R₁ is part of an additional structure that is a cyclic structure, wherein the additional cyclic structure comprises R₁ or a portion of R₁, and may also comprise R₂ or a portion of R₂, and the carbon between R₂ and the alkene double bond.

In some embodiments, R₁ and R₂ are linked via at least one direct bond so as to form a cyclic structure comprising
i) a portion of R₁,
ii) a portion of R₂,
iii) the carbon between R₂ and the alkene double bond, and
iv) the alkene double bond.

In some embodiments, R₁ is selected from the group consisting of:

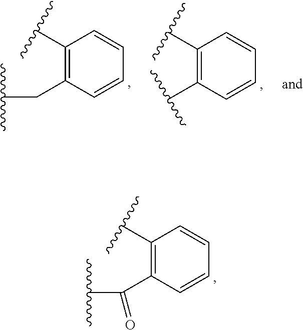 [SMILES: *Cc1ccccc1* ; *c1ccccc1* ; *C(=O)c1ccccc1*]

which is optionally substituted at any position.

In some embodiments, R₁ is

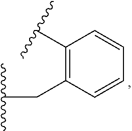 [SMILES: *Cc1ccccc1*]

which is optionally substituted at any position.

In some embodiments, R₁ is

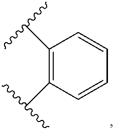 [SMILES: *c1ccccc1*]

which is optionally substituted at any position.

In some embodiments, R₁ is

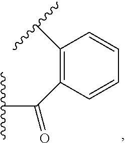 [SMILES: *C(=O)c1ccccc1*]

which is optionally substituted at any position.

In some embodiments, the carbon between R₂ and the alkene double bond is:
(i) directly bonded to R₂ with a single bond and substituted with two substituents independently selected from the group consisting of hydrogen, halogen, optionally substituted benzyl, optionally substituted alkyl or optionally substituted alkoxy; or
(ii) directly bonded to R₂ via a double bond and a single bond.

In some embodiments, the carbon between R₂ and the alkene double bond is substituted with two hydrogens and directly bonded to R₂ with a single bond.

In some embodiments, the carbon between R₂ and the alkene double bond is directly bonded to R₂ via a double bond and a single bond.

In some embodiments, the carbon between R₂ and the alkene double bond is directly bonded to R₂ via a double bond and a single bond so as to form a phenyl ring which is optionally substituted at any position.

In some embodiments, R₂ is

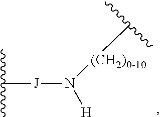

wherein R₂ is attached to A or B via J, and
wherein J is a bond or an organic structure comprising or consisting of a chain of 1, 2, 3, 4, 5, 6, 7, 8, 9, 10 or more moieties, wherein each moiety is independently selected from the group consisting of [PEG(y)]z, polyalkylene glycol, polyoxyalkylated polyol, polyvinyl alcohol, polyvinyl alkyl ether, poly(lactic acid), poly(lactic-glycolic acid), polysaccharide, a branched residue, C₁-C₁₀ alkyl, C₃-C₁₀ cycloalkane, C₂-C₁₀ alkene, C₅-C₁₀ cycloalkene, amine, sulfur, oxygen, succinimide, maleimide, glycerol, triazole, isoxazolidine, C₂-C₅ acyl, C₂-C₅ acylamino, C₂-C₅ acyloxy, succinyl, malonyl, glutaryl, phthalyl, adipoyl, an amino acid, an aryl group, a heteroaryl group, a carbamate, a chemical structure containing a cyclooctane fused to a dihydropyridazine, a chemical structure containing a cyclooctene fused to a triazole, a chemical structure containing a cyclooctene fused to a isoxazolidine, a dibenzocyclooctene, a dibenzoazacyclooctene,

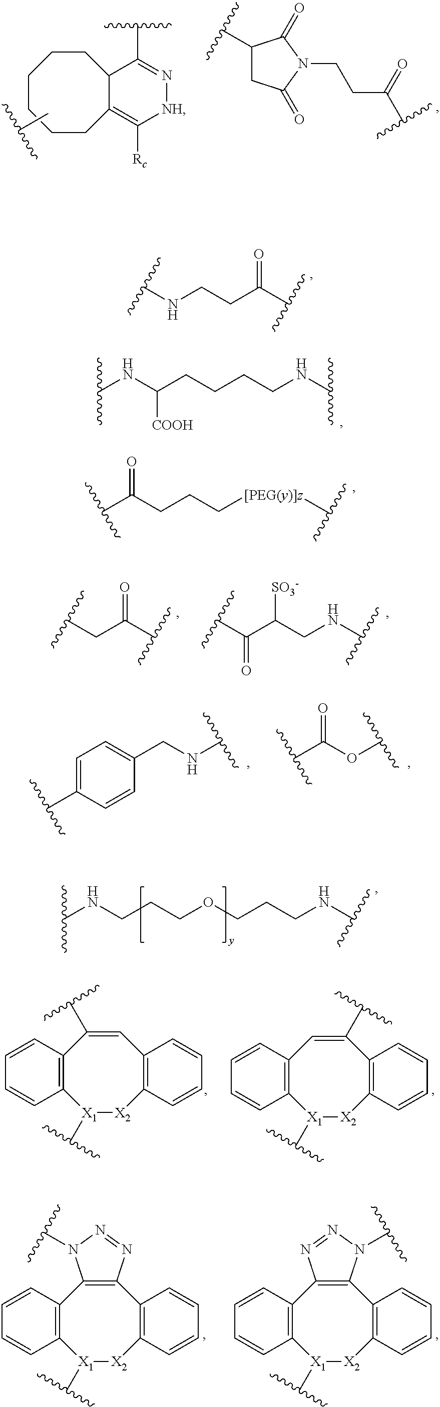

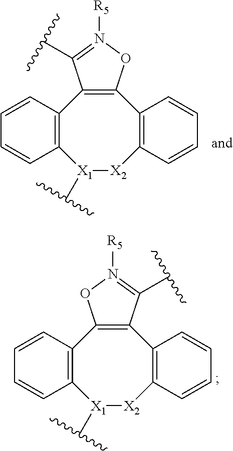

wherein X₁ is CH or N, X₂ is CH₂ or a carbonyl group, and R₅ is an aryl or alkyl group;
wherein [PEG(y)]z is:

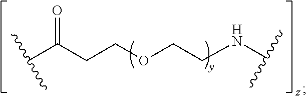

wherein y=1-100 and z=1-10.

In some embodiments, R₂ is

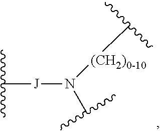

wherein R₂ is attached to A or B via J, and
wherein R₂ is attached to R₁ via the nitrogen atom of R₂, and
wherein J is a bond or an organic structure comprising or consisting of a chain of 1, 2, 3, 4, 5, 6, 7, 8, 9, 10 or more moieties, wherein each moiety is independently selected from the group consisting of [PEG(y)]z, polyalkylene glycol, polyoxyalkylated polyol, polyvinyl alcohol, polyvinyl alkyl ether, poly(lactic acid), poly(lactic-glycolic acid), polysaccharide, a branched residue, C₁-C₁₀ alkyl, C₃-C₁₀ cycloalkane, C₂-C₁₀ alkene, C₅-C₁₀ cycloalkene, amine, sulfur, oxygen, succinimide, maleimide, glycerol, triazole, isoxazolidine, C₂-C₅ acyl, C₂-C₅ acylamino, C₂-C₅ acyloxy, succinyl, malonyl, glutaryl, phthalyl, adipoyl, an amino acid, an aryl group, a heteroaryl group, a carbamate, a chemical structure containing a cyclooctane fused to a dihydropyridazine, a chemical structure containing a cyclooctene fused to a triazole, a chemical structure containing a cyclooctene fused to a isoxazolidine, a dibenzocyclooctene, a dibenzoazacyclooctene,

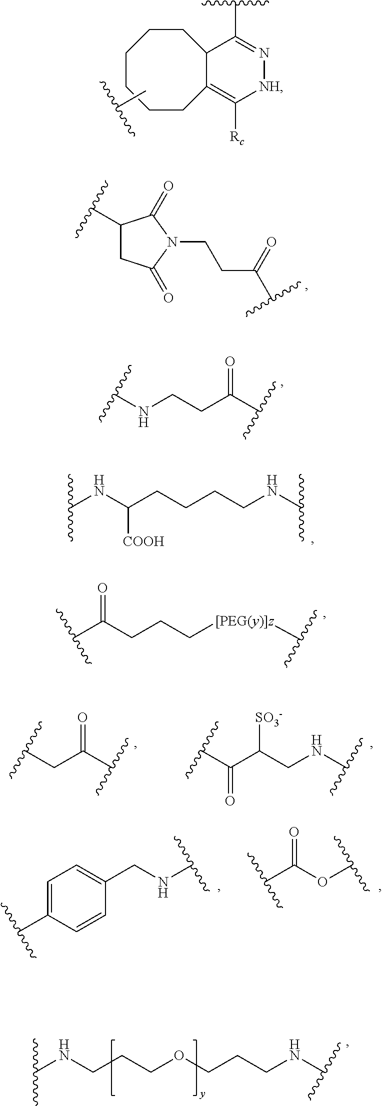

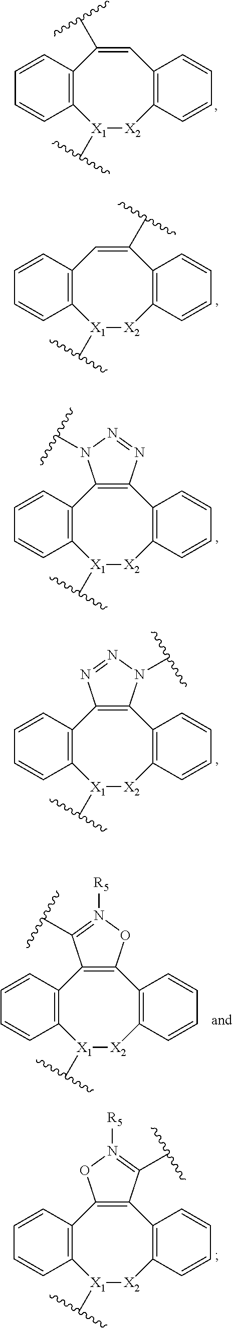

wherein X₁ is CH or N, X₂ is CH₂ or a carbonyl group, and R₅ is an aryl or alkyl group;
wherein [PEG(y)]z is:

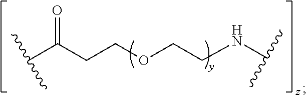

wherein y=1-100 and z=1-10.

In some embodiments, R₂ is

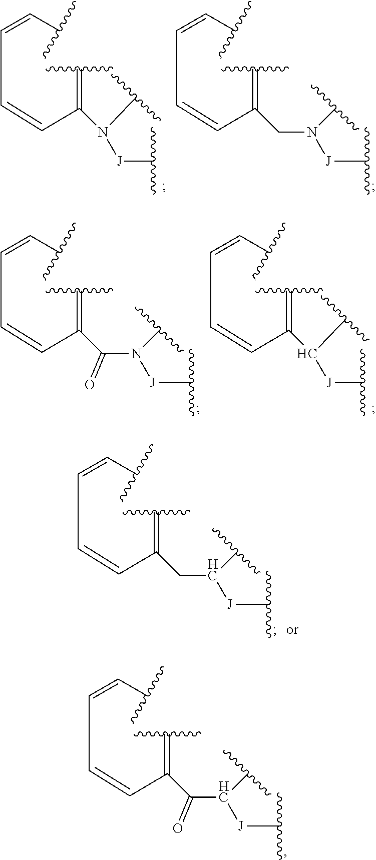

which is optionally substituted at any position,
wherein R₂ is attached to R₁ via the nitrogen or carbon atom of R₂,
wherein R₂ is attached to A or B via J,
and
wherein J is a bond or an organic structure comprising or consisting of a chain of 1, 2, 3, 4, 5, 6, 7, 8, 9, 10 or more moieties, wherein each moiety is independently selected from the group consisting of [PEG(y)]z, polyalkylene glycol, polyoxyalkylated polyol, polyvinyl alcohol, polyvinyl alkyl ether, poly(lactic acid), poly(lactic-glycolic acid), polysaccharide, a branched residue, C₁-C₁₀ alkyl, C₃-C₁₀ cycloalkane, C₂-C₁₀ alkene, C₅-C₁₀ cycloalkene, amine, sulfur, oxygen, succinimide, maleimide, glycerol, triazole, isoxazolidine, C₂-C₅ acyl, C₂-C₅ acylamino, C₂-C₅ acyloxy, succinyl, malonyl, glutaryl, phthalyl, adipoyl, an amino acid, an aryl group, a heteroaryl group, a carbamate, a chemical structure containing a cyclooctane fused to a dihydropyridazine, a chemical structure containing a cyclooctene fused to a triazole, a chemical structure containing a cyclooctene fused to a isoxazolidine, a dibenzocyclooctene, a dibenzoazacyclooctene,

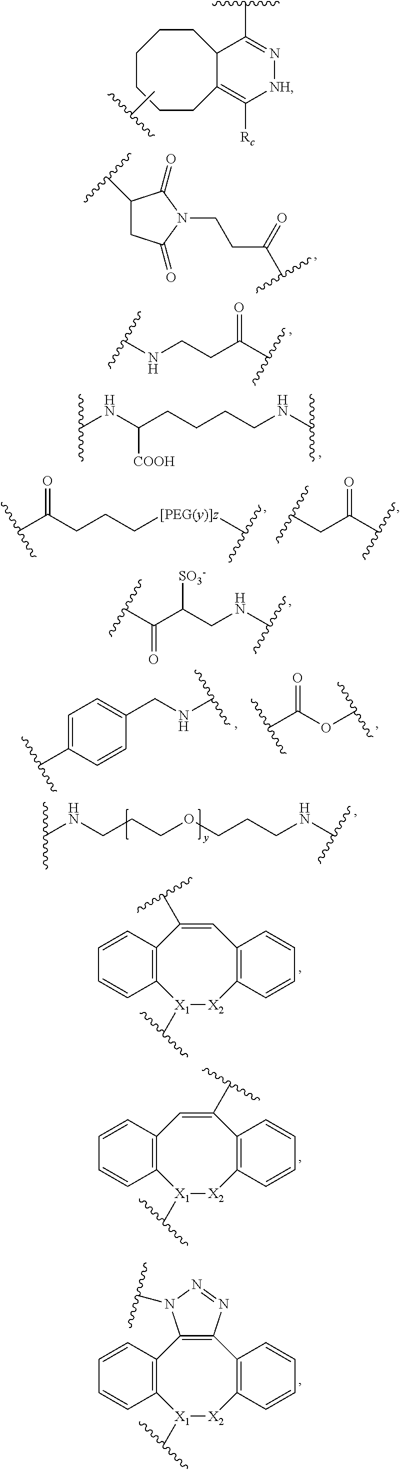

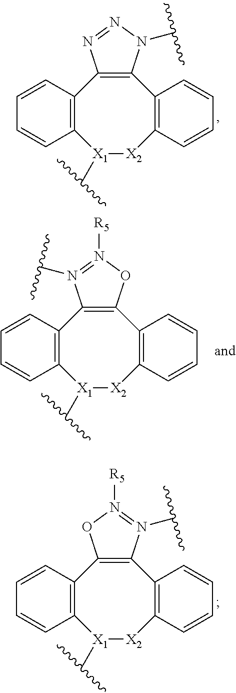

wherein X₁ is CH or N, X₂ is CH₂ or a carbonyl group, and R₅ is an aryl or alkyl group;
wherein [PEG(y)]z is:

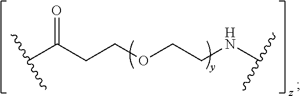

wherein y=1-100 and z=1-10.

In some embodiments, R₂ is

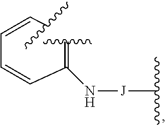

which is optionally substituted at any position.

In some embodiments, R₂ is

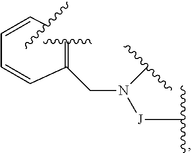

which is optionally substituted at any position.

In some embodiments, R₂ is

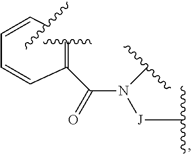

which is optionally substituted at any position.

In some embodiments, R₂ is

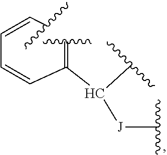

which is optionally substituted at any position.

In some embodiments, R₂ is

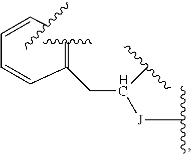

which is optionally substituted at any position.

In some embodiments, R₂ is

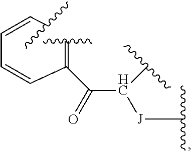

which is optionally substituted at any position.

In some embodiments, R₁ and R₂ taken together are:

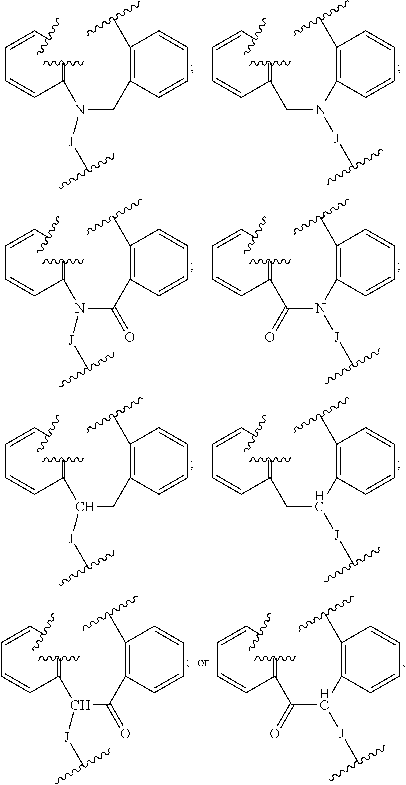

which is optionally substituted at any position,
wherein J is a bond or an organic structure comprising or consisting of a chain of 1, 2, 3, 4, 5, 6, 7, 8, 9, 10 or more moieties, wherein each moiety is independently selected from the group consisting of [PEG(y)]z, polyalkylene glycol, polyoxyalkylated polyol, polyvinyl alcohol, polyvinyl alkyl ether, poly(lactic acid), poly(lactic-glycolic acid), polysaccharide, a branched residue, C₁-C₁₀ alkyl, C₃-C₁₀ cycloalkane, C₂-C₁₀ alkene, C₅-C₁₀ cycloalkene, amine, sulfur, oxygen, succinimide, maleimide, glycerol, triazole, isoxazolidine, C₂-C₅ acyl, C₂-C₅ acylamino, C₂-C₅ acyloxy, succinyl, malonyl, glutaryl, phthalyl, adipoyl, an amino acid, an aryl group, a heteroaryl group, a carbamate, a chemical structure containing a cyclooctane fused to a dihydropyridazine, a chemical structure containing a cyclooctene fused to a triazole, a chemical structure containing a cyclooctene fused to a isoxazolidine, a dibenzocyclooctene, a dibenzoazacyclooctene,

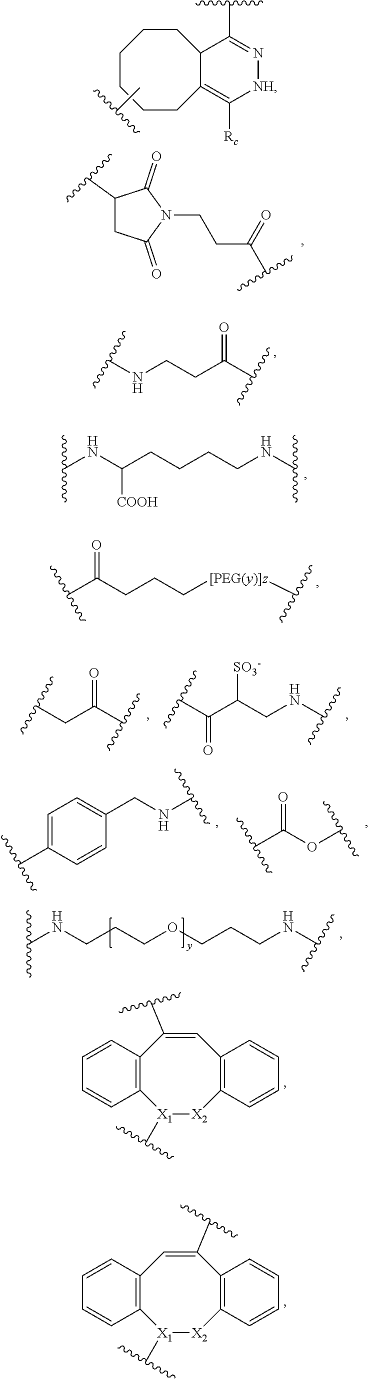

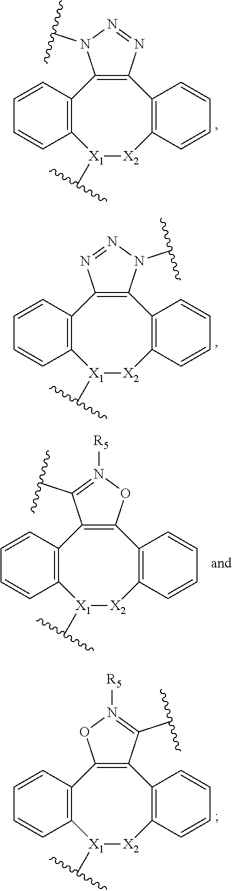

wherein X₁ is CH or N, X₂ is CH₂ or a carbonyl group, and R₅ is an aryl or alkyl group;
wherein [PEG(y)]z is:

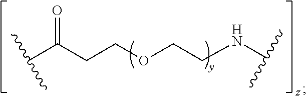

wherein y=1-100 and z=1-10.

In some embodiments, R₁ and R₂ taken together are

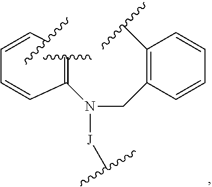

which is optionally substituted at any position.

In some embodiments, R₁ and R₂ taken together are

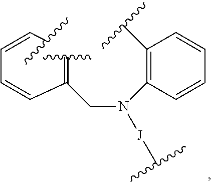

which is optionally substituted at any position.

In some embodiments, the solid line between A and B represents a nonpeptidyl linkage comprising the structure:

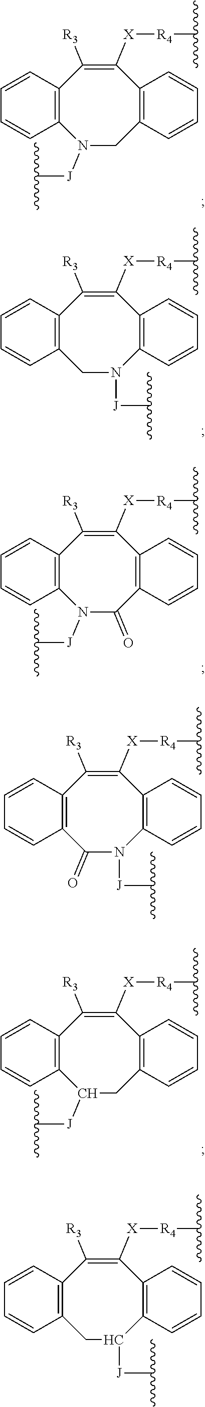

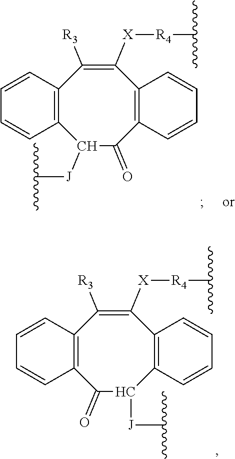

which is optionally substituted at any position,
wherein J is a bond or an organic structure comprising or consisting of a chain of 1, 2, 3, 4, 5, 6, 7, 8, 9, 10 or more moieties, wherein each moiety is independently selected from the group consisting of [PEG(y)]z, polyalkylene glycol, polyoxyalkylated polyol, polyvinyl alcohol, polyvinyl alkyl ether, poly(lactic acid), poly(lactic-glycolic acid), polysaccharide, a branched residue, C₁-C₁₀ alkyl, C₃-C₁₀cycloalkane, C₂-C₁₀ alkene, C₅-C₁₀ cycloalkene, amine, sulfur, oxygen, succinimide, maleimide, glycerol, triazole, isoxazolidine, C₂-C₅ acyl, C₂-C₅ acylamino, C₂-C₅ acyloxy, succinyl, malonyl, glutaryl, phthalyl, adipoyl, an amino acid, an aryl group, a heteroaryl group, a carbamate, a chemical structure containing a cyclooctane fused to a dihydropyridazine, a chemical structure containing a cyclooctene fused to a triazole, a chemical structure containing a cyclooctene fused to a isoxazolidine, a dibenzocyclooctene, a dibenzoazacyclooctene,

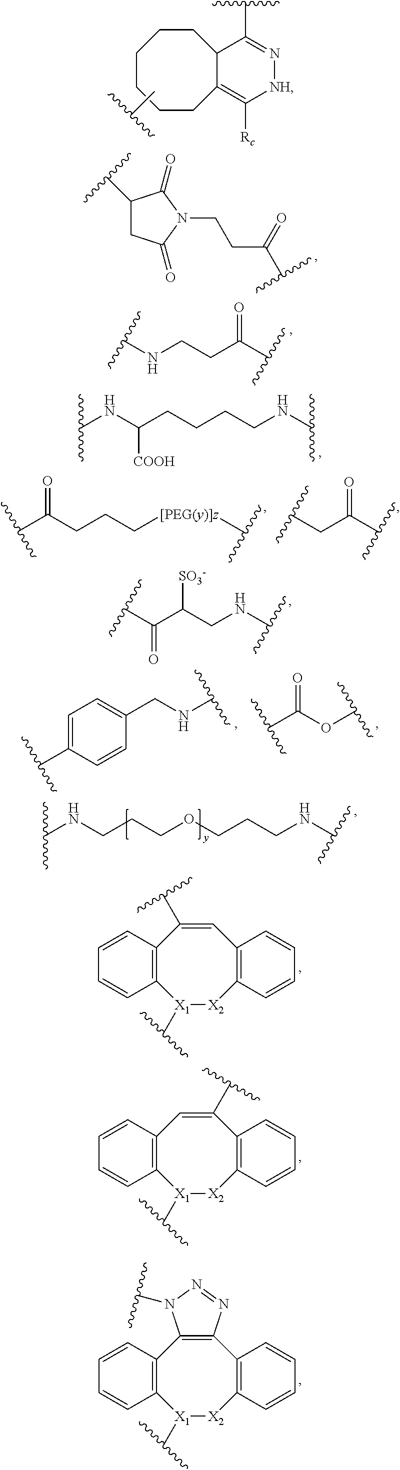

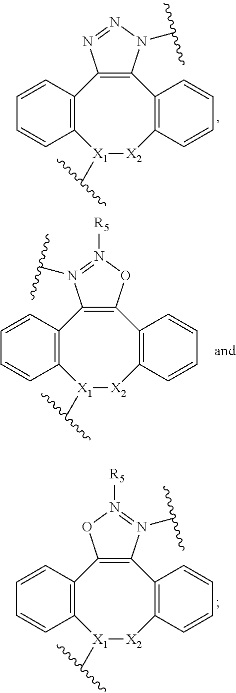

wherein X₁ is CH or N, X₂ is CH₂ or a carbonyl group, and R₅ is an aryl or alkyl group;
wherein [PEG(y)]z is:

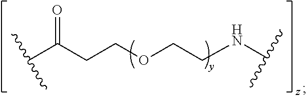

wherein y=1-100 and z=1-10.

In some embodiments, the solid line between A and B represents a nonpeptidyl linkage comprising the structure:

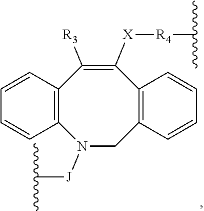

which is optionally substituted at any position.

In some embodiments, the solid line between A and B represents a nonpeptidyl linkage comprising the structure:

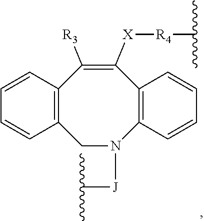

which is optionally substituted at any position.

In some embodiments, the solid line between A and B represents a nonpeptidyl linkage comprising the structure:

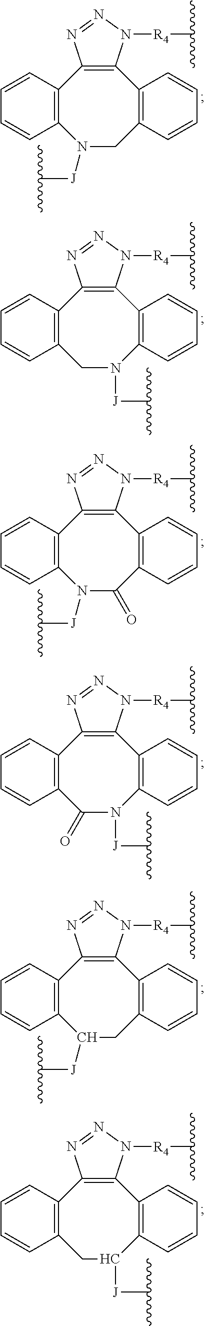

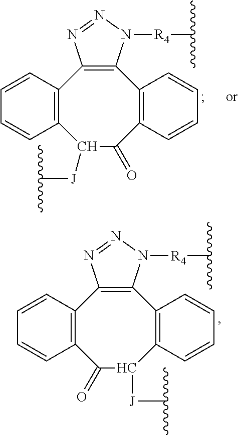

which is optionally substituted at any position,
wherein J is a bond or an organic structure comprising or consisting of a chain of 1, 2, 3, 4, 5, 6, 7, 8, 9, 10 or more moieties, wherein each moiety is independently selected from the group consisting of [PEG(y)]z, polyalkylene glycol, polyoxyalkylated polyol, polyvinyl alcohol, polyvinyl alkyl ether, poly(lactic acid), poly(lactic-glycolic acid), polysaccharide, a branched residue, C₁-C₁₀ alkyl, C₃-C₁₀ cycloalkane, C₂-C₁₀ alkene, C₅-C₁₀ cycloalkene, amine, sulfur, oxygen, succinimide, maleimide, glycerol, triazole, isoxazolidine, C₂-C₅ acyl, C₂-C₅ acylamino, C₂-C₅ acyloxy, succinyl, malonyl, glutaryl, phthalyl, adipoyl, an amino acid, an aryl group, a heteroaryl group, a carbamate, a chemical structure containing a cyclooctane fused to a dihydropyridazine, a chemical structure containing a cyclooctene fused to a triazole, a chemical structure containing a cyclooctene fused to a isoxazolidine, a dibenzocyclooctene, a dibenzoazacyclooctene,

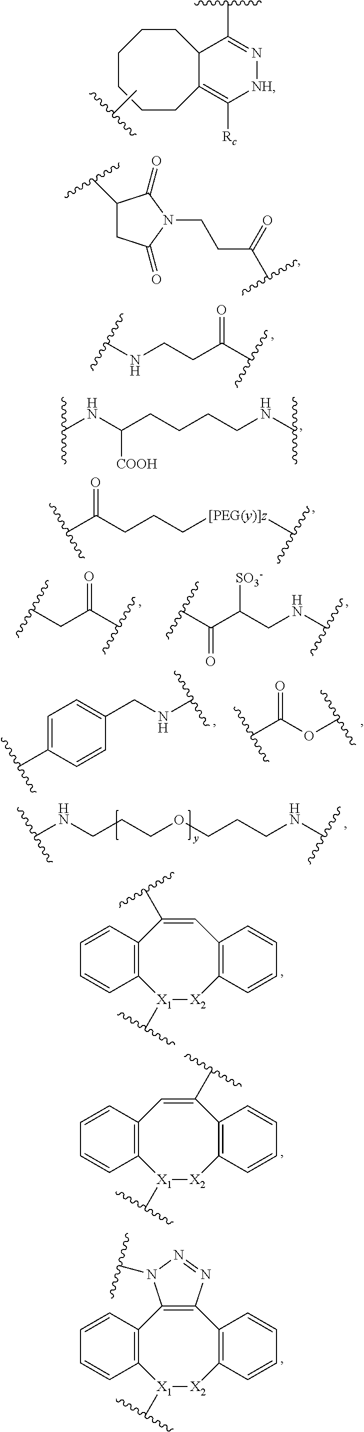

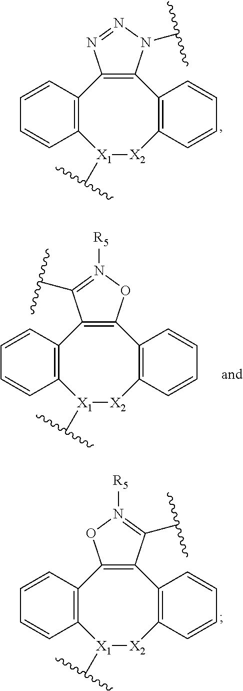

wherein X₁ is CH or N, X₂ is CH₂ or a carbonyl group, and R₅ is an aryl or alkyl group;
wherein [PEG(y)]z is:

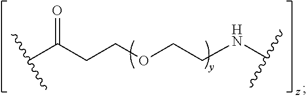

wherein y=1-100 and z=1-10.

In some embodiments, the solid line between A and B represents a monopeptidyl linkage comprising the structure:

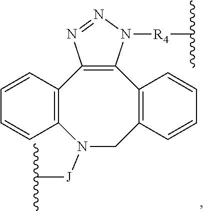

which is optionally substituted at any position.

In some embodiments, the solid line between A and B represents a nonpeptidyl linkage comprising the structure:

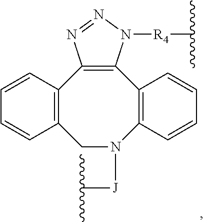

which is optionally substituted at any position.

In some embodiments, the solid line between A and B represents a nonpeptidyl linkage comprising the structure:

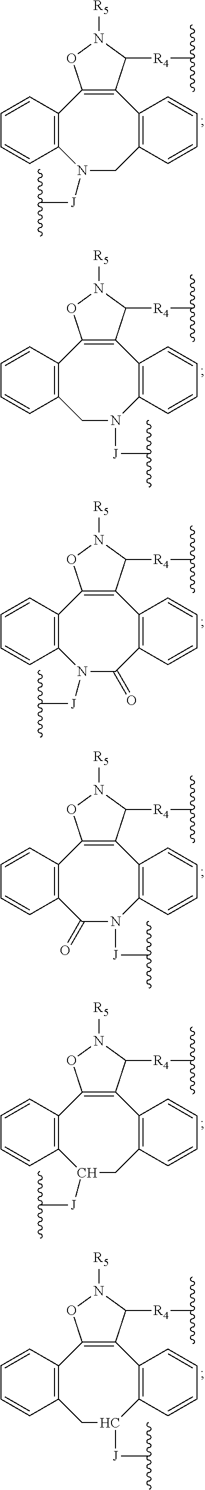

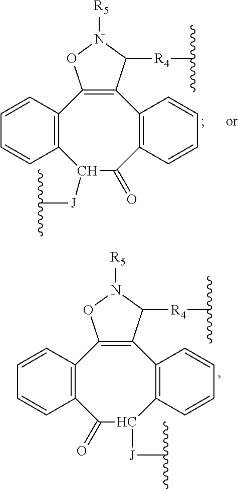

which is optionally substituted at any position,
wherein J is a bond or an organic structure comprising or consisting of a chain of 1, 2, 3, 4, 5, 6, 7, 8, 9, 10 or more moieties, wherein each moiety is independently selected from the group consisting of [PEG(y)]z, polyalkylene glycol, polyoxyalkylated polyol, polyvinyl alcohol, polyvinyl alkyl ether, poly(lactic acid), poly(lactic-glycolic acid), polysaccharide, a branched residue, C₁-C₁₀ alkyl, C₃-C₁₀ cycloalkane, C₂-C₁₀ alkene, C₅-C₁₀ cycloalkene, amine, sulfur, oxygen, succinimide, maleimide, glycerol, triazole, isoxazolidine, C₂-C₅ acyl, C₂-C₅ acylamino, C₂-C₅ acyloxy, succinyl, malonyl, glutaryl, phthalyl, adipoyl, an amino acid, an aryl group, a heteroaryl group, a carbamate, a chemical structure containing a cyclooctane fused to a dihydropyridazine, a chemical structure containing a cyclooctene fused to a triazole, a chemical structure containing a cyclooctene fused to a isoxazolidine, a dibenzocyclooctene, a dibenzoazacyclooctene,

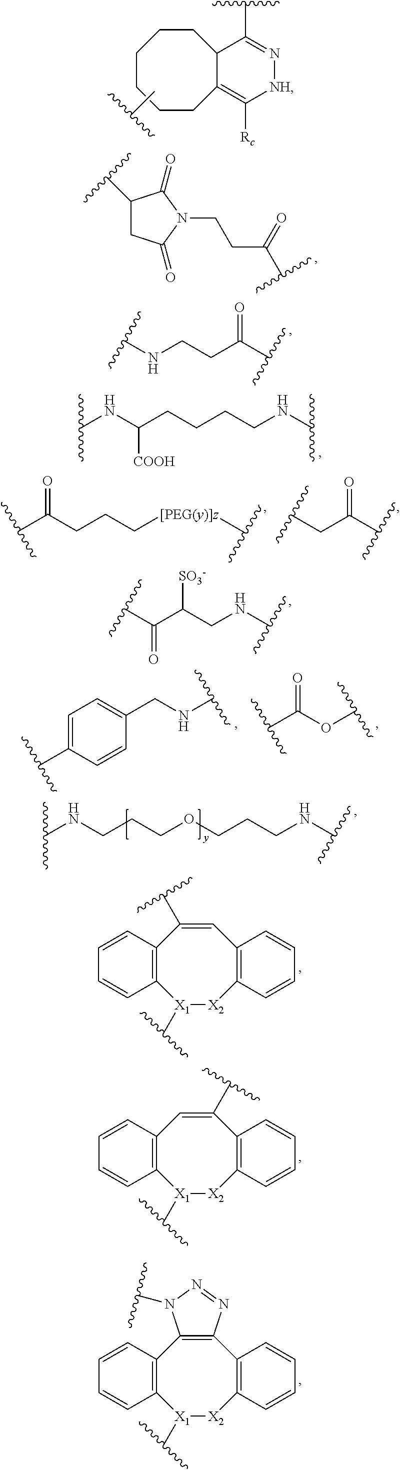

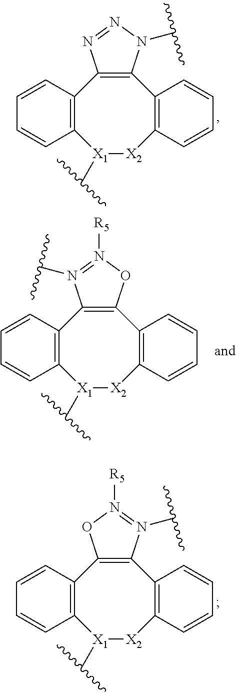

wherein X₁ is CH or N, X₂ is CH₂ or a carbonyl group, and R₅ is an aryl or alkyl group;
wherein [PEG(y)]z is:

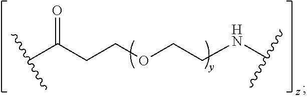

wherein y=1-100 and z=1-10.

In some embodiments, the solid line between A and B represents a nonpeptidyl linkage comprising the structure:

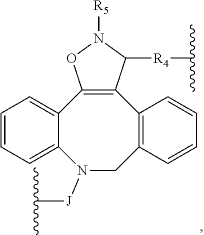

which is optionally substituted at any position.

In some embodiments, the solid line between A and B represents a nonpeptidyl linkage comprising the structure:

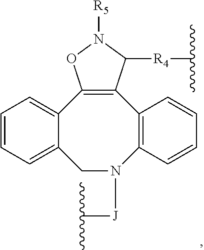

which is optionally substituted at any position.

In some embodiments, the solid line between A and B represents a nonpeptidyl linkage comprising the structure:

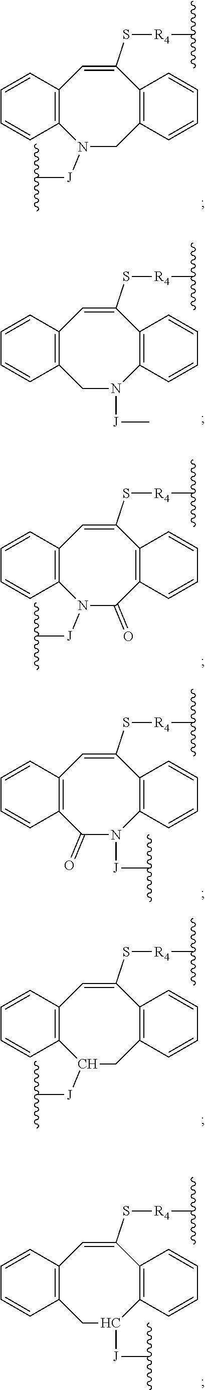

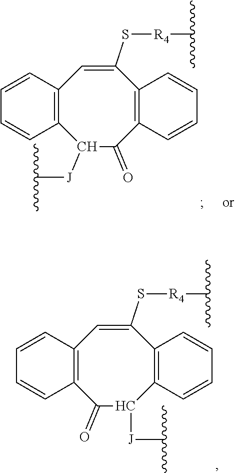

which is optionally substituted at any position,
wherein J is a bond or an organic structure comprising or consisting of a chain of 1, 2, 3, 4, 5, 6, 7, 8, 9, 10 or more moieties, wherein each moiety is independently selected from the group consisting of [PEG(y)]z, polyalkylene glycol, polyoxyalkylated polyol, polyvinyl alcohol, polyvinyl alkyl ether, poly(lactic acid), poly(lactic-glycolic acid), polysaccharide, a branched residue, C₁-C₁₀ alkyl, C₃-C₁₀ cycloalkane, C₂-C₁₀ alkene, C₅-C₁₀ cycloalkene, amine, sulfur, oxygen, succinimide, maleimide, glycerol, triazole, isoxazolidine, C₂-C₅ acyl, C₂-C₅ acylamino, C₂-C₅ acyloxy, succinyl, malonyl, glutaryl, phthalyl, adipoyl, an amino acid, an aryl group, a heteroaryl group, a carbamate, a chemical structure containing a cyclooctane fused to a dihydropyridazine, a chemical structure containing a cyclooctene fused to a triazole, a chemical structure containing a cyclooctene fused to a isoxazolidine, a dibenzocyclooctene, a dibenzoazacyclooctene,

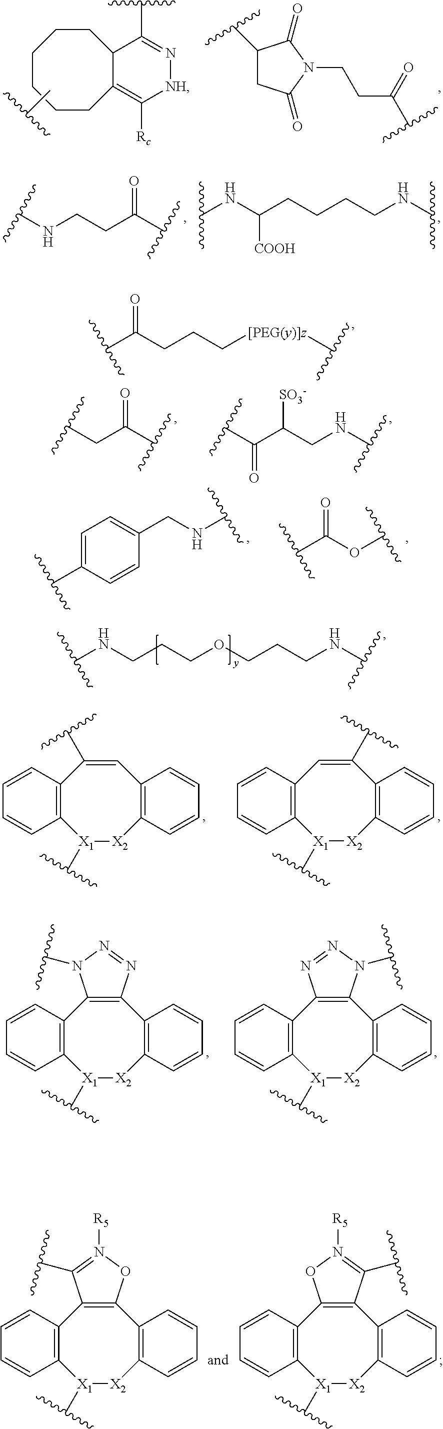

wherein X₁ is CH or N, X₂ is CH₂ or a carbonyl group, and R₅ is an aryl or alkyl group;
wherein [PEG(y)]z is:

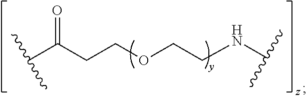

wherein y=1-100 and z=1-10.

In some embodiments, the solid line between A and B represents a nonpeptidyl linkage comprising the structure:

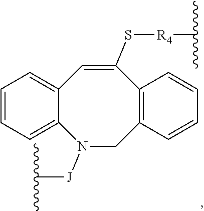

which is optionally substituted at any position.

In some embodiments, the solid line between A and B represents a nonpeptidyl linkage comprising the structure:

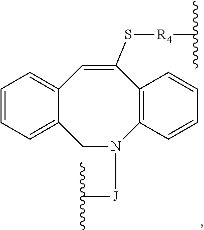

which is optionally substituted at any position.

In some embodiments, the solid line between A and B represents a nonpeptidyl linkage comprising the structure:

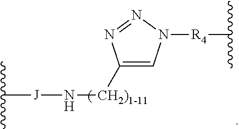

In some embodiments, R₁ is H.

In some embodiments, wherein J is an organic structure comprising a [PEG(y)]z group.

In some embodiments, wherein J is an organic structure comprising a polyalkylene glycol, polyoxyalkylated polyol, polyvinyl alcohol, polyvinyl alkyl ether, poly(lactic acid), poly(lactic-glycolic acid), or polysaccharide group.

In some embodiments, J is an organic structure comprising a C₁-C₄ alkyl group.

In some embodiments, J is an organic structure comprising a succinimide.

In some embodiments, J is an organic structure comprising amine.

In some embodiments, J is an organic structure comprising a succinyl, malonyl, glutaryl, phthalyl or adipoyl.

In some embodiments, J is an organic structure comprising a malonyl.

In some embodiments, J is an organic structure comprising an amino acid.

In some embodiments, J is an organic structure comprising a cysteine.

In some embodiments, J is an organic structure comprising a lysine.

In some embodiments, J is an organic structure consisting of a chain of 3 moieties selected from the group consisting of [PEG(y)]z, polyalkylene glycol, polyoxyalkylated polyol, polyvinyl alcohol, polyvinyl alkyl ether, poly(lactic acid), poly(lactic-glycolic acid), polysaccharide, a branched residue, C₁-C₁₀ alkyl, C₃-C₁₀ cycloalkane, C₂-C₁₀ alkene, C₅-C₁₀ cycloalkene, amine, sulfur, oxygen, succinimide, maleimide, glycerol, triazole, isoxazolidine, C₂-C₅ acyl, C₂-C₅ acylamino, C₂-C₅ acyloxy, succinyl, malonyl, glutaryl, phthalyl, adipoyl, an amino acid, an aryl group, a heteroaryl group, a carbamate, a chemical structure containing a cyclooctane fused to a dihydropyridazine, a chemical structure containing a cyclooctene fused to a triazole, a chemical structure containing a cyclooctene fused to a isoxazolidine, a dibenzocyclooctene, a dibenzoazacyclooctene,

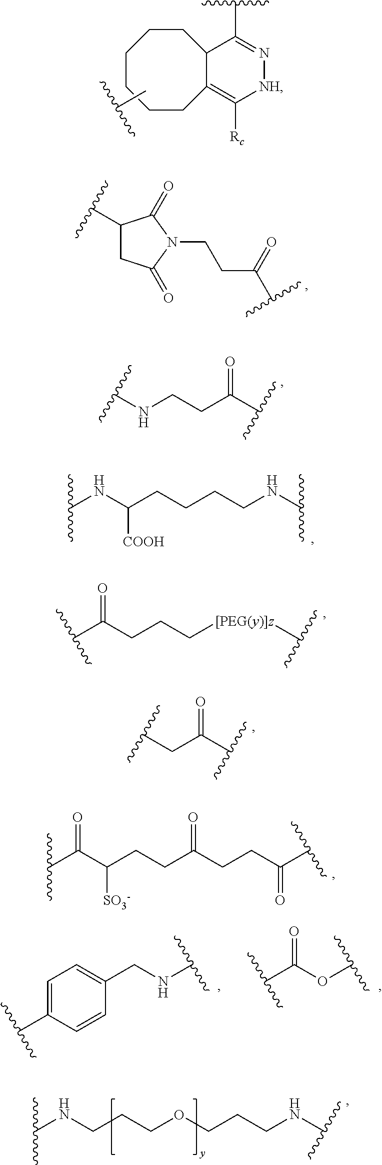

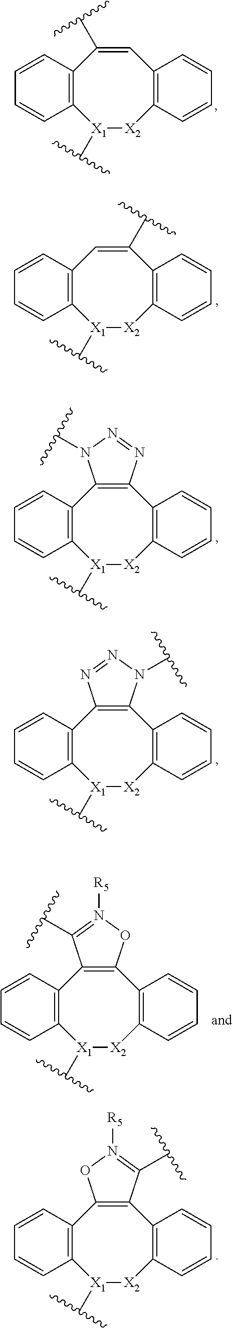

In some embodiments, J is an organic structure consisting of a chain of four moieties selected from the group consisting of [PEG(y)]z, polyalkylene glycol, polyoxyalkylated polyol, polyvinyl alcohol, polyvinyl alkyl ether, poly(lactic acid), poly(lactic-glycolic acid), polysaccharide, a branched residue, C₁-C₁₀ alkyl, C₃-C₁₀ cycloalkane, C₂-C₁₀ alkene, C₅-C₁₀ cycloalkene, amine, sulfur, oxygen, succinimide, maleimide, glycerol, triazole, isoxazolidine, C₂-C₅ acyl, C₂-C₅ acylamino, C₂-C₅ acyloxy, succinyl, malonyl, glutaryl, phthalyl, adipoyl, an amino acid, an aryl group, a heteroaryl group, a carbamate, a chemical structure containing a cyclooctane fused to a dihydropyridazine, a chemical structure containing a cyclooctene fused to a triazole, a chemical structure containing a cyclooctene fused to a isoxazolidine, a dibenzocyclooctene, a dibenzoazacyclooctene,

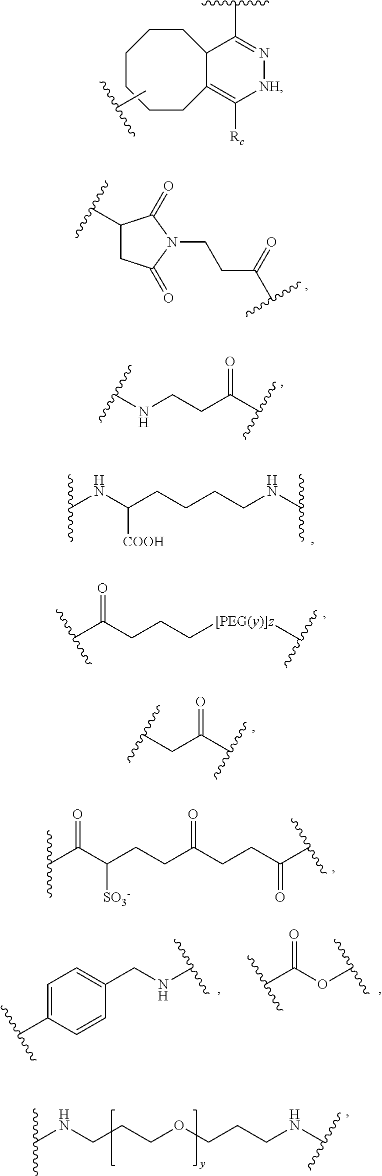

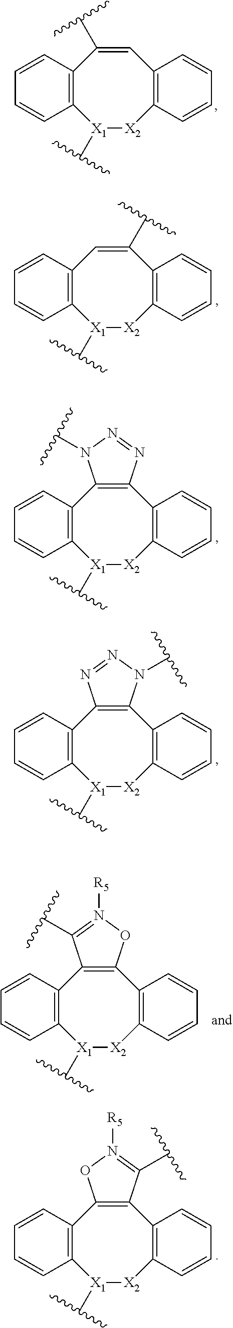

In some embodiments, J is an organic structure consisting of a chain of five moieties selected from the group consisting of [PEG(y)]z, polyalkylene glycol, polyoxyalkylated polyol, polyvinyl alcohol, polyvinyl alkyl ether, poly(lactic acid), poly(lactic-glycolic acid), polysaccharide, a branched residue, C₁-C₁₀ alkyl, C₃-C₁₀ cycloalkane, C₂-C₁₀ alkene, C₅-C₁₀ cycloalkene, amine, sulfur, oxygen, succinimide, maleimide, glycerol, triazole, isoxazolidine, C₂-C₅ acyl, C₂-C₅ acylamino, C₂-C₅ acyloxy, succinyl, malonyl, glutaryl, phthalyl, adipoyl, an amino acid, an aryl group, a heteroaryl group, a carbamate, a chemical structure containing a cyclooctane fused to a dihydropyridazine, a chemical structure containing a cyclooctene fused to a triazole, a chemical structure containing a cyclooctene fused to a isoxazolidine, a dibenzocyclooctene, a dibenzoazacyclooctene,

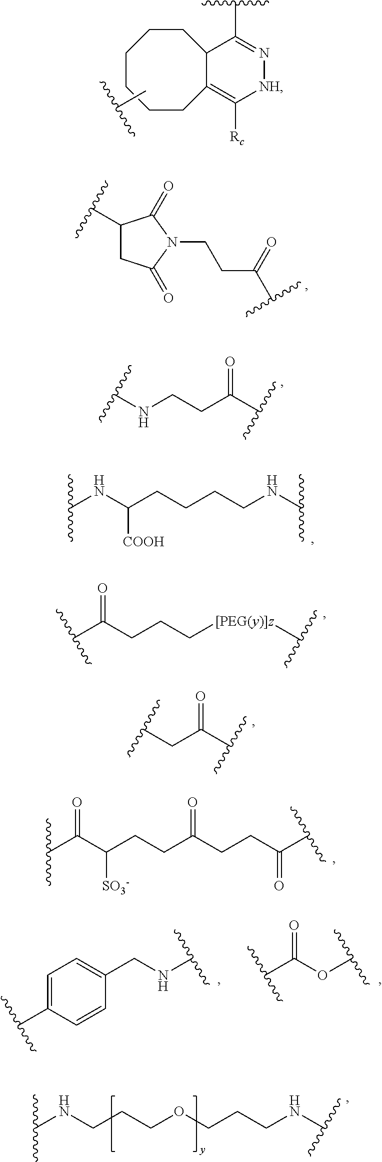

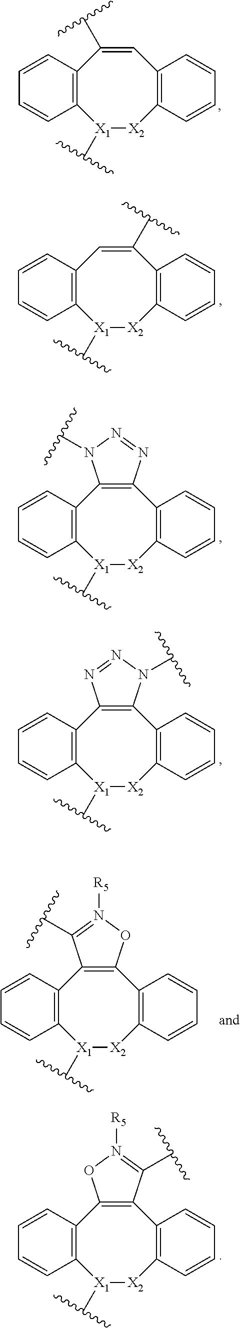

In some embodiments, J comprises a [PEG(y)]z group bonded to a lysine.

In some embodiments, J comprises a C₁-C₄ acyl group bonded to a succinimide group.

In some embodiments, J comprises a lysine bonded to a C₁-C₄ acyl.

In some embodiments, J comprises a [PEG(y)]z group, which is bonded to a glutaryl.

In some embodiments, J is an organic structure consisting of a chain of five moieties selected from the group consisting of [PEG(y)]z, C₂-C₅ acyl, succinyl, malonyl, glutaryl, an amino acid, a chemical structure containing a cyclooctane fused to a dihydropyridazine, a chemical structure containing a cyclooctene fused to a triazole, a chemical structure containing a cyclooctene fused to a isoxazolidine, a dibenzocyclooctene, a dibenzoazacyclooctene,

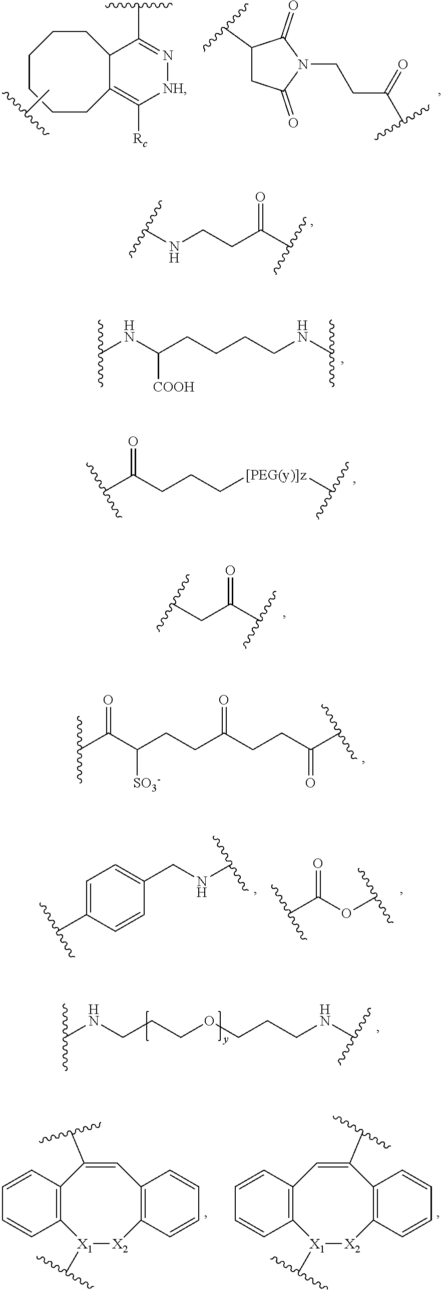

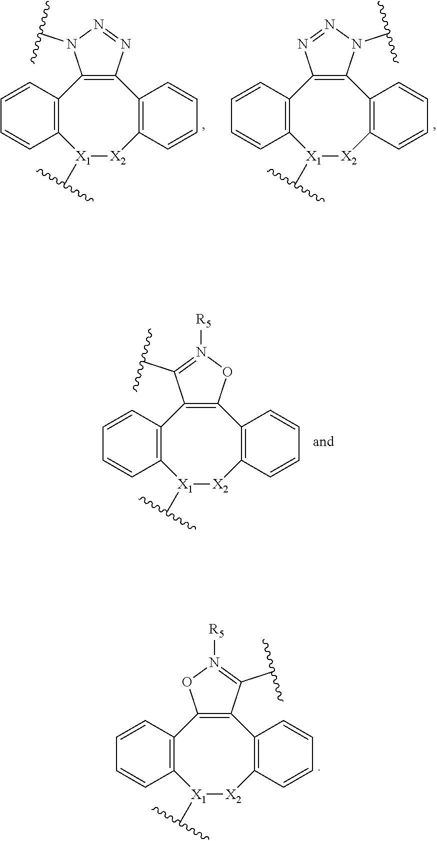

In some embodiments, J is a bond.

In some embodiments, J is a cysteine.

In some embodiments, J has the structure:

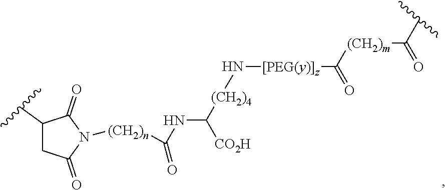

wherein n 1-3, m is 1-4, y is 1-100 and z is 1-10.

In some embodiments, J has a linear structure.

In some embodiments, J has a branched structure.

In some embodiments, R₂ is

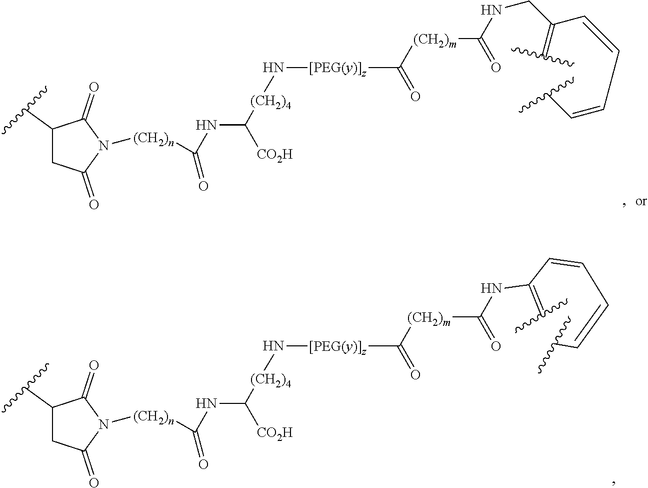

wherein n 1-3, m is 1-4, y is 1-100 and z is 1-10.

In some embodiments, R₂ is

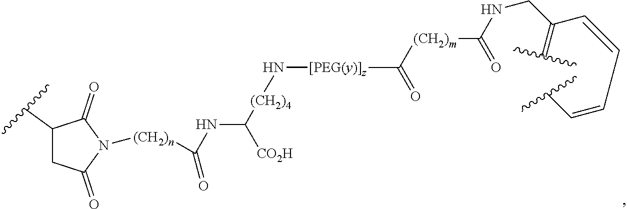

wherein n 1-3, m is 1-4, y is 1-100 and z is 1-10.

In some embodiments, R₂ is

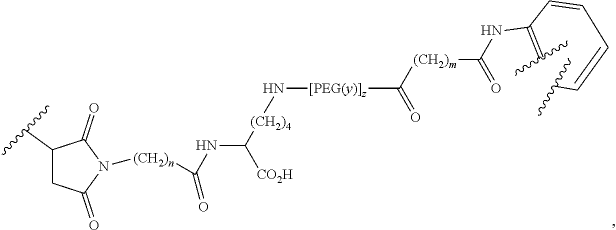

wherein n 1-3, m is 1-4, y is 1-100 and z is 1-10.

In some embodiments, R₁ and R₂ taken together are:

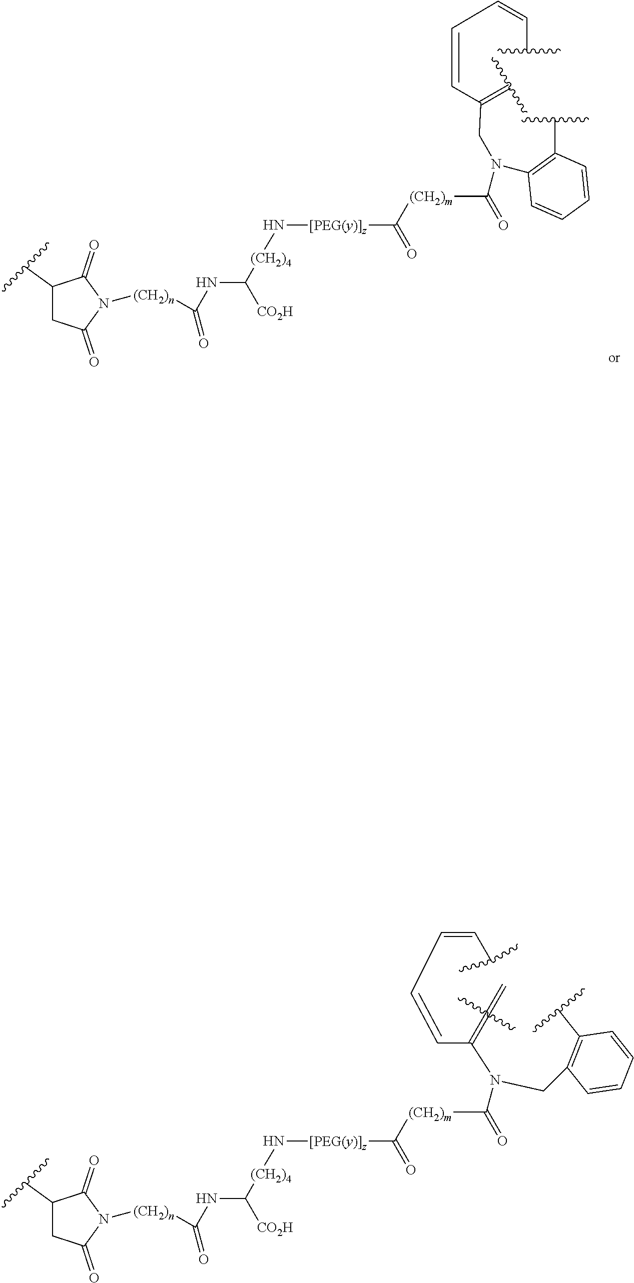

wherein n 1-3, m is 1-4, y is 1-100 and z is 1-10.

In some embodiments, R₁ and R₂ taken together are:

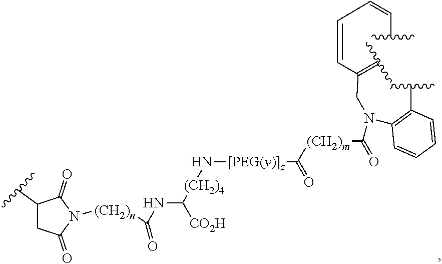

wherein n 1-3, m is 1-4, y is 1-100 and z is 1-10.

In some embodiments, R₁ and R₂ taken together are:

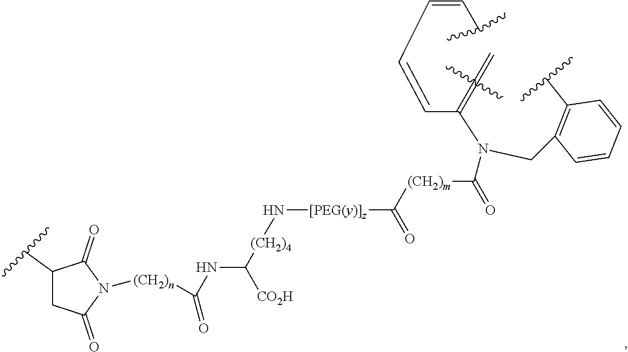

wherein n 1-3, m is 1-4, y is 1-100 and z is 1-10.

In some embodiments, the solid line between A and B represents a nonpeptidyl linkage comprising the structure:

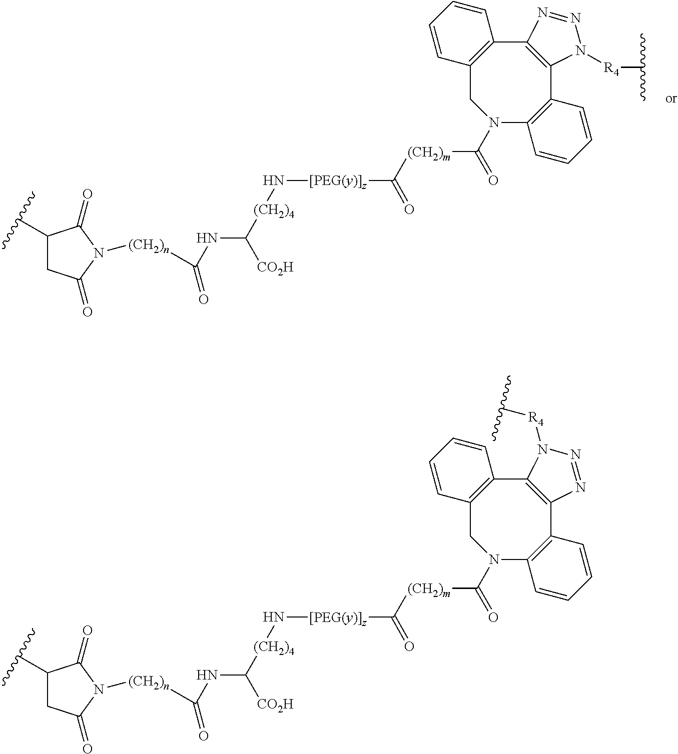

wherein [PEG(y)]z is:

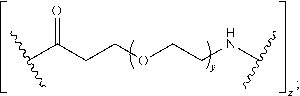

wherein y=1-100 and z=1-10.

In some embodiments, the solid line between A and B represents a nonpeptidyl linkage comprising the structure:

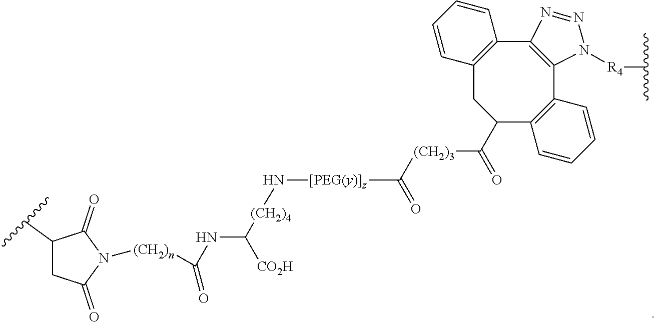

In some embodiments, the solid line between A and B represents a monopeptidyl linkage comprising the structure:

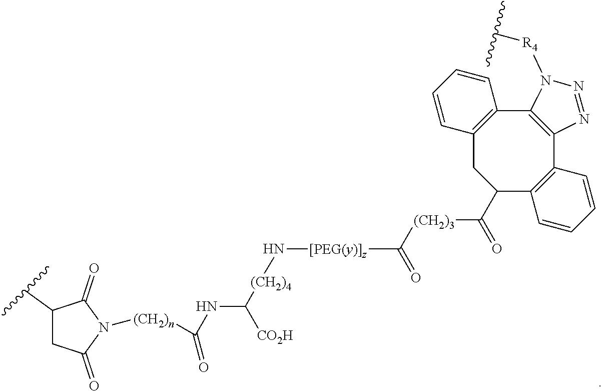

In some embodiments, the solid line between A and B represents a nonpeptidyl linkage comprising the structure:

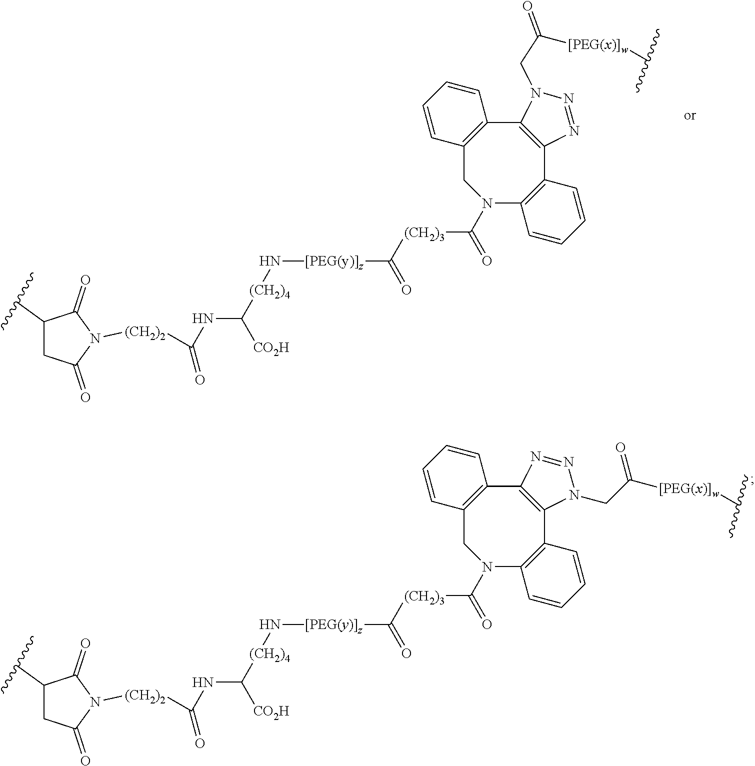

wherein [PEG(y)]z is:

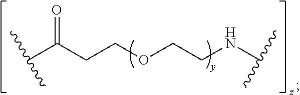

wherein y=1-100 and z=1-10;
wherein [PEG(x)]w is:

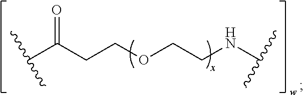

wherein x=1-100 and w=1-10.

In some embodiments, y is 1-20. In some embodiments, y is 21-40. In some embodiments, y is 41-60. In some embodiments, y is 61-80. In some embodiments, y is 30-50. In some embodiments, y is 12, 24, 36 or 48. In some embodiments, z is 1. In some embodiments, z is 0.

In some embodiments, the terminal carbonyl is of the [PEG(y)]z group is part of an amide bond.

In some embodiments, the terminal amine of the [PEG(y)]z group is part of an amide bond.

In some embodiments, R₄ is

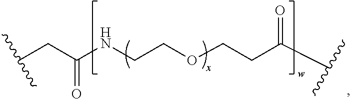

wherein x is 1-100, and w is 0-5.

In some embodiments, x is 1-20. In some embodiments, x is 21-40.

In some embodiments, x is 41-60. In some embodiments, x is 61-80. In some embodiments, x is 30-50. In some embodiments, x is 12, 24, 36 or 48.

In some embodiments, w is 1. In some embodiments, w is 0.

In some embodiments, R₄ has the structure:

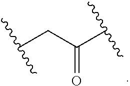 [SMILES: *CC(*)=O]

In some embodiments, R₂ is attached A to via J, and R₄ is attached to B.

In some embodiments, R₂ is attached B to via J, and R₄ is attached to A.

In some embodiments, R₄ is attached to B via the terminal carbonyl carbon.

In some embodiments, the solid line between A and B represents a nonpeptidyl linkage comprising the structure:

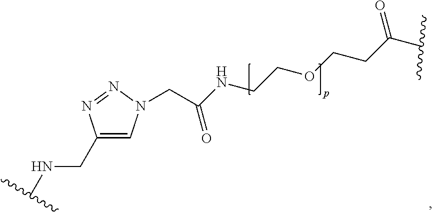

wherein p=0-5, 0-10, 0-50, or 0-100.

In some embodiments, wherein R₂ is attached to A via a carbon-nitrogen bond or a carbon-sulfur bond.

In some embodiments, R₂ is attached to A via a carbon-nitrogen bond.

In some embodiments, the carbon-nitrogen bond is an amide bond.

In some embodiments, R₂ is attached to A via a biologically labile bond.

In some embodiments, R₂ is attached to A via an amide bond between the C-terminal amino acid of A and an amino group in B.

In some embodiments, the terminal amino acid is cysteine.

In some embodiments, R₂ is attached to A via a carbon-sulfur bond.

In some embodiments, R₂ is attached to A via a carbon-sulfur bond formed between R₂ and a free thiol.

In some embodiments, R₂ is attached to A via a succinimide-sulfur bond.

In some embodiments, J comprises a branched residue.

In some embodiments, J is attached to more than one A via the branched residue.

In some embodiments, B comprises a branched residue.

In some embodiments, B is linked to more than one A, each via a nonpeptidyl linkage with the branched residue.

In some embodiments, the solid line between A and B represents a nonpeptidyl linkage comprising the structure:

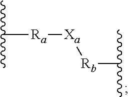

wherein Xₐ is a chemical structure containing a cyclooctane fused to a dihydropyridazine; and
wherein Rₐ represents an organic structure which connects to one of A or B and R_{b} represents an organic structure which connects to the other of A or B.

In some embodiments, Xa has the structure:

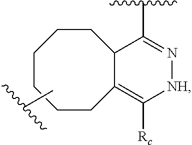

wherein R_{c} is H, alkyl or aryl;
or a tautomer thereof.

In some embodiments, Xₐ has the structure:

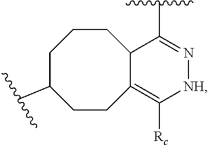

wherein R_{c} is H, alkyl or aryl;
or a tautomer thereof.

In some embodiments, Rₐ is connected to the cyclooctane and R_{b} is connected to the dihydropyridazine.

In some embodiments, the solid line between A and B represents a nonpeptidyl linkage comprising the structure:

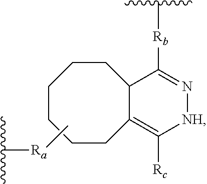

wherein R_{c} is H, alkyl or aryl;
or a tautomer thereof.

In some embodiments, Xa has the structure:

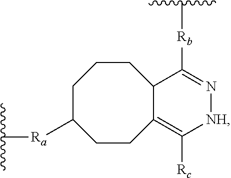

wherein R_{c} is H, alkyl or aryl;
or a tautomer thereof.

In some embodiments, R_{c} is methyl.

In some embodiments, Rₐ and R_{b} are independently a bond or an organic structure comprising or consisting of a chain of 1, 2, 3, 4, 5, 6, 7, 8, 9, 10 or more moieties, wherein each moiety is independently selected from the group consisting of [PEG(y)]z, polyalkylene glycol, polyoxyalkylated polyol, polyvinyl alcohol, polyvinyl alkyl ether, poly(lactic acid), poly(lactic-glycolic acid), polysaccharide, a branched residue, C₁-C₁₀ alkyl, C₃-C₁₀ cycloalkane, C₂-C₁₀ alkene, C₅-C₁₀ cycloalkene, amine, sulfur, oxygen, succinimide, maleimide, glycerol, triazole, isoxazolidine, C₂-C₅ acyl, C₂-C₅ acylamino, C₂-C₅ acyloxy, succinyl, malonyl, glutaryl, phthalyl, adipoyl, an amino acid, an aryl group, a heteroaryl group, a carbamate, a chemical structure containing a cyclooctane fused to a dihydropyridazine, a chemical structure containing a cyclooctene fused to a triazole, a chemical structure containing a cyclooctene fused to a isoxazolidine, a dibenzocyclooctene, a dibenzoazacyclooctene,

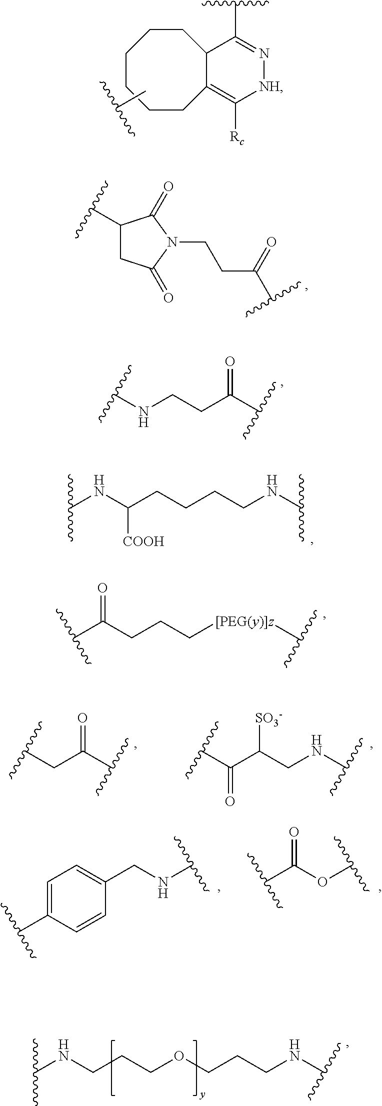

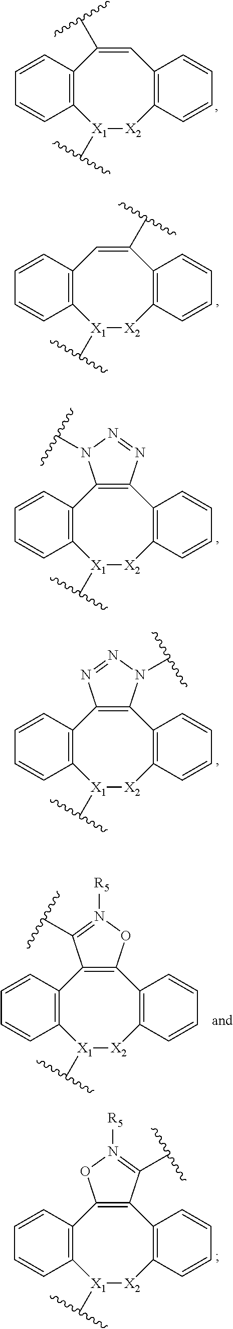

wherein X₁ is CH or N, X₂ is CH₂ or a carbonyl group, and R₅ is an aryl or alkyl group;
wherein [PEG(y)]z is:

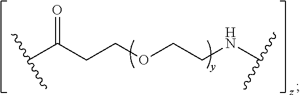

wherein y=1-100 and z=1-10.

In some embodiments, Rₐ and R_{b} are independently a bond or an organic structure comprising or consisting of a chain of 1, 2, 3, 4, 5, 6, 7, 8, 9, 10 or more moieties, wherein each moiety is independently selected from the group consisting of [PEG(y)]z, C₂-C₅ acyl, succinyl, malonyl, glutaryl, an amino acid, a chemical structure containing a cyclooctane fused to a dihydropyridazine, a chemical structure containing a cyclooctene fused to a triazole, a chemical structure containing a cyclooctene fused to a isoxazolidine, a dibenzocyclooctene, a dibenzoazacyclooctene,

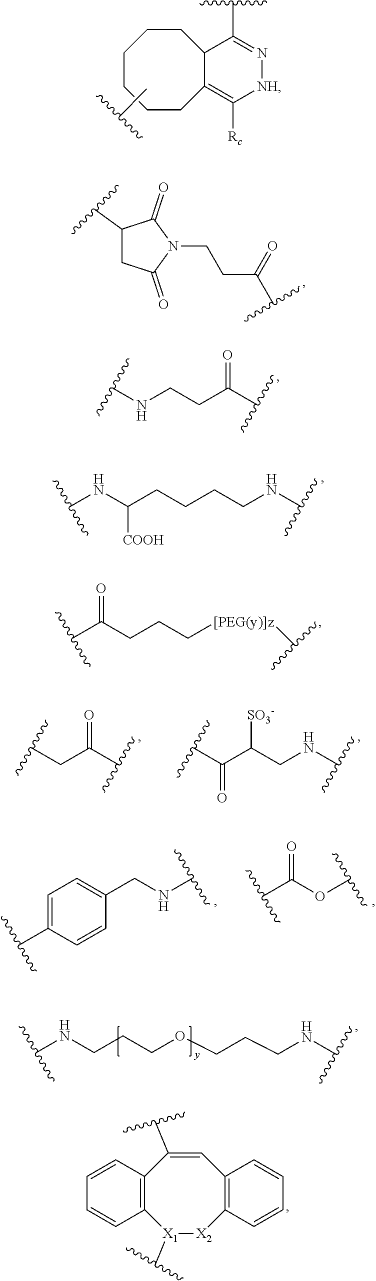

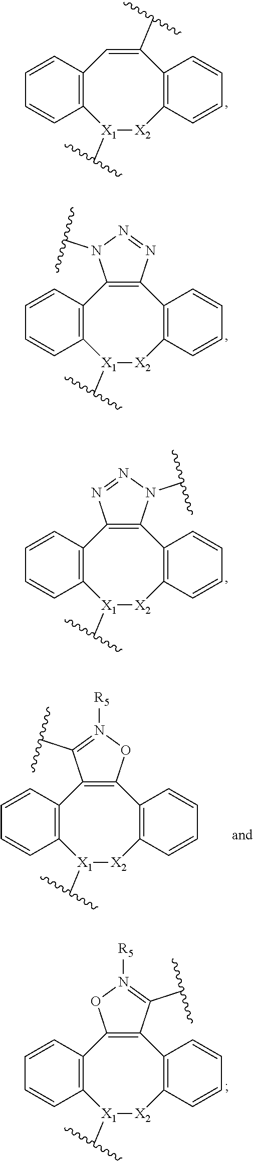

wherein X₁ is CH or N, X₂ is CH₂ or a carbonyl group, and R₅ is an aryl or alkyl group;
wherein [PEG(y)]z is:

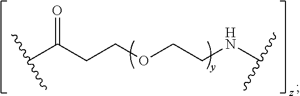

wherein y=1-100 and z=1-10.

In some embodiments, Ra or Rb is attached to A via a carbon-nitrogen bond or a carbon-sulfur bond.

In some embodiments, Ra or Rb is attached to A via a carbon-nitrogen bond.

In some embodiments, the carbon-nitrogen bond is an amide bond.

In some embodiments, Ra or Rb is attached to A via a biologically labile bond.

In some embodiments, Ra or Rb is is attached to A via an amide bond between the C-terminal amino acid of A and an amino group in B.

In some embodiments, the terminal amino acid is cysteine.

In some embodiments, Ra or Rb is is attached to A via a carbon-sulfur bond.

In some embodiments, Ra or Rb is is attached to A via a carbon-sulfur bond formed between R₂ and a free thiol.

In some embodiments, wherein Ra or Rb is is attached to A via a succinimide-sulfur bond.

In some embodiments, Ra or Rb comprises a branched residue.

In some embodiments, Ra or Rb is is attached to more than one A via the branched residue.

In some embodiments, the biological activity of A is increased when it is part of a compound or dimer of the invention compared to the biological activity of A when it is not linked to any other structure.

In some embodiments, A comprises the structure of a compound that is a drug approved for treating a subject afflicted with a disease.

In some embodiments, the subject is a mammalian subject.

In some embodiments, the mammalian subject is a human subject.

In some embodiments, A comprises the structure of an organic compound having a molecular weight less than 1000 Daltons, a DNA aptamer, an RNA aptamer, an oligonucleotide, or a protein that is biologically active.

In some embodiments, the oligonucleotide is an antisense oligonucleotide.

In some embodiments, the oligonucleotide is an RNA interference inducing molecule.

In some embodiments, the oligonucleotide encodes an RNA interference inducing molecule.

In some embodiments, A comprises a primary or a secondary amine.

In some embodiments, A is linked to B via the primary or secondary amine.

In some embodiments, A comprises a primary amine.

In some embodiments, A is aripiprazole or oseltamivir.

In some embodiments, A comprises a secondary amine.

In some embodiments, A is a respiratory drug, an antiasthmatic agent, an analgesic agent, an antidepressant, an antianginal agent, an antiarrhythmic agent, an antihypertensive agent, an antidiabetic agent, an antihistamine, an anti-infective agent, an antibiotic, an antiinflammatory agent, an antiparkinsonism drug, an antipsychotics, an antipyretic agent, an antiulcer agent, an attention deficit hyperactivity disorder (ADHD) drug, a central nervous system stimulant, a decongestant, or a psychostimulant.

In some embodiments, A is alprenolol, acebutolol, amidephrine, amineptine, amosulalol, amoxapine, amphetaminil, atenolol, atomoxetine, balofloxacin, bamethan, befunolol, benazepril, benfluorex, benzoctamine, betahistine, betaxolol, bevantolol, bifemelane, bisoprolol, brinzolamide, bufeniode, butethamine, camylofine, carazolol, carticaine, carvedilol, cephaeline, ciprofloxacin, cloZapine, clobenZorex, clorprenaline, cyclopentamine, delapril, demexiptiline, denopamine, desipramine, desloratadine, diclofenac, dimetofrine, dioxadrol, dobutamine, dopexamine, doripenem, dorzolamide, droprenilamine, duloxetine, eltopraZine, enalapril, enoxacin, epinephrine, ertapenem, esapraZole, esmolol, etoxadrol, fasudil, fendiline, fenethylline, fenfluramine, fenoldopam, fenoterol, fenproporex, flecamide, fluoxetine, formoterol, frovatriptan, gaboxadol, garenoxacin, gatifloxacin, grepafloxacin, hexoprenaline, imidapril, indalpine, indecainide, indeloxazine hydrochloride, isoxsuprine, ispronicline, labetalol, landiolol, lapatinib, levophacetoperane, lisinopril, lomefloxacin, lotrafiban, maprotiline, mecamylamine, mefloquine, mepindolol, meropenem, metapramine, metaproterenol, methoxyphenamine, dextrorotary methylphenidate, methylphenidate, metipranolol, metoprolol, mitoxantrone, mivazerol, moexipril, moprolol, moxifloxacin, nebivolol, nifenalol, nipradilol, norfloxacin, nortriptyline, nylidrin, olanZapine, oxamniquine, oxprenolol, oxyfedrine, paroxetine, perhexyline, phenmetrazine, phenylephrine, phenylpropylmethylamine, pholedrine, picilorex, pimethylline, pindolol, pipemidic acid, piridocaine, practolol, pradofloxacin, pramipexole, pramiverin, prenalterol, prenylamine, prilocalne, procaterol, pronethalol, propafenone, propranolol, propylhexedrine, protokylol, protriptyline, pseudoephedrine, reboxetine, rasagiline, (r)-rasagiline, repinotan, reproterol, rimiterol, ritodrine, safinamide, salbutamol/albuterol, salmeterol, sarizotan, sertraline, silodosin, sotalol, soterenol, sparfloxacin, spirapril, sulfinalol, synephrine, tamsulosin, tebanicline, tianeptine, tirofiban, tretoquinol, trimetazidine, troxipide, varenicline, vildagliptin, viloxazine, viquidil or xamoterol.

In some embodiments, A comprises a protein that is biologically active.

In some embodiments, A comprises a secreted protein.

In some embodiments, A comprises an extracellular domain of a protein.

In some embodiments, A is biologically active such that it has target-binding activity.

In some embodiments, the A is an independently-folding protein or a portion thereof.

In some embodiments, A is a glycosylated protein.

In some embodiments, A comprises intra-chain disulfide bonds.

In some embodiments, A binds a cytokine.

In some embodiments, the cytokine is TNFα.

In some embodiments, A comprises Atrial Natriuretic Peptide (ANP), Calcitonin, Corticotropin Releasing Hormone (CRH), Endothelin, Exenatide, Gastric Inhibitory Peptide (GIP), Glucagon-Like Peptide-1 (GLP-1), Glucagon-Like Peptide-2 (GLP-2), an analog of GLP-1 or GLP-2, Glucagon Vasoactive Intestinal Peptide (GVIP), Ghrelin, Peptide YY or Secretin, or a portion thereof.

In some embodiments, A comprises a stretch of consecutive amino acids in the sequence HGEGTFTSDVSSYLEEQAAKEFIAWLVKGRG (SEQ ID NO:202).

In some embodiments, A comprises at least one stretch of consecutive amino acids which are identical to a stretch of consecutive amino acids present in the heavy chain of a Fab or a Fab′ of an antibody.

In some embodiments, A comprises at least one at least one stretch of consecutive amino acids which are identical to a stretch of consecutive amino acids present in the light chain of a Fab or a Fab′ of an antibody.

In some embodiments, A comprises at least one Fab or Fab′ of an antibody, or a portion of the at least one Fab or Fab′.

In some embodiments, A comprises at least 2, 3, 4, 5, 6, 7, 8, 9, or 10 copies of the Fab or Fab′ or portion thereof.

In some embodiments, A comprises Fab-1 or Fab′1, or a portion thereof of the antibody.

In some embodiments, A comprises Fab-2 or Fab′2, or a portion thereof of the antibody.

In some embodiments, A comprises two Fab or Fab′ hands of the antibody.

In some embodiments, the Fab or Fab′ is present in adalimumab

In some embodiments, A comprises at least one stretch of consecutive amino acids which are identical to a stretch of consecutive amino acids present in a single chain antibody.

In some embodiments, A comprises at least one stretch of consecutive amino acids which are identical to a stretch of consecutive amino acids present in a TNFα receptor.

In some embodiments, the TNFα receptor is TNR1B.

In some embodiments, a compound of the invention forms part of a homodimer.

In some embodiments, a compound of the invention forms part of a heterodimer.

The present invention provides homodimers comprising compounds of the invention.

The present invention provides heterodimers comprising compounds of the invention.

In some embodiments, each compound of the dimer is capable of binding to the other by at least one disulfide bond.

In some embodiments, each compound of the dimer is capable of binding to the other by at least one disulfide bond between the Z of each compound.

In some embodiments, each compound of the dimer is bound to the other by at least one disulfide bond.

In some embodiments, each compound of the dimer is bound to the other by at least one disulfide bond between the Z of each compound.

In some embodiments, each compound of the dimer is non-covalently bound to the other.

The present invention provides a compound having the structure:

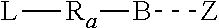

wherein Z is a protein component of the compound, which protein component comprises one or more polypeptides, wherein at least one of the one or more polypeptides comprises consecutive amino acids which (i) are identical to a stretch of consecutive amino acids present in a chain of an F_{c} domain of an antibody; (ii) bind to an F_{c} receptor; and (iii) have at their N-terminus a cysteine or a selenocysteine;
wherein B is a chemical structure linking Rₐ and C;
wherein the dashed line between B and Z represents a peptidyl linkage;
wherein L is selected from the group consisting of: —N₃, an alkyne, a

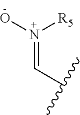

group in which R₅ is an alkyl or aryl group, a

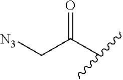 [SMILES: *C(=O)CN=[N+]=[N-]]

group, a tetrazine and a trans-cyclooctene; and
wherein Rₐ is a bond or an organic structure comprising or consisting of a chain of 1, 2, 3, 4, 5, 6, 7, 8, 9, 10 or more moieties, wherein each moiety is independently selected from the group consisting of [PEG(y)]z, polyalkylene glycol, polyoxyalkylated polyol, polyvinyl alcohol, polyvinyl alkyl ether, poly(lactic acid), poly(lactic-glycolic acid), polysaccharide, a branched residue, C₁-C₁₀ alkyl, C₃-C₁₀ cycloalkane, C₂-C₁₀ alkene, C₅-C₁₀ cycloalkene, amine, sulfur, oxygen, succinimide, maleimide, glycerol, triazole, isoxazolidine, C₂-C₅ acyl, C₂-C₅ acylamino, C₂-C₅ acyloxy, succinyl, malonyl, glutaryl, phthalyl, adipoyl, an amino acid, an aryl group, a heteroaryl group, a carbamate, a chemical structure containing a cyclooctane fused to a dihydropyridazine, a chemical structure containing a cyclooctene fused to a triazole, a chemical structure containing a cyclooctene fused to a isoxazolidine, a dibenzocyclooctene, a dibenzoazacyclooctene,

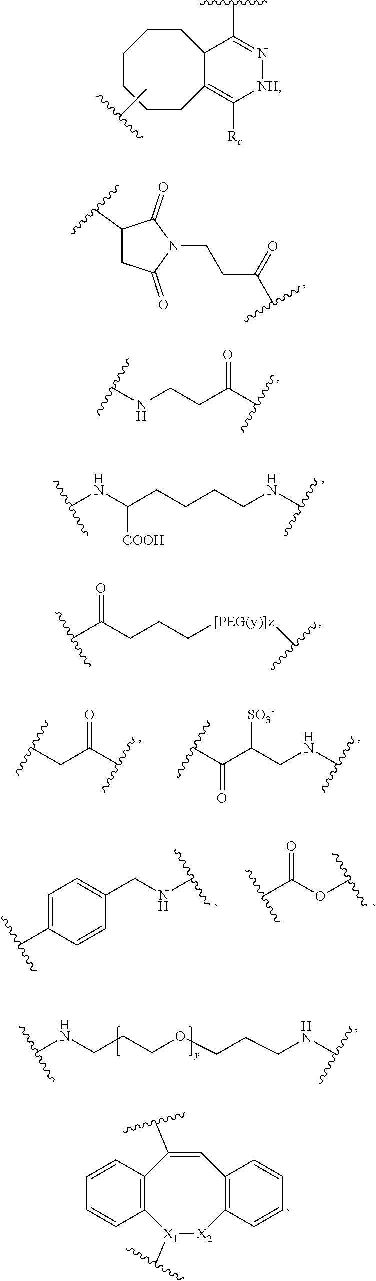

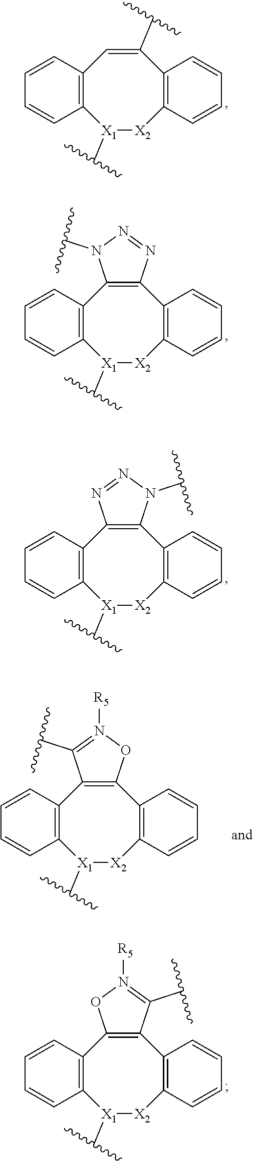

wherein X₁ is CH or N, X₂ is CH₂ or a carbonyl group, and R₅ is an aryl or alkyl group;
wherein [PEG(y)]z is:

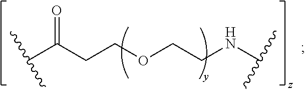

wherein y=1-100 and z=1-10.

In some embodiments, the cysteine or selenocysteine naturally occurs in the stretch of consecutive amino acids. In some embodiments, the cysteine or selenocysteine does not naturally occur in the stretch of consecutive amino acids.

In some embodiments, the consecutive amino acids have at their N-terminus a sequence selected from the group consisting of a cysteine, selenocysteine, CP, CPXCP (where X=P, R, or S) (SEQ ID NOs: 206-208), CDKTHTCPPCP (SEQ ID NO: 209), CVECPPCP (SEQ ID NO: 210), CCVECPPCP (SEQ ID NO: 211) and CDTPPPCPRCP (SEQ ID NO: 212).

In some embodiments, the F_{c} domain of an antibody is a naturally occurring F_{c} domain of an antibody.

In some embodiments, the F_{c} domain of an antibody is a variant F_{c} domain of an antibody.

In some embodiments, the variant F_{c} domain of an antibody is a mutated F_{c} domain of an antibody.

In some embodiments, the mutated F_{c} domain is a substitution mutant.

In some embodiments, the substitution mutant has an amino acid substitution at the N-terminus, the C-terminus, or at a position of the F_{c} domain other than the N-terminus or the C-terminus.

In some embodiments, the substitution mutant has 1, 2, 3, 4, 5, 6, 7, 8, 9, 10, 10-15, 15-20, 10-20, or 20-50 amino acid substitutions in the stretch of consecutive amino acids thereof.

In some embodiments, the substitutions are conservative amino acid substitutions.

In some embodiments, the mutated mutated F_{c} domain is an amino acid addition mutant.

In some embodiments, the amino acid addition mutant has 1, 2, 3, 4, 5, 6, 7, 8, 9, 10, 10-15, 15-20, 10-20, or 20-50 added amino acids in the stretch of consecutive amino acids thereof.

In some embodiments, the mutated F_{c} domain is an amino acid deletion mutant.

In some embodiments, the amino acid deletion mutant has 1, 2, 3, 4, 5, 6, 7, 8, 9, 10, 10-15, 15-20, 10-20, or 20-50 deleted amino acids in the stretch of consecutive amino acids thereof.

In some embodiments, the consecutive amino acids are identical to a stretch of at least 0, 20, 30, 40, 50, 60, 70, 80, 90, 100, 110, 120, 130, 140, 150, 160, 170, 180, or 190 consecutive amino acids present in the chain of the F_{c} domain of the antibody.

In some embodiments, the consecutive amino acids are identical to the stretch of amino acids in the hinge region, the CH2 region or the CH3 region of the Fc domain, or a portion thereof.

In some embodiments, L is —N₃.

In some embodiments, L is

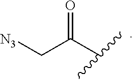 [SMILES: *C(=O)CN=[N+]=[N-]]

In some embodiments, L is an alkyne.

In some embodiments, the alkyne is a propargyl group.

In some embodiments, the alkyne is a cyclooctyne group.

In some embodiments, the alkyne has the structure:

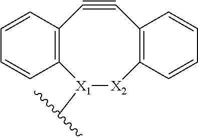

wherein X₁ is CH or N, X₂ is CH₂ or a carbonyl group, and R₅ is an aryl or alkyl group.

In some embodiments, the alkyne has the structure:

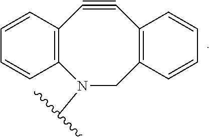 [SMILES: *N1Cc2ccccc2C#Cc2ccccc21]

In some embodiments, L is a tetrazine.

In some embodiments, the tetrazine has the structure:

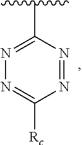

wherein Rc is H, alkyl or aryl.

In some embodiments, the tetrazine has the structure:

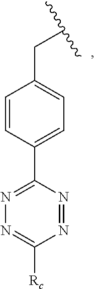

wherein Rc is H, alkyl or aryl.

In some embodiments, the tetrazine has the structure:

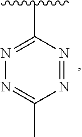 [SMILES: *c1nnc(C)nn1]

In some embodiments, the tetrazine has the structure:

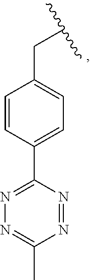 [SMILES: *Cc1ccc(-c2nnc(C)nn2)cc1]

In some embodiments, L is trans-cyclooctene.

In some embodiments, the trans-cyclooctene has the structure:

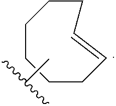

In some embodiments, the trans-cyclooctene has the structure:

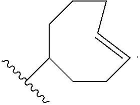 [SMILES: *C1CC/C=C/CCC1]

In some embodiments, Ra or Rb is an organic structure comprising a [PEG(y)]z group.

In some embodiments, Ra or Rb is an organic structure comprising a polyalkylene glycol, polyoxyalkylated polyol, polyvinyl alcohol, polyvinyl alkyl ether, poly(lactic acid), poly(lactic-glycolic acid), or polysaccharide group.

In some embodiments, Ra or Rb is an organic structure comprising a C₁-C₄ alkyl group.

In some embodiments, Ra or Rb is an organic structure comprising a succinimide.

In some embodiments, Ra or Rb is an organic structure comprising an amine.

In some embodiments, Ra or Rb is an organic structure comprising a succinyl, malonyl, glutaryl, phthalyl or adipoyl.

In some embodiments, Ra or Rb is an organic structure comprising a malonyl.

In some embodiments, Ra or Rb is an organic structure comprising an amino acid.

In some embodiments, Ra or Rb is an organic structure comprising a cysteine.

In some embodiments, Ra or Rb is an organic structure comprising a lysine.

In some embodiments, Ra or Rb is an organic structure consisting of a chain of 3 moieties selected from the group consisting of [PEG(y)]z, polyalkylene glycol, polyoxyalkylated polyol, polyvinyl alcohol, polyvinyl alkyl ether, poly(lactic acid), poly(lactic-glycolic acid), polysaccharide, a branched residue, C₁-C₁₀ alkyl, C₃-C₁₀ cycloalkane, C₂-C₁₀ alkene, C₅-C₁₀ cycloalkene, amine, sulfur, oxygen, succinimide, maleimide, glycerol, triazole, isoxazolidine, C₂-C₅ acyl, C₂-C₅ acylamino, C₂-C₅ acyloxy, succinyl, malonyl, glutaryl, phthalyl, adipoyl, an amino acid, an aryl group, a heteroaryl group, a carbamate, a chemical structure containing a cyclooctane fused to a dihydropyridazine, a chemical structure containing a cyclooctene fused to a triazole, a chemical structure containing a cyclooctene fused to a isoxazolidine, a dibenzocyclooctene, a dibenzoazacyclooctene,

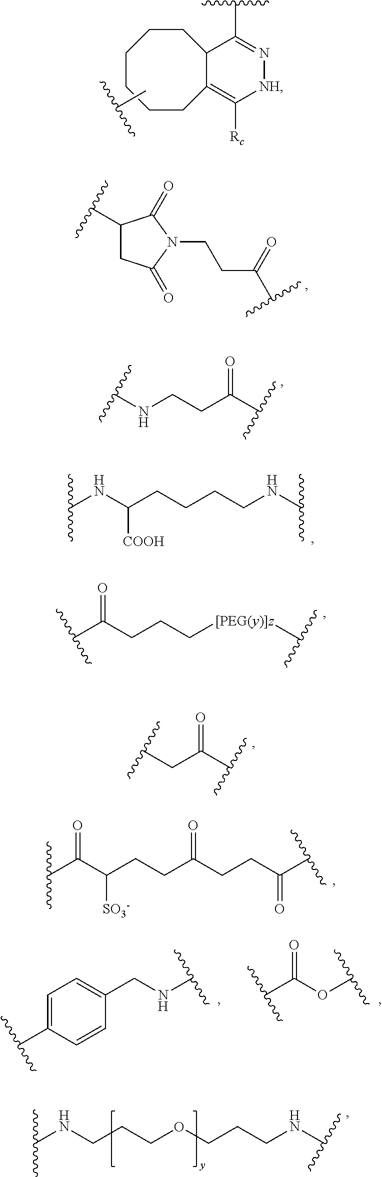

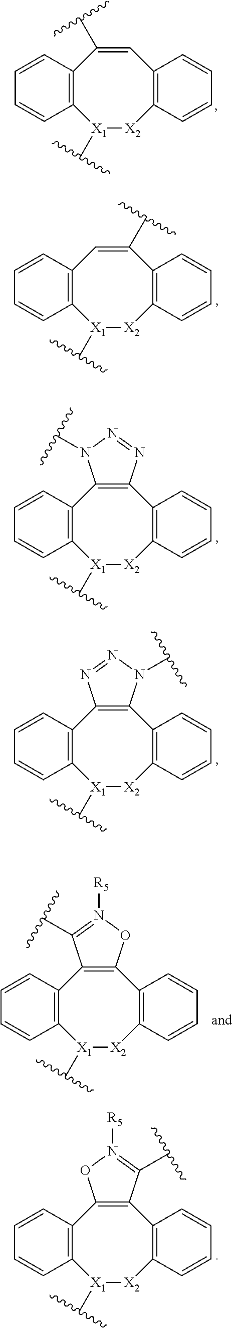

In some embodiments, Ra or Rb is an organic structure consisting of a chain of four moieties selected from the group consisting of [PEG(y)]z, polyalkylene glycol, polyoxyalkylated polyol, polyvinyl alcohol, polyvinyl alkyl ether, poly(lactic acid), poly(lactic-glycolic acid), polysaccharide, a branched residue, C₁-C₁₀ alkyl, C₃-C₁₀ cycloalkane, C₂-C₁₀ alkene, C₅-C₁₀ cycloalkene, amine, sulfur, oxygen, succinimide, maleimide, glycerol, triazole, isoxazolidine, C₂-C₅ acyl, C₂-C₅ acylamino, C₂-C₅ acyloxy, succinyl, malonyl, glutaryl, phthalyl, adipoyl, an amino acid, an aryl group, a heteroaryl group, a carbamate, a chemical structure containing a cyclooctane fused to a dihydropyridazine, a chemical structure containing a cyclooctene fused to a triazole, a chemical structure containing a cyclooctene fused to a isoxazolidine, a dibenzocyclooctene, a dibenzoazacyclooctene,

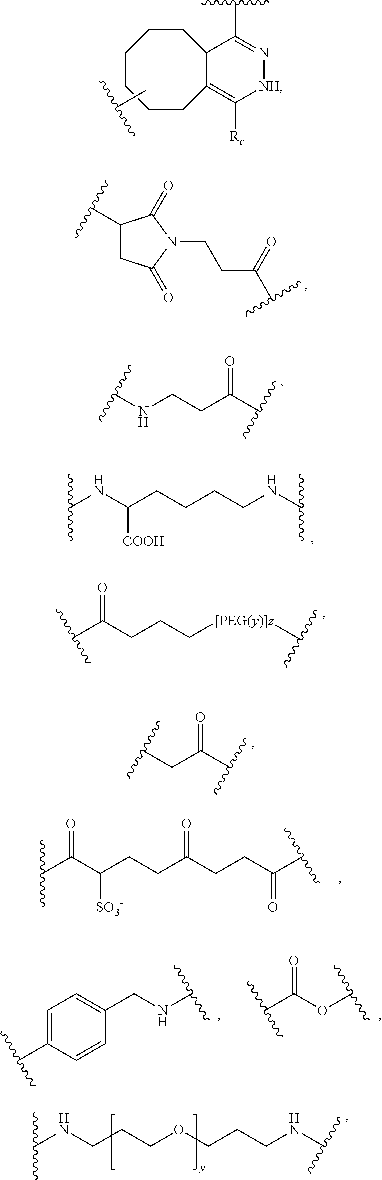

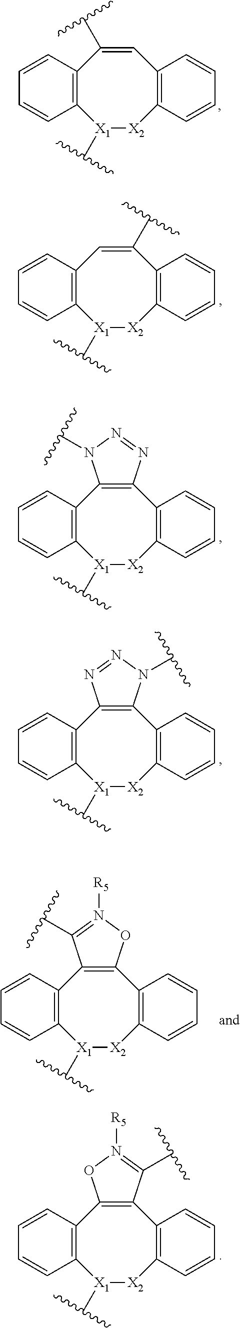

In some embodiments, Ra or Rb is an organic structure consisting of a chain of five moieties selected from the group consisting of [PEG(y)]z, polyalkylene glycol, polyoxyalkylated polyol, polyvinyl alcohol, polyvinyl alkyl ether, poly(lactic acid), poly(lactic-glycolic acid), polysaccharide, a branched residue, C₁-C₁₀ alkyl, C₃-C₁₀ cycloalkane, C₂-C₁₀ alkene, C₅-C₁₀ cycloalkene, amine, sulfur, oxygen, succinimide, maleimide, glycerol, triazole, isoxazolidine, C₂-C₅ acyl, C₂-C₅ acylamino, C₂-C₅ acyloxy, succinyl, malonyl, glutaryl, phthalyl, adipoyl, an amino acid, an aryl group, a heteroaryl group, a carbamate, a chemical structure containing a cyclooctane fused to a dihydropyridazine, a chemical structure containing a cyclooctene fused to a triazole, a chemical structure containing a cyclooctene fused to a isoxazolidine, a dibenzocyclooctene, a dibenzoazacyclooctene,

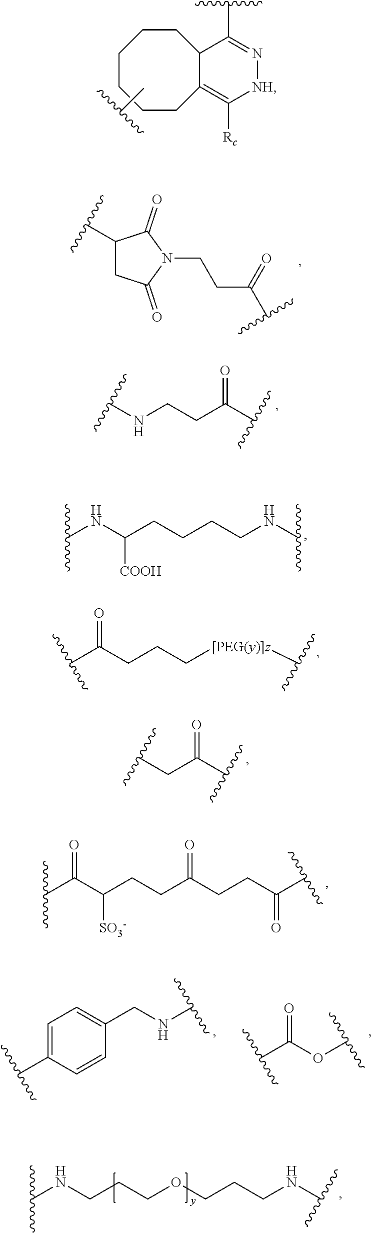

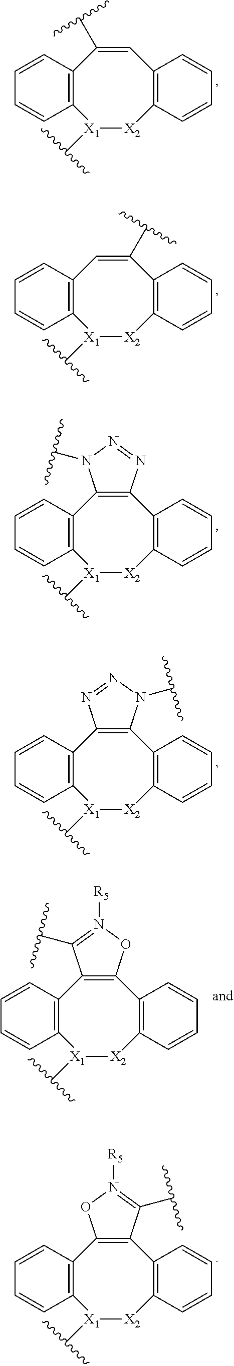

In some embodiments, Ra or Rb comprises a [PEG (y)]z group bonded to a lysine.

In some embodiments, Ra or Rb comprises a C₁-C₄ acyl group bonded to a succinimide group.

In some embodiments, Ra or Rb comprises a lysine bonded to a C₁-C₄ acyl.

In some embodiments, Ra or Rb comprises a [PEG(y)]z group, which is bonded to a glutaryl.

In some embodiments, Ra or Rb is an organic structure consisting of a chain of three, four or five moieties selected from the group consisting of [PEG(y)]z, C₂-C₅ acyl, succinyl, malonyl, glutaryl, an amino acid, a chemical structure containing a cyclooctane fused to a dihydropyridazine, a chemical structure containing a cyclooctene fused to a triazole, a chemical structure containing a cyclooctene fused to a isoxazolidine, a dibenzocyclooctene, a dibenzoazacyclooctene,

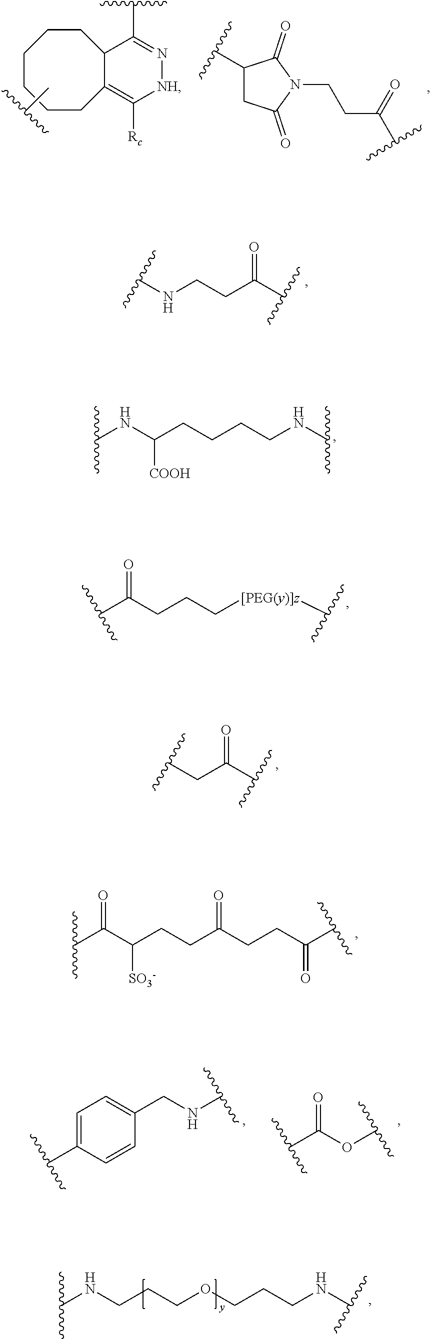

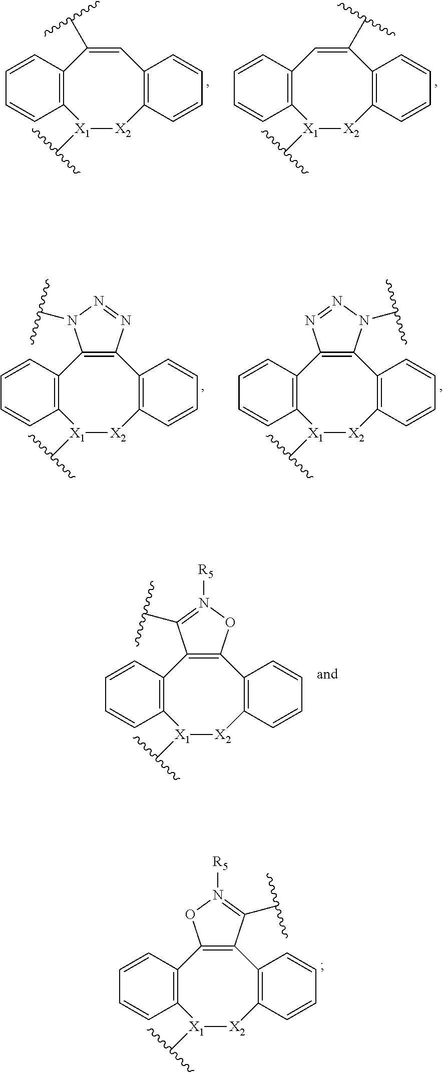

wherein X₁ is CH or N, X₂ is CH₂ or a carbonyl group, and R₅ is an aryl or alkyl group;
wherein [PEG(y)]z is:

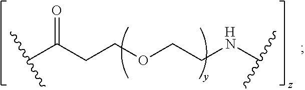

wherein y=1-100 and z=1-10.

In some embodiments, Ra or Rb is a bond.

In some embodiments, Ra or Rb is a cysteine.

In some embodiments, Ra or Rb has a linear structure.

In some embodiments, Ra or Rb has a branched structure.

In some embodiments, y is 1-20. In some embodiments, y is 21-40. In some embodiments, y is 41-60. In some embodiments, y is 61-80. In some embodiments, y is 30-50. In some embodiments, y is 12, 24, 36 or 48.

In some embodiments, z is 1.

In some embodiments, the terminal carbonyl of the [PEG(y)]z group is part of an amide bond.

In some embodiments, the terminal amine of the [PEG(y)]z group is part of an amide bond.

In some embodiments, Ra or Rb is

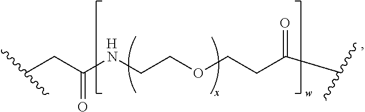

wherein x is 1-100, and w is 0-5.

In some embodiments, x is 1-20. In some embodiments, x is 21-40. In some embodiments, x is 41-60. In some embodiments, x is 61-80. In some embodiments, x is 30-50. In some embodiments, x is 12, 24, 36 or 48.

In some embodiments, w is 1. In some embodiments, w is 0.

In some embodiments, Ra or Rb has the structure:

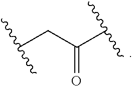 [SMILES: *CC(*)=O]

In some embodiments, Ra or Rb is:

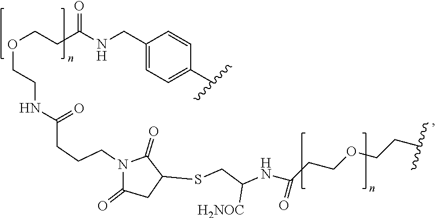

wherein n is 1, 2, 3, 4, 5, 6, 7, 8, 9, 10, 11, 12, 13, 14, 15, 16, 17, 18, 19, 20, 1-30, 1-40, or 1-50.

In some embodiments, Ra or Rb is:

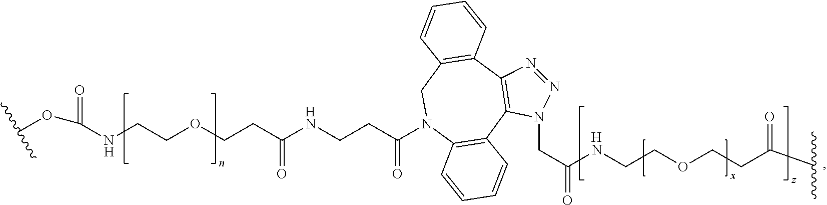

wherein n is 1, 2, 3, 4, 5, 6, 7, 8, 9, 10, 11, 12, 13, 14, 15, 16, 17, 18, 19, 20, 1-30, 1-40, or 1-50, x is 1, 2, 3, 4, 5, 6, 7, 8, 9, 10, 11, 12, 13, 14, 15, 16, 17, 18, 19, 20, 1-30, 1-40, or 1-50 and z is 1, 2, 3, 4, 5, 6, 7, 8, 9, 10, 11, 12, 13, 14, 15, 16, 17, 18, 19, 20, 1-30, 1-40, or 1-50.

In some embodiments, Ra or Rb is:

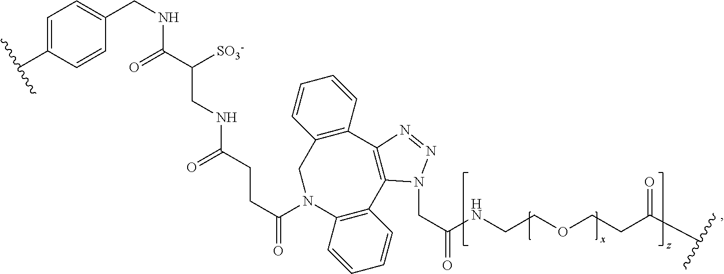

wherein x is 1, 2, 3, 4, 5, 6, 7, 8, 9, 10, 11, 12, 13, 14, 15, 16, 17, 18, 19, 20, 1-30, 1-40, or 1-50 and z is 1, 2, 3, 4, 5, 6, 7, 8, 9, 10, 11, 12, 13, 14, 15, 16, 17, 18, 19, 20, 1-30, 1-40, or 1-50.

In some embodiments, Ra or Rb is:

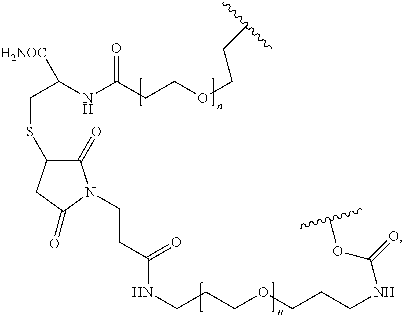

wherein n is 1, 2, 3, 4, 5, 6, 7, 8, 9, 10, 11, 12, 13, 14, 15, 16, 17, 18, 19, 20, 1-30, 1-40, or 1-50.

In some embodiments, the solid line between A and B represents a nonpeptidyl linkage comprising the structure:

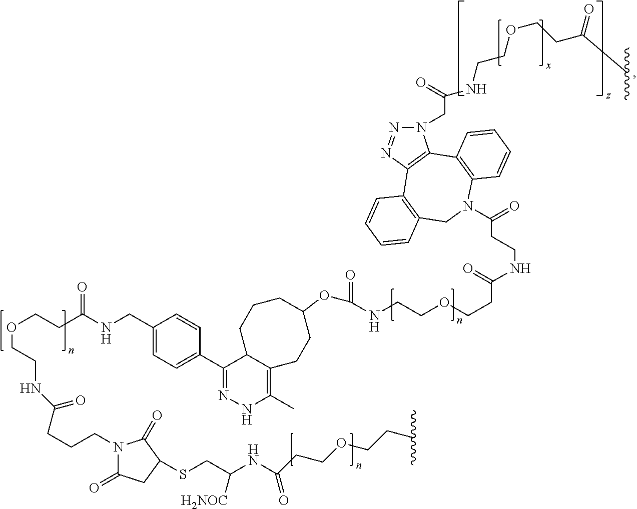

wherein n is 1, 2, 3, 4, 5, 6, 7, 8, 9, 10, 11, 12, 13, 14, 15, 16, 17, 18, 19, 20, 1-30, 1-40, or 1-50, x is 1, 2, 3, 4, 5, 6, 7, 8, 9, 10, 11, 12, 13, 14, 15, 16, 17, 18, 19, 20, 1-30, 1-40, or 1-50 and z is 1, 2, 3, 4, 5, 6, 7, 8, 9, 10, 11, 12, 13, 14, 15, 16, 17, 18, 19, 20, 1-30, 1-40, or 1-50.

In some embodiments, the solid line between A and B represents a nonpeptidyl linkage comprising the structure:

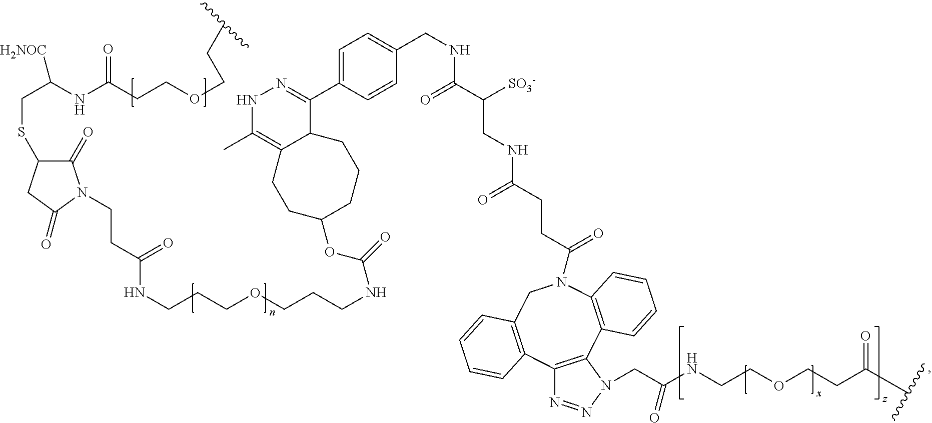

wherein n is 1, 2, 3, 4, 5, 6, 7, 8, 9, 10, 11, 12, 13, 14, 15, 16, 17, 18, 19, 20, 1-30, 1-40, or 1-50, x is 1, 2, 3, 4, 5, 6, 7, 8, 9, 10, 11, 12, 13, 14, 15, 16, 17, 18, 19, 20, 1-30, 1-40, or 1-50 and x is 1, 2, 3, 4, 5, 6, 7, 8, 9, 10, 11, 12, 13, 14, 15, 16, 17, 18, 19, 20, 1-30, 1-40, or 1-50.

The present invention provides a process for producing a compound having the structure:

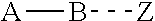

wherein A is a biologically active structure of the compound;
wherein Z is a protein component of the compound, which protein component comprises one or more polypeptides, wherein at least one of the one or more polypeptides comprises consecutive amino acids which (i) are identical to a stretch of consecutive amino acids present in a chain of an F_{c} domain of an antibody; (ii) bind to an F_{c} receptor; and (iii) have at their N-terminus a cysteine or a selenocysteine;
wherein B is a chemical structure linking A and Z;
wherein the dashed line between B and Z represents a peptidyl linkage;
wherein the solid line between A and B represents a nonpeptidyl linkage;
which comprises the following steps:
a) obtaining an A′ which comprises A or a derivative of A, and a first terminal reactive group;
b) obtaining a B′ which comprises B or a derivative of B, a second terminal reactive group and a third terminal reactive group, wherein the second terminal reactive group is capable of reacting with the first terminal reactive group to form a non-peptidyl linkage;
c) obtaining a Z′ which comprises Z or a derivative of Z, and a fourth terminal reactive group, wherein the fourth terminal reactive group is capable of reacting with the third terminal reactive group to form a peptidyl linkage; and
d) reacting A′, B′ and Z′ in any order to produce the compound.

In some embodiments, the cysteine or selenocysteine naturally occurs in the stretch of consecutive amino acids. In some embodiments, the cysteine or selenocysteine does not naturally occur in the stretch of consecutive amino acids.

In some embodiments, the consecutive amino acids have at their N-terminus a sequence selected from the group consisting of a cysteine, selenocysteine, CP, CPXCP (where X=P, R, or S) (SEQ ID NOs: 206-208), CDKTHTCPPCP (SEQ ID NO: 209), CVECPPCP (SEQ ID NO: 210), CCVECPPCP (SEQ ID NO: 211) and CDTPPPCPRCP (SEQ ID NO: 212).

In some embodiments, the F_{c} domain of an antibody is a naturally occurring F_{c} domain of an antibody.

In some embodiments, the F_{c} domain of an antibody is a variant F_{c} domain of an antibody.

In some embodiments, the variant F_{c} domain of an antibody is a mutated F_{c} domain of an antibody.

In some embodiments, the mutated F_{c} domain is a substitution mutant.

In some embodiments, the substitution mutant has an amino acid substitution at the N-terminus, the C-terminus, or at a position of the F_{c} domain other than the N-terminus or the C-terminus.

In some embodiments, the substitution mutant has 1, 2, 3, 4, 5, 6, 7, 8, 9, 10, 10-15, 15-20, 10-20, or 20-50 amino acid substitutions in the stretch of consecutive amino acids thereof.

In some embodiments, the substitutions are conservative amino acid substitutions.

In some embodiments, the mutated mutated F_{c} domain is an amino acid addition mutant.

In some embodiments, the amino acid addition mutant has 1, 2, 3, 4, 5, 6, 7, 8, 9, 10, 10-15, 15-20, 10-20, or 20-50 added amino acids in the stretch of consecutive amino acids thereof.

In some embodiments, the mutated F_{c} domain is an amino acid deletion mutant.

In some embodiments, the amino acid deletion mutant has 1, 2, 3, 4, 5, 6, 7, 8, 9, 10, 10-15, 15-20, 10-20, or 20-50 deleted amino acids in the stretch of consecutive amino acids thereof.

In some embodiments, the consecutive amino acids are identical to a stretch of at least 0, 20, 30, 40, 50, 60, 70, 80, 90, 100, 110, 120, 130, 140, 150, 160, 170, 180, or 190 consecutive amino acids present in the chain of the F_{c} domain of the antibody.

In some embodiments, the consecutive amino acids are identical to the stretch of amino acids in the hinge region, the CH2 region or the CH3 region of the Fc domain, or a portion thereof.

In some embodiments, the solid line between A and B represents a nonpeptidyl linkage comprising the structure:

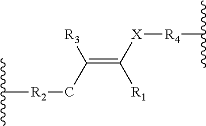

wherein

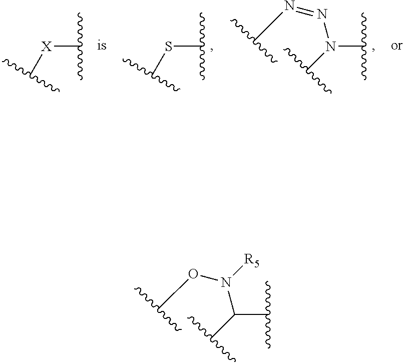

in which R₅ is an alkyl or aryl group
wherein R₁ is H or is part of an additional structure that is a cyclic structure, wherein the additional cyclic structure comprises R₁ or a portion of R₁, and may also comprise R₂ or a portion of R₂, and the carbon between R₂ and the alkene double bond;
   with the proviso that if

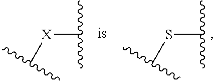

R₃ is a H; if

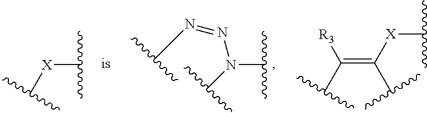

is a triazole ring that comprises

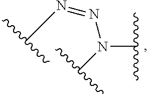 [SMILES: */N=N\N(*)*]

and if

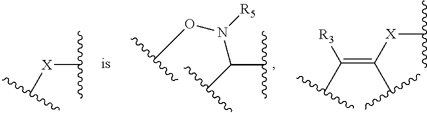

is a N-alkyl or aryl substituted isoxazoline ring that comprises

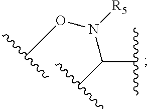

and
wherein R₂ represents an organic structure which connects to one of A or B and R₄ represents an organic structure which connects to the other of A or B;
which comprises the following steps:
a) obtaining an A′ which comprises A or a derivative of A, and a first terminal reactive group;
b) obtaining a B′ which comprises B or a derivative of B, a second terminal reactive group and a third terminal reactive group, wherein the second terminal reactive group is capable of reacting with the first terminal reactive group to form a non-peptidyl linkage;
c) obtaining a C′ which comprises C or a derivative of C, and a fourth terminal reactive group, wherein the fourth terminal reactive group is capable of reacting with the third terminal reactive group to form a peptidyl linkage; and
d) reacting A′, B′ and C′ in any order to produce the compound.

In some embodiments step d) is performed by first reacting A′ and B′ to produce

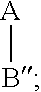

B″; wherein B″ comprises B and the third terminal reactive group, and the solid line between B″ and A represents a non-peptidyl linkage; and then reacting

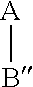

B″ with C′ to produce the compound.

In some embodiments, step d) is performed by first reacting C′ and B′ to produce

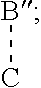

C; wherein B″ comprises B and the second terminal reactive group, and the dashed line between B″ and C represents a peptidyl linkage; and then reacting

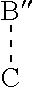

with A′ to produce the compound.

In some embodiments, the first terminal reactive group is an azide, a thiol, a nitrone or an alkyne.

In some embodiments, the first terminal reactive group is an alkyne.

In some embodiments, the alkyne is a cycloalkyne

In some embodiments, the alkyne is an eight-membered ring.

In some embodiments, the alkyne is an azacyclooctyne.

In some embodiments, the cycloalkyne is a biarylazacyclooctyne.

In some embodiments, the cycloalkyne is a cyclooctyne.

In some embodiments, the alkyne is a terminal alkyne.

In some embodiments, the first terminal reactive group is an azide, thiol or nitrone.

In some embodiments, the first terminal reactive group is an azide.

In some embodiments, the first terminal reactive group is a thiol.

In some embodiments, the first terminal reactive group is a nitrone.

In some embodiments, the first terminal reactive group is an N-alkyl nitrone.

In some embodiments, the first terminal reactive group is an N-aryl nitrone.

In some embodiments, the second terminal reactive group is an azide, a thiol, a nitrone or an alkyne.

In some embodiments, the second terminal reactive group is an alkyne.

In some embodiments, the alkyne is a cycloalkyne

In some embodiments, the alkyne is an eight-membered ring.

In some embodiments, the alkyne is an azacyclooctyne.

In some embodiments, the cycloalkyne is a biarylazacyclooctyne.

In some embodiments, the cycloalkyne is a cyclooctyne.

In some embodiments, the alkyne is a terminal alkyne.

In some embodiments, the second terminal reactive group is an azide, thiol or nitrone.

In some embodiments, the second terminal reactive group is an azide.

In some embodiments, the second terminal reactive group is a thiol.

In some embodiments, the second terminal reactive group is a nitrone.

In some embodiments, the second terminal reactive group is an N-alkyl nitrone.

In some embodiments, the second terminal reactive group is an N-aryl nitrone.

In some embodiments, the first terminal reactive group is a terminal alkyne and the second terminal reactive group is an azide, thiol or nitrone.

In some embodiments, the second terminal reactive group is an azide.

In some embodiments, the second terminal reactive group is a thiol.

In some embodiments, the second terminal reactive group is a nitrone.

In some embodiments, the nitrone is an N-alkyl or N-aryl nitrone.

In some embodiments, the first terminal reactive group is an azide, thiol or nitrone, and the second terminal reactive group is a terminal alkyne.

In some embodiments, the first terminal reactive group is an azide.

In some embodiments, the first terminal reactive group is a thiol.

In some embodiments, the first terminal reactive group is a nitrone.

In some embodiments, the nitrone is an N-alkyl or N-aryl nitrone.

In some embodiments, the first terminal reactive group is a cycloalkyne and the second terminal reactive group is an azide, thiol or nitrone.

In some embodiments, the first terminal reactive group is an azide.

In some embodiments, the first terminal reactive group is a thiol.

In some embodiments, the first terminal reactive group is a nitrone.

In some embodiments, the nitrone is an N-alkyl or N-aryl nitrone.

In some embodiments, the first terminal reactive group is an azide, thiol or nitrone, and the second terminal reactive group is a cycloalkyne.

In some embodiments, the first terminal reactive group is an azide.

In some embodiments, the first terminal reactive group is a thiol.

In some embodiments, the first terminal reactive group is a nitrone.

In some embodiments, the nitrone is an N-alkyl or N-aryl nitrone.

In some embodiments, the cycloalkyne is an eight-membered ring.

In some embodiments, the cycloalkyne is an azacyclooctyne.

In some embodiments, the cycloalkyne is a biarylazacyclooctyne.

In some embodiments, the cycloalkyne is a cyclooctyne.

In some embodiments, the first terminal reactive group is an azide and the second terminal reactive group is a terminal alkyne; or the first terminal reactive group is an azide and the second terminal reactive group is a cycloalkyne; or the first terminal reactive group is a thiol and the second terminal reactive group is a cycloalkyne; or the first terminal reactive group is a N-alkyl nitrone or N-aryl nitrone and the second terminal reactive group is a cyclooctyne.

In some embodiments, the second terminal reactive group is an azide and the first terminal reactive group is a terminal alkyne; or the second terminal reactive group is an azide and the first terminal reactive group is a cycloalkyne; or the second terminal reactive group is a thiol and the first terminal reactive group is a cycloalkyne; or the second terminal reactive group is a N-alkyl nitrone or N-aryl nitrone and the first terminal reactive group is a cyclooctyne.

In some embodiments, the first terminal reactive group and the second terminal reactive group react to produce a triazole, thiolene, N-alkyl isoxazoline or N-aryl isoxazoline.

In some embodiments, the first terminal reactive group and the second terminal reactive group react to produce a triazole.

In some embodiments, the first terminal reactive group and the second terminal reactive group react to produce a thiolene.

In some embodiments, the first terminal reactive group and the second terminal reactive group react to produce a N-alkyl isoxazoline or N-aryl isoxazoline.

In some embodiments, reacting the first reactive group with the second reactive group results in at least an 80%, 85% or 90% yield of the reaction in less than 3, 6, 12, 18, 24, 30, 36, 42, 48 or 72 hours.

In some embodiments, the solid line between A and B represents a nonpeptidyl linkage comprising the structure:

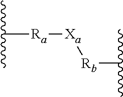

wherein Xₐ is a chemical structure containing a cyclooctane fused to a dihydropyridazine; and
wherein Rₐ represents an organic structure which connects to one of A or B and R_{b} represents an organic structure which connects to the other of A or B.

In some embodiments, the first terminal reactive group is a trans-cyclooctene or a tetrazine.

In some embodiments, the first terminal reactive group is a trans-cyclooctene.

In some embodiments, the alkyne is a tetrazine.

In some embodiments, the second terminal reactive group is a trans-cyclooctene or a tetrazine.

In some embodiments, the second terminal reactive group is a trans-cyclooctene.

In some embodiments, the second terminal reactive group has the structure:

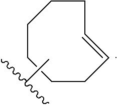

In some embodiments, the second terminal reactive group has the structure:

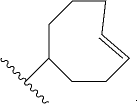 [SMILES: *C1CC/C=C/CCC1]

In some embodiments, the second terminal reactive group is a tetrazine.

In some embodiments, the second terminal reactive group has the structure:

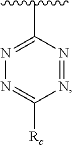

wherein R_{c} is H, alkyl or aryl.

In some embodiments, the second terminal reactive group has the structure:

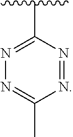 [SMILES: *c1nnc(C)nn1]

In some embodiments, the first terminal reactive group is a trans-cyclooctene and the second terminal reactive group is a tetrazine.

In some embodiments, the first terminal reactive group is a tetrazine and the second terminal reactive group is a trans-cyclooctene.

In some embodiments, the first terminal reactive group and the second terminal reactive group react to produce a chemical structure containing a cyclooctane fused to a dihydropyridazine.

In some embodiments, the first terminal reactive group and the second terminal reactive group react to produce the chemical structure:

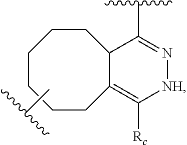

wherein R_{c} is H, alkyl or aryl;
or a tautomer thereof.

In some embodiments, the the third reactive group and the fourth terminal reactive group are each independently an amino acid or amino acid derivative.

In some embodiments, the third reactive group is a threonine or threonine derivative.

In some embodiments, the third reactive group is a thioester derivative of an amino acid.

In some embodiments, the fourth reactive group is cysteine, selenocysteine, homocysteine, or homoselenosysteine, or a derivative of cysteine, selenocysteine, homocysteine, or homoselenosysteine.

In some embodiments, the fourth reactive group is cysteine or a derivative of cysteine.

In some embodiments, the fourth reactive group is cysteine.

In some embodiments, A′ is prepared by the following steps:
i) obtaining an A″ which comprises A or a derivative of A, and a stretch of consecutive amino acids comprising an intein;
ii) obtaining a substituted cysteine, selenocysteine, homocysteine, or homoselenosysteine residue, or a substituted derivative of a cysteine, selenocysteine, homocysteine, or homoselenosysteine residue, wherein the cysteine residue is substituted at the C-terminus with an organic structure containing an alkyne, an azide, a thiol, or a nitrone; and
iii) reacting A″ with the substituted cysteine residue to produce A′.

In some embodiments, the organic structure containing an alkyne is N-propargyl amine.

In some embodiments, A′ is prepared by the following steps:
i) obtaining an A″ which comprises A or a derivative of A, and which comprises at least one free thiol group;
ii) obtaining a compound which comprises a first terminal reactive group and a terminal maleimide; and
iii) reacting A″ with the compound of step ii) to produce A′.

In some embodiments, A″ is prepared by the following steps:
a) obtaining an A′″, wherein A′″ is a polypeptide which comprises A or a derivative of A, and which comprises at least one disulfide bond; and
b) treating A′″ with mercaptoethylamine (MEA) to produce A″.

In some embodiments, the A′″ is prepared by the following steps:
a) obtaining a monoclonal antibody which comprises A or derivative of A, and which comprises at least one disulfide bond; and
b) treating the polypeptide of step a) with IdeS to produce A′″.

In some embodiments, the monoclonal antibody binds TNFα.

In some embodiments, the monoclonal antibody is adalimumab.

In some embodiments, if R1 is hydrogen and the first terminal reactive group is alkyne, then in step d) B′ is reacted in the presence of a metal catalyst.

In some embodiments, if R1 is hydrogen and the second terminal reactive group is alkyne, then in step d) B′ is reacted in the presence of a metal catalyst.

In some embodiments, the metal catalyst is Ag(I) or Cu(I).

In some embodiments, A′ comprises one or more branched residue, wherein each branched residue comprises an additional first terminal reactive group.

In some embodiments, B′ comprises one or more branched residue, wherein each branched residue comprises an additional second terminal reactive group.

In some embodiments, B′ comprises one or more branched residue, wherein each branched residue comprises an additional third terminal reactive group.

In some embodiments, the branched residue is an amino acid residue.

In some embodiments, the amino acid residue is a lysine or a lysine derivative, arginine or an arginine derivative, aspartic acid or an aspartic acid derivative, glutamic acid or a glutamic acid derivative, asparagines or a asparagines derivative, glutamine or glutamine derivative, tyrosine or tyrosine derivative, cysteine or cysteine derivative or ornithine or ornithine derivative.

In some embodiments, the amino acid residue is substituted at the N-position with a residue containing a terminal amino or carbony reactive group.

In some embodiments, the branched residue is an organic residue containing two or more terminal amino groups or two or more terminal carbonyl groups.

In some embodiments, the organic residue is iminodipropionic acid, iminodiacetic acid, 4-amino-pimelic acid, 4-amino-heptanedioic acid, 3-aminohexanedioic acid, 3-aminoadipic acid, 2-aminooctanedioic acid, or 2-amino-6-carbonyl-heptanedioic acid.

In some embodiments, the process is performed in the absence of a non-thiol reducing agent.

In some embodiments, the process is performed in the absence of a thiol reducing agent.

In some embodiments, the process is performed in the presence of a thiol reducing agent.

In some embodiments, the process is performed at an overall yield of 80% or higher.

In some embodiments, the process is performed at an overall yield of 90% or higher.

In some embodiments, reacting the first reactive group with the second reactive group results in at least a 50%, 55%, 60%, 65%, 70%, 80%, 85% or 90% yield of the reaction in less than 1, 2, 3, 4, 5, 6, 7, 8, 9, 10, or 60 minutes.

In some embodiments, B is an organic acid residue.

In some embodiments, B is a stretch of 1-50 amino acid residues, and optionally, an organic acid residue.

In some embodiments, B is a stretch of 1-10 consecutive amino acids.

In some embodiments, B comprises a stretch of consecutive amino acids in the sequence, or a portion thereof, EPKSCDKTHTCPPCP (SEQ ID NO:213), ERKCCVECPPCP (SEQ ID NO:214), ELKTPLGDTTHTCPRCP(EPKSCDTPPPCPRCP)3 (SEQ ID NO:215), ESKYGPPCPSCP (SEQ ID NO:216).

In some embodiments, B has a threonine at its C-terminus.

In some embodiments, Z comprises one C, wherein C is a first polypeptide, which first polypeptide comprises consecutive amino acids which (i) are identical to a stretch of consecutive amino acids present in a chain of an F_{c} domain of an antibody; (ii) bind to an F_{c} receptor; and (iii) have at their N-terminus a sequence selected from the group consisting of a cysteine, selenocysteine, CP, CPXCP (where X=P, R, or S) (SEQ ID NOs: 206-208), CDKTHTCPPCP (SEQ ID NO: 209), CVECPPCP (SEQ ID NO: 210), CCVECPPCP (SEQ ID NO: 211) and CDTPPPCPRCP (SEQ ID NO: 212).

In some embodiments, C comprises consecutive amino acids which (i) are identical to a stretch of consecutive amino acids present in a chain of an F_{c} domain of an antibody; (ii) bind to an F_{c} receptor; and (iii) have at their N-terminus a sequence comprising a naturally occurring cysteine selected from the group consisting of CP, CPXCP (where X=P, R, or S) (SEQ ID NOs: 206-208), CDKTHTCPPCP (SEQ ID NO: 209), CVECPPCP (SEQ ID NO: 210), CCVECPPCP (SEQ ID NO: 211) and CDTPPPCPRCP (SEQ ID NO: 212).

In some embodiments, C is a polypeptide component of the compound, which polypeptide component comprises consecutive amino acids which (i) are identical to a stretch of consecutive amino acids present in a chain of an F_{c} domain of an antibody; (ii) bind to an F_{c} receptor; and (iii) have at their N-terminus a sequence comprising a non-naturally occurring cysteine or selenocysteine.

In some embodiments, C comprises consecutive amino acids which are identical to a stretch of consecutive amino acids present in the chain of an Fc domain of an antibody selected from the group consisting of IgG, IgM, IgA, IgD, and IgE.

In some embodiments, C comprises consecutive amino acids which are identical to a stretch of consecutive amino acids present in the chain of an Fc6 domain of an antibody.

In some embodiments, C further comprises consecutive amino acids which are identical to a stretch of consecutive amino acids present in a chain of an antibody other than a chain of a Fc domain of the antibody.

In some embodiments, C comprises consecutive amino acids which are identical to a stretch of consecutive amino acids present in a heavy chain of a Fab or a Fab′ of an antibody.

In some embodiments, C comprises consecutive amino acids which are identical to a stretch of consecutive amino acids present in the light chain of a Fab or a Fab′ of an antibody.

In some embodiments, Z further comprises a second polypeptide, which second polypeptide comprises consecutive amino acids which are identical to a stretch of consecutive amino acids present in a chain of an antibody other than a chain of a Fc domain of the antibody.

In some embodiments, the second polypeptide comprises consecutive amino acids which are identical to a stretch of consecutive amino acids present in a heavy chain of a Fab or a Fab′ of an antibody.

In some embodiments, the second polypeptide comprises consecutive amino acids which are identical to a stretch of consecutive amino acids present in the light chain of a Fab or a Fab′ of an antibody.

In some embodiments, Z comprises an antibody or a portion thereof.

In some embodiments, Z comprises at least one Fab or Fab′ of an antibody, or a portion of the at least one Fab or Fab′.

In some embodiments, Z comprises at least 2, 3, 4, 5, 6, 7, 8, 9, or 10 copies of the Fab or Fab′ or portion thereof.

In some embodiments, Z comprises Fab-1 or Fab′1, or a portion thereof of an antibody.

In some embodiments, Z comprises Fab-2 or Fab′2, or a portion thereof of an antibody.

In some embodiments, Z comprises two Fab or Fab′ hands of an antibody.

In some embodiments, the antibody is an IgG, IgM, IgA, IgD or IgE antibody.

In some embodiments, the Fab or Fab′ is present in adalimumab.

In some embodiments, Z comprises at least one stretch of consecutive amino acids which are identical to a stretch of consecutive amino acids present in a single chain antibody.

In some embodiments, the C-terminus of C comprises a stretch of consecutive amino acids present in a chain of an F_{c} domain of an antibody that has been modified.

In some embodiments, the C-terminus of C is a cysteine, selenocysteine, homocysteine, or homoselenosysteine, or a derivative of cysteine, selenocysteine, homocysteine, or homoselenosysteine.

In some embodiments, B is linked to Z via a peptidyl linkage between an N-terminal cysteine or selenocysteine of a polypeptide of Z and an amino acid residue or an organic acid residue of B.

In some embodiments, Z comprises a second polypeptide, and B is linked to Z via a peptidyl linkage between the N-terminal cysteine or selenocysteine of the second polypeptide of Z and an amino acid residue or an organic acid residue of B.

In some embodiments, B is linked to C via a peptidyl linkage between the N-terminal cysteine or selenocysteine of C and an amino acid residue or an organic acid residue of B.

In some embodiments, Z comprises one polypeptide, which is C.

In some embodiments, Z comprises two polypeptides, which are C and a second polypeptide.

The present invention provides homodimers and heterodimers comprising compounds of the invention.

In some embodiments, each compound of the dimer is capable of binding to the other by at least one disulfide bond.

In some embodiments, each compound of the dimer is capable of binding to the other by at least one disulfide bond between the C or the second polypeptide of each compound.

In some embodiments, each compound of the dimer is bound to the other by at least one disulfide bond.

In some embodiments, each compound of the dimer is bound to the other by at least one disulfide bond between the C or the second polypeptide of each compound.

In some embodiments, each compound of the dimer is non-covalently bound to the other.

In some embodiments, the dimer is:

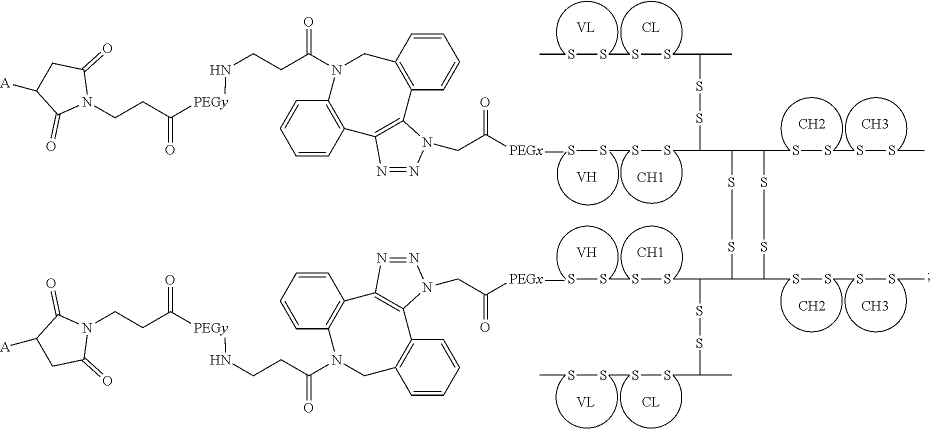

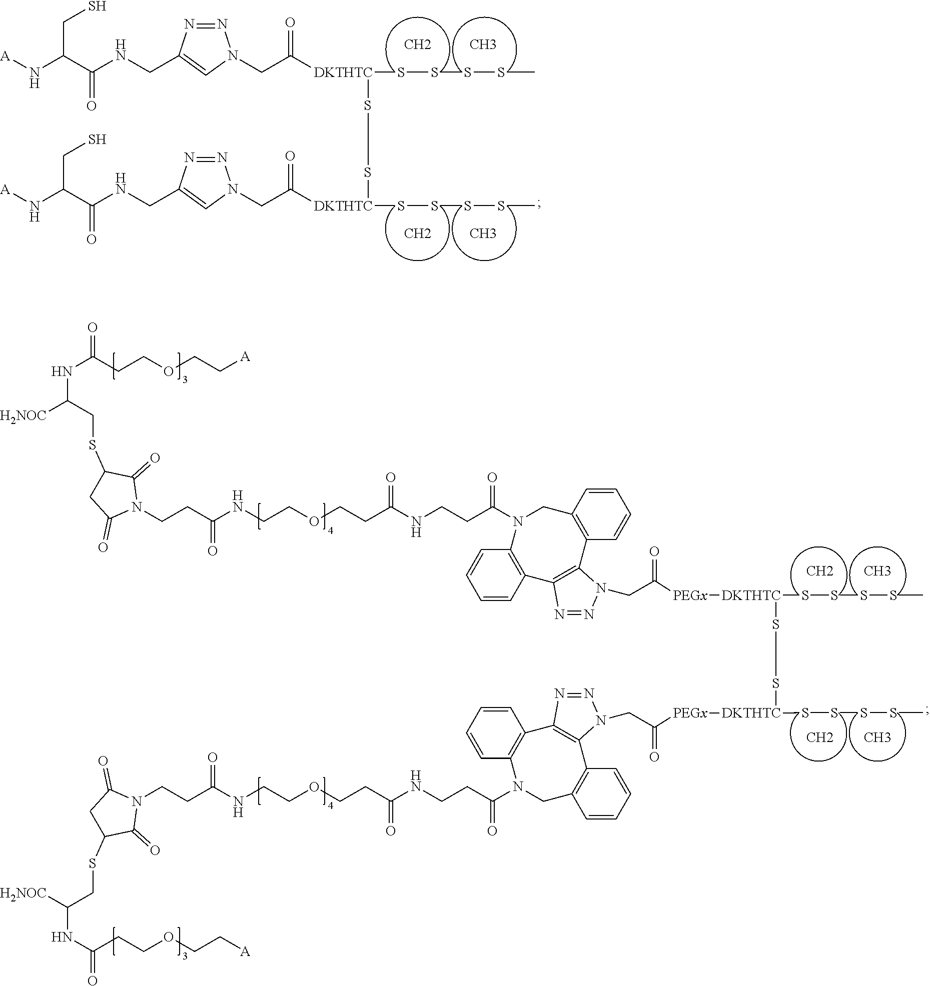

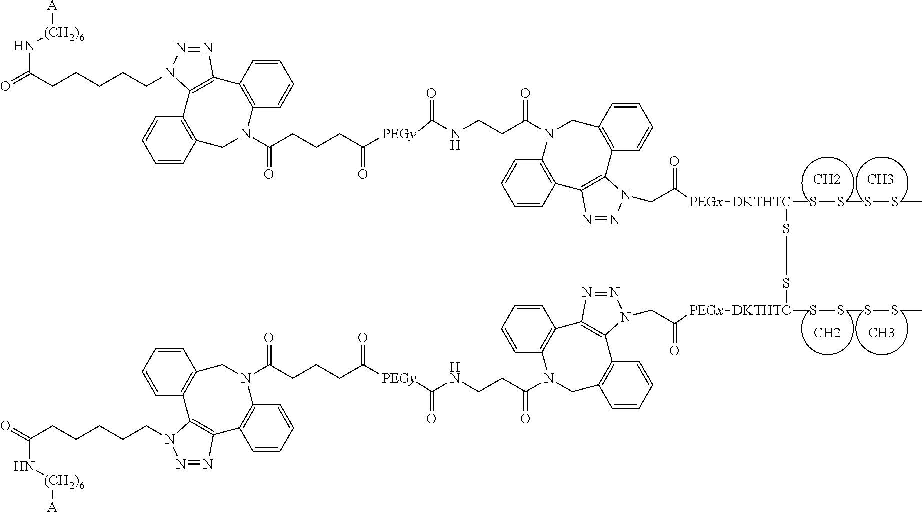

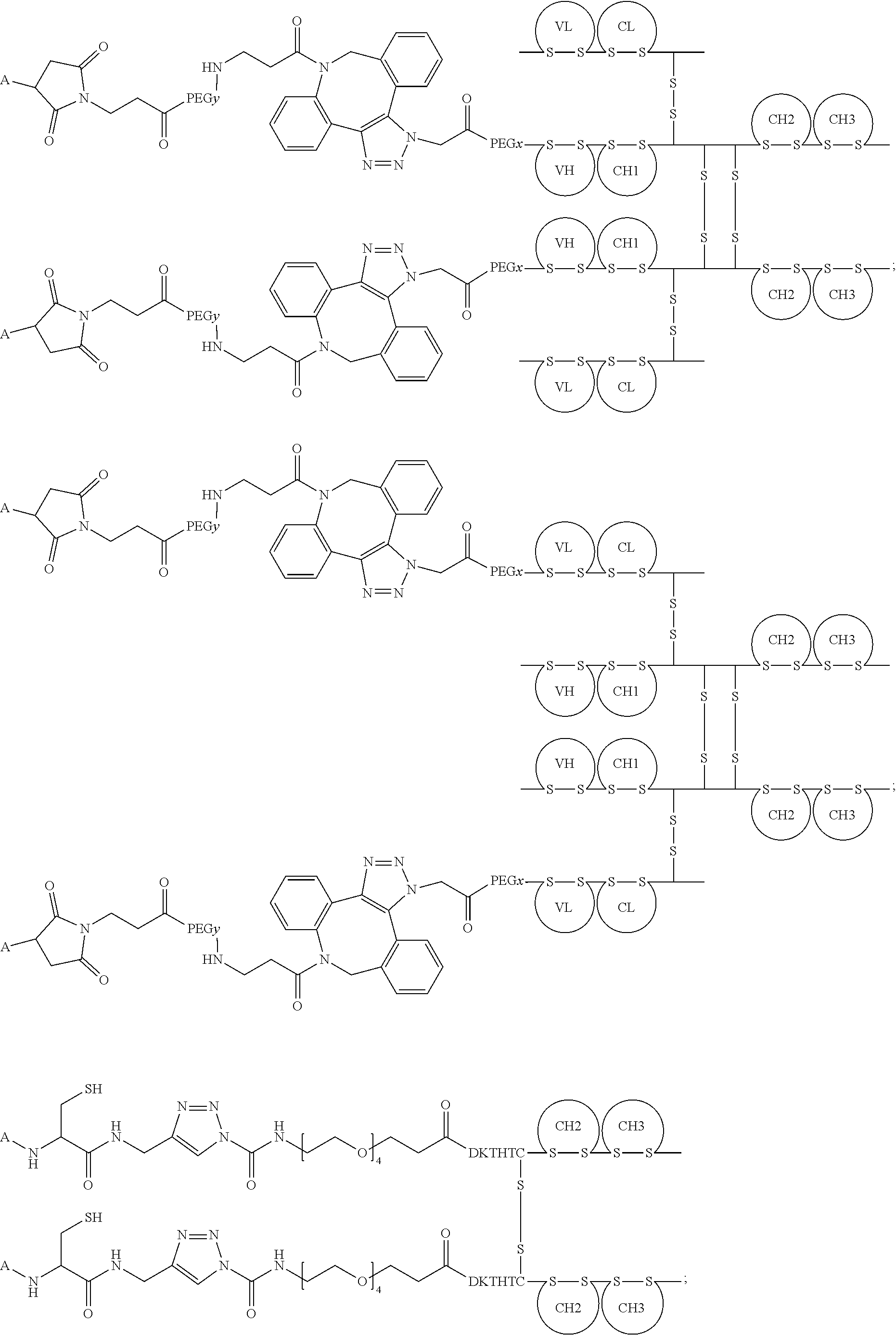

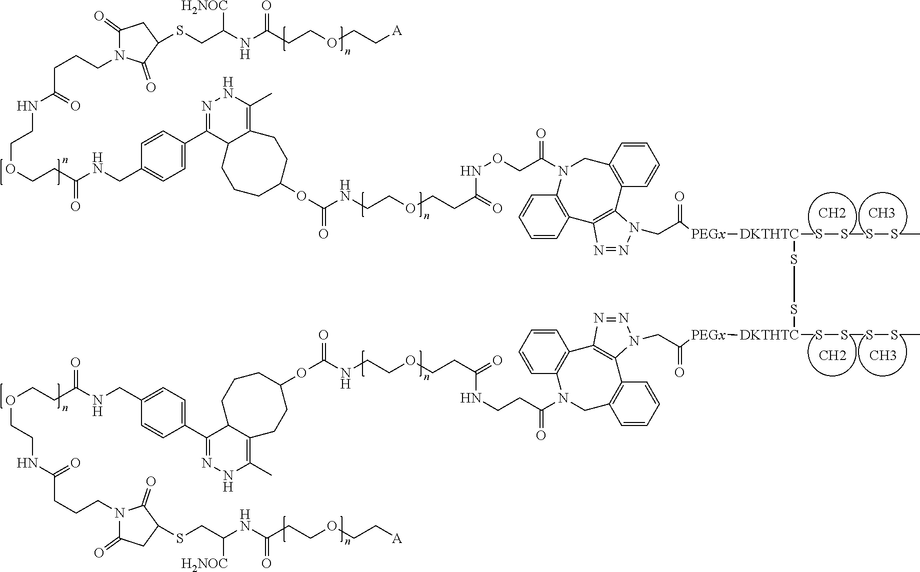

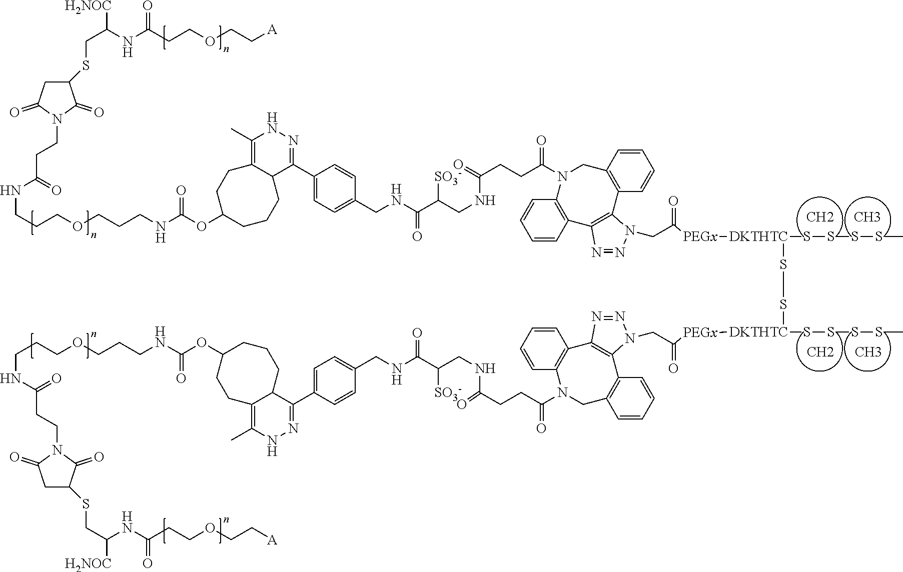

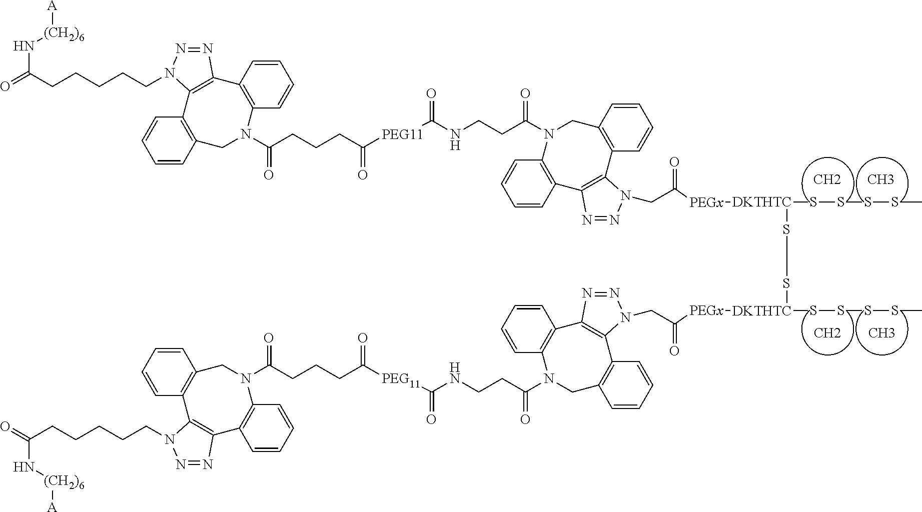

Additional non-limiting examples of dimers of compounds of the present invention are shown in the Figures.

In some embodiments, the branched residue is a lysine or a lysine derivative, arginine or an arginine derivative, aspartic acid or an aspartic acid derivative, glutamic acid or a glutamic acid derivative, asparagines or a asparagines derivative, glutamine or glutamine derivative, tyrosine or tyrosine derivative, cysteine or cysteine derivative or ornithine or ornithine derivative.

In some embodiments, the branched residue is an amino acid substituted at the N-position with a residue containing a terminal amino or carbonyl reactive group. In some embodiments, the branched residue is an organic residue containing two or more terminal amino groups or two or more terminal carbonyl groups.

In some embodiments, the branched residue is an organic residue containing two or more terminal amino groups. In some embodiments, the branched residue is an organic residue containing two or more terminal carbonyl groups. In some embodiments, the branched residue is a diaminopropionic acid. In some embodiments, the branched residue is a diaminopropionic carbonyl compound.

In some embodiments, the branched residue is 4-(carbonylmethoxy)phenylalanine, 2-amino-6-(carbonylmethylamino)hexanoic acid, S-(carbonylpropyl)cysteine, S-(carbonylethyl)cysteine, S-(carbonylmethyl)cysteine, N-(carbonylethyl)glycine, N-(carbonylmethyl)glycine, iminodipropionic acid, iminodiacetic acid, 4-amino-pimelic acid, 4-amino-heptanedioic acid, 3-aminohexanedioic acid, 3-aminoadipic acid, 2-aminooctanedioic acid, or 2-amino-6-carbonyl-heptanedioic acid.

In some embodiments, the branched residue is prepared from Fmoc-L-Asp-AMC, Fmoc-L-Asp-pNA, Fmoc-L-Glu-AMC, Fmoc-L-Glu-pNA, Fmoc-L-Glu(Edans)-OH, Fmoc-L-Glu(PEG-biotinyl)-OH, (S)-Fmoc-2-amino-hexanedioic acid-6-tert-butyl ester, (S)-Fmoc-2-amino-adipic acid-6-tert-butyl ester, (S)-Fmoc-Aad(OtBu)-OH, (S)-Fmoc-2-amino-5-tert-butoxycarbonyl-hexanedioic acid-6-tert-butyl ester, (S)-Fmoc-2-amino-heptanedioic acid-7-tert-butyl ester, (S)-Fmoc-2-amino-pimelic acid-7-tert-butyl ester, (S)-Fmoc-2-amino-6-tert-butoxycarbonyl-heptanedioic acid-7-tert-butyl ester, (S)-Fmoc-2-amino-octanedioic acid-8-tert-butyl ester, (S)-Fmoc-2-amino-suberic acid-8-tert-butyl ester, (S)-Fmoc-Asu(OtBu)-OH, (R)-Fmoc-3-amino-hexanedioic acid-1-tert-butyl ester, (R)-Fmoc-3-amino-adipic acid-1-tert-butyl ester, (R)-Fmoc-4-amino-heptanedioic acid-1-tert-butyl ester, (R)-Fmoc-4-amino-pimelic acid-1-tert-butyl ester, Boc-iminodiacetic acid, Fmoc-iminodiacetic acid, Boc-iminodipropionic acid, Fmoc-iminodipropionic acid, Fmoc-N-(tert-butoxycarbonylmethyl)-glycine, Fmoc-N-(tert-butoxycarbonylethyl)-glycine, Fmoc-L-Cys(tert-butoxycarbonylmethyl)-OH (R)-Fmoc-2-amino-3-(tert-butoxycarbonylmethylsulfanyl)-propionic acid, Fmoc-L-Cys(tert-butoxycarbonylpropyl)-OH (R)-Fmoc-2-amino-3-(3-tert-butoxycarbonylpropylsulfanyl)-propionic acid, Fmoc-L-Cys(tert-butoxycarbonylethyl)-OH (R)-Fmoc-2-amino-3-(2-tert-butoxycarbonylethylsulfanyl)-propionic acid, Fmoc-4-(tert-butoxycarbonylmethoxy)-L-phenylalanine, or (S)-Fmoc-2-amino-6-(Boc-tert-butoxycarbonylmethylamino)-hexanoic acid.

In some embodiments, the branched residue is prepared from N-α-Boc-DL-diaminopropionic acid, N-α-Boc-D-diaminopropionic acid, N-α-Boc-L-diaminopropionic acid, N-α-Fmoc-L-diaminopropionic acid, N-α-Boc-N-β-Alloc-D-diaminopropionic acid, N-α-Boc-N-β-Alloc-L-diaminopropionic acid, N-α-Fmoc-N-β-alloc-L-diaminopropionic acid, N-α-N-β-Bis-Boc-L-diaminopropionic acid, N-α-Fmoc-N-β-Boc-D-diaminopropionic acid, N-α-Fmoc-N-β-Boc-L-diaminopropionic acid, N-α-Z—N-β-Boc-L-diaminopropionic acid, N-α-Boc-N-β-Fmoc-D-diaminopropionic acid, N-α-Boc-N-β-Fmoc-L-diaminopropionic acid, N-α-N-β-Bis-Fmoc-L-diaminopropionic acid, N-α-Z—N-β-Fmoc-L-diaminopropionic acid, N-α-Boc-N-β-Z-L-diaminopropionic acid, N-α-Fmoc-N-β-Z-L-diaminopropionic acid, N-α-Fmoc-N-β-(Boc-aminooxyacetyl)-L-diaminopropionic acid, N-α-Boc-N-gamma-Fmoc-D-diaminobutyric acid, N-α-Boc-N-gamma-Fmoc-L-diaminobutyric acid, N-α-Boc-N-gamma-Fmoc-L-diaminobutyric acid, N-α-Fmoc-N-gamma-Boc-D-diaminobutyric acid, N-α-Fmoc-N-gamma-Boc-L-diaminobutyric acid, N-α-Fmoc-N-gamma-Alloc-L-diaminobutyric acid, (S)—N-b-Fmoc-N-gamma-Boc-3,4-diaminobutyric acid, H-L-ornithine, N-a-Boc-N-delta-Alloc-L-ornithine, N-a-Fmoc-N-delta-Alloc-L-ornithine, N-a-Fmoc-N-delta-Boc-L-ornithine, (S)-Boc-2-amino-5-azido-pentanoic acid·DCHA, (S)-Fmoc-2-amino-5-azido-pentanoic acid, N-a-N-delta-bis-Boc-N-a-N-delta-bis(3-Boc-aminopropyl)-L-ornithine, N-α-Boc-N-β-N-delta-N-delta-tris(3-Boc-aminopropyl)-L-ornithine, Fmoc-L-Lys(Biotin)-OH, Fmoc-L-Lys(Dabcyl)-OH, Fmoc-L-Lys(Boc) (Me)-OH, Fmoc-L-Lys(Boc) (iPr)-OH, (2S,5R)-Fmoc-2-amino-4-(3-Boc-2,2-dimethyl-oxazolidin-5-yl)-butyric acid, (S)-Fmoc-2-amino-6-(Boc-tert-butoxycarbonylmethyl-amino)-hexanoic acid, (S)-Fmoc-2-amino-7-(Boc-amino)-heptanoic acid, Fmoc-L-Arg(Me) (Pbf)-OH, Fmoc-L-Arg(Me)2(Pbf)-OH, Fmoc-L-Arg(Me)2-OH, (S)-Fmoc-3-amino-5-[(N′-Pbf-pyrrolidine-1-carboximidoyl)-amino]-pentanoic acid, Fmoc-L-Homoarg(Et)2-OH, Boc-3-amino-5-(Fmoc-amino)-benzoic acid, 3,5-bis[2-(Boc-amino)ethoxy]-benzoic acid, Fmoc-4-[2-(Boc-amino)ethoxy]-L-phenylalanine, N,N-bis(N′-Fmoc-3-aminopropyl)-glycine potassium hemisulfate, N,N-bis(N′-Fmoc-3-aminopropyl)-glycine potassium hemisulfate, Fmoc-N-(2-Boc-aminoethyl)-glycine, Fmoc-N-(3-Boc-aminopropyl)-glycine, Fmoc-N-(4-Boc-aminobutyl)-glycine, (R,S)—N-α-Fmoc-N-a′-Boc-diaminoacetic acid, N,N′-bis-Fmoc-diaminoacetic acid, (S)—N-4-Fmoc-N-8-Boc-diaminooctanoic acid, (R,S)—N-Fmoc-N′-Boc-imidazolidine-2-carboxylic acid, Fmoc-p(NH-Boc)-L-Phe-OH, Boc-p(NH-Fmoc)-L-Phe-OH, or Boc-p(NH—Z)-L-Phe-OH.

Each embodiment disclosed herein is contemplated as being applicable to each of the other disclosed embodiments. Thus, all combinations of the various elements described herein are within the scope of the invention.

It is understood that where a parameter range is provided, all integers within that range, and tenths thereof, are also provided by the invention. For example, “0.2-5 mg/kg/day” is a disclosure of 0.2 mg/kg/day, 0.3 mg/kg/day, 0.4 mg/kg/day, 0.5 mg/kg/day, 0.6 mg/kg/day etc. up to 5.0 mg/kg/day.

### Terms

As used herein, and unless stated otherwise, each of the following terms shall have the definition set forth below.

Peptidyl linkage: the structure

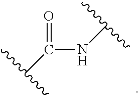 [SMILES: *NC(*)=O]

A peptidyl linkage may be a peptide bond.

Stretch of consecutive amino acids: a plurality of amino acids arranged in a chain, each of which is joined to a preceding amino acid by a peptide bond, excepting that the first amino acid in the chain may optionally not be joined to a preceding amino acid. The amino acids of the chain may be naturally or non-naturally occurring, or may comprise a mixture thereof. The amino acids, unless otherwise indicated, may be genetically encoded, naturally-occurring but not genetically encoded, or non-naturally occurring, and any selection thereof.

N-terminal amino acid residue: the terminal residue of a stretch of two or more consecutive amino acids having a free α-amino (NH₂) functional group, or a derivative of an α-amino (NH₂) functional group.

N-terminus: the free α-amino (NH₂) group (or derivative thereof) of a N-terminal amino acid residue.

C-terminal amino acid residue: the terminal residue of a stretch of two or more consecutive amino acids having a free α-carboxyl (COOH) functional group, or a derivative of a α-carboxyl (COOH) functional group.

C-terminus: the free α-carboxyl (COOH) group (or derivative thereof) of a C-terminal amino acid residue.

A “biologically active structure”, as used herein, means a structure of a molecule or fragment thereof, capable of treating a disease or condition or localizing or targeting a compound of the invention to a site of a disease or condition in the body by performing a function or an action, or stimulating or responding to a function, an action or a reaction, in a biological context (e.g. in an organism, a cell, or an in vitro model thereof). Biologically active structures may comprise a structure of at least one of polypeptides, nucleic acids, small molecules such as small organic or inorganic molecules.

A “bond”, unless otherwise specified, or contrary to context, is understood to include a covalent bond, a dipole-dipole interaction such as a hydrogen bond, and intermolecular interactions such as van der Waals forces.

A “Signal Sequence” is a short (3-60 amino acids long) peptide chain that directs the post-translational transport of a polypeptide.

“Amino acid” as used herein, in one embodiment, means a L or D isomer of the genetically encoded amino acids, i.e. isoleucine, alanine, leucine, asparagine, lysine, aspartate, methionine, cysteine, phenylalanine, glutamate, threonine, glutamine, tryptophan, glycine, valine, proline, arginine, serine, histidine, tyrosine, selenocysteine, pyrrolysine and also includes homocysteine and homoselenocysteine.

Other examples of amino acids include an L or D isomer of taurine, gaba, dopamine, lanthionine, 2-aminoisobutyric acid, dehydroalanine, ornithine and citrulline, as well as non-natural homologues and synthetically modified forms thereof including amino acids having alkylene chains shortened or lengthened by up to two carbon atoms, amino acids comprising optionally substituted aryl groups, and amino acids comprising halogenated groups, including halogenated alkyl and aryl groups as well as beta or gamma amino acids, and cyclic analogs.

Due to the presence of ionizable amino and carboxyl groups, the amino acids in these embodiments may be in the form of acidic or basic salts, or may be in neutral forms. Individual amino acid residues may also be modified by oxidation or reduction. Other contemplated modifications include hydroxylation of proline and lysine, phosphorylation of hydroxyl groups of seryl or threonyl residues, and methylation of the alpha-amino groups of lysine, arginine, and histidine side chains.

Covalent derivatives may be prepared by linking particular functional groups to the amino acid side chains or at the N- or C-termini.

Compounds comprising amino acids with R-group substitutions are within the scope of the invention. It is understood that substituents and substitution patterns on the compounds of the instant invention can be selected by one of ordinary skill in the art to provide compounds that are chemically stable from readily available starting materials.

“Natural amino acid” as used herein means a L or D isomer of the genetically encoded amino acids, i.e. isoleucine, alanine, leucine, asparagine, lysine, aspartate, methionine, cysteine, phenylalanine, glutamate, threonine, glutamine, tryptophan, glycine, valine, proline, arginine, serine, histidine, tyrosine, selenocysteine, pyrrolysine and homocysteine and homoselenocysteine.

“Non-natural amino acid” as used herein means a chemically modified L or D isomer of isoleucine, alanine, leucine, asparagine, lysine, aspartate, methionine, cysteine, phenylalanine, glutamate, threonine, glutamine, tryptophan, glycine, valine, proline, arginine, serine, histidine, tyrosine, selenocysteine, pyrrolysine, homocysteine, homoselenocysteine, taurine, gaba, dopamine, lanthionine, 2-aminoisobutyric acid, dehydroalanine, ornithine or citrulline, including cysteine and selenocysteine derivatives having C₃-C₁₀ aliphatic side chains between the alpha carbon and the S or Se. In one embodiment the aliphatic side chain is an alkylene. In another embodiment, the aliphatic side chain is an alkenylene or alkynylene.

In addition to the stretches of consecutive amino acid sequences described herein, it is contemplated that variants thereof can be prepared by introducing appropriate nucleotide changes into the encoding DNA, and/or by synthesis of the desired consecutive amino acid sequences. Those skilled in the art will appreciate that amino acid changes may alter post-translational processes of the stretches of consecutive amino acids described herein when expression is the chosen method of synthesis (rather than chemical synthesis for example), such as changing the number or position of glycosylation sites or altering the membrane anchoring characteristics.

Variations in the sequences described herein, can be made, for example, using any of the techniques and guidelines for conservative and non-conservative mutations set forth, for instance, in U.S. Pat. No. 5,364,934. Variations may be a substitution, deletion or insertion of one or more codons encoding the consecutive amino acid sequence of interest that results in a change in the amino acid sequence as compared with the native sequence. Optionally the variation is by substitution of at least one amino acid with any other amino acid in one or more of the domains. Guidance in determining which amino acid residue may be inserted, substituted or deleted without adversely affecting the desired activity may be found by comparing the sequence with that of homologous known protein molecules and minimizing the number of amino acid sequence changes made in regions of high homology. Amino acid substitutions can be the result of replacing one amino acid with another amino acid having similar structural and/or chemical properties, such as the replacement of a leucine with a serine, i.e., conservative amino acid replacements. Insertions or deletions may optionally be in the range of about 1 to 5 amino acids. The variation allowed may be determined by systematically making insertions, deletions or substitutions of amino acids in the sequence and testing the resulting variants for activity exhibited by the full-length or mature native sequence. It is understood that any terminal variations are made within the context of the invention disclosed herein.

Amino acid sequence variants of the binding partner are prepared with various objectives in mind, including increasing the affinity of the binding partner for its ligand, facilitating the stability, purification and preparation of the binding partner, modifying its plasma half life, improving therapeutic efficacy, and lessening the severity or occurrence of side effects during therapeutic use of the binding partner.

Amino acid sequence variants of these sequences are also contemplated herein including insertional, substitutional, or deletional variants. Such variants ordinarily can prepared by site-specific mutagenesis of nucleotides in the DNA encoding the target-binding monomer, by which DNA encoding the variant is obtained, and thereafter expressing the DNA in recombinant cell culture. Fragments having up to about 100-150 amino acid residues can also be prepared conveniently by in vitro synthesis. Such amino acid sequence variants are predetermined variants and are not found in nature. The variants exhibit the qualitative biological activity (including target-binding) of the nonvariant form, though not necessarily of the same quantative value. While the site for introducing an amino acid sequence variation is predetermined, the mutation per se need not be predetermined. For example, in order to optimize the performance of a mutation at a given site, random or saturation mutagenesis (where all 20 possible residues are inserted) is conducted at the target codon and the expressed variant is screened for the optimal combination of desired activities. Such screening is within the ordinary skill in the art.

Amino acid insertions usually will be on the order of about from 1 to 10 amino acid residues; substitutions are typically introduced for single residues; and deletions will range about from 1 to 30 residues. Deletions or insertions preferably are made in adjacent pairs, i.e. a deletion of 2 residues or insertion of 2 residues. It will be amply apparent from the following discussion that substitutions, deletions, insertions or any combination thereof are introduced or combined to arrive at a final construct.

In an aspect, the invention concerns a compound comprising a stretch of consecutive amino acids having at least about 80% sequence identity, preferably at least about 81% sequence identity, more preferably at least about 82% sequence identity, yet more preferably at least about 83% sequence identity, yet more preferably at least about 84% sequence identity, yet more preferably at least about 85% sequence identity, yet more preferably at least about 86% sequence identity, yet more preferably at least about 87% sequence identity, yet more preferably at least about 88% sequence identity, yet more preferably at least about 89% sequence identity, yet more preferably at least about 90% sequence identity, yet more preferably at least about 91% sequence identity, yet more preferably at least about 92% sequence identity, yet more preferably at least about 93% sequence identity, yet more preferably at least about 94% sequence identity, yet more preferably at least about 95% sequence identity, yet more preferably at least about 96% sequence identity, yet more preferably at least about 97% sequence identity, yet more preferably at least about 98% sequence identity and yet more preferably at least about 99% sequence identity to an amino acid sequence disclosed in the specification, a figure, a SEQ ID NO. or a sequence listing of the present application.

The % amino acid sequence identity values can be readily obtained using, for example, the WU-BLAST-2 computer program (Altschul et al., Methods in Enzymology 266:460-480 (1996)).

Fragments of native sequences are provided herein. Such fragments may be truncated at the N-terminus or C-terminus, or may lack internal residues, for example, when compared with a full length native protein. Again, it is understood that any terminal variations are made within the context of the invention disclosed herein.

Certain fragments lack amino acid residues that are not essential for a desired biological activity of the sequence of interest.

Any of a number of conventional techniques may be used. Desired peptide fragments or fragments of stretches of consecutive amino acids may be chemically synthesized. An alternative approach involves generating fragments by enzymatic digestion, e.g. by treating the protein with an enzyme known to cleave proteins at sites defined by particular amino acid residues, or by digesting the DNA with suitable restriction enzymes and isolating the desired fragment. Yet another suitable technique involves isolating and amplifying a DNA fragment encoding a desired polypeptide/sequence fragment, by polymerase chain reaction (PCR). Oligonucleotides that define the desired termini of the DNA fragment are employed at the 5′ and 3′ primers in the PCR.

In particular embodiments, conservative substitutions of interest are shown in Table A under the heading of preferred substitutions. If such substitutions result in a change in biological activity, then more substantial changes, denominated exemplary substitutions in Table A, or as further described below in reference to amino acid classes, are introduced and the products screened.

TABLE A

| Original | Exemplary | Preferred |
|---|---|---|
| Ala (A) | val; leu; ile | val |
| Arg (R) | lys; gln; asn | lys |
| Asn (N) | gln; his; lys; arg | gln |
| Asp (D) | glu | glu |
| Cys (C) | ser | ser |
| Gln (Q) | asn | asn |
| Glu (E) | asp | asp |
| Gly (G) | pro; ala | ala |
| His (H) | asn; gln; lys; arg | arg |
| Ile (I) | leu; val; met; ala; phe; norleucine | leu |
| Leu (L) | norleucine; ile; val; met; ala; phe | ile |
| Lys (K) | arg; gln; asn | arg |
| Met (M) | leu; phe; ile | leu |
| Phe (F) | leu; val; ile; ala; tyr | leu |
| Pro (P) | ala | ala |
| Ser (S) | thr | thr |
| Thr (T) | ser | ser |
| Trp (W) | tyr; phe | tyr |
| Tyr (Y) | trp; phe; thr; ser | phe |
| Val (V) | ile; leu; met; phe; ala; norleucine | leu |

Substantial modifications in function or immunological identity of the sequence are accomplished by selecting substitutions that differ significantly in their effect on maintaining (a) the structure of the polypeptide backbone in the area of the substitution, for example, as a sheet or helical conformation, (b) the charge or hydrophobicity of the molecule at the target site, or (c) the bulk of the side chain. Naturally occurring residues are divided into groups based on common side-chain properties:
(1) hydrophobic: norleucine, met, ala, val, leu, ile;
(2) neutral hydrophilic: cys, ser, thr;
(3) acidic: asp, glu;
(4) basic: asn, gln, his, lys, arg;
(5) residues that influence chain orientation: gly, pro;
(6) aromatic: trp, tyr, phe.

Non-conservative substitutions will entail exchanging a member of one of these classes for another class. Such substituted residues also may be introduced into the conservative substitution sites or, more preferably, into the remaining (non-conserved) sites.

The variations can be made using methods known in the art such as oligonucleotide-mediated (site-directed) mutagenesis, alanine scanning, and PCR mutagenesis. Site-directed mutagenesis (Carter et al., Nucl. Acids Res., 13:4331 (1986); Zoller et al., Nucl. Acids Res., 10:6487 (1987)), cassette mutagenesis (Wells et al., Gene, 34:315 (1985)), restriction selection mutagenesis (Wells et al., Philos. Trans. R. Soc. London SerA, 317:415 (1986)) or other known techniques can be performed on the cloned DNA to produce the variant DNA.

Scanning amino acid analysis can also be employed to identify one or more amino acids along a contiguous sequence. Among the preferred scanning amino acids are relatively small, neutral amino acids. Such amino acids include alanine, glycine, serine, and cysteine. Alanine is typically a preferred scanning amino acid among this group because it eliminates the side-chain beyond the beta-carbon and is less likely to alter the main-chain conformation of the variant (Cunningham and Wells, Science, 244:1081-1085 (1989)). Alanine is also typically preferred because it is the most common amino acid. Further, it is frequently found in both buried and exposed positions (Creighton, The Proteins, (W.H. Freeman & Co., N.Y.); Chothia, J. Mol. Biol., 150:1 (1976)). If alanine substitution does not yield adequate amounts of variant, an isoteric amino acid can be used.

Covalent modifications: The stretch of consecutive amino acids may be covalently modified. One type of covalent modification includes reacting targeted amino acid residues with an organic derivatizing agent that is capable of reacting with selected side chains or the N- or C-terminal residues that are not involved in an -x-x- bond. Derivatization with bifunctional agents is useful, for instance, for crosslinking to a water-insoluble support matrix or surface for use in the method for purifying anti-sequence of interest antibodies, and vice-versa. Commonly used crosslinking agents include, e.g., 1,1-bis(diazoacetyl)-2-phenylethane, glutaraldehyde, N-hydroxysuccinimide esters, for example, esters with 4-azidosalicylic acid, homobifunctional imidoesters, including disuccinimidyl esters such as 3,3′-dithiobis(succinimidylpropionate), bifunctional maleimides such as bis-N-maleimido-1,8-octane and agents such as methyl-3-((p-azidophenyl)dithio)propioimidate.

Other modifications include deamidation of glutaminyl and asparaginyl residues to the corresponding glutamyl and aspartyl residues, respectively, hydroxylation of proline and lysine, phosphorylation of hydroxyl groups of seryl or threonyl residues, methylation of the .alpha.-amino groups of lysine, arginine, and histidine side chains (T. E. Creighton, Proteins: Structure and Molecular Properties, W.H. Freeman & Co., San Francisco, pp. 79-86 (1983)), acetylation of the N-terminal amine, and amidation of any C-terminal carboxyl group.

Another type of covalent modification comprises altering the native glycosylation pattern of the stretch of consecutive amino acids. “Altering the native glycosylation pattern” is intended for purposes herein to mean deleting one or more carbohydrate moieties found in amino acid sequences (either by removing the underlying glycosylation site or by deleting the glycosylation by chemical and/or enzymatic means), and/or adding one or more glycosylation sites that are not present in the native sequence. In addition, the phrase includes qualitative changes in the glycosylation of the native proteins, involving a change in the nature and proportions of the various carbohydrate moieties present.

Addition of glycosylation sites to the amino acid sequence may be accomplished by altering the amino acid sequence. The alteration may be made, for example, by the addition of, or substitution by, one or more serine or threonine residues to the native sequence (for O-linked glycosylation sites). The amino acid sequence may optionally be altered through changes at the DNA level, particularly by mutating the DNA encoding the amino acid sequence at preselected bases such that codons are generated that will translate into the desired amino acids.

Another means of increasing the number of carbohydrate moieties on the amino acid sequence is by chemical or enzymatic coupling of glycosides to the polypeptide. Such methods are described in the art, e.g., in WO 87/05330 published Sep. 11, 1987, and in Aplin and Wriston, CRC Crit. Rev. Biochem., pp. 259-306 (1981).

Removal of carbohydrate moieties present on the amino acid sequence may be accomplished chemically or enzymatically or by mutational substitution of codons encoding for amino acid residues that serve as targets for glycosylation. Chemical deglycosylation techniques are known in the art and described, for instance, by Hakimuddin, et al., Arch. Biochem. Biophys., 259:52 (1987) and by Edge et al., Anal. Biochem., 118:131 (1981). Enzymatic cleavage of carbohydrate moieties on polypeptides can be achieved by the use of a variety of endo- and exo-glycosidases as described by Thotakura et al., Meth. Enzymol., 138:350 (1987).

Another type of covalent modification comprises linking the amino acid sequence to one of a variety of nonproteinaceous polymers, e.g., polyethylene glycol (PEG), polypropylene glycol, or polyoxyalkylenes, in the manner set forth in U.S. Pat. Nos. 4,640,835; 4,496,689; 4,301,144; 4,670,417; 4,791,192 or 4,179,337.

The term “substitution”, “substituted” and “substituent” refers to a functional group in which one or more bonds to a hydrogen atom contained therein are replaced by a bond to non-hydrogen atoms, provided that normal valencies are maintained and that the substitution results in a stable compound. Substituted groups also include groups in which one or more bonds to a carbon(s) or hydrogen(s) atom are replaced by one or more bonds, including double or triple bonds, to a heteroatom. Examples of substituent groups include halogens (i.e., F, Cl, Br, and I); alkyl groups, such as methyl, ethyl, n-propyl, isopropryl, n-butyl, tert-butyl, and trifluoromethyl; aryl groups, such as phenyl; heteroaryl groups, such as triazole, dihydropyridazine and tetrazole; hydroxyl; alkoxy groups, such as methoxy, ethoxy, n-propoxy, and isopropoxy; aryloxy groups, such as phenoxy; arylalkyloxy, such as benzyloxy (phenylmethoxy) and p-trifluoromethylbenzyloxy (4-trifluoromethylphenylmethoxy); heteroaryloxy groups; sulfonyl groups, such as sulfonate, trifluoromethanesulfonyl, methanesulfonyl, and p-toluenesulfonyl; sulfnitro, nitrosyl; mercapto; sulfanyl groups, such as methylsulfanyl, ethylsulfanyl and propylsulfanyl; cyano; amino groups, such as amino, methylamino, dimethylamino, ethylamino, and diethylamino; and carboxyl. Where multiple substituent moieties are disclosed or claimed, the substituted compound can be independently substituted by one or more of the disclosed or claimed substituent moieties, singly or plurally.

By independently substituted, it is meant that the (two or more) substituents can be the same or different. In the compounds used in the method of the present invention, alkyl, heteroalkyl, monocycle, bicycle, aryl, heteroaryl and heterocycle groups can be substituted by replacing one or more hydrogen atoms with alternative non-hydrogen groups. These include, but are not limited to, halo, hydroxy, mercapto, amino, carboxy, cyano and carbamoyl.

It is understood that substituents and substitution patterns on the compounds used in the method of the present invention can be selected by one of ordinary skill in the art to provide compounds that are chemically stable and that can be readily synthesized by techniques known in the art from readily available starting materials. If a substituent is itself substituted with more than one group, it is understood that these multiple groups may be on the same carbon or on different carbons, so long as a stable structure results.

In choosing the compounds used in the method of the present invention, one of ordinary skill in the art will recognize that the various substituents, i.e. R₁, R₂, etc. are to be chosen in conformity with well-known principles of chemical structure connectivity.

As used herein, “alkyl” includes both branched and straight-chain saturated aliphatic hydrocarbon groups having the specified number of carbon atoms and may be unsubstituted or substituted. Thus, C₁-Cₙ as in “C₁-Cₙ alkyl” is defined to include groups having 1, 2, . . . , n−1 or n carbons in a linear or branched arrangement. For example, C₁-C₆, as in “C₁-C₆ alkyl” is defined to include groups having 1, 2, 3, 4, 5, or 6 carbons in a linear or branched arrangement, and specifically includes methyl, ethyl, n-propyl, isopropyl, n-butyl, t-butyl, pentyl, and hexyl. Unless otherwise specified contains one to twelve carbons. Alkyl groups can be unsubstituted or substituted with one or more substituents, including but not limited to halogen, alkoxy, alkylthio, trifluoromethyl, difluoromethyl, methoxy, and hydroxyl. An embodiment can be C₁-C₁₂ alkyl, C₂-C₁₂ alkyl, C₃-C₁₂ alkyl, C₄-C₁₂ alkyl and so on. An embodiment can be C₁-C₈ alkyl, C₂-C₈ alkyl, C₃-C₈ alkyl, C₄-C₈ alkyl and so on. Alkyl is intended to include moieties that are monovalent, divalent, trivalent, etc.

As used herein, “C₁-C₄ alkyl” includes both branched and straight-chain C₁-C₄ alkyl.

As used herein, the term “cycloalkane” refers to a monocyclic or bicyclic ring system, which may be unsaturated or partially unsaturated, i.e. possesses one or more double bonds. Monocyclic ring systems are exemplified by a saturated cyclic hydrocarbon group containing from 3 to 8 carbon atoms. Examples of monocyclic ring systems include cyclopropyl, cyclobutyl, cyclopentyl, cyclopentenyl, cyclohexyl, cyclohexenyl, cycloheptyl and cyclooctyl. Bicyclic fused ring systems are exemplified by a cycloalkyl ring fused to another cycloalkyl ring. Examples of bicyclic fused ring systems include, but are not limited to, decalin, 1,2,3,7,8,8a-hexahydro-naphthalene, and the like. Thus, C₃-C₁₀ cycloalkane includes cyclic rings of alkanes of three to eight total carbon atoms, (e.g., cyclopropyl, cyclobutyl, cyclopentyl, cyclohexyl, cycloheptyl or cyclooctyl and so on). Cycloalkane groups can be unsubstituted or substituted with one or more substituents, including but not limited to halogen, alkoxy, alkylthio, trifluoromethyl, difluoromethyl, methoxy, and hydroxyl. Cycloalkane is intended to include moieties that are monovalent, divalent, trivalent, etc.

As used herein, the term “cycloalkene” refers to a cycloalkane which possesses one or more double bonds. Thus, C₅-C₁₀ cycloalkene includes cyclic rings of alkanes of five to ten total carbon atoms, (e.g., cyclopentenyl, cyclohexenyl, cycloheptenyl, cyclohexadienyl, cyclooctenyl or cyclooctadienyl and so on). Cycloalkene is intended to moieties that are monovalent, divalent, trivalent, etc. Cycloalkene is intended to include moieties that are monovalent, divalent, trivalent, etc.

As used herein, “alkene” includes both branched and straight-chain aliphatic hydrocarbon groups having one or more double bond and the specified number of carbon atoms and may be unsubstituted or substituted. Thus, C₂-Cₙ as in “C₂-Cₙ alkene” is defined to include groups having 2, 3, . . . , n−1 or n carbons in a linear or branched arrangement. For example, C₂-C₁₀, as in “C₂-C₁₀ alkene” is defined to include groups having 2, 3, 4, 5 . . . 10 carbons in a linear or branched arrangement, and specifically includes vinyl, allyl, 1-butene, 2-butene, iso-butene, 1-pentene, 2-pentene, etc. Alkyene groups can be unsubstituted or substituted with one or more substituents, including but not limited to halogen, alkoxy, alkylthio, trifluoromethyl, difluoromethyl, methoxy, and hydroxyl. An embodiment can be C₂-C₃ alkene, C₂-C₄ alkene, C₂-C₅ alkene, and so on. Alkene is intended to include moieties that are monovalent, divalent, trivalent, etc.

As used herein, an “acyl” refers to an alkyl group having a ketone at the first position. For example, an “acyl” embodiment can be acetyl, propionyl, butyryl and valeryl. As another example, an “acyl” embodiment can be:

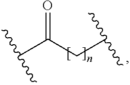

wherein n is 1-10. In another embodiment, n is 1-4.

Thus, a “C₂-C₅ acyl” can be acetyl, propionyl, butyryl, or and valeryl. Acyl is intended to include moieties that are monovalent, divalent, trivalent, etc.

C₂-C₅ acylamino is an acyl group as defined above further substituted with an amine. The amine may be linked to the carbonyl portion of the acyl group so as to form an amide or the amine may linked to a non-carbonyl portion of the acyl group. For example, the amino group may be at the alpha-position, the beta-position, the gamma-position, the delta-position, etc. As further examples, acylamino includes both alpha-aminoacetyl and acetamido groups. Acylamino includes beta-aminopropionyl).

C₂-C₅ acyloxy is an acyl group as defined above further substituted with an oxygen. The oxygen may be linked to the carbonyl portion of the acyl group so as to form an amide or the oxygen may linked to a non-carbonyl portion of the acyl group. For example, the oxygen group may be at the alpha-position, the beta-position, the gamma-position, the delta-position, etc. As further examples, acyloxy includes both alpha-oxyacetyl and acetate groups. Acyloxy includes beta-oxypropionyl).

As used herein, “amino” includes primary, secondary, tertiary and quarternary amines. Thus, amino includes a —NH— group, a —NH₂ group, a —NR— group, a —NR₂⁺— group, a —NRH⁺— group, a —NH₂⁺— group, a —NH₃⁺ group and a —NR₃⁺ group, wherein R is alkyl or aryl. Amino is intended to include moieties that are monovalent, divalent, trivalent, etc.

As used herein, “sulfur” includes a —S— group and a —SH group. The term sulfur is intended to include moieties that are monovalent, divalent, trivalent, etc.

As used herein, “oxygen” includes a —O— group and a —OH group. The term sulfur is intended to moieties that are monovalent and divalent.

As used herein, “succinyl” is derived from succinic acid by removal of one or both hydroxyl groups. An embodiment can be —C(O)—CH₂—CH₂—C(O)—. Succinyl is intended to include moieties that are monovalent, divalent, trivalent, etc.

As used herein, a “malonyl” is derived from malonic acid by removal of one or both hydroxyl groups. An embodiment can be —C(O)—CH₂—C(O)—. Malonyl is intended to include moieties that are monovalent, divalent, trivalent, etc.

As used herein, a “glutaryl” is derived from glutaric acid by removal of one or both hydroxyl groups. An embodiment can be —C(O)—CH₂—CH₂—CH₂—C(O)—. Glutaryl is intended to include moieties that are monovalent, divalent, trivalent, etc.

As used herein, an “adipoyl” is derived from adipic acid by removal of one or both hydroxyl groups. An embodiment can be —C(O)—CH₂—CH₂—CH₂—CH₂—(O)—. Adipoyl is intended to include moieties that are monovalent, divalent, trivalent, etc.

A “polyalkylene glycol” is derived from polyalkylene glycol by removal of both hydrogens from the hydroxyl groups. An embodiment can be derived from polyethylene glycol, polypropylene glycol, or polybutylene glycol.

An “polyalkylene glycol” embodiment can be

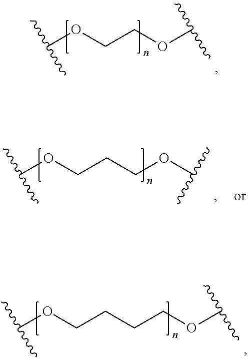

wherein n is 1-10.

As used herein, “aryl” is intended to mean any stable monocyclic, bicyclic or polycyclic carbon ring of up to 10 atoms in each ring, wherein at least one ring is aromatic, and may be unsubstituted or substituted. Examples of such aryl elements include but are not limited to: phenyl, p-toluenyl (4-methylphenyl), naphthyl, tetrahydro-naphthyl, indanyl, phenanthryl, anthryl or acenaphthyl. In cases where the aryl substituent is bicyclic and one ring is non-aromatic, it is understood that attachment is via the aromatic ring.

As used herein, “aryl” is intended to mean any stable monocyclic, bicyclic or polycyclic carbon ring of up to 10 atoms in each ring, wherein at least one ring is aromatic, and may be unsubstituted or substituted. Examples of such aryl elements include but are not limited to: phenyl, p-toluenyl (4-methylphenyl), naphthyl, tetrahydro-naphthyl, indanyl, phenanthryl, anthryl or acenaphthyl. In cases where the aryl substituent is bicyclic and one ring is non-aromatic, it is understood that attachment is via the aromatic ring.

The term “heteroaryl”, as used herein, represents a stable monocyclic, bicyclic or polycyclic ring of up to 10 atoms in each ring, wherein at least one ring is aromatic and contains from 1 to 4 heteroatoms selected from the group consisting of O, N and S. Bicyclic aromatic heteroaryl groups include phenyl, pyridine, pyrimidine or pyridizine rings that are (a) fused to a 6-membered aromatic (unsaturated) heterocyclic ring having one nitrogen atom; (b) fused to a 5- or 6-membered aromatic (unsaturated) heterocyclic ring having two nitrogen atoms; (c) fused to a 5-membered aromatic (unsaturated) heterocyclic ring having one nitrogen atom together with either one oxygen or one sulfur atom; or (d) fused to a 5-membered aromatic (unsaturated) heterocyclic ring having one heteroatom selected from O, N or S. Heteroaryl groups within the scope of this definition include but are not limited to: benzoimidazolyl, benzofuranyl, benzofurazanyl, benzopyrazolyl, benzotriazolyl, benzothiophenyl, benzoxazolyl, carbazolyl, carbolinyl, cinnolinyl, dihydropyridizine, furanyl, indolinyl, indolyl, indolazinyl, indazolyl, isobenzofuranyl, isoindolyl, isoquinolyl, isothiazolyl, isoxazolyl, naphthpyridinyl, oxadiazolyl, oxazolyl, oxazoline, isoxazoline, oxetanyl, pyranyl, pyrazinyl, pyrazolyl, pyridazinyl, pyridopyridinyl, pyridazinyl, pyridyl, pyrimidyl, pyrrolyl, quinazolinyl, quinolyl, quinoxalinyl, tetrazolyl, tetrazolopyridyl, thiadiazolyl, thiazolyl, thienyl, triazolyl, azetidinyl, aziridinyl, 1,4-dioxanyl, hexahydroazepinyl, dihydrobenzoimidazolyl, dihydrobenzofuranyl, dihydrobenzothiophenyl, dihydrobenzoxazolyl, dihydrofuranyl, dihydroimidazolyl, dihydroindolyl, dihydroisooxazolyl, dihydroisothiazolyl, dihydrooxadiazolyl, dihydrooxazolyl, dihydropyrazinyl, dihydropyrazolyl, dihydropyridinyl, dihydropyrimidinyl, dihydropyrrolyl, dihydroquinolinyl, dihydrotetrazolyl, dihydrothiadiazolyl, dihydrothiazolyl, dihydrothienyl, dihydrotriazolyl, dihydroazetidinyl, methylenedioxybenzoyl, tetrahydrofuranyl, tetrahydrothienyl, acridinyl, carbazolyl, cinnolinyl, quinoxalinyl, pyrrazolyl, indolyl, benzotriazolyl, benzothiazolyl, benzoxazolyl, isoxazolyl, isothiazolyl, furanyl, thienyl, benzothienyl, benzofuranyl, quinolinyl, isoquinolinyl, oxazolyl, isoxazolyl, indolyl, pyrazinyl, pyridazinyl, pyridinyl, pyrimidinyl, pyrrolyl, tetra-hydroquinoline. In cases where the heteroaryl substituent is bicyclic and one ring is non-aromatic or contains no heteroatoms, it is understood that attachment is via the aromatic ring or via the heteroatom containing ring, respectively. If the heteroaryl contains nitrogen atoms, it is understood that the corresponding N-oxides thereof are also encompassed by this definition.

The term “phenyl” is intended to mean an aromatic six membered ring containing six carbons, and any substituted derivative thereof.

The term “benzyl” is intended to mean a methylene attached directly to a benzene ring. A benzyl group is a methyl group wherein a hydrogen is replaced with a phenyl group, and any substituted derivative thereof.

The term “triazole” is intended to mean a heteraryl having a five-membered ring containing two carbon atoms and three nitrogen atoms, and any substituted derivative thereof.

Dihydropyradizine is optionally substituted and includes 1,2-dihydropyridazines,

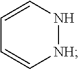

1,4-dihydropyridazines,

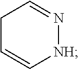

1,6-dihydropyridazines,

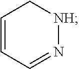

and 4,5-dihydropyridazines,

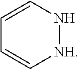

A chemical structure containing a cyclooctane fused to a dihydropyridazine includes, but is not limited to, a chemical structure which contains a cyclooctane fused to the 3rd and 4th position of a dihydropyridazine or a chemical structure which contains a saturated cycloocta[d]pyridazine, any of which are optionally substituted. For example, the chemical structure containing a cyclooctane fused to a dihydropyridazine includes, but is not limited to, a chemical structure which contains a 2,4a,5,6,7,8,9,10-octahydrocycloocta[d]pyridazine,

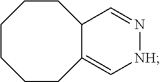 [SMILES: C1=NNC=C2CCCCCCC12]

a 4a,5,6,7,8,9,10,10a-octahydrocycloocta[d]pyridazine,

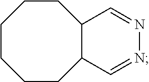 [SMILES: C1=NN=CC2CCCCCCC12]

a 2,3,5,6,7,8,9,10-octahydrocycloocta[d]pyridazine,

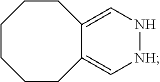 [SMILES: C1=C2CCCCCCC2=CNN1]

or a 1,2,5,6,7,8,9,10-octahydrocycloocta[d]pyridazine,

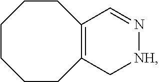 [SMILES: C1=NNCC2=C1CCCCCC2]

each of which may be optionally substituted.

Tautomers of

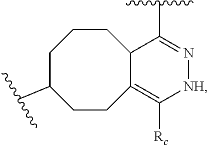

include, but are not limited to:

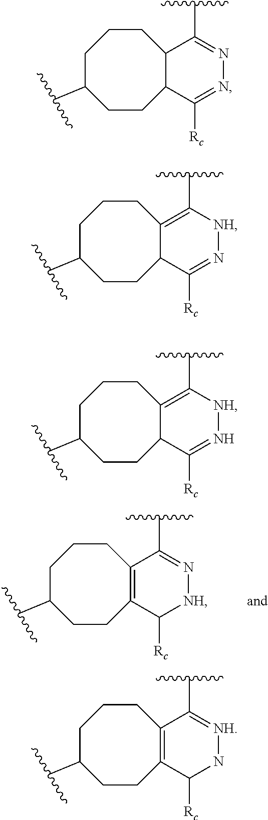

In some embodiments, the dihydropyridazine is oxidized to a pyridazine.

In some embodiments, the dihydropyridazine is reduced to result in an open ring structure having a 1,4-dicarbonyl compound.

The compounds used in the method of the present invention may be prepared by techniques well know in organic synthesis and familiar to a practitioner ordinarily skilled in the art. However, these may not be the only means by which to synthesize or obtain the desired compounds.

Compounds of the subject invention can be converted to prodrugs to optimize absorption and bioavailability. Formation of a prodrug include, but is not limited to, reaction of a free hydroxyl group with a carboxylic acid to form an ester, reaction of a free hydroxyl group with an phosphorus oxychloride followed by hydrolysis to form a phosphate, or reaction of a free hydroxyl group with an amino acid to form an amino acid ester, the process of which has been described previously by Chandran in WO 2005/046575. The substituents are chosen and resulting analogs are evaluated according to principles well known in the art of medicinal and pharmaceutical chemistry, such as quantification of structure-activity relationships, optimization of biological activity and ADMET (absorption, distribution, metabolism, excretion, and toxicity) properties.

The various R groups attached to the aromatic rings of the compounds disclosed herein may be added to the rings by standard procedures, for example those set forth in Advanced Organic Chemistry: Part B: Reaction and Synthesis, Francis Carey and Richard Sundberg, (Springer) 5th ed. Edition. (2007), the content of which is hereby incorporated by reference.

The compounds of present invention may be prepared by techniques described in Vogel's Textbook of Practical Organic Chemistry, A. I. Vogel, A. R. Tatchell, B. S. Furnis, A. J. Hannaford, P. W. G. Smith, (Prentice Hall) 5^{th} Edition (1996), March's Advanced Organic Chemistry: Reactions, Mechanisms, and Structure, Michael B. Smith, Jerry March, (Wiley-Interscience) 5^{th} Edition (2007), and references therein, which are incorporated by reference herein. However, these may not be the only means by which to synthesize or obtain the desired compounds.

A person having ordinary skill in the art will immediately understand that the definitions of the substituents and moieties (e.g. the moieties of J, Ra and Rb) provided herein are intended to obey the standard rules of chemical valency. For example, where a structure provided herein requires a particular substituent or moiety to be divalent, (e.g. a moiety in a linear chain of moieties) a person having ordinary skill in the art will immediately understand that the definitions of that substituent or moiety are divalent in order to obey the standard rules of chemical valency.

A person having ordinary skill in the art will immediately understand that some divalent moieties depicted in the present invention may be linked to other chemical structures in more than one way, e.g., the depicted structures may be linked to other chemical structures when rotated or flipped.

In some embodiments of the present invention, a compound comprises a nonproteinaceous polymer. In some embodiments, the nonproteinaceous polymer may be is a hydrophilic synthetic polymer, i.e., a polymer not otherwise found in nature. However, polymers which exist in nature and are produced by recombinant or in vitro methods are useful, as are polymers which are isolated from nature. Hydrophilic polyvinyl polymers fall within the scope of this invention, e.g. polyvinylalcohol and polyvinylpyrrolidone. Particularly useful are polyalkylene ethers such as polyethylene glycol, polypropylene glycol, polyoxyethylene esters or methoxy polyethylene glycol; polyoxyalkylenes such as polyoxyethylene, polyoxypropylene, and block copolymers of polyoxyethylene and polyoxypropylene (Pluronics); polymethacrylates; carbomers; branched or unbranched polysaccharides which comprise the saccharide monomers D-mannose, D- and L-galactose, fucose, fructose, D-xylose, L-arabinose, D-glucuronic acid, sialic acid, D-galacturontc acid, D-mannuronic acid (e.g. polymannuronic acid, or alginic acid), D-glucosamine, D-galactosamine, D-glucose and neuraminic acid including homopolysaccharides and heteropolysaccharides such as lactose, amylopectin, starch, hydroxyethyl starch, amylose, dextran sulfate, dextran, dextrins, glycogen, or the polysaccharide subunit of acid mucopolysaccharides, e.g. hyaluronic acid; polymers of sugar alcohols such as polysorbitol and polymannitol; and heparin or heparon.

### Salts

Salts of the compounds disclosed herein are within the scope of the invention. As used herein, a “salt” is salt of the instant compounds which has been modified by making acid or base salts of the compounds.

### Fc Domains

The term “Fc domain”, as used herein, generally refers to a monomer or dimer complex, comprising the C-terminal polypeptide sequences of an immunoglobulin heavy chain. The Fc domain may comprise native or variant Fc sequences. Although the boundaries of the Fc domain of an immunoglobulin heavy chain might vary, the human IgG heavy chain Fc domain is usually defined to stretch from an amino acid residue in the hinge region to the carboxyl terminus of the Fc sequence. The Fc sequence of an immunoglobulin generally comprises two constant regions, a CH2 region and a CH3 region, and optionally comprises a CH4 region. A human Fc domain may be obtained from any suitable immunoglobulin, such as the IgG1, IgG2, IgG3, or IgG4 subtypes, IgA, IgE, IgD or IgM.

Suitable Fc domains are prepared by recombinant DNA expression of pre-Fc chimeric polypeptides comprising 1) a signal peptide, obtained from a secreted or transmembrane protein, that is cleaved in front of a mature polypeptide having an N-terminal cysteine residue, contiguous with 2) an Fc domain polypeptide having an N-terminal cysteine residue.

Suitable examples of signal peptides are sonic hedgehog (SHH) (GenBank Acc. No. NM000193), IFNalpha-2 (IFN) (GenBank Acc. No. NP000596), and cholesterol ester transferase (CETP) (GenBank Accession No. NM000078). Other suitable examples include Indian hedgehog (Genbank Acc. No. NM002181), desert hedgehog (Genbank Acc. No. NM021044), IFNalpha-1 (Genbank Acc. No. NP076918), IFNalpha-4 (Genbank Acc. No. NM021068), IFNalpha-5 (Genbank Acc. No. NM002169), IFNalpha-6 (Genbank Acc. No. NM021002), IFNalpha-7 (Genbank Acc. No. NM021057), IFNalpha-8 (Genbank Acc. No. NM002170), IFNalpha-10 (Genbank Acc. No. NM002171), IFNalpha-13 (Genbank Acc. No. NM006900), IFNalpha-14 (Genbank Acc. No. NM002172), IFNalpha-16 (Genbank Acc. No. NM002173), IFNalpha-17 (Genbank Acc. No. NM021268) and IFNalpha-21 (Genbank Acc. No. NM002175).

Suitable examples of Fc domains and their pre-Fc chimeric polypeptides are shown in SEQ ID NO: 1 through SEQ ID NO: 96. The Fc domains are obtained by expressing the pre-Fc chimeric polypeptides in cells under conditions leading to their secretion and cleavage of the signal peptide. The pre-Fc polypeptides may be expressed in either prokaryotic or eukaryotic host cells. Preferably, mammalian host cells are transfected with expression vectors encoding the pre-Fc polypeptides.

Human IgG1 Fc domains having the N-terminal sequence CDKTHTCPPCPAPE, CPPCPAPE, and CPAPE are shown in SEQ ID NO: 1, SEQ ID NO: 9, and SEQ ID NO: 17, respectively, and the DNA sequences encoding them are shown in SEQ ID NO: 2, SEQ ID NO: 10, and SEQ ID NO: 18, respectively. The IgG1 domain of SEQ ID NO: 1 is obtained by expressing the pre-Fc chimeric polypeptides shown in SEQ ID NO: 3 (SHH signal peptide), SEQ ID NO: 5 (IFN signal peptide), and SEQ ID NO: 7 (CETP signal peptide), using the DNA sequences shown in SEQ ID NO: 4, SEQ ID NO: 6, and SEQ ID NO: 8, respectively. The IgG1 domain of SEQ ID NO: 9 is obtained by expressing the pre-Fc chimeric polypeptides shown in SEQ ID NO: 11 (SHH signal peptide), SEQ ID NO: 13 (IFN signal peptide), and SEQ ID NO: 15 (CETP signal peptide), using the DNA sequences shown in SEQ ID NO: 12, SEQ ID NO: 14, and SEQ ID NO: 16, respectively. The IgG1 domain of SEQ ID NO: 17 is obtained by expressing the pre-Fc chimeric polypeptides shown in SEQ ID NO: 19 (SHH signal peptide), SEQ ID NO: 21 (IFN signal peptide), and SEQ ID NO: 23 (CETP signal peptide), using the DNA sequences shown in SEQ ID NO: 20, SEQ ID NO: 22, and SEQ ID NO: 24, respectively.

Human IgG2 Fc domains having the N-terminal sequence CCVECPPCPAPE, CVECPPCPAPE, CPPCPAPE, and CPAPE are shown in SEQ ID NO: 25, SEQ ID NO: 33, SEQ ID NO: 41, and SEQ ID NO: 49, respectively, and the DNA sequences encoding them are shown in SEQ ID NO: 26, SEQ ID NO: 34, SEQ ID NO: 42, and SEQ ID NO: 50, respectively. The IgG2 domain of SEQ ID NO: 25 is obtained by expressing the pre-Fc chimeric polypeptides shown in SEQ ID NO: 27 (SHH signal peptide), SEQ ID NO: 29 (IFN signal peptide), and SEQ ID NO: 31 (CETP signal peptide), using the DNA sequences shown in SEQ ID NO: 28, SEQ ID NO: 30, and SEQ ID NO: 32, respectively. The IgG2 domain of SEQ ID NO: 33 is obtained by expressing the pre-Fc chimeric polypeptides shown in SEQ ID NO: 35 (SHH signal peptide), SEQ ID NO: 37 (IFN signal peptide), and SEQ ID NO: 39 (CETP signal peptide) using the DNA sequences shown in SEQ ID NO: 36, SEQ ID NO: 38, and SEQ ID NO: 40, respectively. The IgG2 domain of SEQ ID NO: 41 is obtained from the pre-Fc chimeric polypeptides shown in SEQ ID NO: 43 (SHH signal peptide), SEQ ID NO: 45 (IFN signal peptide), and SEQ ID NO: 47 (CETP signal peptide), using the DNA sequences shown in SEQ ID NO: 44, SEQ ID NO: 46, and SEQ ID NO: 48, respectively. The IgG2 domain of SEQ ID NO: 49 is obtained from the pre-Fc chimeric polypeptides shown in SEQ ID NO: 51 (SHH signal peptide), SEQ ID NO: 53 (IFN signal peptide), and SEQ ID NO: 55 (CETP signal peptide), using the DNA sequences shown in SEQ ID NO: 52, SEQ ID NO: 54, and SEQ ID NO: 56, respectively.

Human IgG3 Fc domains having the N-terminal sequence (CPRCPEPKSDTPPP)₃-CPRCPAPE, CPRCPAPE, and CPAPE are shown in SEQ ID NO: 57, SEQ ID NO: 65, and SEQ ID NO: 73, respectively, and the DNA sequences encoding them are shown in SEQ ID NO: 58, SEQ ID NO: 66, SEQ ID NO: 42, and SEQ ID NO: 74, respectively. The IgG3 domain of SEQ ID NO: 57 is obtained by expressing the pre-Fc chimeric polypeptides shown in SEQ ID NO: 59 (SHH signal peptide), SEQ ID NO: 61 (IFN signal peptide), and SEQ ID NO: 63 (CETP signal peptide), using the DNA sequences shown in SEQ ID NO: 60, SEQ ID NO: 62, and SEQ ID NO: 64, respectively. The IgG3 domain of SEQ ID NO: 65 is obtained by expressing the pre-Fc chimeric polypeptides shown in SEQ ID NO: 67 (SHH signal peptide), SEQ ID NO: 69 (IFN signal peptide), and SEQ ID NO: 71 (CETP signal peptide), using the DNA sequences shown in SEQ ID NO: 68, SEQ ID NO: 70, and SEQ ID NO: 72, respectively. The IgG3 domain of SEQ ID NO: 73 is obtained by expressing the pre-Fc chimeric polypeptides shown in SEQ ID NO: 75 (SHH signal peptide), SEQ ID NO: 77 (IFN signal peptide), and SEQ ID NO: 79 (CETP signal peptide), using the DNA sequences shown in SEQ ID NO: 76, SEQ ID NO: 78, and SEQ ID NO: 80, respectively.

The sequences of human IgG4 Fc domains having the N-terminal sequence CPSCPAPE and CPAPE are shown in SEQ ID NO: 81 and SEQ ID NO: 89, respectively, and the DNA sequences encoding them are shown in SEQ ID NO: 82 and SEQ ID NO: 90, respectively. The IgG4 domain of SEQ ID NO: 81 is obtained by expressing the pre-Fc chimeric polypeptides shown in SEQ ID NO: 83 (SHH signal peptide), SEQ ID NO: 85 (IFN signal peptide), and SEQ ID NO: 87 (CETP signal peptide), using the DNA sequences shown in SEQ ID NO: 84, SEQ ID NO: 86, and SEQ ID NO: 88, respectively. The IgG4 domain of SEQ ID NO: 89 is obtained by expressing the pre-Fc chimeric polypeptides shown in SEQ ID NO: 91 (SHH signal peptide), SEQ ID NO: 93 (IFN signal peptide), and SEQ ID NO: 95 (CETP signal peptide), using the DNA sequences shown in SEQ ID NO: 92, SEQ ID NO: 94, and SEQ ID NO: 96, respectively.

Suitable antibody variants having at their heavy chain N-terminus a cysteine residue are prepared by recombinant DNA expression of pre-heavy chain chimeric polypeptides comprising 1) a signal peptide, obtained from a secreted or transmembrane protein, that is cleaved in front of a mature polypeptide having an N-terminal cysteine residue, contiguous with 2) a antibody heavy chain polypeptide having an N-terminal cysteine residue.

Suitable antibody variants having at their light chain N-terminus a cysteine residue are prepared by recombinant DNA expression of pre-light chain chimeric polypeptides comprising 1) a signal peptide, obtained from a secreted or transmembrane protein, that is cleaved in front of a mature polypeptide having an N-terminal cysteine residue, contiguous with 2) a antibody light chain polypeptide having an N-terminal cysteine residue.

Trastuzumab heavy and light chains are obtained by expressing the pre-heavy and pre-light chimeric polypeptides in cells under conditions leading to their secretion and cleavage of the signal peptide. The pre-heavy chain and pre-light chain polypeptides may be expressed in either prokaryotic or eukaryotic host cells. Preferably, mammalian host cells are transfected with expression vectors encoding the pre-heavy chain and pre-light chain polypeptides.

Protein sequences added to the N-terminus of the aforementioned antibody heavy chain, pre-heavy chain, light chain, and pre-light chain variants are illustrated herein for the recombinant antibody trastuzumab, but are generally applicable to any recombinant antibody. DNA sequences encoding trastuzumab and its variants may be constructed and expressed in mammalian cells by cotransfecting DNA vectors for its heavy and light chains, and variants derived thereof, as described in U.S. Pat. No. 5,821,337 (“Immunoglobulin Variants”) which is hereby incorporated by reference. The amino acid sequence of the wild-type trastuzumab light and heavy chains are shown in SEQ ID NO: 128 and SEQ ID NO: 129, respectively.

Suitable examples of trastuzumab light chains with N-terminal cysteine residues and their pre-Fc chimeric polypeptides are shown in SEQ ID NO: 130 through SEQ ID NO: 165. Suitable examples of trastuzumab heavy chains with N-terminal cysteine residues and their pre-Fc chimeric polypeptides are shown in SEQ ID NO: 166 through SEQ ID NO: 201.

Trastuzumab light chains having the N-terminal sequence C, CP, CPP, CPR, CPS, CDKT, CDKTHT, CVE, and CDTPPP are shown in SEQ ID NO: 130, SEQ ID NO: 134, SEQ ID NO: 138, SEQ ID NO: 142, SEQ ID NO: 146, SEQ ID NO: 150, SEQ ID NO: 154, SEQ ID NO: 158, and SEQ ID NO: 162, respectively. The light chain of SEQ ID NO: 130 is obtained by expressing the pre-light chimeric polypeptides shown in SEQ ID NO: 131 (SHH signal peptide), SEQ ID NO: 132 (IFN signal peptide), and SEQ ID NO: 133 (CETP signal peptide). The light chain of SEQ ID NO: 134 is obtained by expressing the pre-light chain chimeric polypeptides shown in SEQ ID NO: 135 (SHH signal peptide), SEQ ID NO: 136 (IFN signal peptide), and SEQ ID NO: 137 (CETP signal peptide). The light chain of SEQ ID NO: 138 is obtained by expressing the pre-light chimeric polypeptides shown in SEQ ID NO: 139 (SHH signal peptide), SEQ ID NO: 140 (IFN signal peptide), and SEQ ID NO: 141 (CETP signal peptide). The light chain of SEQ ID NO: 142 is obtained by expressing the pre-light chimeric polypeptides shown in SEQ ID NO: 143 (SHH signal peptide), SEQ ID NO: 144 (IFN signal peptide), and SEQ ID NO: 145 (CETP signal peptide). The light chain of SEQ ID NO: 146 is obtained by expressing the pre-heavy light chimeric polypeptides shown in SEQ ID NO: 147 (SHH signal peptide), SEQ ID NO: 148 (IFN signal peptide), and SEQ ID NO: 149 (CETP signal peptide). The light chain of SEQ ID NO: 150 is obtained by expressing the pre-light chimeric polypeptides shown in SEQ ID NO: 151 (SHH signal peptide), SEQ ID NO: 152 (IFN signal peptide), and SEQ ID NO: 153 (CETP signal peptide). The light chain of SEQ ID NO: 154 is obtained by expressing the pre-light chimeric polypeptides shown in SEQ ID NO: 155 (SHH signal peptide), SEQ ID NO: 156 (IFN signal peptide), and SEQ ID NO: 157 (CETP signal peptide). The light chain of SEQ ID NO: 158 is obtained by expressing the pre-light chimeric polypeptides shown in SEQ ID NO: 159 (SHH signal peptide), SEQ ID NO: 160 (IFN signal peptide), and SEQ ID NO: 161 (CETP signal peptide). The light chain of SEQ ID NO: 162 is obtained by expressing the pre-light chimeric polypeptides shown in SEQ ID NO: 163 (SHH signal peptide), SEQ ID NO: 164 (IFN signal peptide), and SEQ ID NO: 165 (CETP signal peptide).

Trastuzumab heavy chains having the N-terminal sequence C, CP, CPP, CPR, CPS, CDKT, CDKTHT, CVE, and CDTPPP are shown in SEQ ID NO: 166, SEQ ID NO: 170, SEQ ID NO: 174, SEQ ID NO: 178, SEQ ID NO: 182, SEQ ID NO: 186, SEQ ID NO: 190, SEQ ID NO: 194, and SEQ ID NO: 198, respectively. The heavy chain of SEQ ID NO: 166 is obtained by expressing the pre-heavy chain chimeric polypeptides shown in SEQ ID NO: 167 (SHH signal peptide), SEQ ID NO: 168 (IFN signal peptide), and SEQ ID NO: 169 (CETP signal peptide). The heavy chain of SEQ ID NO: 170 is obtained by expressing the pre-heavy chain chimeric polypeptides shown in SEQ ID NO: 171 (SHH signal peptide), SEQ ID NO: 172 (IFN signal peptide), and SEQ ID NO: 173 (CETP signal peptide) The heavy chain of SEQ ID NO: 174 is obtained from the pre-heavy chain chimeric polypeptides shown in SEQ ID NO: 175 (SHH signal peptide), SEQ ID NO: 176 (IFN signal peptide), and SEQ ID NO: 177 (CETP signal peptide). The heavy chain of SEQ ID NO: 178 is obtained from the pre-heavy chain chimeric polypeptides shown in SEQ ID NO: 179 (SHH signal peptide), SEQ ID NO: 180 (IFN signal peptide), and SEQ ID NO: 181 (CETP signal peptide). The heavy chain of SEQ ID NO: 182 is obtained by expressing the pre-heavy chain chimeric polypeptides shown in SEQ ID NO: 183 (SHH signal peptide), SEQ ID NO: 184 (IFN signal peptide), and SEQ ID NO: 185 (CETP signal peptide) The heavy chain of SEQ ID NO: 186 is obtained by expressing the pre-heavy chain chimeric polypeptides shown in SEQ ID NO: 187 (SHH signal peptide), SEQ ID NO: 188 (IFN signal peptide), and SEQ ID NO: 189 (CETP signal peptide). The heavy chain of SEQ ID NO: 190 is obtained from the pre-heavy chain chimeric polypeptides shown in SEQ ID NO: 191 (SHH signal peptide), SEQ ID NO: 192 (IFN signal peptide), and SEQ ID NO: 193 (CETP signal peptide). The heavy chain of SEQ ID NO: 194 is obtained from the pre-heavy chain chimeric polypeptides shown in SEQ ID NO: 195 (SHH signal peptide), SEQ ID NO: 196 (IFN signal peptide), and SEQ ID NO: 197 (CETP signal peptide). The heavy chain of SEQ ID NO: 198 is obtained from the pre-heavy chain chimeric polypeptides shown in SEQ ID NO: 199 (SHH signal peptide), SEQ ID NO: 200 (IFN signal peptide), and SEQ ID NO: 201 (CETP signal peptide).

Suitable host cells include 293 human embryonic cells (ATCC CRL-1573) and CHO-K1 hamster ovary cells (ATCC CCL-61) obtained from the American Type Culture Collection (Rockville, Md.). Cells are grown at 37° C. in an atmosphere of air, 95%; carbon dioxide, 5%. 293 cells are maintained in Minimal essential medium (Eagle) with 2 mM L-glutamine and Earle's BSS adjusted to contain 1.5 g/L sodium bicarbonate, 0.1 mM non-essential amino acids, and 1.0 mM sodium pyruvate, 90%; fetal bovine serum, 10%. CHO-K1 cells are maintained in Ham's F12K medium with 2 mM L-glutamine adjusted to contain 1.5 g/L sodium bicarbonate, 90%; fetal bovine serum, 10%. Other suitable host cells include CV1 monkey kidney cells (ATCC CCL-70), COS-7 monkey kidney cells (ATCC CRL-1651), VERO-76 monkey kidney cells (ATCC CRL-1587), HELA human cervical cells (ATCC CCL-2), W138 human lung cells (ATCC CCL-75), MDCK canine kidney cells (ATCC CCL-34), BRL3A rat liver cells (ATCC CRL-1442), BHK hamster kidney cells (ATCC CCL-10), MMT060562 mouse mammary cells (ATCC CCL-51), and human CD8.sup.+T lymphocytes (described in U.S. Ser. No. 08/258,152 incorporated herein in its entirety by reference).

Examples of a suitable expression vectors are pCDNA3.1(+) shown in SEQ ID NO: 97 and pSA shown in SEQ ID NO: 98. Plasmid pSA contains the following DNA sequence elements: 1) pBluescriptIIKS(+) (nucleotides 912-2941/1-619, GenBank Accession No. X52327), 2) a human cytomegalovirus promoter, enhancer, and first exon splice donor (nucleotides 63-912, GenBank Accession No. K03104), 3) a human alpha1-globin second exon splice acceptor (nucleotides 6808-6919, GenBank Accession No. J00153), 4) an SV40 T antigen polyadenylation site (nucleotides 2770-2533, Reddy et al. (1978) Science 200, 494-502), and 5) an SV40 origin of replication (nucleotides 5725-5578, Reddy et al., ibid). Other suitable expression vectors include plasmids pSVeCD4DHFR and pRKCD4 (U.S. Pat. No. 5,336,603), plasmid pIK.1.1 (U.S. Pat. No. 5,359,046), plasmid pVL-2 (U.S. Pat. No. 5,838,464), plasmid pRT43.2F3 (described in U.S. Ser. No. 08/258,152 incorporated herein in its entirety by reference).

Suitable expression vectors for human IgG pre-Fc polypeptides may be constructed by the ligation of a HindIII-PspOM1 vector fragment prepared from SEQ ID NO: 98, with a HindIII-EagI insert fragment prepared from SEQ ID NOS: 4, 6, 8, 12, 14, 16, 20, 22, 24, 28, 30, 32, 36, 38, 40, 44, 46, 48, 52, 54, 56, 60, 62, 64, 68, 70, 72, 76, 78, 80, 84, 86, 88, 92, 94, and 96.

Suitable selectable markers include the Tn5 transposon neomycin phosphotransferase (NEO) gene (Southern and Berg (1982) J. Mol. Appl. Gen. 1, 327-341), and the dihydrofolate reductase (DHFR) cDNA (Lucas et al. (1996) Nucl. Acids Res. 24, 1774-1779). One example of a suitable expression vector that incorporates a NEO gene is plasmid pSA-NEO, which is constructed by ligating a first DNA fragment, prepared by digesting SEQ ID NO: 99 with EcoRI and BglII, with a second DNA fragment, prepared by digesting SEQ ID NO:98 with EcoRI and BglII. SEQ ID NO:99 incorporates a NEO gene (nucleotides 1551 to 2345, Genbank Accession No. U00004) preceded by a sequence for translational initiation (Kozak (1991) J. Biol. Chem, 266, 19867-19870). Another example of a suitable expression vector that incorporates a NEO gene and a DHFR cDNA is plasmid pSVe-NEO-DHFR, which is constructed by ligating a first DNA fragment, prepared by digesting SEQ ID NO:99 with EcoRI and BglII, with a second DNA fragment, prepared by digesting pSVeCD4DHFR with EcoRI and BglII. Plasmid pSVe-NEO-DHFR uses SV40 early promoter/enhancers to drive expression of the NEO gene and the DHFR cDNA. Other suitable selectable markers include the XPGT gene (Mulligan and Berg (1980) Science 209, 1422-1427) and the hygromycin resistance gene (Sugden et al. (1985) Mol. Cell. Biol. 5, 410-413).

In one embodiment, cells are transfected by the calcium phosphate method of Graham et al. (1977) J. Gen. Virol. 36, 59-74. A DNA mixture (10 ug) is dissolved in 0.5 ml of 1 mM Tris-HCl, 0.1 mM EDTA, and 227 mM CaCl₂. The DNA mixture contains (in a ratio of 10:1:1) the expression vector DNA, the selectable marker DNA, and a DNA encoding the VA RNA gene (Thimmappaya et al. (1982) Cell 31, 543-551). To this mixture is added, dropwise, 0.5 mL of 50 mM Hepes (pH 7.35), 280 mM NaCl, and 1.5 mM NaPO₄. The DNA precipitate is allowed to form for 10 minutes at 25° C., then suspended and added to cells grown to confluence on 100 mm plastic tissue culture dishes. After 4 hours at 37° C., the culture medium is aspirated and 2 ml of 20% glycerol in PBS is added for 0.5 minutes. The cells are then washed with serum-free medium, fresh culture medium is added, and the cells are incubated for 5 days.

In another embodiment, cells are transiently transfected by the dextran sulfate method of Somparyrac et al. (1981) Proc. Nat. Acad. Sci. 12, 7575-7579. Cells are grown to maximal density in spinner flasks, concentrated by centrifugation, and washed with PBS. The DNA-dextran precipitate is incubated on the cell pellet. After 4 hours at 37° C., the DEAE-dextran is aspirated and 20% glycerol in PBS is added for 1.5 minutes. The cells are then washed with serum-free medium, re-introduced into spinner flasks containing fresh culture medium with 5 micrograms/ml bovine insulin and 0.1 micrograms/ml bovine transferring, and incubated for 4 days.

Following transfection by either method, the conditioned media is centrifuged and filtered to remove the host cells and debris. The sample contained the Fc domain is then concentrated and purified by any selected method, such as dialysis and/or column chromatography (see below). To identify the Fc domain in the cell culture supernatant, the culture medium is removed 24 to 96 hours after transfection, concentrated, and analyzed by SDS-polyacrylamide gel electrophoresis (SDS-PAGE) in the presence or absence of a reducing agent such as dithiothreitol.

For unamplified expression, plasmids are transfected into human 293 cells (Graham et al., J. Gen. Virol. 36:59 74 (1977)), using a high efficiency procedure (Gorman et al., DNA Prot. Eng. Tech. 2:3 10 (1990)). Media is changed to serum-free and harvested daily for up to five days. For unamplified expression, plasmids are transfected into human 293 cells (Graham et al., J. Gen. Virol. 36:59 74 (1977)), using a high efficiency procedure (Gorman et al., DNA Prot. Eng. Tech. 2:3 10 (1990)). Media is changed to serum-free and harvested daily for up to five days. The Fc domains are purified from the cell culture supernatant using HiTrap Protein A HP (Pharmacia). The eluted Fc domains are buffer-exchanged into PBS using a Centricon-30 (Amicon), concentrated to 0.5 ml, sterile filtered using a Millex-GV (Millipore) at 4° C.

### Stretches of Consecutive Amino Acids

Examples of stretches of consecutive amino acids as referred to herein include, but are not limited to, consecutive amino acids including binding domains such as secreted or transmembrane proteins, intracellular binding domains and antibodies (whole or portions thereof) and modified versions thereof. The following are some non-limiting examples:

### 1) Immunoglobulins

The term “antibody” is used in the broadest sense and specifically covers monoclonal antibodies (including full length monoclonal antibodies), polyclonal antibodies, multispecific antibodies (e.g., bispecific antibodies), monovalent antibodies, multivalent antibodies, and antibody fragments so long as they exhibit the desired biological activity (e.g., Fab and/or single-armed antibodies).

The “class” of an antibody refers to the type of constant domain or constant region possessed by its heavy chain. There are five major classes of antibodies: IgA, IgD, IgE, IgG, and IgM, and several of these may be further divided into subclasses (isotypes), e.g., IgG1, IgG2, IgG3, IgG4, IgA1, and IgA2. The heavy chain constant domains that correspond to the different classes of immunoglobulins are called α, δ, ε, γ, and μ, respectively.

An “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)₂; diabodies; linear antibodies; single-chain antibody molecules (e.g., scFv); and multispecific antibodies formed from antibody fragments.

The terms “full length antibody,” “intact antibody,” and “whole antibody” are used herein interchangeably to refer to an antibody having a structure substantially similar to a native antibody structure or having heavy chains that contain an Fc region as defined herein.

A “blocking” antibody or an “antagonist” antibody is one which significantly inhibits (either partially or completely) a biological activity of the antigen it binds.

An “antibody that binds to the same epitope” as a reference antibody refers to an antibody that blocks binding of the reference antibody to its antigen in a competition assay by 50% or more, and conversely, the reference antibody blocks binding of the antibody to its antigen in a competition assay by 50% or more. An exemplary competition assay is provided herein.

The term “variable region” or “variable domain” refers to the domain of an antibody heavy or light chain that is involved in binding the antibody to antigen. The variable domains of the heavy chain and light chain (VH and VL, respectively) of a native antibody generally have similar structures, with each domain comprising four conserved framework regions (FRs) and three hypervariable regions (HVRs). (See, e.g., Kindt et al. Kuby Immunology, 6^{th} ed., W. H. Freeman and Co., page 91 (2007).) A single VH or VL domain may be sufficient to confer antigen-binding specificity. Furthermore, antibodies that bind a particular antigen may be isolated using a VH or VL domain from an antibody that binds the antigen to screen a library of complementary VL or VH domains, respectively. See, e.g., Portolano et al., J. Immunol. 150:880-887 (1993); Clarkson et al., Nature 352:624-628 (1991).

The term “hypervariable region” or “HVR,” as used herein, refers to each of the regions of an antibody variable domain which are hypervariable in sequence and/or form structurally defined loops (“hypervariable loops”). Generally, native four-chain antibodies comprise six HVRs; three in the VH (H1, H2, H3), and three in the VL (L1, L2, L3). HVRs generally comprise amino acid residues from the hypervariable loops and/or from the “complementarity determining regions” (CDRs), the latter being of highest sequence variability and/or involved in antigen recognition. Exemplary hypervariable loops occur at amino acid residues 26-32 (L1), 50-52 (L2), 91-96 (L3), 26-32 (H1), 53-55 (H2), and 96-101 (H3). (Chothia and Lesk, J. Mol. Biol. 196:901-917 (1987).) Exemplary CDRs (CDR-L1, CDR-L2, CDR-L3, CDR-H1, CDR-H2, and CDR-H3) occur at amino acid residues 24-34 of L1, 50-56 of L2, 89-97 of L3, 31-35B of H1, 50-65 of H2, and 95-102 of H3. (Kabat et al., Sequences of Proteins of Immunological Interest, 5th Ed. Public Health Service, National Institutes of Health, Bethesda, Md. (1991).) With the exception of CDR1 in VH, CDRs generally comprise the amino acid residues that form the hypervariable loops. CDRs also comprise “specificity determining residues,” or “SDRs,” which are residues that contact antigen. SDRs are contained within regions of the CDRs called abbreviated-CDRs, or a-CDRs. Exemplary a-CDRs (a-CDR-L1, a-CDR-L2, a-CDR-L3, a-CDR-H1, a-CDR-H2, and a-CDR-H3) occur at amino acid residues 31-34 of L1, 50-55 of L2, 89-96 of L3, 31-35B of H1, 50-58 of H2, and 95-102 of H3. (See Almagro and Fransson, Front. Biosci. 13:1619-1633 (2008).) Unless otherwise indicated, HVR residues and other residues in the variable domain (e.g., FR residues) are numbered herein according to Kabat et al., supra.

“Framework” or “FR” refers to variable domain residues other than hypervariable region (HVR) residues. The FR of a variable domain generally consists of four FR domains: FR1, FR2, FR3, and FR4. Accordingly, the HVR and FR sequences generally appear in the following sequence in VH (or VL): FR1-H1(L1)-FR2-H2(L2)-FR3-H3(L3)-FR4.

The phrase “N-terminally truncated heavy chain”, as used herein, refers to a polypeptide comprising parts but not all of a full length immunoglobulin heavy chain, wherein the missing parts are those normally located on the N terminal region of the heavy chain. Missing parts may include, but are not limited to, the variable domain, CH1, and part or all of a hinge sequence. Generally, if the wild type hinge sequence is not present, the remaining constant domain(s) in the N-terminally truncated heavy chain would comprise a component that is capable of linkage to another Fc sequence (i.e., the “first” Fc polypeptide as described herein). For example, said component can be a modified residue or an added cysteine residue capable of forming a disulfide linkage.

“Fc receptor” or “FcR” describes a receptor that binds to the Fc region of an antibody. In some embodiments, an FcR is a native human FcR. In some embodiments, an FcR is one which binds an IgG antibody (a gamma receptor) and includes receptors of the FcγRI, FcγRII, and FcγRIII subclasses, including allelic variants and alternatively spliced forms of those receptors. FcγRII receptors include FcγRIIA (an “activating receptor”) and FcγRIIB (an “inhibiting receptor”), which have similar amino acid sequences that differ primarily in the cytoplasmic domains thereof. Activating receptor FcγRIIA contains an immunoreceptor tyrosine-based activation motif (ITAM) in its cytoplasmic domain Inhibiting receptor FcγRIIB contains an immunoreceptor tyrosine-based inhibition motif (ITIM) in its cytoplasmic domain. (see, e.g., Daeron, Annu. Rev. Immunol. 15:203-234 (1997)). FcRs are reviewed, for example, in Ravetch and Kinet, Annu. Rev. Immunol 9:457-92 (1991); Capel et al., Immunomethods 4:25-34 (1994); and de Haas et al., J. Lab. Clin. Med. 126:330-41 (1995). Other FcRs, including those to be identified in the future, are encompassed by the term “FcR” herein.

The term “Fc receptor” or “FcR” also includes the neonatal receptor, FcRn, which is responsible for the transfer of maternal IgGs to the fetus (Guyer et al., J. Immunol. 117:587 (1976) and Kim et al., J. Immunol. 24:249 (1994)) and regulation of homeostasis of immunoglobulins. Methods of measuring binding to FcRn are known (see, e.g., Ghetie and Ward., Immunol. Today 18(12):592-598 (1997); Ghetie et al., Nature Biotechnology, 15(7):637-640 (1997); Hinton et al., J. Biol. Chem. 279(8):6213-6216 (2004); WO 2004/92219 (Hinton et al.).

Binding to human FcRn in vivo and serum half life of human FcRn high affinity binding polypeptides can be assayed, e.g., in transgenic mice or transfected human cell lines expressing human FcRn, or in primates to which the polypeptides with a variant Fc region are administered. WO 2000/42072 (Presta) describes antibody variants with improved or diminished binding to FcRs. See also, e.g., Shields et al. J. Biol. Chem. 9(2):6591-6604 (2001).

The “hinge region,” “hinge sequence”, and variations thereof, as used herein, includes the meaning known in the art, which is illustrated in, for example, Janeway et al., Immuno Biology: the immune system in health and disease, (Elsevier Science Ltd., NY) (4th ed., 1999); Bloom et al., Protein Science (1997), 6:407-415; Humphreys et al., J. Immunol. Methods (1997), 209:193-202.

Unless indicated otherwise, the expression “multivalent antibody” is used throughout this specification to denote an antibody comprising three or more antigen binding sites. The multivalent antibody is preferably engineered to have the three or more antigen binding sites and is generally not a native sequence IgM or IgA antibody.

An “Fv” fragment is an antibody fragment which contains a complete antigen recognition and binding site. This region consists of a dimer of one heavy and one light chain variable domain in tight association, which can be covalent in nature, for example in scFv. It is in this configuration that the three HVRs of each variable domain interact to define an antigen binding site on the surface of the V_{H}-V_{L} dimer. Collectively, the six HVRs or a subset thereof confer antigen binding specificity to the antibody. However, even a single variable domain (or half of an Fv comprising only three HVRs specific for an antigen) has the ability to recognize and bind antigen, although usually at a lower affinity than the entire binding site.

The “Fab” fragment contains a variable and constant domain of the light chain and a variable domain and the first constant domain (CH1) of the heavy chain. F(ab′) 2 antibody fragments comprise a pair of Fab fragments which are generally covalently linked near their carboxy termini by hinge cysteines between them. Other chemical couplings of antibody fragments are also known in the art.

The phrase “antigen binding arm”, as used herein, refers to a component part of an antibody fragment that has an ability to specifically bind a target molecule of interest. Generally and preferably, the antigen binding arm is a complex of immunoglobulin polypeptide sequences, e.g., HVR and/or variable domain sequences of an immunoglobulin light and heavy chain.

“Single-chain Fv” or “scFv” antibody fragments comprise the V_{H} and V_{L} domains of antibody, wherein these domains are present in a single polypeptide chain. Generally the Fv polypeptide further comprises a polypeptide linker between the V_{H} and V_{L} domains, which enables the scFv to form the desired structure for antigen binding. For a review of scFv, see Pluckthun in The Pharmacology of Monoclonal Antibodies, Vol 113, Rosenburg and Moore eds. Springer-Verlag, New York, pp. 269-315 (1994).

The term “diabodies” refers to small antibody fragments with two antigen-binding sites, which fragments comprise a heavy chain variable domain (V_{H}) connected to a light chain variable domain (V_{L}) in the same polypeptide chain (V_{H} and V_{L}). By using a linker that is too short to allow pairing between the two domains on the same chain, the domains are forced to pair with the complementary domains of another chain and create two antigen-binding sites. Diabodies are described more fully in, for example, EP 404,097; WO 93/11161; and Hollinger et al., Proc. Natl. Acad. Sci. USA, 90:6444-6448 (1993).

The expression “linear antibodies” refers to the antibodies described in Zapata et al., Protein Eng., 8(10):1057-1062 (1995). Briefly, these antibodies comprise a pair of tandem Fd segments (V.sub.H-C.sub.H1-V.sub.H-C.sub.H1) which, together with complementary light chain polypeptides, form a pair of antigen binding regions. Linear antibodies can be bispecific or monospecific.

The term “monoclonal antibody” as used herein refers to an antibody obtained from a population of substantially homogeneous antibodies, i.e., the individual antibodies comprising the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies, and is not to be construed as requiring production of the antibody by any particular method. For example, the monoclonal antibodies to be used may be made by a variety of techniques, including but not limited to the hybridoma method, recombinant DNA methods, phage-display methods, and methods utilizing transgenic animals containing all or part of the human immunoglobulin loci, such methods and other exemplary methods for making monoclonal antibodies being described herein.

The term “chimeric” antibody refers to an antibody in which a portion of the heavy and/or light chain is derived from a particular source or species, while the remainder of the heavy and/or light chain is derived from a different source or species.

A “humanized” antibody refers to a chimeric antibody comprising amino acid residues from non-human HVRs and amino acid residues from human FRs. In certain embodiments, a humanized antibody will comprise substantially all of at least one, and typically two, variable domains, in which all or substantially all of the HVRs (e.g., CDRs) correspond to those of a non-human antibody, and all or substantially all of the FRs correspond to those of a human antibody. A humanized antibody optionally may comprise at least a portion of an antibody constant region derived from a human antibody. A “humanized form” of an antibody, e.g., a non-human antibody, refers to an antibody that has undergone humanization.

A “human antibody” is one which possesses an amino acid sequence which corresponds to that of an antibody produced by a human or a human cell or derived from a non-human source that utilizes human antibody repertoires or other human antibody-encoding sequences. This definition of a human antibody specifically excludes a humanized antibody comprising non-human antigen-binding residues.

A “naked antibody” refers to an antibody that is not conjugated to a heterologous moiety (e.g., a cytotoxic moiety) or radiolabel. The naked antibody may be present in a pharmaceutical formulation.

“Native antibodies” refer to naturally occurring immunoglobulin molecules with varying structures. For example, native IgG antibodies are heterotetrameric glycoproteins of about 150,000 Daltons, composed of two identical light chains and two identical heavy chains that are disulfide-bonded. From N- to C-terminus, each heavy chain has a variable region (VH), also called a variable heavy domain or a heavy chain variable domain, followed by three constant domains (CH1, CH2, and CH3). Similarly, from N- to C-terminus, each light chain has a variable region (VL), also called a variable light domain or a light chain variable domain, followed by a constant light (CL) domain. The light chain of an antibody may be assigned to one of two types, called kappa (κ) and lambda (λ), based on the amino acid sequence of its constant domain.

“Affinity” refers to the strength of the sum total of noncovalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (Kd). Affinity can be measured by common methods known in the art, including those described herein. Specific illustrative and exemplary embodiments for measuring binding affinity are described in the following.

An “affinity matured” antibody refers to an antibody with one or more alterations in one or more HVRs, compared to a parent antibody which does not possess such alterations, such alterations resulting in an improvement in the affinity of the antibody for antigen.

An antibody having a “biological characteristic” of a designated antibody is one which possesses one or more of the biological characteristics of that antibody which distinguish it from other antibodies that bind to the same antigen.

A “functional antigen binding site” of an antibody is one which is capable of binding a target antigen. The antigen binding affinity of the antigen binding site is not necessarily as strong as the parent antibody from which the antigen binding site is derived, but the ability to bind antigen must be measurable using any one of a variety of methods known for evaluating antibody binding to an antigen. Moreover, the antigen binding affinity of each of the antigen binding sites of a multivalent antibody herein need not be quantitatively the same. For the multimeric antibodies herein, the number of functional antigen binding sites can be evaluated using ultracentrifugation analysis as described in Example 2 of U.S. Patent Application Publication No. 20050186208. According to this method of analysis, different ratios of target antigen to multimeric antibody are combined and the average molecular weight of the complexes is calculated assuming differing numbers of functional binding sites. These theoretical values are compared to the actual experimental values obtained in order to evaluate the number of functional binding sites.

A “species-dependent antibody” is one which has a stronger binding affinity for an antigen from a first mammalian species than it has for a homologue of that antigen from a second mammalian species. Normally, the species-dependent antibody “binds specifically” to a human antigen (i.e. has a binding affinity (K.sub.d) value of no more than about 1.times.10.sup.-7 M, preferably no more than about 1.times.10.sup.-8 M and most preferably no more than about 1.times.10.sup.-9 M) but has a binding affinity for a homologue of the antigen from a second nonhuman mammalian species which is at least about 50 fold, or at least about 500 fold, or at least about 1000 fold, weaker than its binding affinity for the human antigen. The species-dependent antibody can be any of the various types of antibodies as defined above. In some embodiments, the species-dependent antibody is a humanized or human antibody.

An “isolated” antibody is one which has been separated from a component of its natural environment. In some embodiments, an antibody is purified to greater than 95% or 99% purity as determined by, for example, electrophoretic (e.g., SDS-PAGE, isoelectric focusing (IEF), capillary electrophoresis) or chromatographic (e.g., ion exchange or reverse phase HPLC). For review of methods for assessment of antibody purity, see, e.g., Flatman et al., J. Chromatogr. B 848:79-87 (2007).

### 2) Extracellular Proteins

Extracellular proteins play important roles in, among other things, the formation, differentiation and maintenance of multicellular organisms. A discussion of various intracellular proteins of interest is set forth in U.S. Pat. No. 6,723,535, Ashkenazi et al., issued Apr. 20, 2004, hereby incorporated by reference.

The fate of many individual cells, e.g., proliferation, migration, differentiation, or interaction with other cells, is typically governed by information received from other cells and/or the immediate environment. This information is often transmitted by secreted polypeptides (for instance, mitogenic factors, survival factors, cytotoxic factors, differentiation factors, neuropeptides, and hormones) which are, in turn, received and interpreted by diverse cell receptors or membrane-bound proteins. These secreted polypeptides or signaling molecules normally pass through the cellular secretory pathway to reach their site of action in the extracellular environment.

Secreted proteins have various industrial applications, including as pharmaceuticals, diagnostics, biosensors and bioreactors. Most protein drugs available at present, such as thrombolytic agents, interferons, interleukins, erythropoietins, colony stimulating factors, and various other cytokines, are secretory proteins. Their receptors, which are membrane proteins, also have potential as therapeutic or diagnostic agents. Efforts are being undertaken by both industry and academia to identify new, native secreted proteins. Many efforts are focused on the screening of mammalian recombinant DNA libraries to identify the coding sequences for novel secreted proteins. Examples of screening methods and techniques are described in the literature (see, for example, Klein et al., Proc. Natl. Acad. Sci. 93:7108-7113 (1996); U.S. Pat. No. 5,536,637)).

Membrane-bound proteins and receptors can play important roles in, among other things, the formation, differentiation and maintenance of multicellular organisms. The fate of many individual cells, e.g., proliferation, migration, differentiation, or interaction with other cells, is typically governed by information received from other cells and/or the immediate environment. This information is often transmitted by secreted polypeptides (for instance, mitogenic factors, survival factors, cytotoxic factors, differentiation factors, neuropeptides, and hormones) which are, in turn, received and interpreted by diverse cell receptors or membrane-bound proteins. Such membrane-bound proteins and cell receptors include, but are not limited to, cytokine receptors, receptor kinases, receptor phosphatases, receptors involved in cell-cell interactions, and cellular adhesin molecules like selectins and integrins. For instance, transduction of signals that regulate cell growth and differentiation is regulated in part by phosphorylation of various cellular proteins. Protein tyrosine kinases, enzymes that catalyze that process, can also act as growth factor receptors. Examples include fibroblast growth factor receptor and nerve growth factor receptor.

Membrane-bound proteins and receptor molecules have various industrial applications, including as pharmaceutical and diagnostic agents. Receptor immunoadhesins, for instance, can be employed as therapeutic agents to block receptor-ligand interactions. The membrane-bound proteins can also be employed for screening of potential peptide or small molecule inhibitors of the relevant receptor/ligand interaction.

### 3) Intein-Based C-Terminal Syntheses

As described, for example, in U.S. Pat. No. 6,849,428, issued Feb. 1, 2005, inteins are the protein equivalent of the self-splicing RNA introns (see Perler et al., Nucleic Acids Res. 22:1125-1127 (1994)), which catalyze their own excision from a precursor protein with the concomitant fusion of the flanking protein sequences, known as exteins (reviewed in Perler et al., Curr. Opin. Chem. Biol. 1:292-299 (1997); Perler, F. B. Cell 92(1):1-4 (1998); Xu et al., EMBO J. 15(19):5146-5153 (1996)).

Studies into the mechanism of intein splicing led to the development of a protein purification system that utilized thiol-induced cleavage of the peptide bond at the N-terminus of the Sce VMA intein (Chong et al., Gene 192(2):271-281 (1997)). Purification with this intein-mediated system generates a bacterially-expressed protein with a C-terminal thioester (Chong et al., (1997)). In one application, where it is described to isolate a cytotoxic protein, the bacterially expressed protein with the C-terminal thioester is then fused to a chemically-synthesized peptide with an N-terminal cysteine using the chemistry described for “native chemical ligation” (Evans et al., Protein Sci. 7:2256-2264 (1998); Muir et al., Proc. Natl. Acad. Sci. USA 95:6705-6710 (1998)).

This technique, referred to as “intein-mediated protein ligation” (IPL), represents an important advance in protein semi-synthetic techniques. However, because chemically-synthesized peptides of larger than about 100 residues are difficult to obtain, the general application of IPL was limited by the requirement of a chemically-synthesized peptide as a ligation partner.

IPL technology was significantly expanded when an expressed protein with a predetermined N-terminus, such as cysteine, was generated, as described for example in U.S. Pat. No. 6,849,428. This allows the fusion of one or more expressed proteins from a host cell, such as bacterial, yeast or mammalian cells. In one non-limiting example the intein a modified RIR1 Methanobacterium thermoautotrophicum is that cleaves at either the C-terminus or N-terminus is used which allows for the release of a bacterially expressed protein during a one-column purification, thus eliminating the need proteases entirely.

Intein technology is one example of one route to obtain components. In one embodiment, the subunits of the compounds of the invention are obtained by transfecting suitable cells, capable of expressing and secreting mature chimeric polypeptides, wherein such polypeptides comprise, for example, an adhesin domain contiguous with an isolatable c-terminal intein domain (see U.S. Pat. No. 6,849,428, Evans et al., issued Feb. 1, 2005, hereby incorporated by reference). The cells, such as mammalian cells or bacterial cells, are transfected using known recombinant DNA techniques. The secreted chimeric polypeptide can then be isolated, e.g. using a chitin-derivatized resin in the case of an intein-chitin binding domain (see U.S. Pat. No. 6,897,285, Xu et al., issued May 24, 2005, hereby incorporated by reference), and is then treated under conditions permitting thiol-mediated cleavage and release of the now C-terminal thioester-terminated subunit. The thioester-terminated adhesion subunit is readily converted to a C-terminal cysteine terminated subunit.

For example, following an intein autocleavage reaction, a thioester intermediate is generated that permits the facile addition of cysteine, selenocysteine, homocysteine, or homoselenocysteine, or a derivative of cysteine, selenocysteine, homocysteine, homoselenocysteine, to the C-terminus by native chemical ligation. Methods of adding a cysteine, selenocysteine, homocysteine, or homoselenocysteine, or a derivative of cysteine, selenocysteine, homocysteine, homoselenocysteine, to the C-terminus by native chemical ligation which are useful in aspects of the present invention are described in U.S. Patent Application No. 2008/0254512, Capon, published Oct. 16, 2008, the entire contents of which are hereby incorporated herein by reference.

### Kits

Another aspect of the present invention provides kits comprising the compounds disclosed herein and the pharmaceutical compositions comprising these compounds. A kit may include, in addition to the compound or pharmaceutical composition, diagnostic or therapeutic agents. A kit may also include instructions for use in a diagnostic or therapeutic method. In a diagnostic embodiment, the kit includes the compound or a pharmaceutical composition thereof and a diagnostic agent. In a therapeutic embodiment, the kit includes the antibody or a pharmaceutical composition thereof and one or more therapeutic agents, such as an additional antineoplastic agent, anti-tumor agent or chemotherapeutic agent.

### General Techniques

The description below relates primarily to production of stretches of consecutive amino acids or polypeptides of interest by culturing cells transformed or transfected with a vector containing an encoding nucleic acid. It is, of course, contemplated that alternative methods, which are well known in the art, may be employed. For instance, the amino acid sequence, or portions thereof, may be produced by direct peptide synthesis using solid-phase techniques (see, e.g., Stewart et al., Solid-Phase Peptide Synthesis, W.H. Freeman Co., San Francisco, Calif. (1969); Merrifield, J. Am. Chem. Soc., 85:2149-2154 (1963)). In vitro protein synthesis may be performed using manual techniques or by automation. Automated synthesis may be accomplished, for instance, using an Applied Biosystems Peptide Synthesizer (Foster City, Calif.) using manufacturer's instructions. Various portions of the stretches of consecutive amino acids or polypeptides of interest may be chemically synthesized separately and combined using chemical or enzymatic methods to produce the full-length stretches of consecutive amino acids or polypeptides of interest.

### 1. Selection and Transformation of Host Cells

Host cells are transfected or transformed with expression or cloning vectors described herein for production and cultured in conventional nutrient media modified as appropriate for inducing promoters, selecting transformants, or amplifying the genes encoding the desired sequences. The culture conditions, such as media, temperature, pH and the like, can be selected by the skilled artisan without undue experimentation. In general, principles, protocols, and practical techniques for maximizing the productivity of cell cultures can be found in Mammalian Cell Biotechnology: a Practical Approach, M. Butler, ed. (IRL Press, 1991) and Sambrook et al., supra.

Methods of eukaryotic cell transfection and prokaryotic cell transformation are known to the ordinarily skilled artisan, for example, CaCl₂, CaPO₄, liposome-mediated and electroporation. Depending on the host cell used, transformation is performed using standard techniques appropriate to such cells. The calcium treatment employing calcium chloride, as described in Sambrook et al., supra, or electroporation is generally used for prokaryotes. Infection with Agrobacterium tumefaciens is used for transformation of certain plant cells, as described by Shaw et al., Gene, 23:315 (1983) and WO 89/05859 published Jun. 29, 1989. For mammalian cells without such cell walls, the calcium phosphate precipitation method of Graham and van der Eb, Virology, 52:456-457 (1978) can be employed. General aspects of mammalian cell host system transfections have been described in U.S. Pat. No. 4,399,216. Transformations into yeast are typically carried out according to the method of Van Solingen et al., J. Bact., 130:946 (1977) and Hsiao et al., Proc. Natl. Acad. Sci. (USA), 76:3829 (1979). However, other methods for introducing DNA into cells, such as by nuclear microinjection, electroporation, bacterial protoplast fusion with intact cells, or polycations, e.g., polybrene, polyornithine, may also be used. For various techniques for transforming mammalian cells, see Keown et al., Methods in Enzymology, 185:527-537 (1990) and Mansour et al., Nature, 336:348-352 (1988).

Suitable host cells for cloning or expressing the DNA in the vectors herein include prokaryote, yeast, or higher eukaryote cells. Suitable prokaryotes include but are not limited to eubacteria, such as Gram-negative or Gram-positive organisms, for example, Enterobacteriaceae such as E. coli. Various E. coli strains are publicly available, such as E. coli K12 strain MM294 (ATCC 31,446); E. coli X1776 (ATCC 31,537); E. coli strain W3110 (ATCC 27,325) and K5772 (ATCC 53,635). Other suitable prokaryotic host cells include Enterobacteriaceae such as Escherichia, e.g., E. coli, Enterobacter, Erwinia, Klebsiella, Proteus, Salmonella, e.g., Salmonella typhimurium, Serratia, e.g., Serratia marcescans, and Shigella, as well as Bacilli such as B. subtilis and B. licheniformis (e.g., B. licheniformis 41P disclosed in DD 266,710 published Apr. 12, 1989), Pseudomonas such as P. aeruginosa, and Streptomyces. These examples are illustrative rather than limiting. Strain W3110 is one particularly preferred host or parent host because it is a common host strain for recombinant DNA product fermentations. Preferably, the host cell secretes minimal amounts of proteolytic enzymes. For example, strain W3110 may be modified to effect a genetic mutation in the genes encoding proteins endogenous to the host, with examples of such hosts including E. coli W3110 strain 1A2, which has the complete genotype tonA; E. coli W3110 strain 9E4, which has the complete genotype tonA ptr3; E. coli W3110 strain 27C7 (ATCC 55,244), which has the complete genotype tonAptr3phoA E15 (argF-lac)169 degP ompT kan.sup.r; E. coli W3110 strain 37D6, which has the complete genotype tonA ptr3 phoA E15 (argF-lac)169 degP ompT rbs7 ilvG kan.sup.r, E. coli W3110 strain 40B4, which is strain 37D6 with a non-kanamycin resistant degP deletion mutation; and an E. coli strain having mutant periplasmic protease disclosed in U.S. Pat. No. 4,946,783 issued Aug. 7, 1990. Alternatively, in vitro methods of cloning, e.g., PCR or other nucleic acid polymerase reactions, are suitable.

In addition to prokaryotes, eukaryotic microbes such as filamentous fungi or yeast are suitable cloning or expression hosts for encoding vectors. Saccharomyces cerevisiae is a commonly used lower eukaryotic host microorganism. Others include Schizosaccharomyces pombe (Beach and Nurse, Nature, 290:140 (1981); EP 139,383 published May 2, 1985); Kluyveromyces hosts (U.S. Pat. No. 4,943,529; Fleer et al., Bio/Technology, 9:968-975 (1991)) such as, e.g., K. lactis (MW98-8C, CBS683, CBS4574; Louvencourt et al., J. Bacteriol., 737 (1983)), K. fragilis (ATCC 12,424), K. bulgaricus (ATCC 16,045), K. wickeramii (ATCC 24,178), K. waltii (ATCC 56,500), K. drosophilarum (ATCC 36,906; Van den Berg et al., Bio/Technology, 8:135 (1990)), K. thermotolerans, and K. marxianus; yarrowia (EP 402,226); Pichia pastoris (EP 183,070; Sreekrishna et al., J. Basic Microbiol., 28:265-278 (1988)); Candida; Trichoderma reesia (EP 244,234); Neurospora crassa (Case et al., Proc. Natl. Acad. Sci. USA, 76:5259-5263 (1979)); Schwanniomyces such as Schwanniomyces occidentalis (EP 394,538 published Oct. 31, 1990); and filamentous fungi such as, e.g., Neurospora, Penicillium, Tolypocladium (WO 91/00357 published Jan. 10, 1991), and Aspergillus hosts such as A. nidulans (Ballance et al., Biochem. Biophys. Res. Commun., 112:284-289 (1983); Tilburn et al., Gene, 26:205-221 (1983); Yelton et al., Proc. Natl. Acad. Sci. USA, 81:1470-1474 (1984)) and A. niger (Kelly and Hynes, EMBO J., 4:475479 (1985)). Methylotropic yeasts are suitable herein and include, but are not limited to, yeast capable of growth on methanol selected from the genera consisting of Hansenula, Candida, Kloeckera, Pichia, Saccharomyces, Torulopsis, and Rhodotorula. A list of specific species that are exemplary of this class of yeasts may be found in C. Anthony, The Biochemistry of Methylotrophs, 269 (1982).

Suitable host cells for the expression of glycosylated stretches of consecutive amino acids or polypeptides of interest are derived from multicellular organisms. Examples of invertebrate cells include insect cells such as Drosophila S2 and Spodoptera Sf9, as well as plant cells. Examples of useful mammalian host cell lines include Chinese hamster ovary (CHO) and COS cells. More specific examples include monkey kidney CV1 line transformed by SV40 (COS-7, ATCC CRL 1651); human embryonic kidney line (293 or 293 cells subcloned for growth in suspension culture, Graham et al., J. Gen Virol., 36:59 (1977)); Chinese hamster ovary cells/-DHFR (CHO, Urlaub and Chasin, Proc. Natl. Acad. Sci. USA, 77:4216 (1980)); mouse sertoli cells (TM4, Mather, Biol. Reprod., 23:243-251 (1980)); human lung cells (W138, ATCC CCL 75); human liver cells (Hep G2, HB 8065); and mouse mammary tumor (MMT 060562, ATCC CCL51). The selection of the appropriate host cell is deemed to be within the skill in the art.

### 2. Selection and Use of a Replicable Vector

The nucleic acid (e.g., cDNA or genomic DNA) encoding the stretch of consecutive amino acids or polypeptides of interest may be inserted into a replicable vector for cloning (amplification of the DNA) or for expression. Various vectors are publicly available. The vector may, for example, be in the form of a plasmid, cosmid, viral particle, or phage. The appropriate nucleic acid sequence may be inserted into the vector by a variety of procedures. In general, DNA is inserted into an appropriate restriction endonuclease site(s) using techniques known in the art. Vector components generally include, but are not limited to, one or more of a signal sequence, an origin of replication, one or more marker genes, an enhancer element, a promoter, and a transcription termination sequence. Construction of suitable vectors containing one or more of these components employs standard ligation techniques which are known to the skilled artisan.

The stretches of consecutive amino acids or polypeptides of interest may be produced recombinantly not only directly, but also as a fusion polypeptide with a heterologous polypeptide, which may be a signal sequence or other polypeptide having a specific cleavage site at the N-terminus of the mature protein or polypeptide. In general, the signal sequence may be a component of the vector, or it may be a part of the encoding DNA that is inserted into the vector. The signal sequence may be a prokaryotic signal sequence selected, for example, from the group of the alkaline phosphatase, penicillinase, 1pp, or heat-stable enterotoxin II leaders. For yeast secretion the signal sequence may be, e.g., the yeast invertase leader, alpha factor leader (including Saccharomyces and Kluyveromyces alpha-factor leaders, the latter described in U.S. Pat. No. 5,010,182), or acid phosphatase leader, the C. albicans glucoamylase leader (EP 362,179 published Apr. 4, 1990), or the signal described in WO 90/13646 published Nov. 15, 1990. In mammalian cell expression, mammalian signal sequences may be used to direct secretion of the protein, such as signal sequences from secreted polypeptides of the same or related species, as well as viral secretory leaders.

Both expression and cloning vectors contain a nucleic acid sequence that enables the vector to replicate in one or more selected host cells. Such sequences are well known for a variety of bacteria, yeast, and viruses. The origin of replication from the plasmid pBR322 is suitable for most Gram-negative bacteria, the 2mu plasmid origin is suitable for yeast, and various viral origins (SV40, polyoma, adenovirus, VSV or BPV) are useful for cloning vectors in mammalian cells.

Expression and cloning vectors will typically contain a selection gene, also termed a selectable marker. Typical selection genes encode proteins that (a) confer resistance to antibiotics or other toxins, e.g., ampicillin, neomycin, methotrexate, or tetracycline, (b) complement auxotrophic deficiencies, or (c) supply critical nutrients not available from complex media, e.g., the gene encoding D-alanine racemase for Bacilli.

An example of suitable selectable markers for mammalian cells are those that enable the identification of cells competent to take up the encoding nucleic acid, such as DHFR or thymidine kinase. An appropriate host cell when wild-type DHFR is employed is the CHO cell line deficient in DHFR activity, prepared and propagated as described by Urlaub et al., Proc. Natl. Acad. Sci. USA, 77:4216 (1980). A suitable selection gene for use in yeast is the trp1 gene present in the yeast plasmid YRp7 (Stinchcomb et al., Nature, 282:39 (1979); Kingsman et al., Gene, 7:141 (1979); Tschemper et al., Gene, 10:157 (1980)). The trp1 gene provides a selection marker for a mutant strain of yeast lacking the ability to grow in tryptophan, for example, ATCC No. 44076 or PEP4-1 (Jones, Genetics, 85:12 (1977)).

Expression and cloning vectors usually contain a promoter operably linked to the encoding nucleic acid sequence to direct mRNA synthesis. Promoters recognized by a variety of potential host cells are well known. Promoters suitable for use with prokaryotic hosts include the beta-lactamase and lactose promoter systems (Chang et al., Nature, 275:615 (1978); Goeddel et al., Nature, 281:544 (1979)), alkaline phosphatase, a tryptophan (trp) promoter system (Goeddel, Nucleic Acids Res., 8:4057 (1980); EP 36,776), and hybrid promoters such as the tac promoter (deBoer et al., Proc. Natl. Acad. Sci. USA, 80:21-25 (1983)). Promoters for use in bacterial systems also will contain a Shine-Dalgarno (S.D.) sequence operably linked to the encoding DNA.

Examples of suitable promoting sequences for use with yeast hosts include the promoters for 3-phosphoglycerate kinase (Hitzeman et al., J. Biol. Chem., 255:2073 (1980)) or other glycolytic enzymes (Hess et al., J. Adv. Enzyme Re.g., 7:149 (1968); Holland, Biochemistry, 17:4900 (1978)), such as enolase, glyceraldehyde-3-phosphate dehydrogenase, hexokinase, pyruvate decarboxylase, phosphofructokinase, glucose-6-phosphate isomerase, 3-phosphoglycerate mutase, pyruvate kinase, triosephosphate isomerase, phosphoglucose isomerase, and glucokinase.

Other yeast promoters, which are inducible promoters having the additional advantage of transcription controlled by growth conditions, are the promoter regions for alcohol dehydrogenase 2, isocytochrome C, acid phosphatase, degradative enzymes associated with nitrogen metabolism, metallothionein, glyceraldehyde-3-phosphate dehydrogenase, and enzymes responsible for maltose and galactose utilization. Suitable vectors and promoters for use in yeast expression are further described in EP 73,657.

Transcription from vectors in mammalian host cells is controlled, for example, by promoters obtained from the genomes of viruses such as polyoma virus, fowlpox virus (UK 2,211,504 published Jul. 5, 1989), adenovirus (such as Adenovirus 2), bovine papilloma virus, avian sarcoma virus, cytomegalovirus, a retrovirus, hepatitis-B virus and Simian Virus 40 (SV40), from heterologous mammalian promoters, e.g., the actin promoter or an immunoglobulin promoter, and from heat-shock promoters, provided such promoters are compatible with the host cell systems.

Transcription of a DNA encoding the stretches of consecutive amino acids or polypeptides of interest by higher eukaryotes may be increased by inserting an enhancer sequence into the vector. Enhancers are cis-acting elements of DNA, usually about from 10 to 300 bp, that act on a promoter to increase its transcription. Many enhancer sequences are now known from mammalian genes (globin, elastase, albumin, alpha-fetoprotein, and insulin). Typically, however, one will use an enhancer from a eukaryotic cell virus. Examples include the SV40 enhancer on the late side of the replication origin (bp 100-270), the cytomegalovirus early promoter enhancer, the polyoma enhancer on the late side of the replication origin, and adenovirus enhancers. The enhancer may be spliced into the vector at a position 5′ or 3′ to the coding sequence, but is preferably located at a site 5′ from the promoter.

Expression vectors used in eukaryotic host cells (yeast, fungi, insect, plant, animal, human, or nucleated cells from other multicellular organisms) will also contain sequences necessary for the termination of transcription and for stabilizing the mRNA. Such sequences are commonly available from the 5′ and, occasionally 3′, untranslated regions of eukaryotic or viral DNAs or cDNAs. These regions contain nucleotide segments transcribed as polyadenylated fragments in the untranslated portion of the mRNA encoding stretches of consecutive amino acids or polypeptides of interest.

Still other methods, vectors, and host cells suitable for adaptation to the synthesis of stretches of consecutive amino acids or polypeptides in recombinant vertebrate cell culture are described in Gething et al., Nature 293:620-625 (1981); Mantei et al., Nature, 281:4046 (1979); EP 117,060; and EP 117,058.

### 3. Detecting Gene Amplification/Expression

Gene amplification and/or expression may be measured in a sample directly, for example, by conventional Southern blotting, Northern blotting to quantitate the transcription of mRNA (Thomas, Proc. Natl. Acad. Sci. USA, 77:5201-5205 (1980)), dot blotting (DNA analysis), or in situ hybridization, using an appropriately labeled probe, based on the sequences provided herein. Alternatively, antibodies may be employed that can recognize specific duplexes, including DNA duplexes, RNA duplexes, and DNA-RNA hybrid duplexes or DNA-protein duplexes. The antibodies in turn may be labeled and the assay may be carried out where the duplex is bound to a surface, so that upon the formation of duplex on the surface, the presence of antibody bound to the duplex can be detected.

Gene expression, alternatively, may be measured by immunological methods, such as immunohistochemical staining of cells or tissue sections and assay of cell culture or body fluids, to quantitate directly the expression of gene product. Antibodies useful for immunohistochemical staining and/or assay of sample fluids may be either monoclonal or polyclonal, and may be prepared in any mammal. Conveniently, the antibodies may be prepared against a native sequence stretches of consecutive amino acids or polypeptides of interest or against a synthetic peptide based on the DNA sequences provided herein or against exogenous sequence fused to DNA encoding a stretch of consecutive amino acids or polypeptide of interest and encoding a specific antibody epitope.

### 4. Purification of Polypeptide

Forms of the stretches of consecutive amino acids or polypeptides of interest may be recovered from culture medium or from host cell lysates. If membrane-bound, it can be released from the membrane using a suitable detergent solution (e.g. Triton-X 100) or by enzymatic cleavage. Cells employed in expression of the stretches of consecutive amino acids or polypeptides of interest can be disrupted by various physical or chemical means, such as freeze-thaw cycling, sonication, mechanical disruption, or cell lysing agents.

It may be desired to purify the stretches of consecutive amino acids or polypeptides of interest from recombinant cell proteins or polypeptides. The following procedures are exemplary of suitable purification procedures: by fractionation on an ion-exchange column; ethanol precipitation; reverse phase HPLC; chromatography on silica or on a cation-exchange resin such as DEAE; chromatofocusing; SDS-PAGE; ammonium sulfate precipitation; gel filtration using, for example, Sephadex G-75; protein A Sepharose columns to remove contaminants such as IgG; and metal chelating columns to bind epitope-tagged forms. Various methods of protein purification may be employed and such methods are known in the art and described for example in Deutscher, Methods in Enzymology, 182 (1990); Scopes, Protein Purification: Principles and Practice, Springer-Verlag, New York (1982). The purification step(s) selected will depend, for example, on the nature of the production process used and the particular stretches of consecutive amino acids or polypeptides of interest produced.

Example of Expression of Stretch of Consecutive Amino Acids or Polypeptide Component of Interest in E. coli

The DNA sequence encoding the desired amino acid sequence of interest or polypeptide is initially amplified using selected PCR primers. The primers should contain restriction enzyme sites which correspond to the restriction enzyme sites on the selected expression vector. A variety of expression vectors may be employed. An example of a suitable vector is pBR322 (derived from E. coli; see Bolivar et al., Gene, 2:95 (1977)) which contains genes for ampicillin and tetracycline resistance. The vector is digested with restriction enzyme and dephosphorylated. The PCR amplified sequences are then ligated into the vector. The vector will preferably include sequences which encode for an antibiotic resistance gene, a trp promoter, a polyhis leader (including the first six STII codons, polyhis sequence, and enterokinase cleavage site), the specific amino acid sequence of interest/polypeptide coding region, lambda transcriptional terminator, and an argU gene.

The ligation mixture is then used to transform a selected E. coli strain using the methods described in Sambrook et al., supra. Transformants are identified by their ability to grow on LB plates and antibiotic resistant colonies are then selected. Plasmid DNA can be isolated and confirmed by restriction analysis and DNA sequencing.

Selected clones can be grown overnight in liquid culture medium such as LB broth supplemented with antibiotics. The overnight culture may subsequently be used to inoculate a larger scale culture. The cells are then grown to a desired optical density, during which the expression promoter is turned on.

After culturing the cells for several more hours, the cells can be harvested by centrifugation. The cell pellet obtained by the centrifugation can be solubilized using various agents known in the art, and the solubilized amino acid sequence of interest or polypeptide can then be purified using a metal chelating column under conditions that allow tight binding of the protein.

The primers can contain restriction enzyme sites which correspond to the restriction enzyme sites on the selected expression vector, and other useful sequences providing for efficient and reliable translation initiation, rapid purification on a metal chelation column, and proteolytic removal with enterokinase. The PCR-amplified, poly-His tagged sequences can be ligated into an expression vector used to transform an E. coli host based on, for example, strain 52 (W3110 fuhA(tonA) Ion galE rpoHts(htpRts) clpP(lacIq). Transformants can first be grown in LB containing 50 mg/ml carbenicillin at 30° C. with shaking until an O.D.600 of 3-5 is reached. Cultures are then diluted 50-100 fold into C RAP media (prepared by mixing 3.57 g (NH₄)₂SO₄, 0.71 g sodium citrate-2H₂O, 1.07 g KCl, 5.36 g Difco yeast extract, 5.36 g Sheffield hycase SF in 500 mL water, as well as 110 mM MPS, pH 7.3, 0.55% (w/v) glucose and 7 mM MgSO₄) and grown for approximately 20-30 hours at 30° C. with shaking. Samples were removed to verify expression by SDS-PAGE analysis, and the bulk culture is centrifuged to pellet the cells. Cell pellets were frozen until purification and refolding.

E. coli paste from 0.5 to 1 L fermentations (6-10 g pellets) was resuspended in 10 volumes (w/v) in 7 M guanidine, 20 mM Tris, pH 8 buffer. Solid sodium sulfite and sodium tetrathionate is added to make final concentrations of 0.1M and 0.02 M, respectively, and the solution was stirred overnight at 4° C. This step results in a denatured protein with all cysteine residues blocked by sulfitolization. The solution was centrifuged at 40,000 rpm in a Beckman Ultracentifuge for 30 min. The supernatant was diluted with 3-5 volumes of metal chelate column buffer (6 M guanidine, 20 mM Tris, pH 7.4) and filtered through 0.22 micron filters to clarify. Depending the clarified extract was loaded onto a 5 mil Qiagen Ni-NTA metal chelate column equilibrated in the metal chelate column buffer. The column was washed with additional buffer containing 50 mM imidazole (Calbiochem, Utrol grade), pH 7.4. The protein was eluted with buffer containing 250 mM imidazole. Fractions containing the desired protein were pooled and stored at 4.degree. C. Protein concentration was estimated by its absorbance at 280 nm using the calculated extinction coefficient based on its amino acid sequence.

### Expression of Stretch of Consecutive Amino Acids or Polypeptides in Mammalian Cells

This general example illustrates a preparation of a glycosylated form of a desired amino acid sequence of interest or polypeptide component by recombinant expression in mammalian cells.

The vector pRK5 (see EP 307,247, published Mar. 15, 1989) can be employed as the expression vector. Optionally, the encoding DNA is ligated into pRK5 with selected restriction enzymes to allow insertion of the DNA using ligation methods such as described in Sambrook et al., supra.

In one embodiment, the selected host cells may be 293 cells. Human 293 cells (ATCC CCL 1573) are grown to confluence in tissue culture plates in medium such as DMEM supplemented with fetal calf serum and optionally, nutrient components and/or antibiotics. About 10 μg of the ligated vector DNA is mixed with about 1 μg DNA encoding the VA RNA gene [Thimmappaya et al., Cell 31:543 (1982)] and dissolved in 500 μl of I mM Tris-HCl, 0.1 mM EDTA, 0.227 M CaCl₂ To this mixture is added, dropwise, 500 μl of 50 mM HEPES (pH 7.35), 280 mM NaCl, 1.5 mM NaPO₄, and a precipitate is allowed to form for 10 minutes at 25° C. The precipitate is suspended and added to the 293 cells and allowed to settle for about four hours at 37° C. The culture medium is aspirated off and 2 ml of 20% glycerol in PBS is added for 30 seconds. The 293 cells are then washed with serum free medium, fresh medium is added and the cells are incubated for about 5 days.

Approximately 24 hours after the transfections, the culture medium is removed and replaced with culture medium (alone) or culture medium containing 200 μCi/ml ³⁵S-cysteine and 200 μCi/ml ³⁵S-methionine. After a 12 hour incubation, the conditioned medium is collected, concentrated on a spin filter, and loaded onto a 15% SDS gel. The processed gel may be dried and exposed to film for a selected period of time to reveal the presence of amino acid sequence of interest or polypeptide component. The cultures containing transfected cells may undergo further incubation (in serum free medium) and the medium is tested in selected bioassays.

In an alternative technique, the nucleic acid amino acid sequence of interest or polypeptide component may be introduced into 293 cells transiently using the dextran sulfate method described by Somparyrac et al., Proc. Natl. Acad. Sci., 12:7575 (1981). 293 cells are grown to maximal density in a spinner flask and 700 μg of the ligated vector is added. The cells are first concentrated from the spinner flask by centrifugation and washed with PBS. The DNA-dextran precipitate is incubated on the cell pellet for four hours. The cells are treated with 20% glycerol for 90 seconds, washed with tissue culture medium, and re-introduced into the spinner flask containing tissue culture medium, 5 μg/ml bovine insulin and 0.1 μg/ml bovine transferrin. After about four days, the conditioned media is centrifuged and filtered to remove cells and debris. The sample containing expressed amino acid sequence of interest or polypeptide component can then be concentrated and purified by any selected method, such as dialysis and/or column chromatography.

In another embodiment, the amino acid sequence of interest or polypeptide component can be expressed in CHO cells. The amino acid sequence of interest or polypeptide component can be transfected into CHO cells using known reagents such as CaPO₄ or DEAE-dextran. As described above, the cell cultures can be incubated, and the medium replaced with culture medium (alone) or medium containing a radiolabel such as ³⁵S-methionine. After determining the presence of amino acid sequence of interest or polypeptide component, the culture medium may be replaced with serum free medium. Preferably, the cultures are incubated for about 6 days, and then the conditioned medium is harvested. The medium containing the expressed amino acid sequence of interest or polypeptide component can then be concentrated and purified by any selected method.

Epitope-tagged amino acid sequence of interest or polypeptide component may also be expressed in host CHO cells. The amino acid sequence of interest or polypeptide component may be subcloned out of a pRK5 vector. The subclone insert can undergo PCR to fuse in frame with a selected epitope tag such as a poly-his tag into a Baculovirus expression vector. The poly-his tagged amino acid sequence of interest or polypeptide component insert can then be subcloned into a SV40 driven vector containing a selection marker such as DHFR for selection of stable clones. Finally, the CHO cells can be transfected (as described above) with the SV40 driven vector. Labeling may be performed, as described above, to verify expression. The culture medium containing the expressed poly-His tagged amino acid sequence of interest or polypeptide component can then be concentrated and purified by any selected method, such as by Ni²⁺-chelate affinity chromatography.

In an embodiment the amino acid sequence of interest or polypeptide component are expressed as an IgG construct (immunoadhesin), in which the coding sequences for the soluble forms (e.g. extracellular domains) of the respective proteins are fused to an IgG1 constant region sequence containing the hinge, CH2 and CH2 domains and/or is a poly-His tagged form.

Following PCR amplification, the respective DNAs are subcloned in a CHO expression vector using standard techniques as described in Ausubel et al., Current Protocols of Molecular Biology, Unit 3.16, John Wiley and Sons (1997). CHO expression vectors are constructed to have compatible restriction sites 5′ and 3′ of the DNA of interest to allow the convenient shuttling of cDNA's. The vector used in expression in CHO cells is as described in Lucas et al., Nucl. Acids Res. 24:9 (1774-1779 (1996), and uses the SV40 early promoter/enhancer to drive expression of the cDNA of interest and dihydrofolate reductase (DHFR). DHFR expression permits selection for stable maintenance of the plasmid following transfection.

### Expression of Stretch of Consecutive Amino Acids or Polypeptides in Yeast

The following method describes recombinant expression of a desired amino acid sequence of interest or polypeptide component in yeast.

First, yeast expression vectors are constructed for intracellular production or secretion of a stretch of consecutive amino acids from the ADH2/GAPDH promoter. DNA encoding a desired amino acid sequence of interest or polypeptide component, a selected signal peptide and the promoter is inserted into suitable restriction enzyme sites in the selected plasmid to direct intracellular expression of the amino acid sequence of interest or polypeptide component. For secretion, DNA encoding the stretch of consecutive amino acids can be cloned into the selected plasmid, together with DNA encoding the ADH2/GAPDH promoter, the yeast alpha-factor secretory signal/leader sequence, and linker sequences (if needed) for expression of the stretch of consecutive amino acids.

Yeast cells, such as yeast strain AB110, can then be transformed with the expression plasmids described above and cultured in selected fermentation media. The transformed yeast supernatants can be analyzed by precipitation with 10% trichloroacetic acid and separation by SDS-PAGE, followed by staining of the gels with Coomassie Blue stain.

Recombinant amino acid sequence of interest or polypeptide component can subsequently be isolated and purified by removing the yeast cells from the fermentation medium by centrifugation and then concentrating the medium using selected cartridge filters. The concentrate containing the amino acid sequence of interest or polypeptide component may further be purified using selected column chromatography resins.

### Expression of Stretches of Stretch of Consecutive Amino Acids or Polypeptides in Baculovirus-Infected Insect Cells

The following method describes recombinant expression of stretches of consecutive amino acids in Baculovirus-infected insect cells.

The desired nucleic acid encoding the stretch of consecutive amino acids is fused upstream of an epitope tag contained with a baculovirus expression vector. Such epitope tags include poly-his tags and immunoglobulin tags (like Fc regions of IgG). A variety of plasmids may be employed, including plasmids derived from commercially available plasmids such as pVL1393 (Novagen). Briefly, the amino acid sequence of interest or polypeptide component or the desired portion of the amino acid sequence of interest or polypeptide component (such as the sequence encoding the extracellular domain of a transmembrane protein) is amplified by PCR with primers complementary to the 5′ and 3′ regions. The 5′ primer may incorporate flanking (selected) restriction enzyme sites. The product is then digested with those selected restriction enzymes and subcloned into the expression vector.

Recombinant baculovirus is generated by co-transfecting the above plasmid and BaculoGold™ virus DNA (Pharmingen) into Spodoptera frugiperda (“Sf9”) cells (ATCC CRL 1711) using lipofectin (commercially available from GIBCO-BRL). After 4-5 days of incubation at 28° C., the released viruses are harvested and used for further amplifications. Viral infection and protein expression is performed as described by O'Reilley et al., Baculovirus expression vectors: A laboratory Manual, Oxford: Oxford University Press (1994).

Expressed poly-his tagged amino acid sequence of interest or polypeptide component can then be purified, for example, by Ni²⁺-chelate affinity chromatography as follows. Extracts are prepared from recombinant virus-infected Sf9 cells as described by Rupert et al., Nature, 362:175-179 (1993). Briefly, Sf9 cells are washed, resuspended in sonication buffer (25 mL Hepes, pH 7.9; 12.5 mM MgCl₂; 0.1 mM EDTA; 10% Glycerol; 0.1% NP40; 0.4 M KCl), and sonicated twice for 20 seconds on ice. The sonicates are cleared by centrifugation, and the supernatant is diluted 50-fold in loading buffer (50 mM phosphate, 300 mM NaCl, 10% Glycerol, pH 7.8) and filtered through a 0.45 μm filter. A Ni²⁺-NTA agarose column (commercially available from Qiagen) is prepared with a bed volume of 5 mL, washed with 25 mL of water and equilibrated with 25 mL of loading buffer. The filtered cell extract is loaded onto the column at 0.5 mL per minute. The column is washed to baseline A₂₈₀ with loading buffer, at which point fraction collection is started. Next, the column is washed with a secondary wash buffer (50 mM phosphate; 300 mM NaCl, 10% Glycerol, pH 6.0), which elutes nonspecifically bound protein. After reaching A₂₈₀ baseline again, the column is developed with a 0 to 500 mM Imidazole gradient in the secondary wash buffer. One mL fractions are collected and analyzed by SDS-PAGE and silver staining or western blot with Ni²⁺-NTA-conjugated to alkaline phosphatase (Qiagen). Fractions containing the eluted His₁₀-tagged sequence are pooled and dialyzed against loading buffer.

Alternatively, purification of the IgG tagged (or Fc tagged) amino acid sequence can be performed using known chromatography techniques, including for instance, Protein A or Protein G column chromatography.

Fc containing constructs of proteins can be purified from conditioned media as follows. The conditioned media is pumped onto a 5 ml Protein A column (Pharmacia) which is equilibrated in 20 mM Na phosphate buffer, pH 6.8. After loading, the column is washed extensively with equilibration buffer before elution with 100 mM citric acid, pH 3.5. The eluted protein is immediately neutralized by collecting 1 ml fractions into tubes containing 275 mL of 1 M Tris buffer, pH 9. The highly purified protein is subsequently desalted into storage buffer as described above for the poly-His tagged proteins. The homogeneity of the proteins is verified by SDS polyacrylamide gel (PEG) electrophoresis and N-terminal amino acid sequencing by Edman degradation.

### Examples of Pharmaceutical Compositions

Non-limiting examples of such compositions and dosages are set forth as follows:

Compositions comprising a compound comprising a stretch of consecutive amino acids which comprises consecutive amino acids having the sequence of etanercept (e.g. Enbrel) may comprise mannitol, sucrose, and tromethamine. In an embodiment, the composition is in the form of a lyophilizate. In an embodiment, the composition is reconstituted with, for example, Sterile Bacteriostatic Water for Injection (BWFI), USP (containing 0.9% benzyl alcohol). In an embodiment the compound is administered to a subject for reducing signs and symptoms, inducing major clinical response, inhibiting the progression of structural damage, and improving physical function in subjects with moderately to severely active rheumatoid arthritis. The compound may be initiated in combination with methotrexate (MTX) or used alone. In an embodiment the compound is administered to a subject for reducing signs and symptoms of moderately to severely active polyarticular-course juvenile rheumatoid arthritis in subjects who have had an inadequate response to one or more DMARDs. In an embodiment the compound is administered to a subject for reducing signs and symptoms, inhibiting the progression of structural damage of active arthritis, and improving physical function in subjects with psoriatic arthritis. In an embodiment the compound is administered to a subject for reducing signs and symptoms in subjects with active ankylosing spondylitis. In an embodiment the compound is administered to a subject for the treatment of chronic moderate to severe plaque psoriasis. In an embodiment wherein the subject has rheumatoid arthritis, psoriatic arthritis, or ankylosing spondylitis the compound is administered at 25-75 mg per week given as one or more subcutaneous (SC) injections. In a further embodiment the compound is administered at 50 mg per week in a single SC injection. In an embodiment wherein the subject has plaque psoriasis the compound is administered at 25-75 mg twice weekly or 4 days apart for 3 months followed by a reduction to a maintenance dose of 25-75 mg per week. In a further embodiment the compound is administered at a dose of at 50 mg twice weekly or 4 days apart for 3 months followed by a reduction to a maintenance dose of 50 mg per week. In an embodiment the dose is between 2× and 100× less than the doses set forth herein. In an embodiment wherein the subject has active polyarticular-course JRA the compound may be administered at a dose of 0.2-1.2 mg/kg per week (up to a maximum of 75 mg per week). In a further embodiment the compound is administered at a dose of 0.8 mg/kg per week (up to a maximum of 50 mg per week). In some embodiments the dose is between 2× and 100× less than the doses set forth hereinabove.

Compositions comprising a compound comprising a stretch of consecutive amino acids which comprises consecutive amino acids having the sequence of infliximab (e.g. Remicade) may comprise sucrose, polysorbate 80, monobasic sodium phosphate, monohydrate, and dibasic sodium phosphate, dihydrate. Preservatives are not present in one embodiment. In an embodiment, the composition is in the form of a lyophilizate. In an embodiment, the composition is reconstituted with, for example, Water for Injection (BWFI), USP. In an embodiment the pH of the composition is 7.2 or is about 7.2. In one embodiment the compound is administered is administered to a subject with rheumatoid arthritis in a dose of 2-4 mg/kg given as an intravenous infusion followed with additional similar doses at 2 and 6 weeks after the first infusion then every 8 weeks thereafter. In a further embodiment the compound is administered in a dose of 3 mg/kg given as an intravenous infusion followed with additional similar doses at 2 and 6 weeks after the first infusion then every 8 weeks thereafter. In an embodiment the dose is adjusted up to 10 mg/kg or treating as often as every 4 weeks. In an embodiment the compound is administered in combination with methotrexate. In one embodiment the compound is administered is administered to a subject with Crohn's disease or fistulizing Crohn's disease at dose of 2-7 mg/kg given as an induction regimen at 0, 2 and 6 weeks followed by a maintenance regimen of 4-6 mg/kg every 8 weeks thereafter for the treatment of moderately to severely active Crohn's disease or fistulizing disease. In a further embodiment the compound is administered at a dose of 5 mg/kg given as an induction regimen at 0, 2 and 6 weeks followed by a maintenance regimen of 5 mg/kg every 8 weeks thereafter for the treatment of moderately to severely active Crohn's disease or fistulizing disease. In an embodiment the dose is adjusted up to 10 mg/kg. In one embodiment the compound is administered to a subject with ankylosing spondylitis at a dose of 2-7 mg/kg given as an intravenous infusion followed with additional similar doses at 2 and 6 weeks after the first infusion, then every 6 weeks thereafter. In a further embodiment the compound is administered at a dose of 5 mg/kg given as an intravenous infusion followed with additional similar doses at 2 and 6 weeks after the first infusion, then every 6 weeks thereafter. In one embodiment the compound is administered to a subject with psoriatic arthritis at a dose of 2-7 mg/kg given as an intravenous infusion followed with additional similar doses at 2 and 6 weeks after the first infusion then every 8 weeks thereafter. In a further embodiment the compound is administered at a dose of 5 mg/kg given as an intravenous infusion followed with additional similar doses at 2 and 6 weeks after the first infusion then every 8 weeks thereafter. In an embodiment the compound is administered with methotrexate. In one embodiment the compound is administered to a subject with ulcerative colitis at a dose of 2-7 mg/kg given as an induction regimen at 0, 2 and 6 weeks followed by a maintenance regimen of 2-7 mg/kg every 8 weeks thereafter for the treatment of moderately to severely active ulcerative colitis. In a further embodiment the compound is administered to a subject with ulcerative colitis at a dose of 5 mg/kg given as an induction regimen at 0, 2 and 6 weeks followed by a maintenance regimen of 5 mg/kg every 8 weeks thereafter. In some embodiments the dose is between 2× and 100× less than the doses set forth hereinabove for treating the individual diseases.

In each of the embodiments of the compositions described herein, the compositions, when in the form of a lyophilizate, may be reconstituted with, for example, sterile aqueous solutions, sterile water, Sterile Water for Injections (USP), Sterile Bacteriostatic Water for Injections (USP), and equivalents thereof known to those skilled in the art.

It is understood that in administration of any of the instant compounds, the compound may be administered in isolation, in a carrier, as part of a pharmaceutical composition, or in any appropriate vehicle.

### Dosage

It is understood that where a dosage range is stated herein, e.g. 1-10 mg/kg per week, the invention disclosed herein also contemplates each integer dose, and tenth thereof, between the upper and lower limits. In the case of the example given, therefore, the invention contemplates 1.0, 1.1, 1.2, 1.3, 1.4, 1.5, 1.6, 1.7, 1.8, 1.9, 2.0, 2.1, 2.2, 2.3, 2.4 etc. mg/kg up to 10 mg/kg.

In embodiments, the compounds of the present invention can be administered as a single dose or may be administered as multiple doses.

In general, the daily dosage for treating a disorder or condition according to the methods described above will generally range from about 0.01 to about 10.0 mg/kg body weight of the subject to be treated.

Variations based on the aforementioned dosage ranges may be made by a physician of ordinary skill taking into account known considerations such as the weight, age, and condition of the person being treated, the severity of the affliction, and the particular route of administration chosen.

It is also expected that the compounds disclosed will effect cooperative binding with attendant consequences on effective dosages required.

### Pharmaceuticals

The term “pharmaceutically acceptable carrier” is understood to include excipients, carriers or diluents. The particular carrier, diluent or excipient used will depend upon the means and purpose for which the active ingredient is being applied.

For parenteral administration, solutions containing a compound of this invention or a pharmaceutically acceptable salt thereof in sterile aqueous solution may be employed. Such aqueous solutions should be suitably buffered if necessary and the liquid diluent first rendered isotonic with sufficient saline or glucose. These particular aqueous solutions are especially suitable for intravenous, intramuscular, subcutaneous and intraperitoneal administration. The sterile aqueous media employed are all readily available by standard techniques known to those skilled in the art.

The compositions of this invention may be in a variety of forms. These include, for example, liquid, semi-solid and solid dosage forms, such as liquid solutions (e.g., injectable and infusible solutions), dispersions or suspensions. The preferred form depends on the intended mode of administration and therapeutic application. Some compositions are in the form of injectable or infusible solutions. A mode of administration is parenteral (e.g., intravenous, subcutaneous, intraperitoneal, intramuscular). In an embodiment, the compound is administered by intravenous infusion or injection. In another embodiment, the compound is administered by intramuscular or subcutaneous injection.

For therapeutic use, the compositions disclosed here can be administered in various manners, including soluble form by bolus injection, continuous infusion, sustained release from implants, oral ingestion, local injection (e.g. intracardiac, intramuscular), systemic injection, or other suitable techniques well known in the pharmaceutical arts. Other methods of pharmaceutical administration include, but are not limited to oral, subcutaneously, transdermal, intravenous, intramuscular and parenteral methods of administration. Typically, a soluble composition will comprise a purified compound in conjunction with physiologically acceptable carriers, excipients or diluents. Such carriers will be nontoxic to recipients at the dosages and concentrations employed. The preparation of such compositions can entail combining a compound with buffers, antioxidants, carbohydrates including glucose, sucrose or dextrins, chelating agents such as EDTA, glutathione and other stabilizers and excipients. Neutral buffered saline or saline mixed with conspecific serum albumin are exemplary appropriate diluents. The product can be formulated as a lyophilizate using appropriate excipient solutions (e.g., sucrose) as diluents.

Other derivatives comprise the compounds/compositions of this invention covalently bonded to a nonproteinaceous polymer. The bonding to the polymer is generally conducted so as not to interfere with the preferred biological activity of the compound, e.g. the binding activity of the compound to a target. The nonproteinaceous polymer ordinarily is a hydrophilic synthetic polymer, i.e., a polymer not otherwise found in nature. However, polymers which exist in nature and are produced by recombinant or in vitro methods are useful, as are polymers which are isolated from nature. Hydrophilic polyvinyl polymers fall within the scope of this invention, e.g. polyvinylalcohol and polyvinylpyrrolidone. Particularly useful are polyalkylene ethers such as polyethylene glycol, polypropylene glycol, polyoxyethylene esters or methoxy polyethylene glycol; polyoxyalkylenes such as polyoxyethylene, polyoxypropylene, and block copolymers of polyoxyethylene and polyoxypropylene (Pluronics); polymethacrylates; carbomers; branched or unbranched polysaccharides which comprise the saccharide monomers D-mannose, D- and L-galactose, fucose, fructose, D-xylose, L-arabinose, D-glucuronic acid, sialic acid, D-galacturontc acid, D-mannuronic acid (e.g. polymannuronic acid, or alginic acid), D-glucosamine, D-galactosamine, D-glucose and neuraminic acid including homopolysaccharides and heteropolysaccharides such as lactose, amylopectin, starch, hydroxyethyl starch, amylose, dextran sulfate, dextran, dextrins, glycogen, or the polysaccharide subunit of acid mucopolysaccharides, e.g. hyaluronic acid; polymers of sugar alcohols such as polysorbitol and polymannitol; as well as heparin or heparon.

The pharmaceutical compositions of the invention may include a “therapeutically effective amount” or a “prophylactically effective amount” of a compound of the invention. A “therapeutically effective amount” refers to an amount effective, at dosages and for periods of time necessary, to achieve the desired therapeutic result. A therapeutically effective amount of the compound may vary according to factors such as the disease state, age, sex, and weight of the individual. A therapeutically effective amount is also one in which any toxic or detrimental effects of the compound are outweighed by the therapeutically beneficial effects. A “prophylactically effective amount” refers to an amount effective, at dosages and for periods of time necessary, to achieve the desired prophylactic result. Typically, since a prophylactic dose is used in subjects prior to or at an earlier stage of disease, the prophylactically effective amount will be less than the therapeutically effective amount.

All combinations of the various elements disclosed herein are within the scope of the invention.

This invention will be better understood by reference to the Experimental Details which follow, but those skilled in the art will readily appreciate that the specific experiments detailed are only illustrative of the invention as described more fully in the claims which follow thereafter.

### Experimental Details

### Example 1: TNR1B-alkyne-azide-Fc6

TNR1B-alkyne-azide-Fc6 was prepared via the reaction of alkyne-modified TNR1B (TNF receptor 1B) with azide-modified Fc6 as follows. TNR1B-azide-alkyne-Fc6 is prepared using the same principles via the reaction of azide-modified TNR1B with alkyne-modified Fc6.

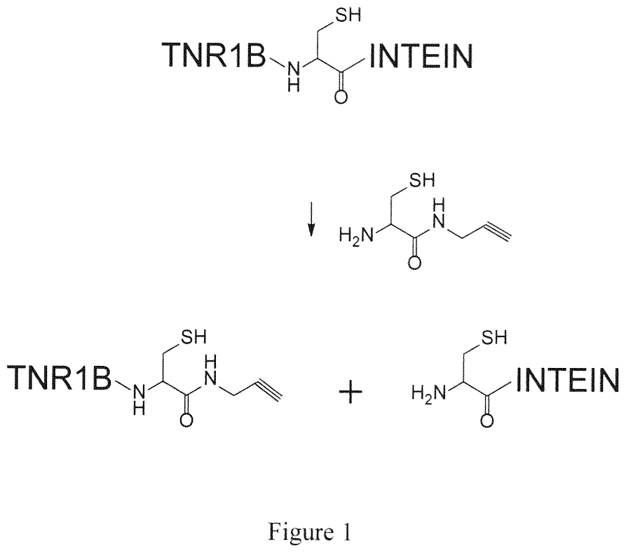

Alkyne-modified TNFR1B (TNR1B-Alk) was prepared by cleavage of TNR1B-intein (TNR1B-Mth fusion protein) with cystyl-propargylamide, HSCH₂CH[NH2]CONHCH₂C≡CH₃ (FIG. 1) and azide-modified TNR1B (TNR1B-Az) was prepared by cleavage of TNR1B-intein with cystyl-3-azidopropylamide, HSCH₂CH[NH₂]CONH(CH₂)₃NH₂.

TNR1B-intein and Fc6 are described in U.S. Ser. No. 11/982,085, published Oct. 16, 2008, the whole of which is incorporated herein by reference.

TNR1B-intein fusion protein was produced using vector pCDNA3-TNR1B-Mth, the sequence of which is shown in SEQ ID NO: 100. The pre-TNR1B-intein chimeric polypeptide that is initially expressed, containing the TNR1B extracellular domain joined at its C-terminus by a peptide bond to the N-terminus of an Mth RIR1 self-splicing intein at the autocleavage site, is shown in SEQ ID NO: 101. Cleavage of the homologous TNR signal sequences by the cellular signal peptidase provides the mature TNR1B-intein fusion protein that is secreted into the cell culture fluid, the sequence of which is shown in SEQ ID NO: 102.

Fc6 protein was expressed using vector pCDNA3-SHH-IgG1-Fc11, the sequence of which is shown in SEQ ID NO: 103. The pre-Fc6 polypeptide that is initially expressed is shown in SEQ ID NO: 104. Cleavage of the heterologous sonic hedgehog (SHH) signal sequences by the cellular signal peptidase provides the mature Fc6 protein that is secreted into the cell culture fluid, the sequence of which is shown in SEQ ID NO: 105.

Protein production was executed by transient expression in CHO-DG44 cells, adapted to serum-free suspension culture. Transient transfections were done with polyethylenimine as transfection agent, complexed with DNA, under high density conditions as described by Rajendra et al., J. Biotechnol. 153, 22-26 (2011). Seed train cultures were maintained in TubeSpin® bioreactor 50 tubes obtained from TPP (Trasadingen, CH) and scaled up in volume to generate sufficient biomass for transfection. Transfections were carried out in cultures of 0.5-1.0 L. Cultures at this scale were maintained in 2 L or 5 L Schott-bottles with a ventilated cap. The bottles were shaken at 180 rpm in a Kühner incubator shaker with humidification and CO₂ control at 5%. The cell culture fluid was harvested after 10 days, centrifuged and sterile-filtered, prior to purification.

Cystyl-propargylamide and cystyl-3-azidopropylamide were prepared as follows. Boc-Cys(Trt)-OH, (C₆H₅)₃CSCH₂CH[NHCO₂C(CH₃)₃]CO₂H; propargylamine, HC≡CCH₂NH; 3-azidopropylamine, NH₂CH₂CH₂CH₂N3; EDC, N-(3-Dimethylaminopropyl)-N′-ethylcarbodiimide hydrochloride; and HOBt, 1-Hydroxybenzotriazole, and were obtained from AnaSpec (Freemont, CA) or CPC Scientific (San Jose, CA). All other chemicals were obtained from Sigma-Aldrich (St. Louis, MO). For the synthesis of cystyl-propargylamide, a solution of Boc-Cys(Trt)-OH (100 mM) and propargylamine (100 mM) in CH2Cl2 was made 100 mM each in EDC, HOBt, and triethylamine. For the synthesis of cystyl-3-azidopropylamide, 3-azidopropylamine (100 mM) was substituted for propargylamine. Both reactions were worked up by the following procedure. After stirring overnight at room temperature, the reaction was stopped with an excess of saturated NaHCO₃ in water, extracted with CH2Cl2, dried over MgSO4, filtered, evaporated, and purified by column chromatography. To remove the Boc/Trt protecting groups, the intermediate product was dissolved at a concentration of 0.05M in TFA/triisopropylsilane/H2O (90:5:5) and stirred for 30 minutes at room temperature. The reaction was then dried by evaporation and extracted with CH2Cl2. The organic layer was then extracted with water, yielding the final cystyl-propargylamide product as a yellowish oil, and the final cystyl-3-azidopropylamide product as a yellowish solid.

To prepare the alkyne-modified TNR1B (FIG. 1) or the azide-modified TNR1B, the TNR1B-intein protein in the cell culture fluid was applied to a column packed with chitin beads obtained from New England BioLabs (Beverley, MA) that was pre-equilibrated with buffer A (20 mM Tris-HCl, 500 mM NaCl, pH 7.5). Unbound protein was washed from the column with buffer A. Cleavage was initiated by rapidly equilibrating the chitin resin in buffer B (20 mM Tris-HCl, 500 mM NaCl, pH 8.0) containing either 50 mM cystyl-propargylamide (for alkyne-modified TNR1B) or 50 mM cystyl-3-azidopropylamide (for azide-modified TNR1B) and incubation was carried out for 24 to 96 hours at room temperature. The cleaved alkyne-modified TNR1B (SEQ ID NO: 106) or azide-modified TNR1B proteins (SEQ ID NO: 107) were eluted from the column with buffer A, concentrated using an Amicon Ultracel-3 Centrifugal Filter Unit from Millipore (Billerica, MA), dialyzed against Dulbecco's phosphate buffered saline without Ca or Mg salts (PBS) obtained from the UCSF Cell Culture Facility (San Francisco, CA), and stored at 4° C. prior to use.

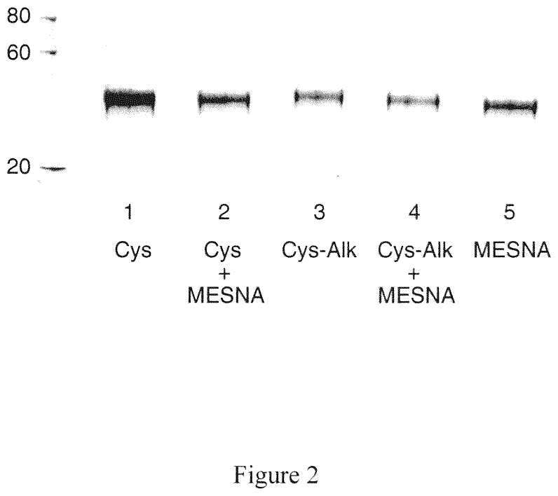

FIG. 2 shows SDS-polyacrylamide gel electrophoresis (SDS-PAGE) analysis of the alkyne-modified TNR1B, compared with cysteine-modified TNR1B (SEQ ID NO: 108) prepared using 50 mM cysteine instead of cystyl-propargylamide. SDS-PAGE was carried out using NuPAGE® Novex Bis-Tris Midi Gels (10%) obtained from Invitrogen (Carlsbad, CA). Proteins were visualized using Silver Stain Plus or Bio-Safe Coomassie Stain obtained from Bio-Rad (Hercules, CA). The alkyne-modified TNR1B (lane 3) and the cysteine-modified TNR1B (lane 1) had the same Mr ~43,000. In addition, the alkyne-modified TNR1B had comparable biological activity to cysteine-modified TNR1B as measured using a Human sTNFRII/TNFRSF1B Quantikine ELISA obtained from R&D Systems (Minneapolis, MN). Preparations of the cysteine-modified TNR1B (lane 2), alkyne-modified TNR1B (lane 4), or thioester-modified TNR1B (SEQ ID NO: 109) (lane 5) made in the presence of 50 mM MESNA had a similar Mr, but had less than 5% of the biological activity observed for preparations made in the absence of MESNA. Thus, alkyne-modified TNR1B prepared in the absence of MESNA was employed in further studies.

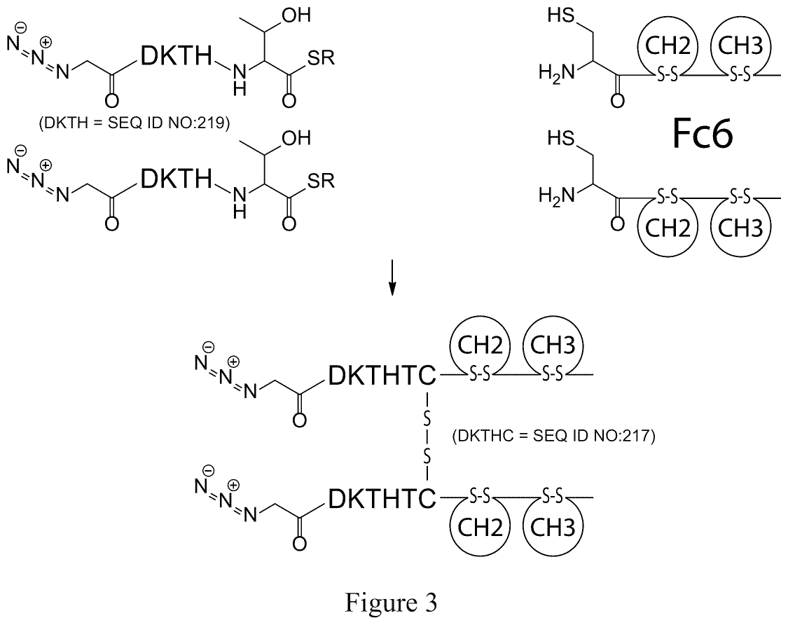

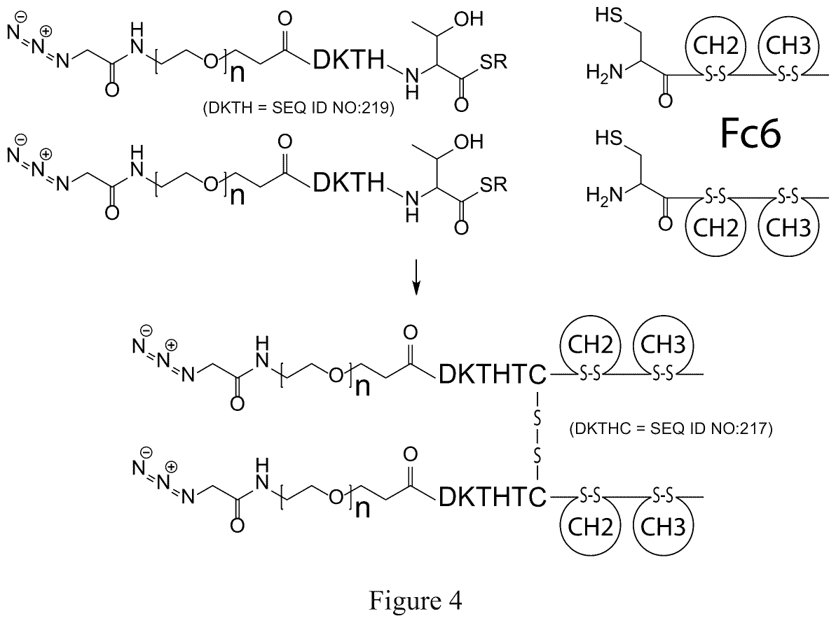

Azide-modified Fc6 (Az-Fc6) was prepared by the reaction of Fc6 protein with various azide-containing peptide thioesters (FIG. 3) and azide-containing PEG thioesters (FIG. 4). Alkyne-modified Fc6 (Alk-Fc6) was prepared by the reaction of alkyne-containing thioesters with Fc6 protein.

Recombinant Fc6 protein was expressed in Chinese hamster ovary (CHO) cells as described for TNR1B-intein (see above) and purified by Protein A affinity chromatography. The culture supernatant was applied to a column packed with rProtein A Fast Flow from Pharmacia (Uppsala, Sweden) pre-equilibrated with PBS. The column was washed extensively with PBS and the Fc6 protein then eluted with 0.1 M glycine buffer pH 2.7. Fractions were collected into tubes containing 0.05 vol/vol of 1.0 M Tris-HCl pH 9.0 (giving a final pH of 7.5), pooled, dialyzed against PBS, and stored at 4° C. prior to use.

Table 1 shows representative azide-containing and alkyne-containing peptide/PEG thioesters. Thioesters were synthesized by an Fmoc/t-Butyl solid-phase strategy on a 2-chlorotrityl chloride resin preloaded with the Fmoc-Thr(tBu)-OH. Amino acid derivatives were obtained from CPC Scientific (Sunnyvale, CA), Fmoc-PEGₙ-OH derivatives were obtained from Quanta BioDesign (Powell, OH), and 2-(1H-benzotriazole-1-yl)-1,1,3,3-tetramethylaminium hexafluorophosphate (HBTU), dichloromethane (DCM), trichloroacetic acid (TFA), N,N′-diisopropylcarbodiimide (DIC), 1-hydroxybenzotriazole (HOBt), N,N′-diisopropylethylamine (DIEA) and triisopropylsilane (TIS) were obtained from Sigma (St. Louis, MO). The standard HBTU activation was employed for peptide elongation. Peptides containing PEG required the insertion of a Fmoc-PEGₙ-OH. As a final step in peptide elongation, the terminal α-Fmoc (9-fluorenylmethoxycarbonyl) protecting group was converted to Boc (tert-butoxycarbonyl). The peptide resin was washed with DCM and cleaved with 1% TFA/DCM to yield the fully protected peptide with a free carboxylic acid on the C-terminus. The thioester of the peptides was formed by treating the crude protected peptide with DIC/HOBt/DIEA and benzyl mercaptan or thiophenol in DCM overnight. After concentration, the crude protected peptide thioester was precipitated by multiple triturations with cold ether followed by centrifugation. Deprotection was carried out by treatment of the crude protected product with 95:2.5:2.5 TFA/TIS/H₂O for 2 hours at room temperature. After precipitation with ice-cold ether the deprotected peptide thioester was purified by preparative RP-HPLC in a H₂O-acetonitrile (0.1% TFA) system to afford the final product with 91-95% purity and the desired MS.

Azide-modified Fc6 and alkyne-modified Fc6 were prepared by native chemical ligation as follows. 2-(N-morpholino)ethanesulfonic acid (MES) was obtained from Acros (Morris Plains, NJ), tris(2-carboxyethyl)phosphine (TCEP) was obtained from Pierce (Rockford, IL), and 4-mercaptophenylacetic acid (MPAA) was obtained from Sigma-Aldrich (St. Louis, MO). Reactions were carried out by ligating the various thioesters shown in Table 1 with the Fc6 protein as follows. Reactions (100 uL) contained 50 mM MES buffer, pH 6.5, 0.8 mM TCEP, 10 mM MPAA, 4 mg/ml of the peptide thioester, and 0.5 mg/ml of the Fc6 protein. Following overnight incubation at room temperature, reactions were adjusted to pH 7.0 with 0.05 vol/vol of 1.0 M Tris-HCl pH 9.0, purified using Protein A Magnetic Beads from New England BioLabs, dialyzed in 0.1 M phosphate pH 8.0, and concentrated.

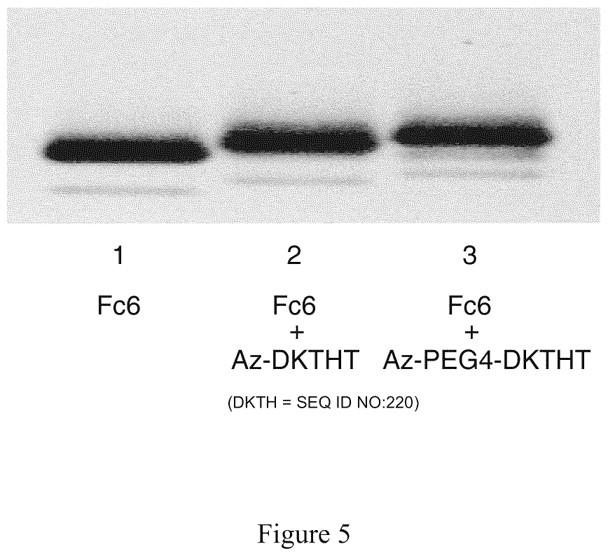

FIG. 5 shows SDS-PAGE analysis demonstrating that Fc6 protein (lane 1) reacted quantitatively with azide-DKTHT-thioester to yield the Az-DKTHT-Fc6 protein (lane 2) and azide-PEG₄-DKTHT-thioester to yield the Az-PEG₄-DKTHT-Fc6 protein (lane 3). DKTHT is SEQ ID NO: 220. The sequence of the Az-DKTHT-Fc6 protein is shown in SEQ ID NO: 110 and the sequence of the Az-PEG₄-DKTHT-Fc6 is shown in SEQ ID NO: 111. The PEG₄ oligomer gave an incremental size increase comparable to the 5 amino acid DKTHT sequence (SEQ ID NO:220). This shows that a single oxyethylene monomer unit makes a contribution to contour length similar to a single amino acid residue, consistent with their having comparable fully extended conformations of ~3.5 to 4 Å (Flory (1969) Statistical Mechanics of Chain Molecules (Interscience Publishers, New York).

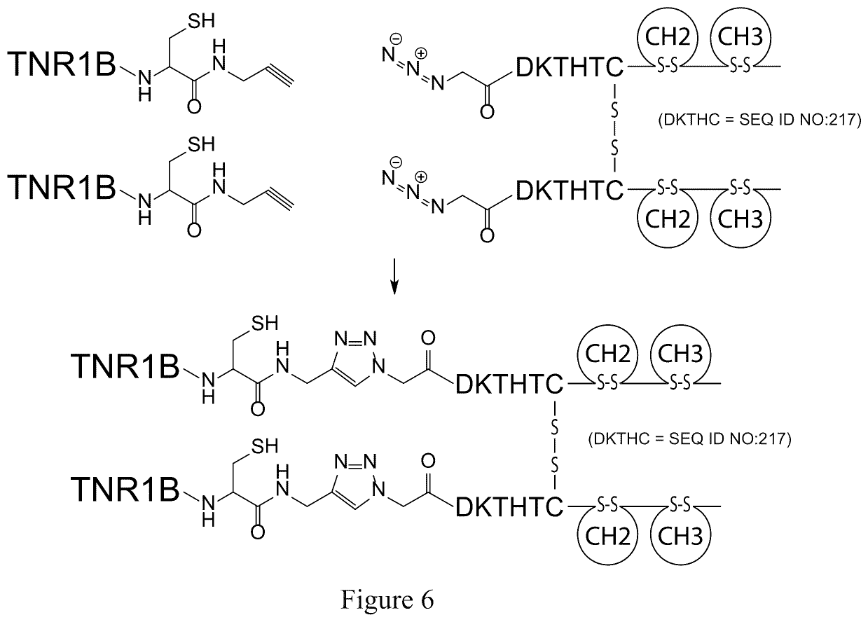

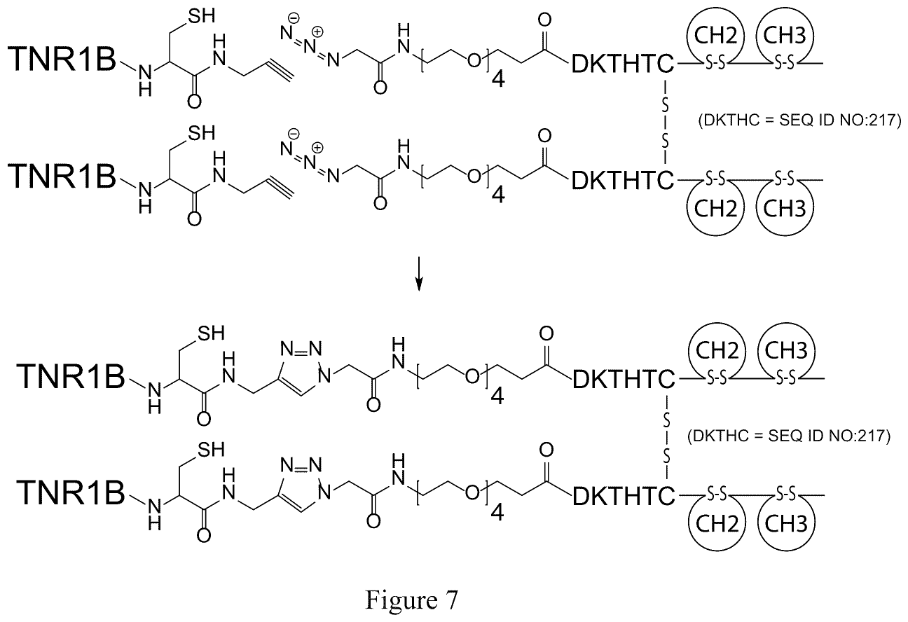

TNR1B-alkyne-azide-Fc6 was prepared via the reaction of the alkyne-modified TNR1B with the Az-DKTHT-Fc6 protein (FIG. 6) and the Az-PEG₄-DKTHT-Fc6 protein (FIG. 7). DKTHT is SEQ ID NO: 220. Sodium phosphate, dibasic (anhydrous) and sodium phosphate, monobasic (monohydrate) were obtained from Acros, TCEP was from Pierce, CuSO₄ (pentahydrate) was from Sigma-Aldrich, and Tris[1-benzyl-1H-1,2,3-triazol-4-yl)methyl]amine (TBTA) from AnaSpec (Freemont, CA). Reactions (60 uL) contained 0.1 M sodium phosphate, pH 8.0, 1.0 mM CuSO₄, 2.0 mM TBTA, the alkyne-modified TNR1B (30 ug), and either the unmodified Fc6 protein, the Az-DKTHT-Fc6 protein, or the Az-PEG₄-DKTHT-Fc6 protein (10 ug). DKTHT is SEQ ID NO: 220. Reactions were initiated by the addition of 2.0 mM TCEP, and incubated overnight at room temperature. The reaction products were purified using Protein A Magnetic Beads to remove any unreacted alkyne-modified TNR1B.

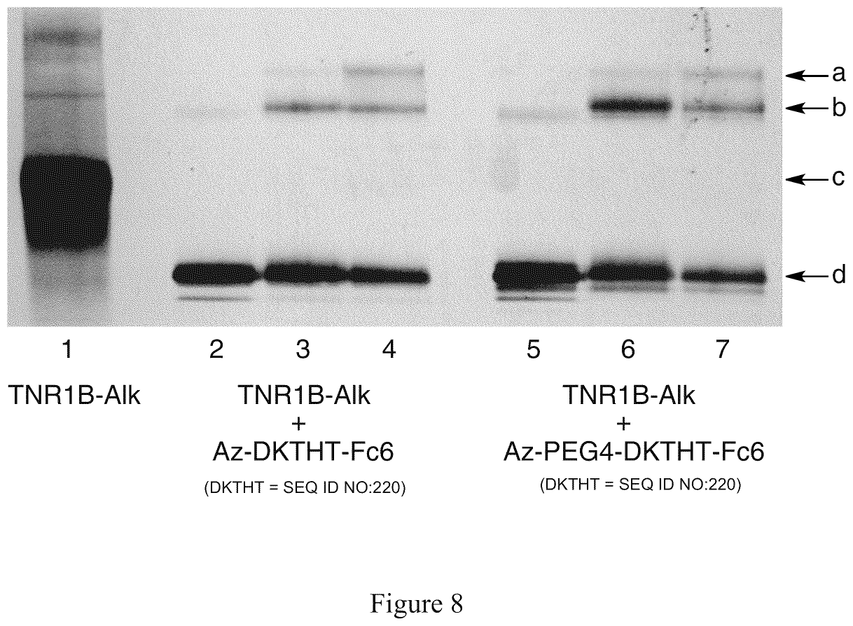

FIG. 8 shows SDS-PAGE analysis of the TNR1B-alkyne-azide-Fc6 products under reducing conditions. In the absence of CuSO₄, TBTA and TCEP, both Az-DKTHT-Fc6 (lane 2) and Az-PEG₄-DKTHT-Fc6 (lane 5) gave a single band of Mr ~28-30,000 daltons (arrow d) corresponding to the input azide-modified Fc6 proteins, with no sign of any product formation. DKTHT is SEQ ID NO: 220. In addition, there was no evidence of any carryover of the input alkyne-modified TNR1B (shown in lane 1) following the Protein A purification. However, in the presence of CuSO₄, TBTA and TCEP, the reaction between alkyne-modified TNR1B and Az-DKTHT-Fc6 (lane 3) and the reaction between alkyne-modified TNR1B and Az-PEG₄-DKTHT-Fc6 (lane 6) both yielded two new products of Mr ~75,000 daltons (arrow a) and ~65,000 daltons (arrow b). DKTHT is SEQ ID NO: 220. Reactions carried out using a preparation of alkyne-modified TNR1B following buffer-exchange in 0.1 M phosphate pH 8.0 to remove salt gave essentially similar reaction products with both Az-DKTHT-Fc6 (lane 4) and Az-PEG₄-DKTHT-Fc6 (lane 6), although there was a significant increase in the yield of the Mr ~75,000 dalton product over the Mr ~65,000 dalton product. DKTHT is SEQ ID NO: 220.

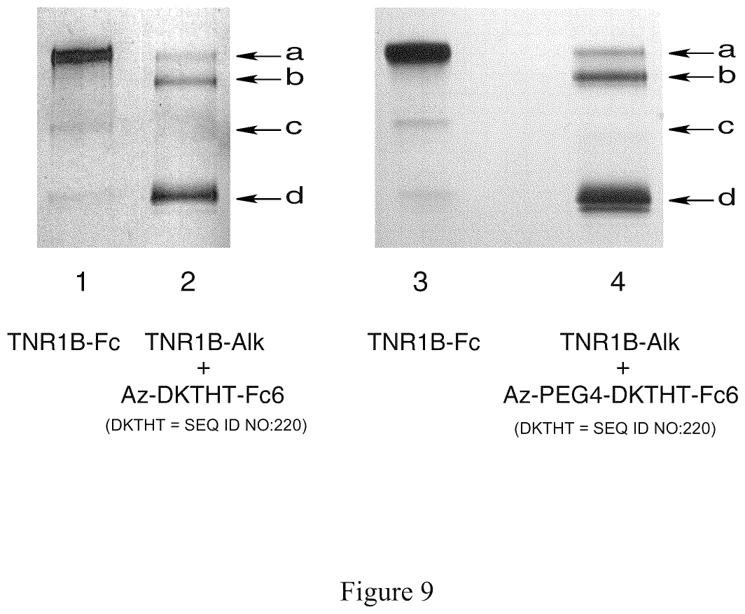

FIG. 9 shows SDS-PAGE analysis comparing the TNR1B-alkyne-azide-Fc6 reaction products (left panel) and the TNR1B-alkyne-azide-PEG₄-Fc6 reaction products (right panel) with TNR1B-Fc fusion protein (etanercept). The TNR1B-alkyne-azide-Fc6 product of Mr ~75,000 daltons (lane 2), having the predicted sequence shown in SEQ ID NO: 112, and the TNR1B-alkyne-azide-PEG₄-Fc6 product of Mr ~75,000 daltons (lane 4), having the predicted sequence of shown in SEQ ID NO: 113, are essentially indistinguishable in size from etanercept (lanes 1, 3), the sequence of which is shown in SEQ ID NO: 114.

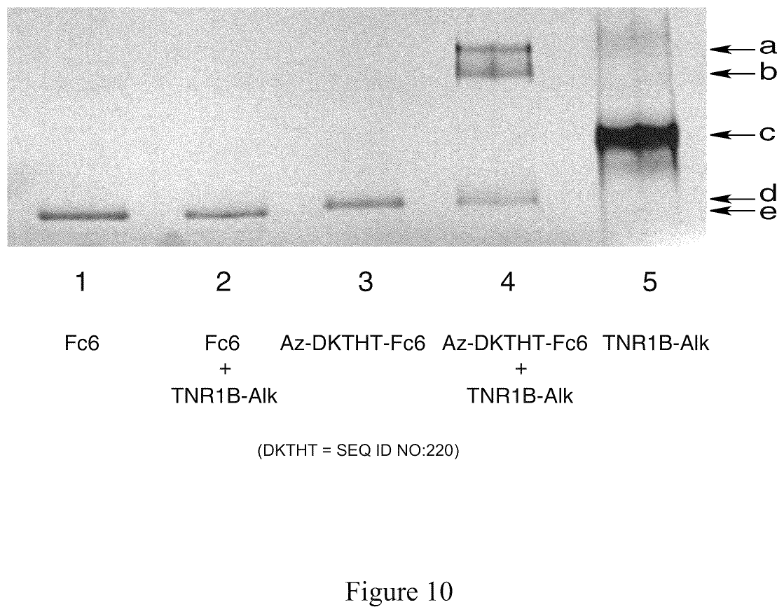

FIG. 10 shows SDS-PAGE analysis providing further evidence confirming the requirement of the alkyne and azide groups for reactivity. Reaction mixtures that contained alkyne-modified TNR1B with unmodified Fc6 protein gave no reaction product (lane 2) compared with Fc6 alone (lane 1), while alkyne-modified TNR1B with Az-DKTHT-Fc6 gave the expected products (lane 4) compared with Az-DKTHT-Fc6 alone (lane 3). Again, no carryover of the input alkyne-modified TNR1B (shown in lane 5) was apparent following the Protein A purification. DKTHT is SEQ ID NO: 220.

The TNR1B-alkyne-azide-Fc6 products of FIG. 10 were further characterized by sequencing of their tryptic peptide by LC-MS. Following SDS-PAGE, the gel was Coomassie stained and four gel regions were excised, corresponding to the Mr ~75,000 product (arrow a), the Mr ~65,000 product (arrow b), the unstained region where alkyne-modified TNR1B would migrate (arrow c), and the unreacted Az-DKTHT-Fc6 protein of Mr ~28,000 (arrow d). DKTHT is SEQ ID NO: 220. The four gel slices were diced into small small pieces (~0.5-1.0 mm³) and processed as follows. Ammonium bicarbonate, acetonitrile, dithiothreitol, and iodoacetamide were obtained from Sigma-Aldrich, formic acid was obtained from Pierce, and porcine trypsin (sequencing grade) was obtained from Promega (Madison, WI). To remove the Coomassie stain, each gel slice was extracted with 200 μL of 25 mM NH₄HCO₃ in 50% acetonitrile by vortexing, centrifuged to remove the supernatant, and dehydrated by adding acetonitrile for a few minutes until the gel pieces shrank and turned white. The acetonitrile was discarded, and the gel slices dried in a Speed Vac (Savant Instruments, Farmingdale, NY). Reduction and alkylation was then carried out by rehydrating the gel slices in 40 ul of 10 mM dithiothreitol in 25 mM NH₄HCO₃, vortexing, and incubated at 56° C. for 45 minutes. The supernatant was then discarded, 40 μL of 55 mM iodoacetamide in 25 mM NH₄HCO₃ was added, the gel slices vortexed and incubated in the dark for 30 minutes at room temperature. The supernatant was discarded, the gel slices again dehydrated in acetonitrile and dried in a Speed Vac. Trypsin digestion was then carried out by rehydrating the gel slices in 25 μL of trypsin (12.5 ug/mL) in 25 mM NH₄HCO₃ on ice for 60 minutes. Excess trypsin solution was then removed, the gel slices covered with 25 mM NH₄HCO₃ and incubated at 37° C. overnight. The supernatant was removed, and the gel then extracted twice with 30 μL of 50% acetonitrile/0.1% formic acid in water. The organic extracts were combined with the aqueous supernatant, reduced to a volume of 10 uL in a Speed Vac, then analysed by LC-MS using a Q-Star Elite mass spectrometer (AB SCIEX, Foster City, CA).

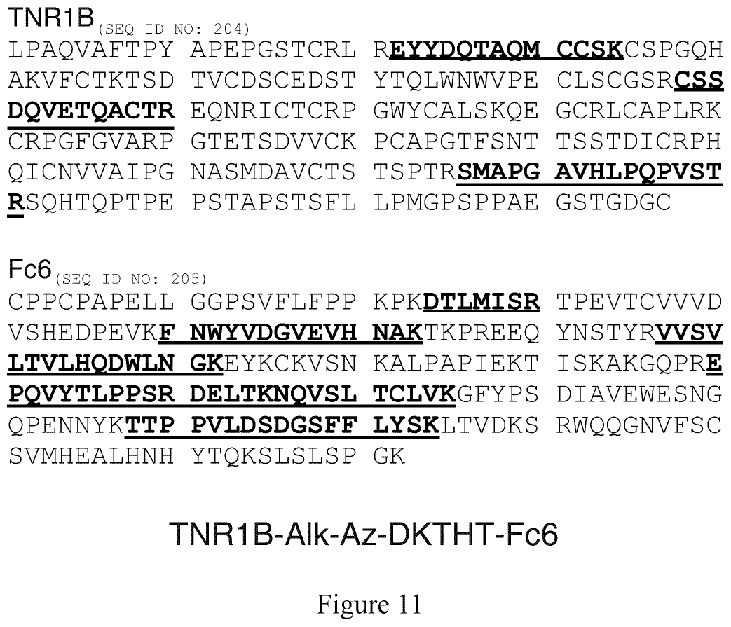

FIG. 11 summarizes the characterization of the structure of the TNR1B-alkyne-azide-Fc6 reaction product by mass spectrometry. The Mr ~75,000 product, as expected for the full-length TNR1B-alkyne-azide-Fc6 product, contained peptides from both the alkyne-modified TNR1B and azide-modified Fc6 parent proteins. In addition, the peptide coverage of the alkyne-modified TNR1B sequence (upper panel) extended from the N-terminal region (EYYDQTAQMCCSK—SEQ ID NO: 221) to the C-terminal region (SMAPGAVHLPQPVST—SEQ ID NO: 222). Similarly, the peptide coverage of the azide-modified Fc6 protein sequence (lower panel) extended from the N-terminal region (DTLMISR—SEQ ID NO: 223) to the C-terminal region (TTPPPVLDSDGSFFLYSK—SEQ ID NO: 224). In contrast, the Mr ~65,000 lacked the EYYDQTAQMCCSK (SEQ ID NO: 221) peptide, suggesting it was an N-terminally deleted version of the expected full-length TNR1B-alkyne-azide-Fc6 product.

Sequences derived from the TNR1B protein were not detected in the unstained region of Mr ~43,000 where the alkyne-modified TNR1B would normally migrate (arrow c), while only sequences derived from the Fc6 protein were detected in the unreacted Az-DKTHT-Fc6 protein of Mr ~28,000 (arrow d).

The TNR1B-alkyne-azide-Fc6 and TNR1B-alkyne-azide-PEG₄-Fc6 products of FIG. 10 were further characterized for their biological activity by measuring their ability to bind TNF-α using surface plasmon resonance (SPR). Recombinant human TNF-α protein (carrier-free) was obtained from R&D Systems and reconstituted in PBS. SPR studies were carried out using a Biacore T100 instrument from Biacore AB (Uppsala, Sweden). The surface-bound ligands, TNR1B-alkyne-azide-Fc6 and TNR1B-alkyne-azide-PEG₄-Fc6, were immobilized onto a CM5 sensor chip, Series S, using a Amine Coupling Kit (BR-1000-50) obtained from GE Healthcare (Piscataway, NJ) according to the manufacturer's instructions. Binding of TNF-α was carried out at 25° C. in 10 mM Hepes buffer pH 7.4, 150 mM NaCl, 3 mM EDTA, and 0.005% Tween-20. Binding was evaluated in duplicate at TNF-α concentrations of 0.156 nM, 0.312 nM, 0.625 nM, 1.25 nM, 2.5 nM, 5.0 nM, 10.0 nM, 20.0 nM and 40 nM. Data was evaluated using Biacore T100 Evaluation Software, version 2.0.3.

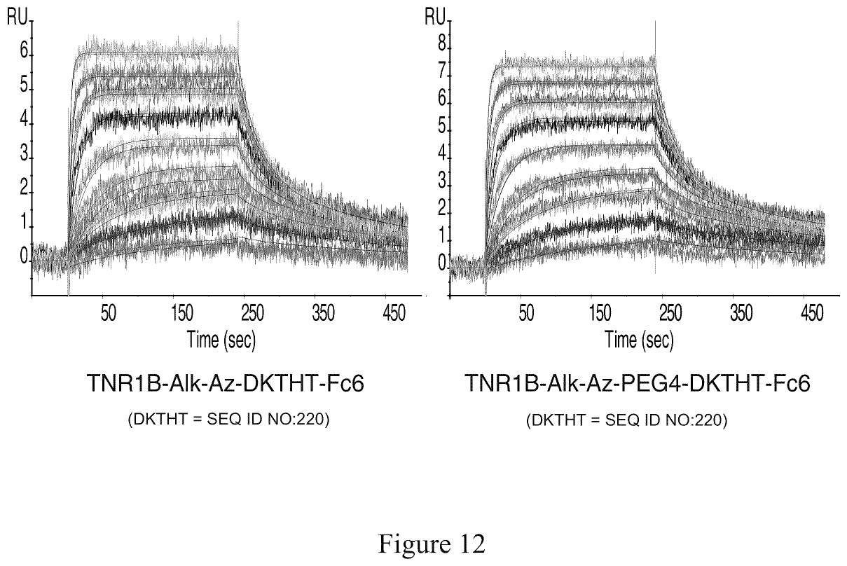

FIG. 12 shows the kinetic binding curves for TNR1B-alkyne-azide-Fc6 (left panel) and TNR1B-alkyne-azide-PEG₄-Fc6 (right panel). Both products showed saturable TNF-α binding, and an excellent fit was obtained employing a two-exponential model (Chi² ~0.05). Table 2 summarizes the kinetic binding data. Approximately 40% of the binding sites for each product were higher affinity, with a 1.6-fold greater dissociation constant for TNR1B-alkyne-azide-PEG₄-Fc6 (K_{D}=1.86×10⁻¹⁰ M) than for TNR1B-alkyne-azide-Fc6 (K_{D}=2.99×10⁻¹⁰ M). The remaining 60% of the binding sites were of lower affinity, with the dissociation constants about the same for TNR1B-alkyne-azide-PEG₄-Fc6 (K_{D}=5.12×10⁻⁹ M) and TNR1B-alkyne-azide-Fc6 (K_{D}=5.17×10⁻⁹ M). The association of the PEG₄ linker with increased high affinity binding, but equal low affinity binding, provides compelling evidence for a higher degree of cooperative (two-handed) binding of TNF-α by TNR1B-alkyne-azide-PEG₄-Fc6 compared with TNR1B-alkyne-azide-Fc6.

TABLE 1
Azide-containing and Alkyne-Containing Thioesters

| Name | Formula | Mr | MH⁺ | Sequence |
|---|---|---|---|---|
| Az-DKTHT | C₃₃H₄₇O₁₀N₁₁S | 789.86 | 780.60 | Azide-DKTHT-thioester |
| Az-PEG₄-DKTHT | C₄₄H₆₈O₁₅N₁₂S | 1037.14 | 1038.20 | Azide-PEG₄-DKTHT-thioester |
| Az-PEG₁₂-DKTHT | C₅₉H₉₈O₂₃N₁₂S | 1375.55 | 1376.26 | Azide-PEG₁₂-DKTHT-thioester |
| Az-PEG₂₄-DKTHT | C₈₃H₁₄₆O₃₅N₁₂S | 1904.18 | 1904.80 | Azide-PEG₂₄-DKTHT-thioester |
| Az-PEG₃₆-DKTHT | C₁₀₇H₁₉₄O₄₇N₁₂S | 2432.82 | 2434.40 | Azide-PEG₃₆-DKTHT-thioester |
| Alk-PEG₁₂ | C₅₃H₇₄O₁₅N₂S | 1011.22 | 1011.80 | DIBAC-PEG₁₂-thioester |

Mr, relative molecular mass;
MH⁺, monoisotypic mass value.
DKTHT = SEQ ID NO: 220

TABLE 2
TNF-α binding measured by surface plasmon resonance

| Surface-bound | ka1 | kd1 | | | ka2 | kd2 | | | |
|---|---|---|---|---|---|---|---|---|---|
| ligand | (1/Ms) | (1/s) | KD1 (M) | Rmax1 | (1/Ms) | (1/s) | KD2 (M) | Rmax2 | Chi² |
| TNR1B-Alk-Az- | 1.252E+7 | 0.003744 | 2.990E-10 | 2.5 | 5.176E+6 | 0.03392 | 6.553E−9 | 3.9 | 0.0514 |
| DKTHT-Fc6 | | | | | | | | | |
| TNR1B-Alk-Az- | 1.400E+7 | 0.002613 | 1.866E-10 | 3.0 | 5.129E+6 | 0.03021 | 5.890E−9 | 4.8 | 0.0503 |
| PEG4-9DKTHT-Fc6 | | | | | | | | | |

Abbreviations:
ka, on-rate (measured);
kd, off-rate (measured):
KD, dissociation constant (calculated).
DKTHT = SEQ ID NO: 220

### Example 2: Fab′-alkyne-azide-Fc6

Fab′-alkyne-azide-Fc6 was prepared via the reaction of cycloalkyne-modified Fab′ with azide-modified Fc6 as follows.

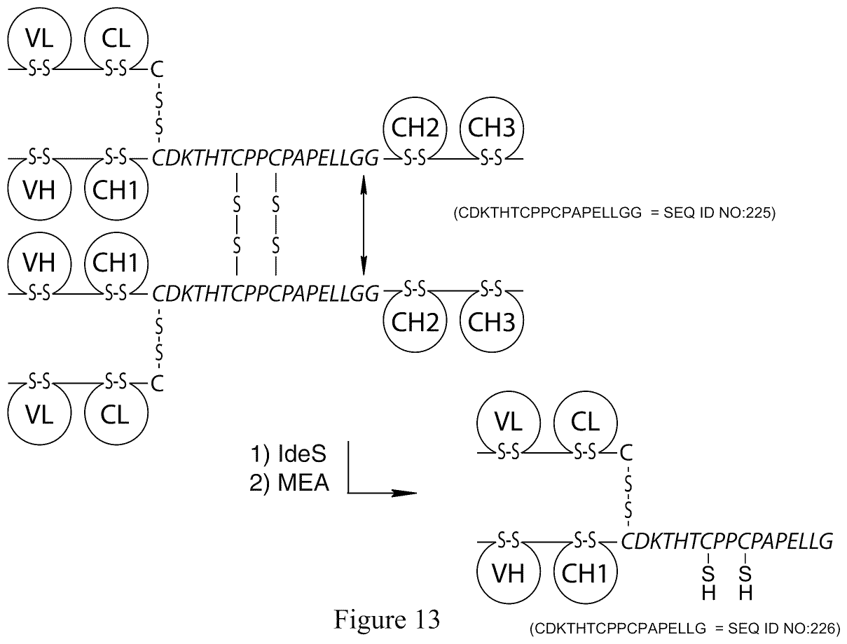

Adalimumab (Humira) was obtained as a liquid formulation (50 mg/ml) from Abbott (Abbott Park, IL). The Fab′ fragment was prepared using IdesS protease to first generate Fab′2 fragment followed by selective reduction of the interchain disulfides with 2-mercaptoethylamine (FIG. 13). Antibody (10 mg) was exchanged into cleavage buffer (50 mM sodium phosphate, 150 mM NaCl, pH 6.6) using a Slide-A-Lyzer Mini Dialysis Unit, 10K MWCO from Pierce (Rockford, IL), then incubated with his-tagged recombinant IdeS immobilized on agarose beads (FragIT MidiSpin column) from Genovis (Lund, Sweden) for 1 hour at room temperature with constant mixing. The beads were removed from the digest solution by centrifugation, washed twice with cleavage buffer, and the digest and wash solutions then combined and applied to a HiTrap Protein A HP column from GE Life Sciences (Piscataway, NJ) to remove Fc′ fragment and undigested antibody. Flow-through fractions containing the Fab′2 fragment were reduced to the Fab′ fragment by adding 1 mL aliquots to a vial containing 6 mg 2-mercaptoethylamine (MEA) from Pierce. Reductions were carried out with 10 mM EDTA to minimize re-oxidation of the interchain disulfides. Following incubation at 37° C. for 110 min, excess MEA was removed by buffer-exchange into PBS containing 10 mM EDTA using a PD-10 desalting column from GE Life Sciences (Piscataway, NJ). The eluate containing the Fab′ fragment was concentrated using an Amicon Ultracel-3 Centrifugal Filter Unit from Millipore (Billerica, MA).

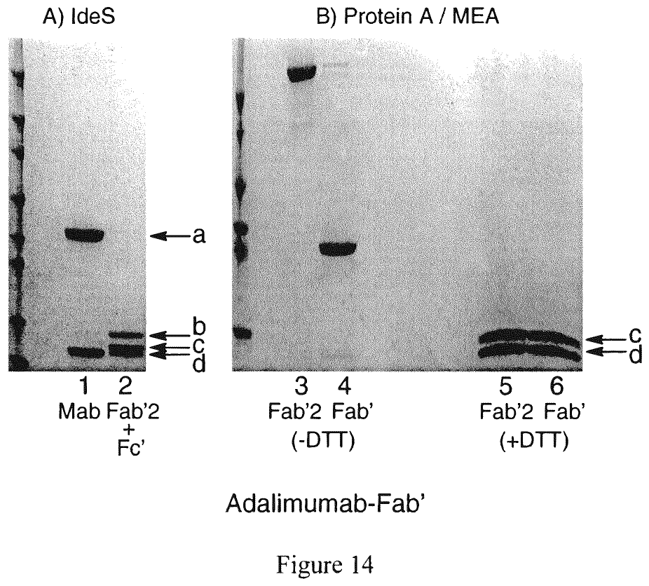

FIG. 14 shows SDS-PAGE analysis of adalimumab after cleavage with IdeS (panel A), followed by Protein A chromatography and mild reduction with MEA (panel B). In the presence of a strong reducing agent (dithiothreitol) in the polyacrylamide gel, the whole antibody (lane 1) migrated as a heavy chain of Mr ~55,000 (arrow a) and a light chain of Mr ~25,000 (arrow d). IdeS cleaved the heavy chain (lane 2) into a C-terminal fragment of Mr ~29,000 (arrow b) and an N-terminal fragment of Mr ~26,000 (arrow c). The light chain and the N-terminal heavy chain fragment comprise the Fab′2 domain, while the C-terminal heavy chain fragment comprises the Fc′ domain. The Protein A column efficiently removed the Fc′ domain from the Fab′ domain (compare lane 2 with lanes 5 and 6). Under non-reducing conditions, the Fab′2 domain migrated as a single species of Mr ~110,000 (lane 3), while the Fab′ domain produced by mild reduction with MEA migrated as a single species of Mr ~55,000 (lane 4). Under reducing conditions, the Fab′2 domain (lane 5) and the Fab′ domain (lane 6) both yielded the same light chain (arrow d) and N-terminal heavy chain fragment (arrow c), as expected. Thus, the Fab′ domain obtained by this procedure was essentially free of the Fab′2 and Fc′ domains.

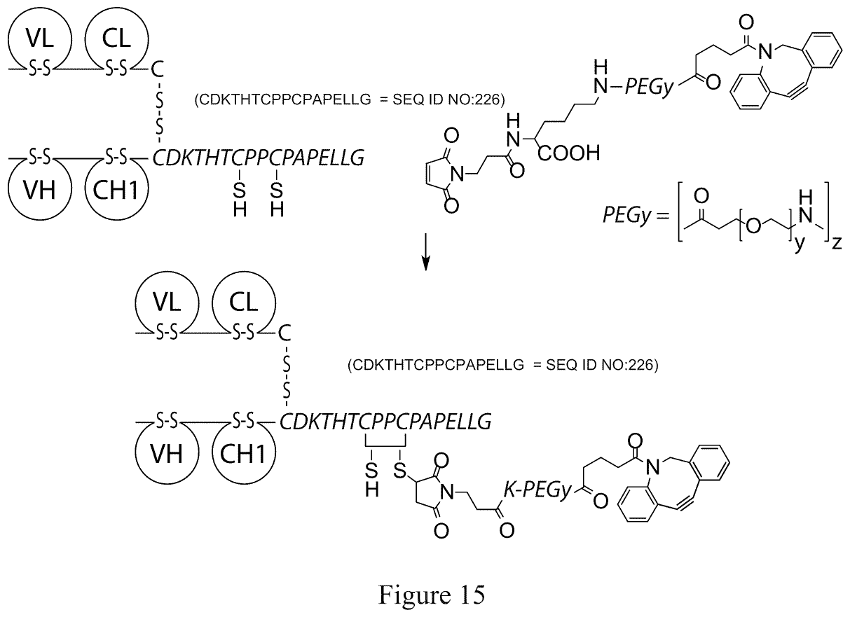

Cycloalkyne-modified Fab′ was prepared from the adalimumab Fab′ domain using a bifunctional linker, DIBAC-PEG₁₂-Lys(Mal), which contains a maleimide group capable of reacting with the free thiol groups on the Fab′ fragment (FIG. 15). DIBAC-PEG₁₂-Lys(Mal) was prepared using an Fmoc solid-phase synthesis strategy. Lys(Mtt)-Wang resin and succinimido 3-maleimidopropanoate (Mpa-OSu) were obtained from CPC Scientific (Sunnyvale, CA), Fmoc-N-amido-dPEG₁₂-acid was obtained from Quanta BioDesign (Powell, OH), and 5-(11,12-Didehydrodibenzo[b,f]azocin-5(6H)-yl)-5-oxopentanoic acid, an acid-functionalised aza-dibenzocyclooctyne (DIBAC-acid), was synthesized as described by Debets, M. F. et al., Chem. Commun. 46, 97-99 (2010). Fmoc-N-amido-dPEG₁₂-acid and DIBAC-acid were sequentially reacted with Lys(Mtt)-Wang resin to obtain DIBAC-PEG₁₂-Lys(Mtt)-Wang resin, then treated with TFA/DCM/TIS (1:96:3) to remove the Mtt group. The deprotected resin was reacted with Mpa-OSu on the free amino group on the lysine side chain to obtain DIBAC-PEG12-Lys(Mpa)-Wang resin. Following cleavage with TFA/water (95:5), the crude product was purified by preparative RP-HPLC to afford the DIBAC-PEG₁₂-Lys(Mal) product (DPKM) with 93% purity and the desired MS spectra.

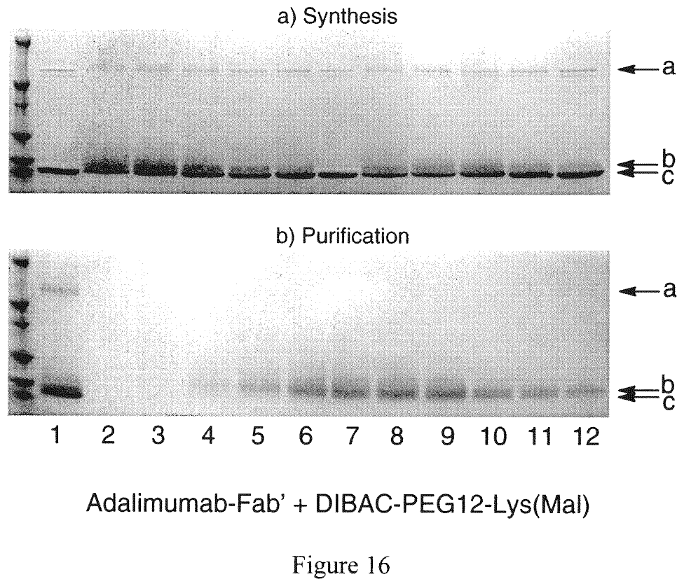

FIG. 16 shows the chemical modification of adalimumab Fab′ fragment with the DIBAC-PEG₁₂-Lys (Mal) linker and the purification of the resulting cycloalkyne-modified Fab′. For purification, reactions (0.535 mL) were carried out in 0.1 M sodium phosphate at pH 7.0 and pH 7.4, each containing 5 mg of Fab′ fragment and 10 mg of DIBAC-PEG₁₂-Lys (Mal). After 30 hours incubation at room temperature, the two reactions were combined and buffered-exchanged into 20 mM sodium acetate, 20 mM NaCl, pH 5.5 using a PD-10 column. The eluate (3.5 mL) was applied to a HiTrap SP HP cation-exchange column from GE Life Sciences which retained all the unmodified Fab′ and residual Fab′2. The flow-through fractions (5.5 mL) containing the purified cycloalkyne-modified Fab′ (FIG. 16b) were pooled, adjusted to pH 7.0 with 10×PBS (0.55 mL), and concentrated by affinity chromatography on a Protein L column (Capto L) from GE Life Sciences. The cycloalkyne-modified Fab′ was eluted from the Protein L column with 0.1 M glycine HCl pH 2.7 (2.4 mL), neutralized with 1/20 volume 1.0 M Tris HCl pH 9.0, buffered-exchanged into PBS using a PD-10 column (3.5 mL) and concentrated using Amicon Ultracel-3 Centrifugal Filter Unit to a final volume of 70 uL at a concentration of 9.5 mg/mL.

Various azide-modified Fc6 proteins with PEG linkers of different lengths were used in the preparation of the adalimumab Fab′-cycloalkyne-azide-Fc6. Az-DKTHT-Fc6 (FIG. 3) and Az-DKTHT-PEGₓ-Fc6 derivatives with x=12, 24, and 36 (FIG. 4) were prepared in reactions (2 ml) that contained 50 mM MES pH 6.5, 0.8 mM TCEP, 10 mM MPAA, 5 mg/ml of each of the four Az-DKTHT-PEGₓ-thioesters, and 2.36 mg/ml of Fc6 protein. DKTHT is SEQ ID NO: 220. After 20 hours at room temperature, the reactions were neutralized with 100 μL of Tris HCl pH 9.0, clarified by centrifugation at 12,000×g, and applied to a 1 ml HiTrap Protein A HP column. The columns were washed with 12 vol of PBS, the azide-modified Fc6 proteins were then eluted with 0.1 M glycine HCl pH 2.7 (2.0 mL), neutralized with 1/20 volume 1.0 M Tris HCl pH 9.0, dialysed against three changes of PBS for 12 hours each using a Slide-A-Lyzer Mini Dialysis Unit, 10K MWCO, and concentrated using Amicon Ultracel-3 Centrifugal Filter Units.

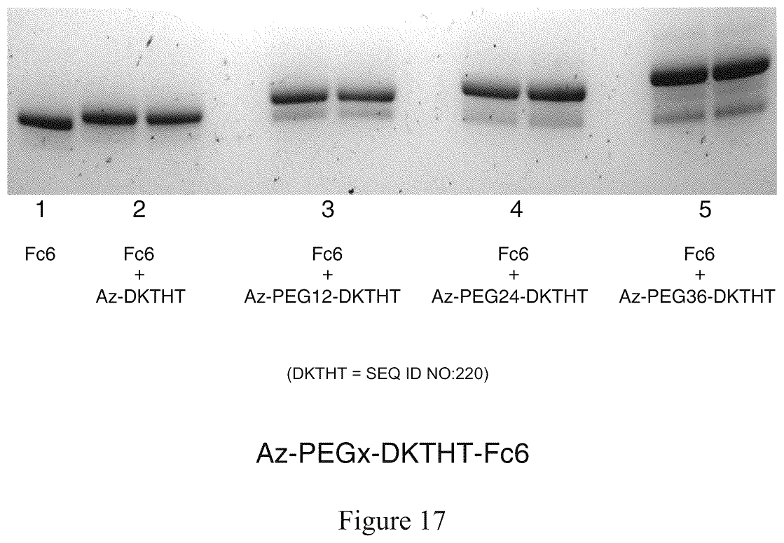

FIG. 17 shows analysis by SDS-PAGE under reducing conditions of the Fc6 (lane 1) Az-DKTHT-Fc6 (lane 2), Az-DKTHT-PEG₁₂-Fc6 (lane 3), Az-DKTHT-PEG₂₄-Fc6 (lane 4), and Az-DKTHT-PEG₃₆-Fc6 (lane 5) proteins by SDS-PAGE. The Fc6 protein reacted quantitatively (>90%) with all four thioesters, yielding a ladder of products of increasing size. DKTHT is SEQ ID NO: 220.

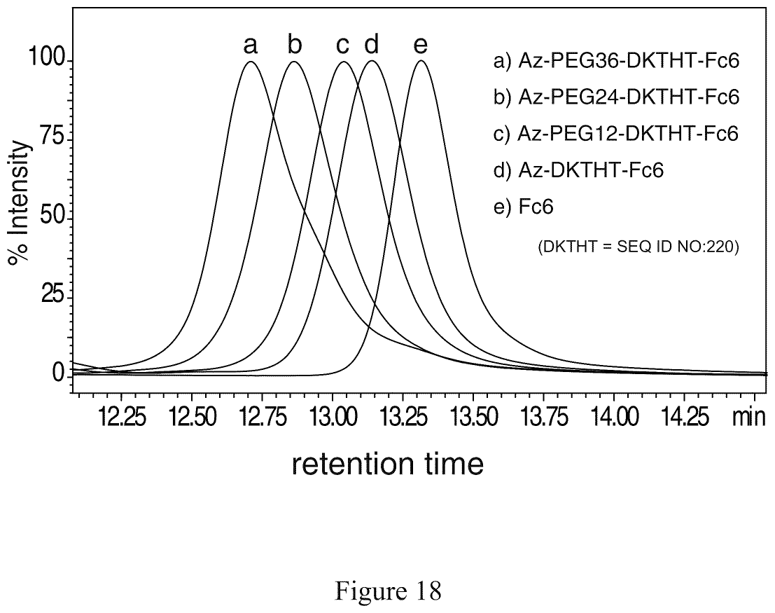

FIG. 18 shows analysis by size-exclusion chromatography (SEC) to confirm that the four azide-modified Fc6 protein products had the same dimeric structure as the parent Fc6 molecule. SEC was carried out using a Prominence HPLC System (Shimadzu Corp, Kyoto, Japan). TSKgel Super SW3000 columns (4.6 mm×30 cm column, 4.6 mm×5 cm guard column) were obtained from TOSOH Bioscience (Tokyo, Japan). Mobile phase, flow rate, column temperature, and detection wavelength used were 50 mM sodium phosphate, 300 mM NaCl, pH 7.4, 0.35 mL/min., 30° C., and 280 nm, respectively. The four azide-modified Fc6 protein products displayed a retention time that decreased as the size of PEG linker increased, confirming their dimer structure. All four products also gave essentially a single peak, demonstrating a two-handed structure in which both N-termini of the parent Fc6 dimer were modified by the PEG linker that was confirmed by SDS-PAGE analysis under non-reducing conditions (see below).

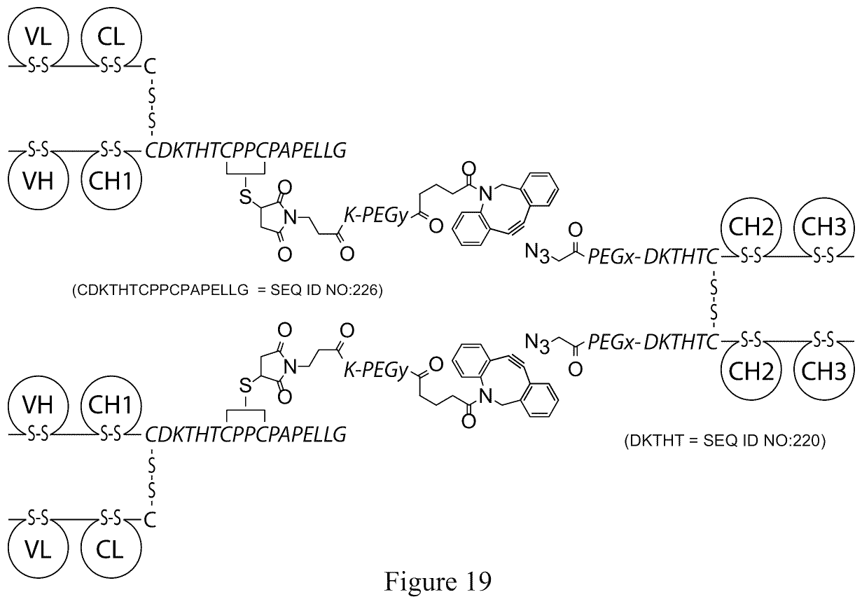

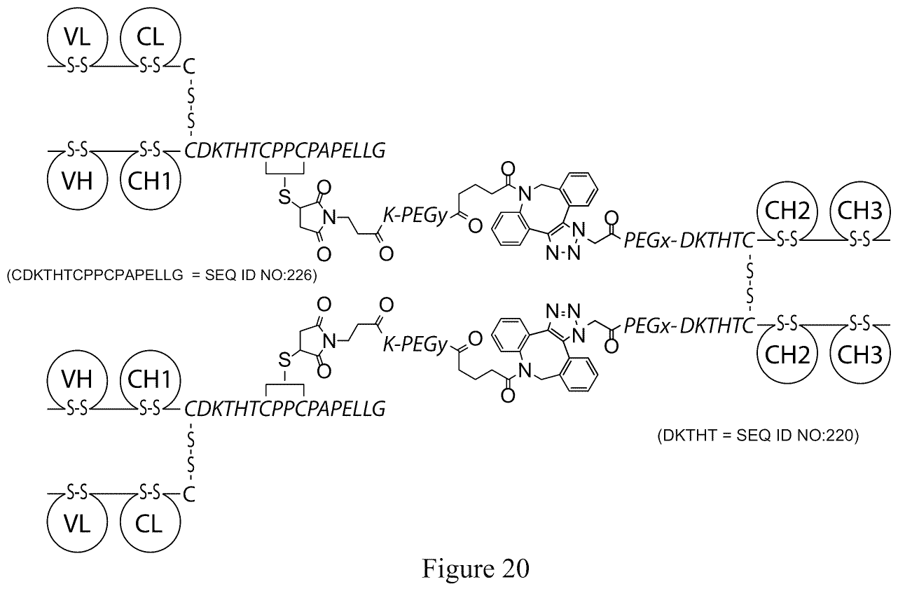

The cyclooctyne-modified Fab′ was reacted with all four azide-modified Fc6 molecules (FIG. 19), yielding a family of Fab′-PEGy-cycloalkyne-azide-PEGₓ-Fc6 structures with arms of increasing length (FIG. 20). The overall lengths of the resulting arms were Fab′-PEG₁₂-Fc6 (for x=0, y=12), Fab′-PEG₂₄-Fc6 (for x=12, y=12), Fab′-PEG₃₆-Fc6 (for x=24, y=12), and Fab′-PEG₄₈-Fc6 (for x=36, y=12). The reactions (8 uL) were carried out in 0.1 M sodium phosphate pH 7.0 overnight at room temperature with each of the four azide-modified Fc6 proteins (2.5 mg/ml) in the presence or the absence of the cycloalkyne-modified Fab′ (5 mg/ml).

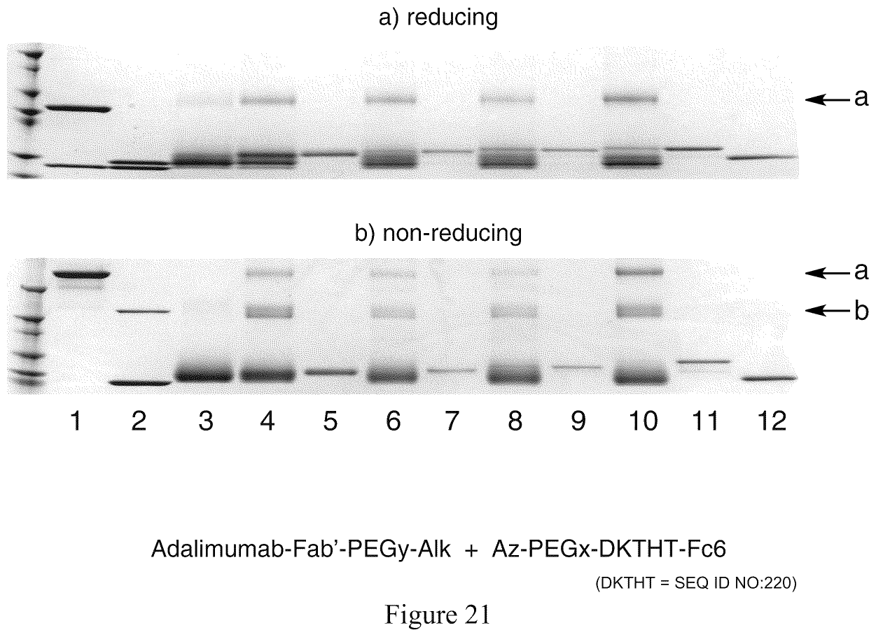

FIG. 21 shows SDS-PAGE analysis of the Fab′-cycloalkyne-azide-Fc6 reaction under reducing and non-reducing conditions. In the absence of the cycloalkyne-modified Fab′ (lanes 5, 7, 9, and 11), all four of the azide-modified Fc6 proteins gave a single band on both reducing and non-reducing gels, confirming their dimeric, two-handed handed structure. In the presence of the cycloalkyne-modified Fab′ (lanes 4, 6, 8, and 10), all four of the azide-modified Fc6 proteins were largely consumed in the resulting reaction. Under reducing conditions, all four reactions gave a product with Mr ~57,000 to 62,000 (arrow a). The size of the Fab′-PEG₁₂-Fc6 product (lane 4) was approximately 1-2 kD greater than the wild-type adalimumab heavy chain (lane 1), while the sizes of the Fab′-PEG₂₄-Fc6 (lane 6), Fab′-PEG₃₆-Fc6 (lane 8), and Fab′-PEG₄₈-Fc6 (lane 10) products further increased with the overall length of the PEG linker. Under non-reducing conditions, two products were observed, a first product of Mr ~155,000 to 160,000 (arrow a), and a second of Mr ~110,000 to 115,000 (arrow b). The larger Fab′-PEG₁₂-Fc6 product (lane 4) was approximately 5 kD greater than the adalimumab whole antibody (lane 1), consistent with the expected two-handed product, while the larger Fab′-PEG₂₄-Fc6 (lane 6), Fab′-PEG₃₆-Fc6 (lane 8), and Fab′-PEG₄₈-Fc6 (lane 10) products still further increased in size as the overall length of the PEG linker increased.

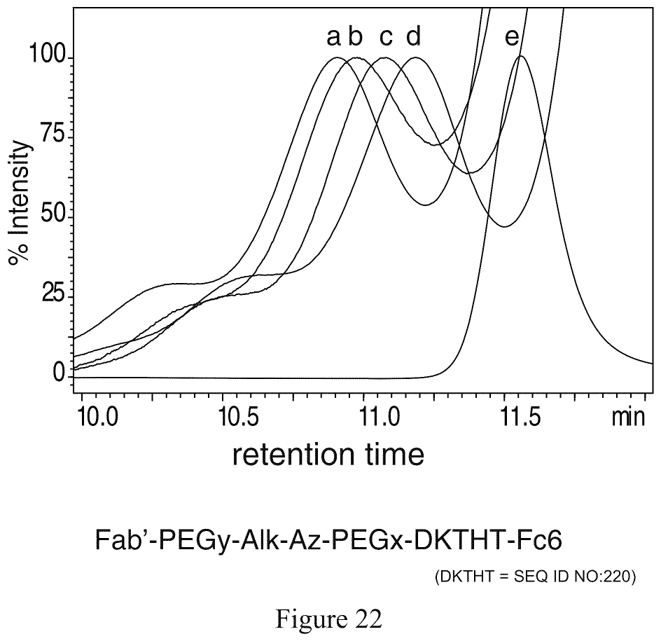

FIG. 22 shows analysis by SEC to confirm the two-handed structure (ie, two Fab′ hands attached to one Fc6 domain) of the larger reaction product with Mr ~155,000 to 160,000 of the Fab′-PEG₁₂-Fc6, Fab′-PEG₂₄-Fc6, Fab′-PEG₃₆-Fc6, and Fab′-PEG₄₈-Fc6 reactions. All four reaction products displayed a shorter retention time than the adalimumab whole antibody that further decreased as the size of PEG linker increased, confirming the two-handed structure observed by SDS-PAGE analysis.

The biological activity of the Fab′-cycloalkyne-azide-Fc6 products evaluated by their ability to neutralize TNF-α-mediated cytotoxicity on murine WEHI cells treated with actinomycin D. The mouse WEHI-13VAR cell line (ATCC CRL-2148) was obtained from the American Type Culture Collection (Rockville, MD) and grown in Gibco RPMI media 1640 (RPMI-1640) supplemented with 10% fetal bovine serum (FBS) and penicillin and streptomycin (10 U/ml), obtained from Life Technologies (Grand Island, NY). TNF-α cytotoxity assays were carried out as follows. WEHI-13VAR cells were plated in 96-well Nunc white cell culture plates obtained from Thermo Fisher (Waltham, MA) at 2×10⁴ cells per well overnight and then treated with actinomycin D (0.5 μg/ml) obtained from Sigma (St Louis, MO) and TNF-α (0.2 ng/ml) in the absence or presence of TNFR-IgG or other inhibitors. After 24 hr of incubation at 37° C./5% CO2, the cell viability was determined with CellTiter-Glo Luminescent Cell Viability Assay Systems (Promega, Madison, WI) measuring the quantity of the ATP present in metabolically active cells and luminescence measured using a POLARstar luminometer (BMG LABTECH Inc., Cary, NC). Each inhibitor was diluted by ten 3-fold serial dilutions starting at 10 μg/ml and measured in duplicate or triplicate. Cytotoxicity data were calculated using the following equations: (1-sample luciferase reading/luciferase reading from cells treated with actinomycin D alone)×100%, and presented as the mean±standard deviation. Enbrel was used as a cytotoxicity positive control and Fc6 as a negative control.

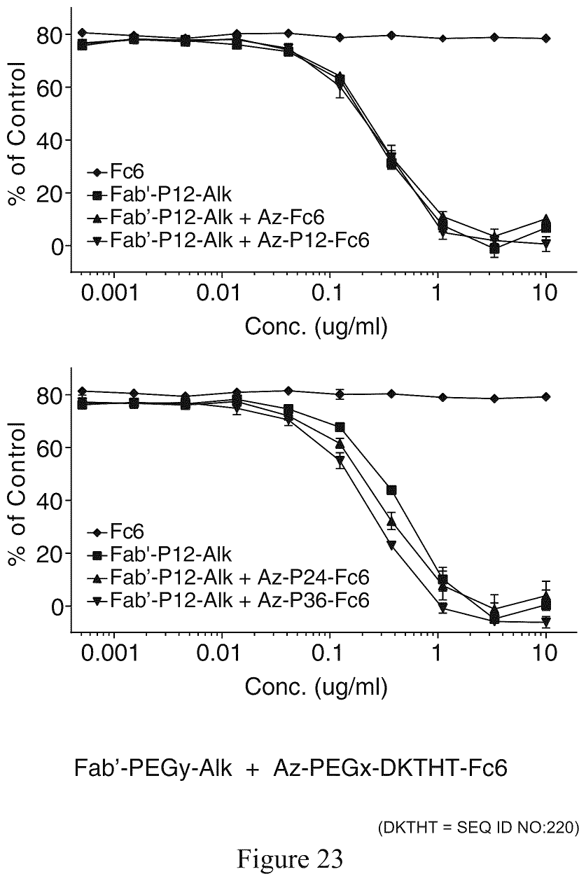

FIG. 23 shows the neutralization of TNF-α-mediated cytotoxicity by Fab′-PEG₁₂-Fc6, Fab′-PEG₂₄-Fc6, Fab′-PEG₃₆-Fc6, and Fab′-PEG₄₈-Fc6 reaction mixtures compared with the cycloalkyne-modified Fab′ (based upon an equal amounts of input cycloalkyne-modified Fab′). The Fab′-PEG₁₂-Fc6 and Fab′-PEG₂₄-Fc6 reaction mixtures both displayed comparable TNF-α neutralization activity compared with that of the input cycloalkyne-modified Fab′ (upper panel), whereas the Fab′-PEG₃₆-Fc6 and Fab′-PEG₄₈-Fc6 reaction mixtures displayed a 1.5-fold and 2.0-fold increase, respectively, in their TNF-α neutralization activity compared with the input cycloalkyne-modified Fab′ (lower panel). Since the amount of two-handed product represented only 10-20% of the total cycloalkyne-modified Fab′ in each reaction as estimated by SDS-PAGE (FIG. 22), the two-handed products of the Fab′-PEG₃₆-Fc6 and Fab′-PEG₄₈-Fc6 reactions are estimated to be at least 7.5-fold and 10-fold greater than the input cycloalkyne-modified Fab′, respectively.

### Example 3: Fab-alkyne-azide-Fc6

Fab-alkyne-azide-Fc6 is prepared by reacting azide-modified Fc6 with an alkyne-modified or cycloalkyne-modified Fab protein that is produced by cleavage of an Fab-intein fusion protein as follows. Similarly, Fab-azide-alkyne-Fc6 is prepared by reacting alkyne-modified or cycloalkyne-modified Fc6 with an azide-modified Fab protein that is produced by cleavage of an Fab-intein fusion protein.

Adalimumab Fab-intein fusion protein is produced by cotransfecting expression vector pFUSE2ss-DE27-Vκ-CLIg-hk (SEQ ID NO: 115) with pPUSEss-DE27-Vγ1-CHIg-hG1-Mth-1 (SEQ ID NO: 116) or pFUSEss-DE27-Vγ1-CHIg-hG1-Mth-2 (SEQ ID NO: 117).

Vector pFUSE2ss-DE27-Vκ-CLIg-hk directs the expression of the pre-kappa light chain of adalimumab shown in SEQ ID NO: 118. Cleavage of the heterologous IL-2 signal sequence by the cellular signal peptidase provides the mature kappa light chain of adalimumab shown in SEQ ID NO: 119.

Vector pFUSEss-DE27-Vγ1-CHIg-hG1-Mth-1 directs the expression of a first type of pre-heavy chain-intein chimeric polypeptide shown in SEQ ID NO: 120, in which the adalimumab heavy chain VH and CH1 domains are joined at their C-terminus to the N-terminus of an RIR1 self-splicing intein at the autocleavage site. Cleavage of the heterologous IL-2 signal sequence by the cellular signal peptidase provides the mature heavy chain-intein fusion protein shown in SEQ ID NO: 121. Together, the proteins of SEQ ID NO: 119 and SEQ ID NO: 121 comprise the adalimumab Fab-1-intein fusion protein that is secreted into the cell culture fluid.

Vector pFUSEss-DE27-Vγ1-CHIg-hG1-Mth-2 directs the expression of a second type of pre-heavy chain-intein chimeric polypeptide shown in SEQ ID NO: 122, in which the adalimumab heavy chain VH and CH1 domains are joined at their C-terminus to the N-terminus of an RIR1 self-splicing intein at the autocleavage site.

Cleavage of the heterologous IL-2 signal sequence by the cellular signal peptidase provides the mature heavy chain-intein fusion protein shown in SEQ ID NO: 123. Together, the proteins of SEQ ID NO: 119 and SEQ ID NO: 123 comprise the adalimumab Fab-2-intein fusion protein that is secreted into the cell culture fluid.

Protein production is executed by transient expression in CHO-DG44 cells essentially as described in Example 1, by the cotransfection of SEQ ID NO: 115 with SEQ ID NO: 116 to produce the adalimumab Fab-1-intein fusion protein, and by cotransfection of SEQ ID NO: 115 with SEQ ID NO: 117 to produce adalimumab Fab-2-intein fusion protein.

Alkyne-modified adalimumab Fab proteins are produced by cleavage of adalimumab Fab-intein fusion proteins with 50 mM cystyl-propargylamide essentially as described in Example 1. The adalimumab Fab-1-intein fusion protein is cleaved with cystyl-propargylamide to produce alkyne-modified adalimumab Fab-1 protein which is a heterodimer protein of SEQ ID NO: 119 and SEQ ID NO: 124. The adalimumab Fab-2-intein fusion protein is cleaved with cystyl-propargylamide to produce alkyne-modified adalimumab Fab-2 protein which is a heterodimer protein of SEQ ID NO: 119 and SEQ ID NO: 125.

Azide-modified adalimumab Fab proteins are produced by cleavage of adalimumab Fab-intein fusion proteins with 50 mM cystyl-3-azidopropylamide essentially as described in Example 1. The adalimumab Fab-1-intein fusion protein is cleaved with cystyl-3-azidopropylamide to produce azide-modified adalimumab Fab-1 protein which is a heterodimer protein of SEQ ID NO: 119 and SEQ ID NO: 126. The adalimumab Fab-2-intein fusion protein is cleaved with cystyl-3-azidopropylamide to produce azide-modified adalimumab Fab-2 protein which is a heterodimer protein of SEQ ID NO: 119 and SEQ ID NO: 127.

Adalimumab Fab-1-alkyne-azide-Fc6 and Adalimumab Fab-2-alkyne-azide-Fc6 are prepared via the reaction of alkyne-modified adalimumab Fab-1 protein or alkyne-modified adalimumab Fab-2 protein with Az-DKTHT-Fc6 protein (FIG. 6) or Az-PEGx-DKTHT-Fc6 proteins (FIG. 7). DKTHT is SEQ ID NO: 220.

Tris(3-hydroxypropyltriazolylmethyl)amine (THTPA) is prepared as described by Hong et al., Angew. Chem. Int. Ed. 48, 1-7 (2009) Reactions are carried out in 0.1 M sodium phosphate, pH 7.0, with the Linker-Fc at a concentration of 5 mgs/mL or greater, and a molar ratio of >2:1 of Fab-A:Linker-Fc. To the reaction is added a final concentration of 0.0001 M CuSO₄, 0.0005 M THTPA. The reaction is initiated by adding to a final concentration 0.005 M aminoguanidine and 0.005 M sodium ascorbate. Following incubation at room temperature for 12-18 hours in a closed tube, the reaction mixture is applied to a chromatographic column packed with Protein A (GE Lifesciences, NJ) to remove excess reagent and unreacted Fab-A, washed with PBS, eluted with 0.1 M Glycine-HCl, pH 2.7, and immediately neutralized by adding 1.0 M Tris-HCl, pH 9.0. The eluted Adalimumab Fab-1-alkyne-azide-Fc6 and Adalimumab Fab-2-alkyne-azide-Fc6 products are dialysed against PBS.

Adalimumab Fab-1-azide-alkyne-Fc6 and Adalimumab Fab-2-azide-alkyne-Fc6 are prepared via the reaction of azide-modified adalimumab Fab-1 protein or azide-modified adalimumab Fab-2 protein with cycloalkyne-modified Fc6 protein.

Cycloalkyne-modified Fc6 proteins are prepared essentially as described in Example 1 using DIBAC-PEG₁₂-thioester (Table 1) and other DIBAC-PEGx-thioesters and DIBAC-PEGx-DKTHT-thioesters similarly prepared. DKTHT is SEQ ID NO: 220.

### Example 4: N-Terminal Azide-Modified FC Proteins

A series of azide-modified Fc proteins (N₃-Px-Fc), each having an azide functional group at its N-terminus, and optionally a PEG linker, was prepared by reacting the Fc6 protein with five thioesters having the sequence azidoacetyl-Px-DKTHT-thiophenol (x=0, 12, 24, 36, 48). Reactions were carried out in the absence of TCEP to minimize any reduction of the azide group to a primary amine. The azidoacetyl-Px-DKTHT-thiophenol thioesters with x=12, 24, 36 are shown in Table 1. Azidoacetyl-DKTHT-thiophenol was prepared as described in Example 1 (calculated for C₃₂H₄₅O₁₀N₁₁S [M+H]⁺ 776.8, found 776.3). DKTHT is SEQ ID NO: 220. Azidoacetyl-PEG48-DKTHT-thiophenol was prepared by solid-phase by the sequential condensation of Fmoc-PEG12-OH and Fmoc-PEG36-OH obtained from Quanta BioDesign (calculated for C₁₃₄H₂₄₇N₁₃O₆₀S [M+H]+ 3032.5, found 3032.8). The structural formulas are as follows:

Each reaction (2 mL) contained 50 mM MES pH 6.5, 10 mM mercaptophenylacetic acid, 2.2 mg of Fc6, and one of the five thioesters as follows: azidoacetyl-DKTHT-thiophenol (5 mg), azidoacetyl-PEG12-DKTHT-thiophenol (5 mg), azidoacetyl-PEG24-DKTHT-thiophenol (10 mg), azidoacetyl-PEG36-DKTHT-thiophenol (10 mg), or azidoacetyl-PEG48-DKTHT-thiophenol (20 mg). DKTHT is SEQ ID NO: 220. Reactions were carried out for 20 hours at room temperature, neutralized with 0.1 mL of Tris HCl pH 9.0, centrifuged at 12,000×g, and applied to a HiTrap Protein A HP column. The columns were washed with 12 vol of PBS, and the N₃-Px-Fc proteins were then eluted with 0.1 M glycine HCl pH 2.7 and neutralized with 1/20 volume of 1.0 M Tris HCl pH 9.0. The peak fractions by A280 were combined, desalted on PD-10 columns, and concentrated using Amicon Ultracel-3 Centrifugal Filter Units.

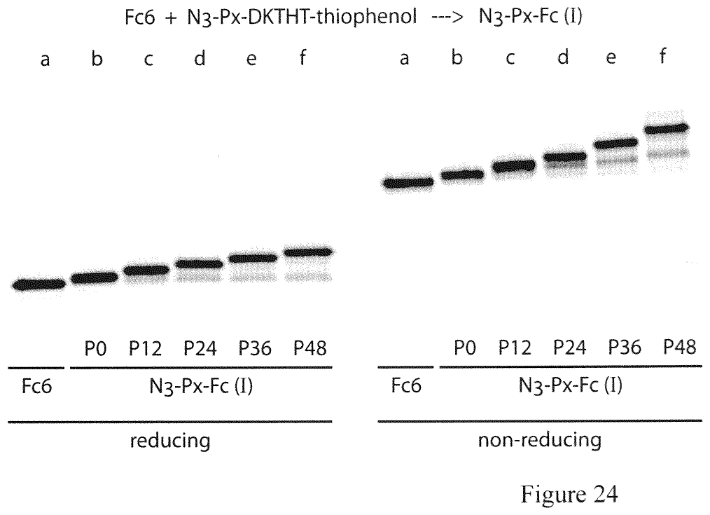

FIG. 24 shows the purified N₃-Px-Fc proteins by SDS-PAGE under reducing (left) and non-reducing conditions (right): Fc6 control (lanes a), N₃-P0-Fc (lanes b), N₃-P12-Fc (lanes c), N₃-P24-Fc (lanes d), N₃-P36-Fc (lanes e), and N₃-P48-Fc (lanes f). The size of N₃-Px-Fc proteins increased with PEG linker length. In addition, the size of N₃-Px-Fc proteins prepared without TCEP (FIG. 24) were indistinguishable by SDS-PAGE from the size of N₃-Px-Fc proteins prepared with TCEP (FIG. 17).

### Example 5: GLP1-triazole-Fc Hybrid Immunoglobulins

A series of GLP1-triazole-Fc hybrid immunoglobulins (GLP1-P4-DN-Px-Fc) were prepared by reacting a GLP-1 (glucagon-like peptide 1) analog, further modified to have a cyclooctyne functional group, with each of the five N₃-Px-Fc proteins of Example 4. The sequence of the GLP-1 analog, HGEGTFTSDVSSYLEEQAAKEFIAWLVKGRG-PEG₃-C—NH₂ (HGEGTFTSDVSSYLEEQAAKEFIAWLVKGRG is SEQ ID NO:202), corresponds to residues 7-37 of the native GLP-1 peptide, in which glycine is substituted for alanine at position 8 and glutamic acid is substituted for glycine at position 22. In addition, the GLP-1 analog has a PEG3 linker and cysteine residue at its C-terminus used to attach the cyclooctyne functional group. This GLP-1 analog, gly8-glu22-GLP-1(7-37)-PEG3-cys-NH₂, was prepared by SPPS (calculated for C₁₆₅H₂₅₃N₄₃O₅₃S [M+H]⁺ 3720.3, found 3721.3).

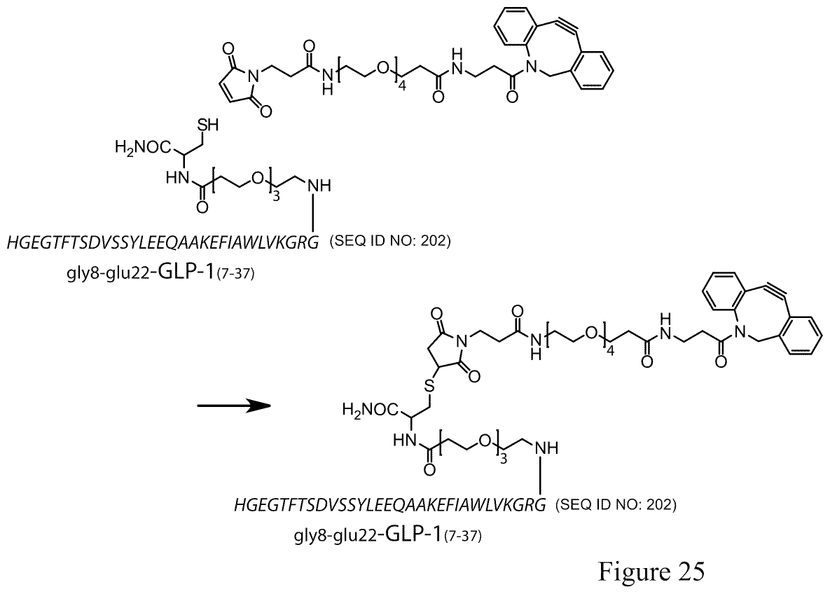

A cyclooctyne functional group was added to gly8-glu22-GLP-1(7-37)-PEG3-cys-NH₂ using a heterobifunctional linker, DBCO-PEG4-Maleimide, containing a maleimide group capable of reacting with the free thiol group on the C-terminal cysteine residue (FIG. 25). DBCO-PEG4-Maleimide (C₅₀H₅₄N₄O₉, mol weight 854.92), was obtained from Click Chemistry Tools (Scottsdale, AZ). Prior to use, the linker was dissolved at a concentration of 25 mg/mL in dimethysulfoxide (DMSO) obtained from Sigma-Aldrich (St. Louis, MO). Reactions (0.4 mL) contained 50 mM MES pH 6.5, 5 mM EDTA, 0.45 mg of gly8-glu22-GLP-1(7-37)-PEG3-cys-NH₂ peptide and 0.9 mg/mL of the DBCO-PEG4-Maleimide linker. Reactions were carried out for 30 minutes at room temperature. Excess unreacted linker was removed using a 5 mL HiTrap Desalting Column obtained from GE Life Sciences. FIG. 25 shows the structure of the resulting cyclooctyne-modified GLP-1 analog reaction product (GLP1-P7-DBCO).

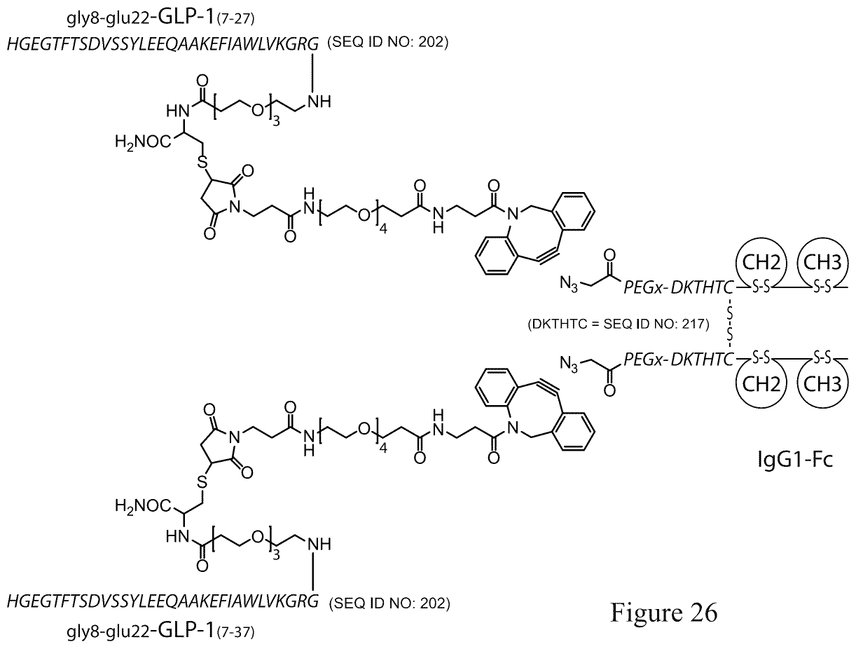

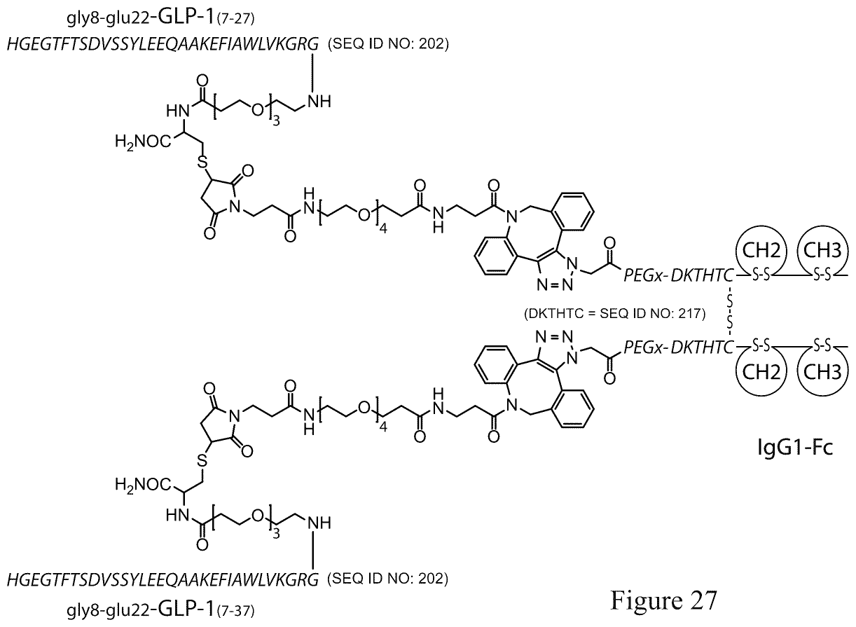

GLP1-P7-DBCO was reacted individually with each one of the five N₃-Px-Fc proteins (FIG. 26), to generate a series of GLP1-P7-triazole-Px-Fc hybrid immunoglobulins (FIG. 27). Each reaction (1.5 mL) contained 0.1 M sodium phosphate pH 7.0, 0.375 mg of the GLP1-P7-DBCO peptide, and 0.5 mg of one of the five N₃-Px-Fc proteins. Reactions were carried out for 3.5 hours at room temperature, the reactions were purified by HiTrap Protein A HP chromatography, desalted and concentrated as described in Example 4.

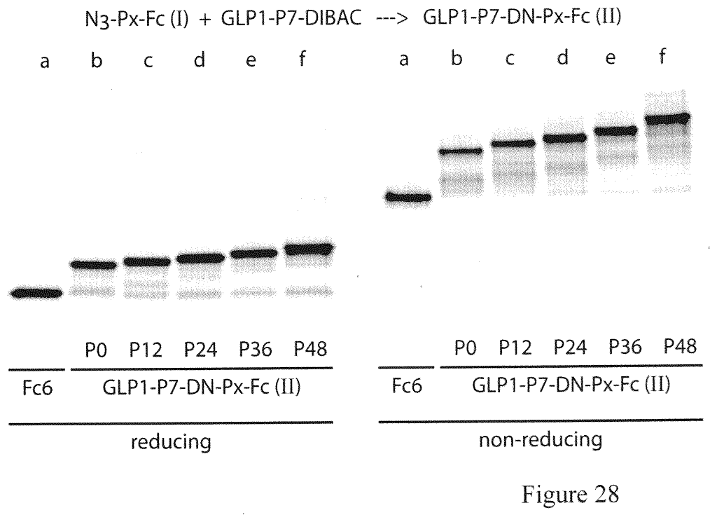

FIG. 28 shows the purified GLP1-triazole-Fc hybrid immunoglobulins by SDS-PAGE under reducing conditions (left) and non-reducing conditions (right): Fc6 control (lanes a), GLP1-P4-DN-P0-Fc (lanes b), GLP1-P4-DN-P12-Fc (lanes c), GLP1-P4-DN-P24-Fc (lanes d), GLP1-P4-DN-P36-Fc (lanes e), and GLP1-P4-DN-P48-Fc (lanes f). The size of GLP1-triazole-Fc hybrid immunoglobulins increased with PEG linker length comparable to the N₃-Px-Fc proteins.

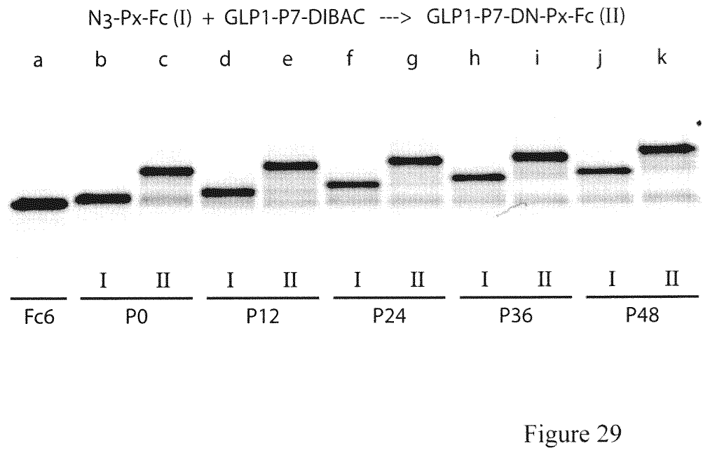

FIG. 29 directly compares the GLP1-triazole-Fc hybrid immunoglobulins and N₃-Px-Fc proteins by SDS-PAGE under reducing conditions: Fc6 control (lane a), N₃-P0-Fc (lane b), GLP1-P4-DN-P0-Fc (lane c), N₃-P12-Fc (lane d), GLP1-P4-DN-P12-Fc (lane e), N₃-P24-Fc (lane f), GLP1-P4-DN-P24-Fc (lane g), N₃-P36-Fc (lane h), GLP1-P4-DN-P36-Fc (lane i), N₃-P48-Fc (lane j), GLP1-P4-DN-P48-Fc (lane k). The conversion of each N₃-Px-Fc protein to the corresponding GLP1-P4-DN-Px-Fc hybrid immunoglobulin was approximately 95%.

### Example 6: Biological Activity of GLP1-triazole-Fc Hybrid Immunoglobulins

The biological activity of the GLP1-P7-triazole-Px-Fc hybrid immunoglobulins was evaluated in a cell-based assay that measured the induction of cAMP synthesis in cells expressing the human GLP-1 receptor (GLP-1R). For isolation of GLP-1R expressing cells, Dulbecco's Modified Eagle Medium (DMEM) from Invitrogen (Grand Island, NY), fetal bovine serum (FBS), penicillin, streptomycin, and geneticin sulfate (G418) were obtained from Corning (Manassas, VA), the CalPhos transfection kit was obtained from Clontech (Mountain View, CA), human GLP-1 receptor expression plasmid was obtained from GeneCopoeia (Rockville, MD), and anti-human GLP-1R-phycoerythrin monoclonal antibody was obtained from R&D Systems (Minneapolis, MN). For cAMP assays, 3-isobutyl-1-methylxanthine (IBMX) was obtained from Sigma-Aldrich (No. 15879), the cAMP dynamic 2 kit was obtained from Cisbio Bioassays (Bedford, MA), and the GLP-1(7-37) peptide was obtained from AnaSpec (No. 20761).

GLP-1R-expressing cells were prepared by transfecting a GLP-1R expression vector (EX-A0510-M02) into human 293T embryonic kidney cells using a CalPho mammalian transfection kit. Transfected cells were grown in DMEM supplemented with 10% FBS and penicillin and streptomycin (10 IU/ml), and selection for stable transfectants was carried out in same media containing 2 mg/ml G418. GLP-1R expression was evaluated by flow cytometric analysis using a anti-human GLP-1R-phycoerythrin monoclonal antibody.

For cAMP assays, 293T-GLP-1R cells were plated overnight into 384-well tissue culture treated white microtiter plates (Corning No. 3704) at a density of 5,000, 8,000 or 10,000 cells/well in 20 uL medium/well. The following day, serial dilutions of agonist (GLP-1 peptide or the GLP1-triazole-Fc hybrid immunoglobulins) in 20 uL PBS containing 0.5 mM IBMX were added to the cells, and the cells then incubated at 37° C. for 1, 4 or 24 hours. Following stimulation, cAMP levels were determined by Homogeneous Time-Resolved Fluorescence (HTRF) in a ClarioStar microplate reader (BMG Labtech) using a Cisbio cAMP dynamic 2 kit according to the manufacturer's instructions. Following addition of HTRF detection reagents, anti-cAMP Mab labeled with Cryptate (20 uL) and the cAMP labeled with d2 dye (20 uL), the plates were incubated for 1 hour at room temperature and the fluorescence ratio (665 nm/620 nm) calculated and used to determine the cAMP concentration in the cell lysates by four-parameter fit to a cAMP standard curve.

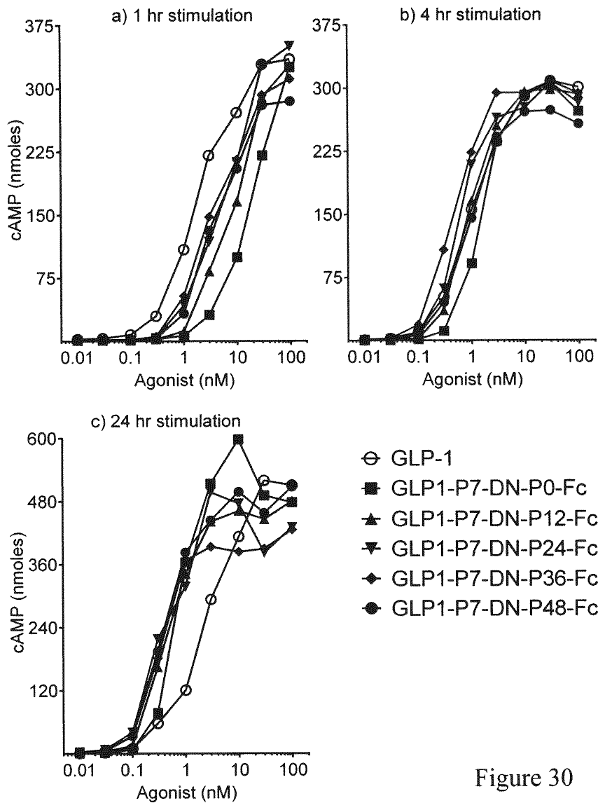

FIG. 30 shows the results for GLP-1(7-37) peptide and the GLP1-P7-DN-Px-Fc proteins (x=0, 12, 24, 36, 48). All five GLP1-triazole-Fc hybrid immunoglobulins induced cAMP levels comparable to GLP-1(7-37) peptide. Stimulation by GLP-1(7-37) was similar whether cells were exposed to agonist for 1, 4 or 24 hours, with an EC50 of ~2 nM at 24 hours, whereas stimulation by the GLP1-triazole-Fc hybrid immunoglobulins increased dramatically as cells were exposed to agonist for longer times, with an EC50 of ~0.4 nM at 24 hours.

### Example 7: N-Terminal Cyclooctyne-Fc Proteins

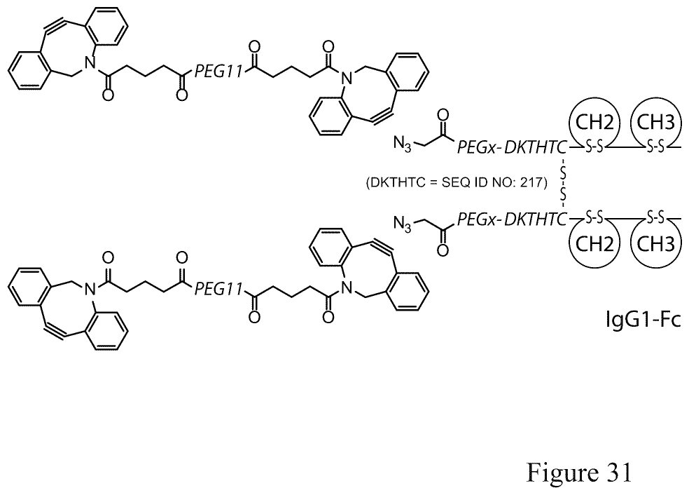

A series of cyclooctyne-modified Fc proteins (DIBAC-P11-DN-Px-Fc), each having a cyclooctyne functional group at its N-terminus is prepared by reacting a homobifunctional cyclooctyne linker with the azide-modified N₃-Px-Fc proteins of Example 4. The linker, DIBAC-PEG11-DIBAC, shown in FIG. 31, was obtained from CPC Scientific (calculated for C₇₄H₁₀₂N₆O₁₇ [M+H]+ 1346.6, found 1346.4). The PEG11 portion of this linker was derived from diamido-dPEG11-diamine (Quanta Biodesigns No. 10361) having the structure: [—NH—CH₂—(CH₂—CH₂—O)₃—(CH₂)₃—CO—NH—(CH₂—CH₂—O)₅—(CH₂)₂—CO—NH—CH₂—(CH₂—CH₂—O)₃—(CH₂)₃—NH—].

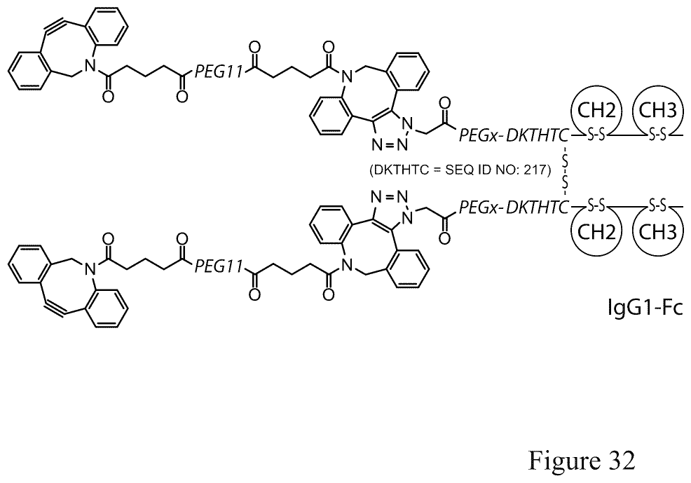

DIBAC-PEG11-DIBAC is reacted individually with each one of the five N₃-Px-Fc proteins (FIG. 31), to generate a series of DIBAC-P11-DN-Px-Fc proteins (FIG. 32). Representative results are shown for the reaction of DIBAC-PEG11-DIBAC with the N₃-P0-Fc protein to generate DIBAC-P11-DN-P0-Fc. The reaction (1 mL) was initiated by adding 84 mg of the N₃-Px-Fc protein to 11.25 mg of the DIBAC-PEG11-DIBAC linker in 0.02 M sodium phosphate pH 7.0 in water-ethanol (0.64:0.36 vol/vol). The reaction was carried out for 12 hours at room temperature, and the DIBAC-PEG11-DIBAC linker was then extracted by adding 1 mL of PBS, mixing well, and centrifuging at 12,000×g which separated out the linker as an denser, oily phase. The desired DIBAC-P11-DN-P0-Fc product contained within the upper aqueous phase was purified by HiTrap Protein A HP chromatography, desalted and concentrated as described in Example 4.

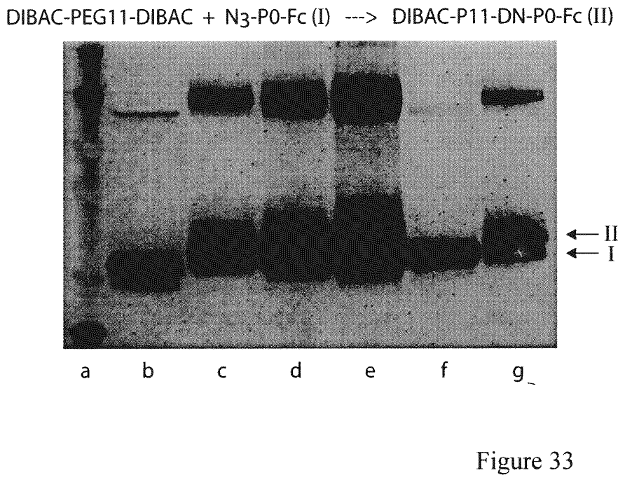

FIG. 33 shows the DIBAC-P11-DN-P0-Fc reaction product by SDS-PAGE under reducing conditions: Fc6 control (lane b), unpurified reaction product (lanes c-e), the purified N₃-P0-Fc protein (lane f), and the purified DIBAC-P11-DN-P0-Fc protein (lane g). Approximately, 70% of the N₃-P0-Fc (I) protein was converted into a product having the expected size of the DIBAC-P11-DN-P0-Fc (II) protein.

### Example 8: DNA-triazole-Fc Hybrid Immunoglobulins

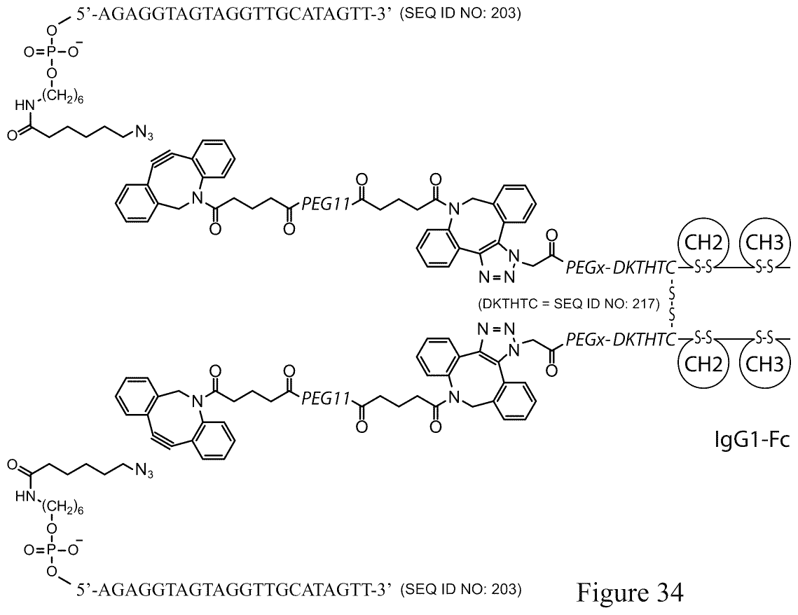

A series of DNA-triazole-Fc hybrid immunoglobulins (DNA-P11-DN-Px-Fc) are prepared by reacting an azide-modified DNA or RNA, with each of the five DIBAC-P11-DN-Px-Fc proteins of Example 7. FIG. 34 shows the structure of the azide-modified DNA, 5AzD-let7d, having the sequence 5′-AGAGGTAGTAGGTTGCATAGTT-3′ (SEQ ID NO:203) of the DNA coding strand for the mature human hsa-let-7d-5p miRNA (www.mirbase.org, Accession No. MIMAT0000065). The 5AzD-let7d oligonucleotide (5AzD-let7d) was obtained from Integrated DNA Technologies (Coralville, IA). Prior to use, 5AzD-let7d (molecular weight 7187.8) was dissolved in 10 mM Tris HCl, 1 mM EDTA.

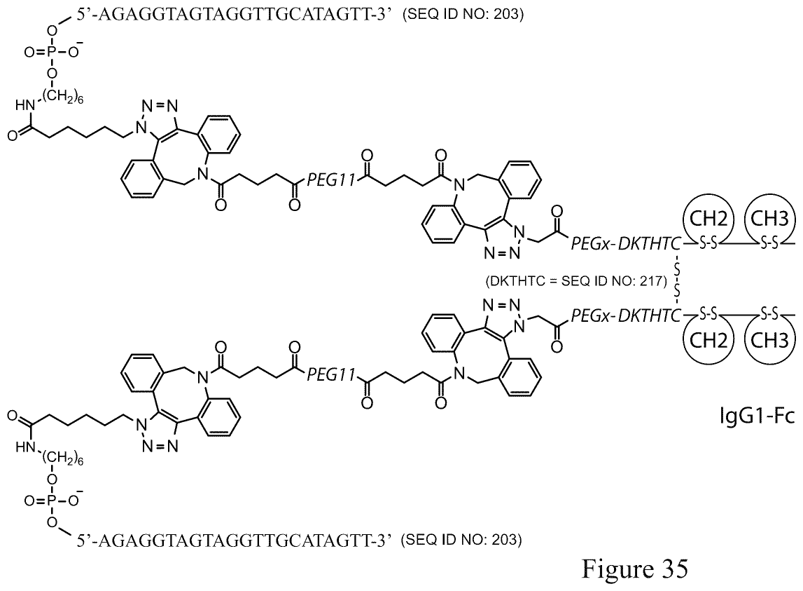

5AzD-let7d was reacted individually with each of the DIBAC-P11-DN-Px-Fc proteins (FIG. 34) to generate a series of DNA-triazole-Fc hybrid immunoglobulins (FIG. 35). Representative results are shown for the reaction of 5AzD-let7d with the DIBAC-P11-DN-P0-Fc protein. Reactions (20 ul) contained 0.1 M sodium phosphate pH 7.0, 50 μg of 5AzD-let7d or a series of two-fold dilutions thereof, and 5.7 ug of the DNA-P11-DN-Px-Fc protein. Reactions were carried out at room temperature for 2 hours.

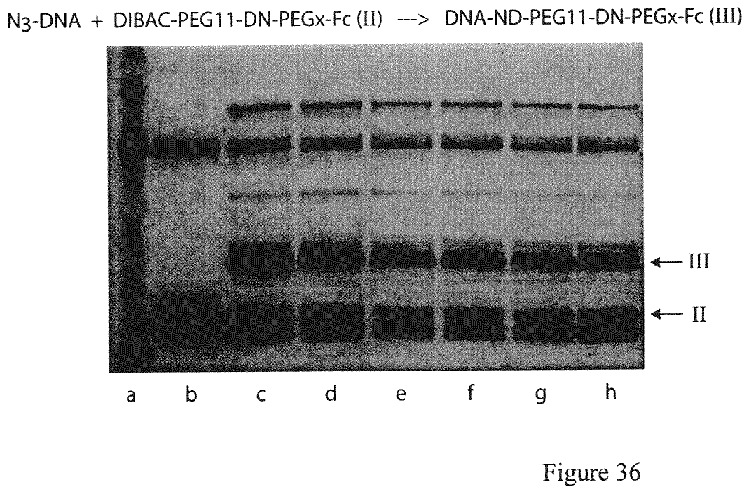

FIG. 36 shows the reaction products by SDS-PAGE under reducing conditions: the 5AzD-let7d oligonucleotide concentration (mg/ml) was as follows: markers (lane a), 0 (lane b), 2.5 (lane c), 1.25 (lane d), 0.063 (lane e), 0.031 (lane f), 0.016 (lane g), 0.08 (lane h). Approximately 90% of the DIBAC-P11-DN-P0-Fc (II) protein was converted into a product having the expected size of the DNA-ND-P11-DN-PEG0-Fc (III) hybrid immunoglobulin.

### Example 9: N-Terminal Azide-Mab Proteins

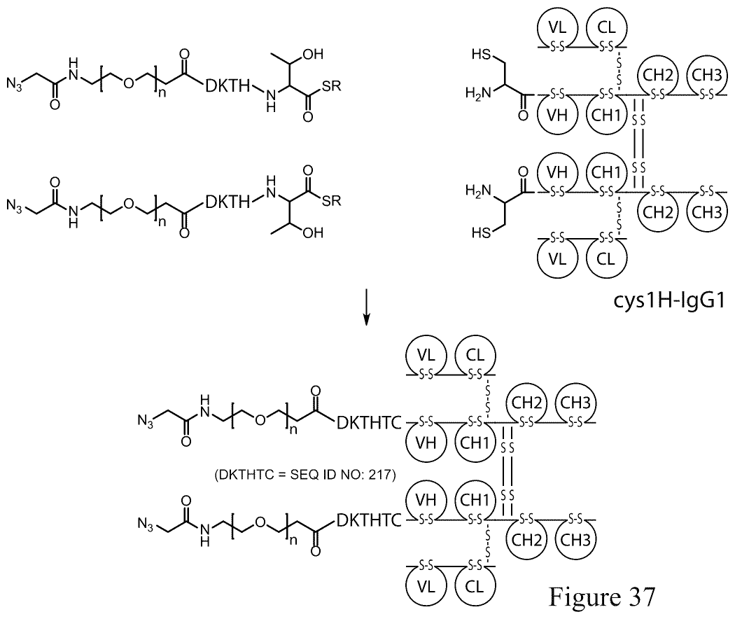

A series of azide-modified trastuzumab proteins (N₃-Px-Hc), each having an azide functional group at the N-terminus of its heavy chain, and optionally a PEG linker, is prepared by reacting a trastuzumab protein variant, cys1H-IgG1, with thioesters having the sequence azidoacetyl-Px-DKTHT-thiophenol (FIG. 37). DKTHT is SEQ ID NO: 220.

Cys1H-IgG1 consists of the wild-type trastuzumab light chain shown in SEQ ID NO: 128, and a variant trastuzumab heavy chain, having at its heavy chain N-terminus a cysteine residue. The cys1H-IgG1 heavy chain is initially initially expressed as the variant trastuzumab pre-heavy chains shown in SEQ ID NO: 167, SEQ ID NO: 168, and SEQ ID NO: 169, having a SHH signal peptide, IFN signal peptide, and CETP signal peptide, respectively. Cleavage of the heterologous signal sequences by the cellular signal peptidase provides the mature heavy chain protein having an N-terminal cysteine, the sequence of which is shown in SEQ ID NO: 166.

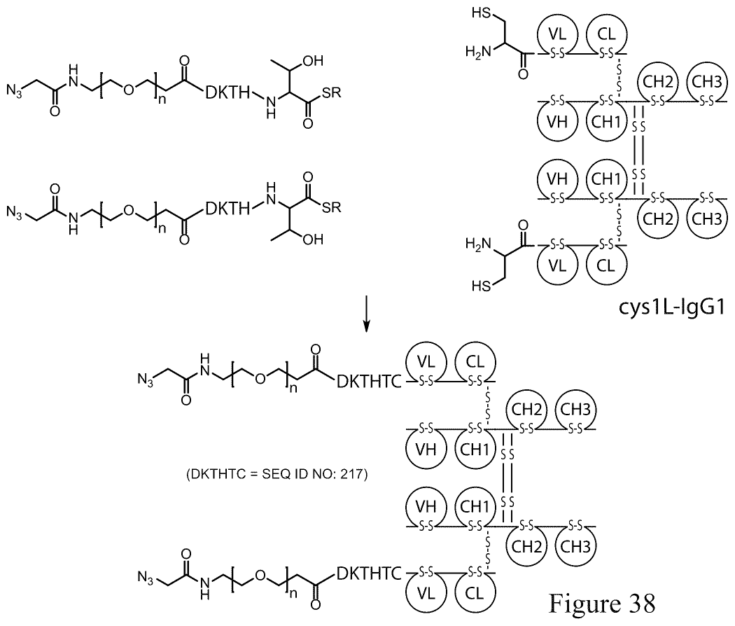

A second series of azide-modified trastuzumab proteins (N3-Px-Lc), each having an azide functional group at the N-terminus of its light chain, and optionally a PEG linker, is prepared by reacting a trastuzumab protein variant, cys1L-IgG1, with thioesters having the sequence azidoacetyl-Px-DKTHT-thiophenol (FIG. 38). DKTHT is SEQ ID NO: 220.

Cys1L-IgG1 consists of the wild-type trastuzumab heavy chain shown in SEQ ID NO: 129, and a variant trastuzumab light chain, having at its heavy chain N-terminus a cysteine residue. The cys1L-IgG1 light chain is initially initially expressed as the variant trastuzumab pre-light chains shown in SEQ ID NO: 131, SEQ ID NO: 132, and SEQ ID NO: 133 having a SHH signal peptide, IFN signal peptide, and CETP signal peptide, respectively. Cleavage of the heterologous signal sequences by the cellular signal peptidase provides the mature light chain protein having an N-terminal cysteine, the sequence of which is shown in SEQ ID NO: 130.

Appropriate light and heavy chain expression vectors are co-transfected to produce the cys1H-IgG1 and cysL-IgG proteins. Protein production is executed by transient expression in CHO-DG44 cells, adapted to serum-free suspension culture followed by Protein A purification, as described in Example 1.

### Example 10: Mertansine-triazole-trastuzumab Hybrid Immunoglobulins

A series of mertansine-triazole-trastuzumab hybrid immunoglobulins are prepared by reacting the maytansinoid DM1 (mertansine), further modified to have a cyclooctyne functional group, with each of the N₃-Px-Hc and N₃-Px-Lc proteins of Example 9.

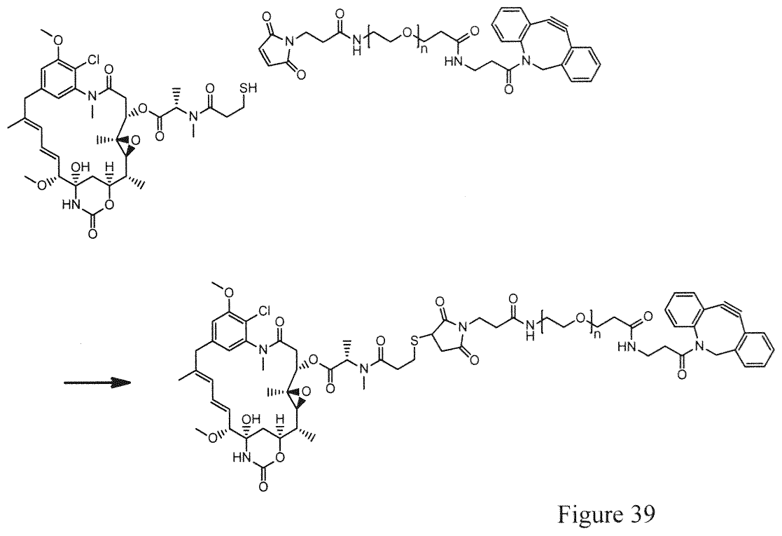

DM1 (free thiol form; M.W. 737.5 g/mole) is prepared as described previously in U.S. Pat. Nos. 5,208,020 and 6,333,410 B1, which are hereby incorporated by reference. A cyclooctyne functional group is added to DM1 using the DBCO-PEG4-Maleimide heterobifunctional linker which contains a maleimide group capable of reacting with the free thiol group of DM1 (FIG. 39). DM1 is reacted with DBCO-PEG4-Maleimide in DMSO using the procedures of Example 5. The cycloctyne-modified-DM1 product (DM1-P4-DBCO) is purified by HPLC, and dissolved in DMSO prior to use.

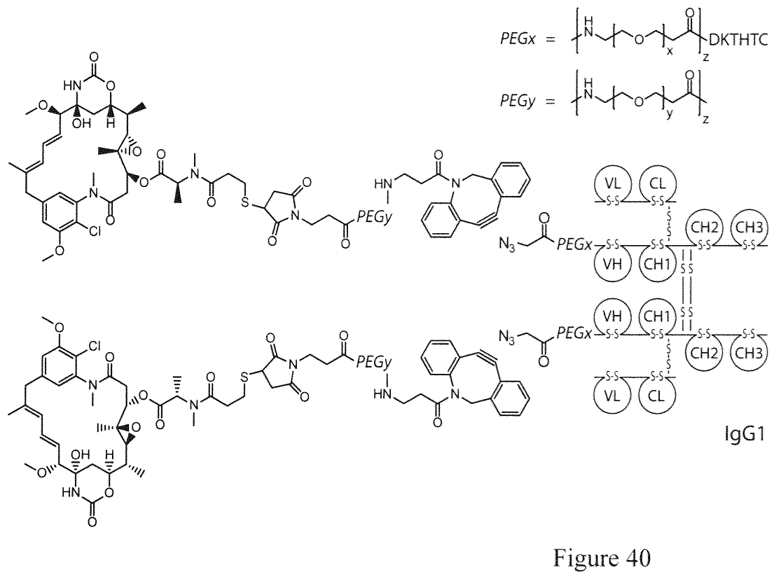

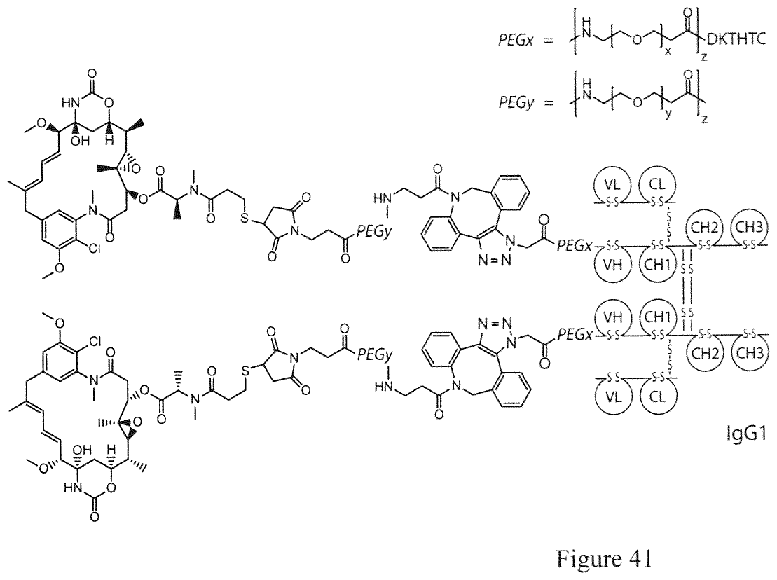

DM1-P4-DBCO is reacted individually with each one of the five N3-Px-Hc proteins (FIG. 40), to generate a series of mertansine-triazole-trastuzumab hybrid immunoglobulins modified with DM1 at the N-terminus of the trastuzumab heavy chain (DM1-P4-triazole-Px-Hc) (FIG. 41).

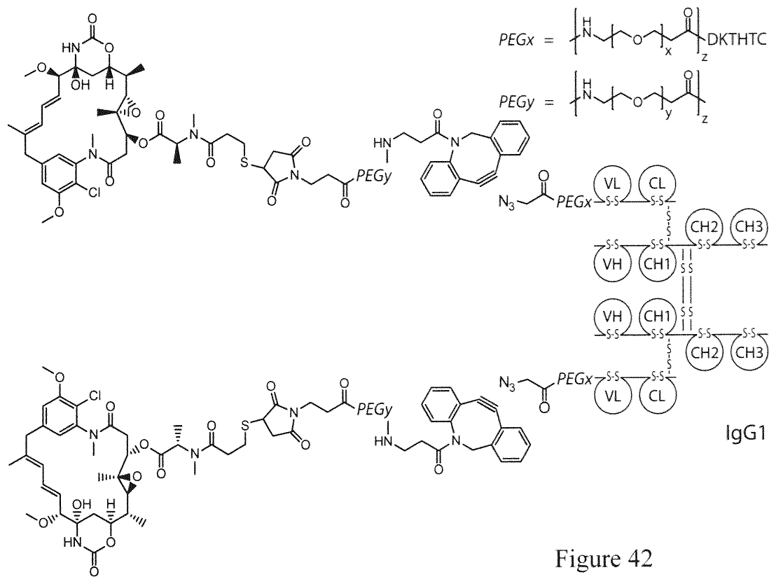

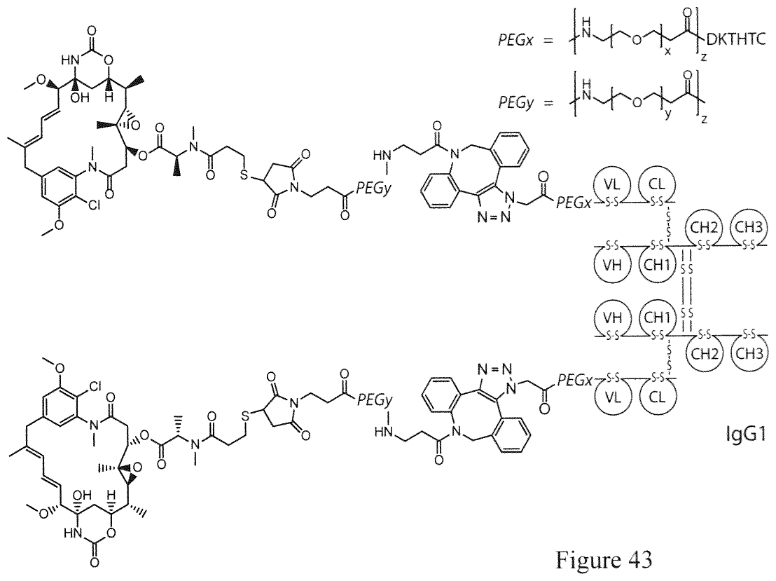

DM1-P4-DBCO is reacted individually with each one of the five N3-Px-Lc proteins (FIG. 42), to generate a series of mertansine-triazole-trastuzumab hybrid immunoglobulins modified with DM1 at the N-terminus of the trastuzumab light chain (DM1-P4-triazole-Px-Lc) (FIG. 43).

The efficacy of the mertansine-triazole-trastuzumab hybrid immunoglobulins as novel antibody drug conjugates is evaluated and compared with ado-trastuzumab emtansine, obtained from Genentech (South San Francisco, CA), using in vitro cell proliferation assays and in vivo tumor growth inhibition assays as described in U.S. Pat. No. 7,521,541B2, which is hereby incorporated by reference.

### Example 11: N-Terminal Tetrazine-Fc Proteins

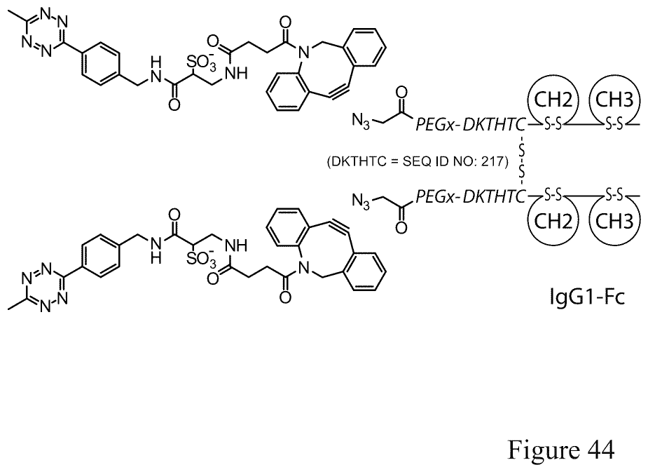

A series of tetrazine-modified Fc proteins (Tet-Px-Fc), each having a tetrazine group at its N-terminus and optionally a PEGx linker was prepared by reacting a heterobifunctional linker with the azide-modified N₃-Px-Fc proteins of Example 4. FIG. 44 shows the Tetrazine-DBCO heterobifunctional linker, which has a cycloctyne group at one end capable reacting with the azide group of the N₃-Px-Fc proteins, and a tetrazine group at the other end. The Tetrazine-DBCO linker was obtained from Click Chemistry Tools (Item No. 1022; C₃₂H₂₉N₇O₆S, protonated; molecular weight 639.68, protonated). Prior to use, Tetrazine-DBCO was dissolved at a concentration of 25 mg/mL in water.

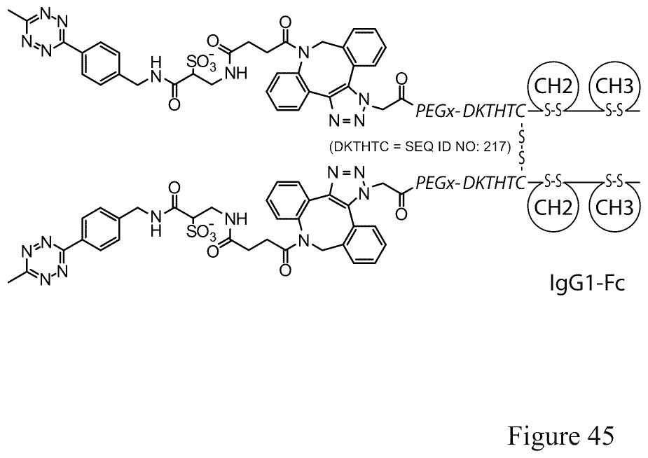

Tetrazine-DBCO was reacted individually with each N₃-Px-Fc protein (FIG. 44) to generate the corresponding series of Tet-Px-Fc proteins (FIG. 45). Reactions (0.72 mL) contained 0.1 M sodium phosphate pH 7.0, 0.1875 mg of the Tetrazine-DBCO linker, and 0.6 mg of the N₃-Px-Fc protein. Reactions were carried out for 3.5 hours at room temperature. Excess unreacted linker was removed by HiTrap ProteinA HP chromatography.

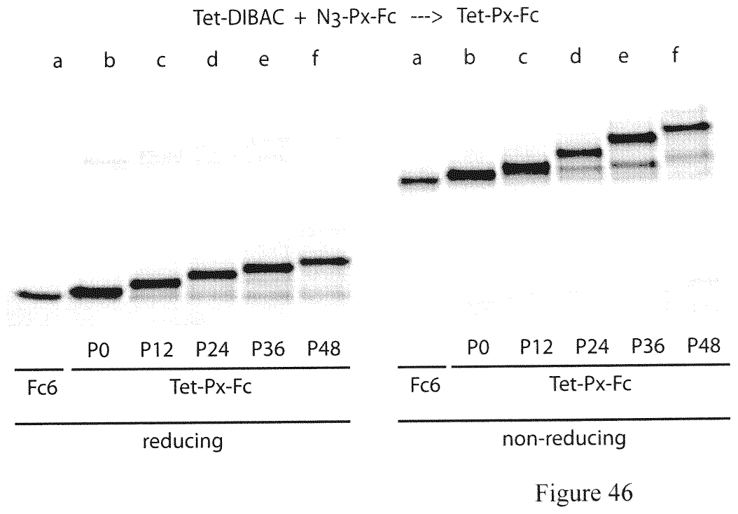

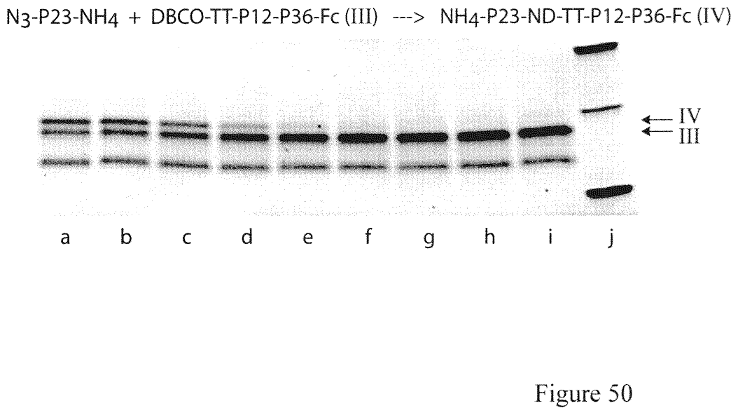

FIG. 46 shows the purified Tet-Px-Fc proteins by SDS-PAGE under reducing (left) and non-reducing conditions (right): Fc6 control (lanes a), Tet-P0-Fc (lanes b), Tet-P12-Fc (lanes c), Tet-P24-Fc (lanes d), Tet-P36-Fc (lanes e), and Tet-P48-Fc (lanes f). The size on SDS-PAGE of the Tet-Px-Fc proteins increased as the PEG linker length increased, under both reducing and non-reducing conditions. In addition, each of the Tet-Px-Fc proteins was larger than the corresponding N₃-Px-Fc protein by SDS-PAGE under reducing conditions (FIG. 50).

### Example 12: N-Terminal Trancyclooctene-Fc Proteins

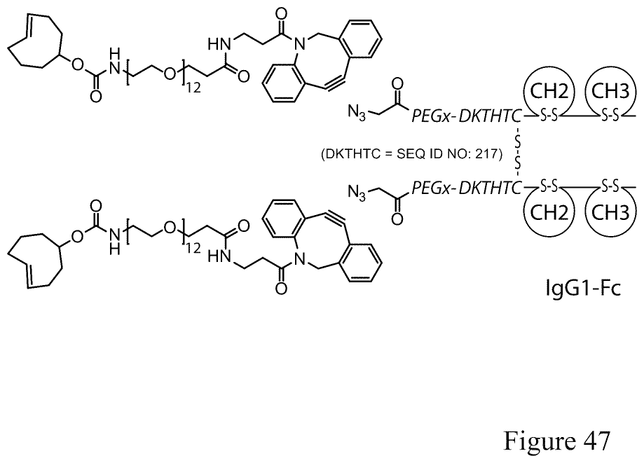

A series of transcyclooctene-modified Fc proteins (Tco-Px-Fc), each having a transcyclooctene group at its N-terminus and optionally a PEGx linker, is prepared by reacting a heterobifunctional linker with the azide-modified N₃-Px-Fc proteins of Example 4. FIG. 47 shows the TCO-PEG12-DBCO heterobifunctional linker, which has a cycloctyne group at one end capable reacting with the azide group of the N₃-Px-Fc proteins, and a transcyclooctene group at the other end. The TCO-PEG12-DBCO linker was obtained from Click Chemistry Tools (Item No. 1005; C₅₄H₈₁N₃O₁₆; molecular weight 1028.23). Prior to use, TCO-PEG12-DBCO was dissolved at a concentration of 100 mg/mL in DMSO.

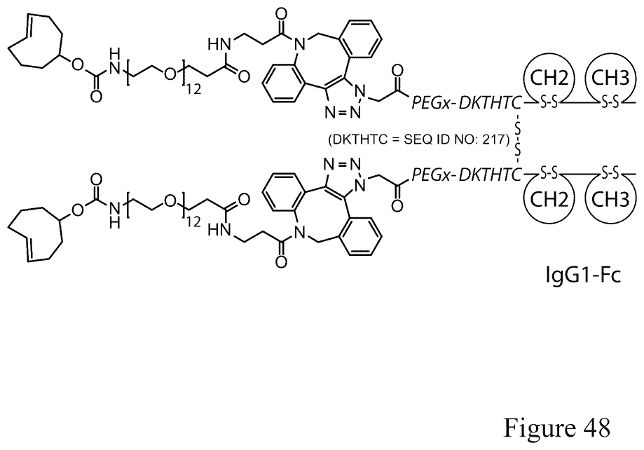

TCO-PEG12-DBCO is reacted individually with each N₃-Px-Fc protein (FIG. 47) to generate the corresponding series of Tco-P12-Px-Fc proteins (FIG. 48). Representative results are shown for the reaction of TCO-PEG12-DBCO with the N₃-P36-Fc protein. Reactions (6 uL) contained 0.1 M sodium phosphate pH 7.0, 0.2 mg of the TCO-PEG12-DBCO linker or a series of two-fold dilutions thereof in DMSO, and 5 μg of the N₃-P36-Fc protein. Reactions were carried out for 3.5 hours at room temperature.

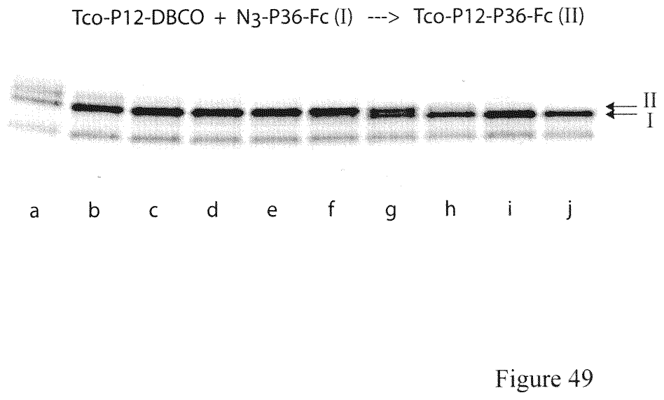

FIG. 49 shows the Tco-P12-Px-Fc proteins by SDS-PAGE under reducing conditions: the Tco-P12-DBCO linker concentration (mg/ml) was as follows: 32 (lane a), 16 (lane b), 8 (lane c), 4 (lane d), 2 (lane e), 1 (lane f), 0.5 (lane g), 0.25 (lane h), 0.125 (lane i), and 0 (lane j). The conversion the N₃-P36-Fc (I) protein into the Tco-P12-P36-Fc (II) protein was essentially complete at a Tco-P12-DBCO linker concentration of 1 mg/ml (lane f). In further studies, the Tco-P12-P36-Fc protein thereby obtained was purified by HiTrap ProteinA HP chromatography, desalted and concentrated as described in Example 4.

To test the reactivity of the Tco-P12-P36-Fc protein with a tetrazine functional group, purified Tco-P12-P36-Fc protein was first reacted with the heterobifunctional Tetrazine-DBCO linker to prepare DBCO-TT-P12-P36-Fc protein, which was purified by Protein A and then tested for its ability to react with an azido-PEG-amine linker, NH₂-PEG23-N₃, obtained from Quanta Biodesigns (Item No. 10525, C₄₈H₉₈N₄O₂₃, molecular weight 1099.30). The test reactions (6 uL) contained 0.1 M sodium phosphate pH 7.0, 0.2 mg of the NH₂-PEG23-N₃ linker or a series of two-fold dilutions thereof, and 5 μg of the DBCO-TT-P12-P36-Fc protein. Reactions were carried out for 1 hour at room temperature.

FIG. 50 shows the reaction products by SDS-PAGE under reducing conditions: the NH₂-PEG23-N₃ linker concentration (mg/ml) was as follows: 0.12 (lane a), 0.06 (lane b), 0.03 (lane c), 0.015 (lane d), 0.0075 (lane e), 0.0038 (lane f), 0.002 (lane g), 0.001 (lane h), 0 (lane i). The DBCO-TT-P12-P36-Fc (III) protein, but not the Tco-P12-P36-Fc protein (not shown), was converted into the expected NH₂-P23-ND-TT-P12-P36-Fc (IV) protein, confirming the reactivity of the Tco-P12-P36-Fc protein with a tetrazine functional group.

### Example 13: GLP1-dihydropyridizine-Fc Hybrid Immunoglobulins

A series of GLP1-dihydropyridizine-Fc hybrid immunoglobulins (GLP1-P3-TT-Px-Fc) were prepared by reacting a transcyclooctene-modified GLP-1 analog with the Tet-Px-Fc proteins of Example 11. GLP1-dihydropyridizine-Fc hybrid immunoglobulins are also prepared by reacting a tetrazine-modified GLP-1 analog with the Tco-Px-Fc proteins of Example 12.

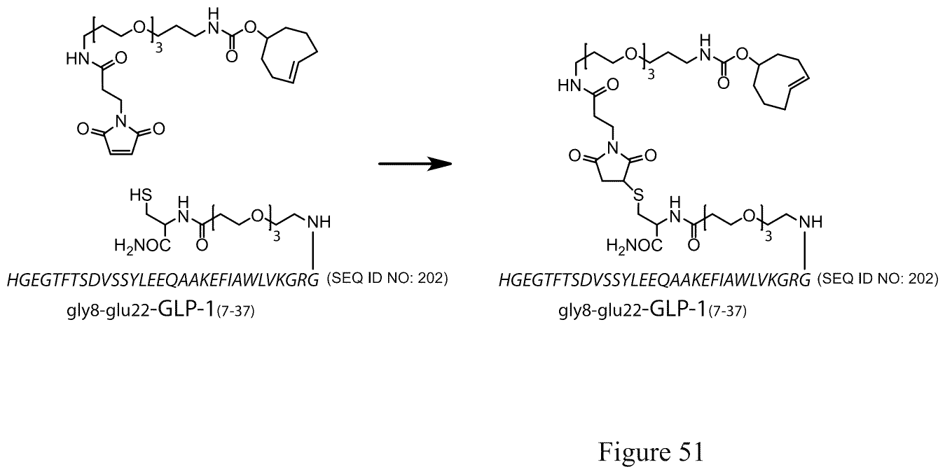

To prepare the transcyclooctene-modified GLP-1 analog, the gly8-glu22-GLP-1(7-37)-PEG3-cys-NH2 peptide was reacted with a heterobifunctional linker, TCO-PEG3-Maleimide, which contains a maleimide group capable of reacting with the free thiol group on the C-terminal cysteine residue (FIG. 51). TCO-PEG3-Maleimide (C₂₆H₄₁N₃O₈, mol weight 523.62) was obtained from Click Chemistry Tools (Item No. 1002). Prior to use, the linker was dissolved at a concentration of 25 mg/mL in DMSO. Reactions (0.42 ml) contained 50 mM MES pH 6.5, 5 mM EDTA, 0.45 mg of gly8-glu22-GLP-1(7-37)-PEG3-cys-NH₂ peptide and 0.375 mg of the TCO-PEG3-Maleimide linker. Reactions were carried out at room temperature for 60 minutes. Excess unreacted linker was removed by buffer-exchange into 0.02 M sodium phosphate pH 7.0 using a 5 mL HiTrap Desalting Column. FIG. 51 shows the structure of the transcyclooctene-modified GLP-1 analog (GLP1-P6-Tco).

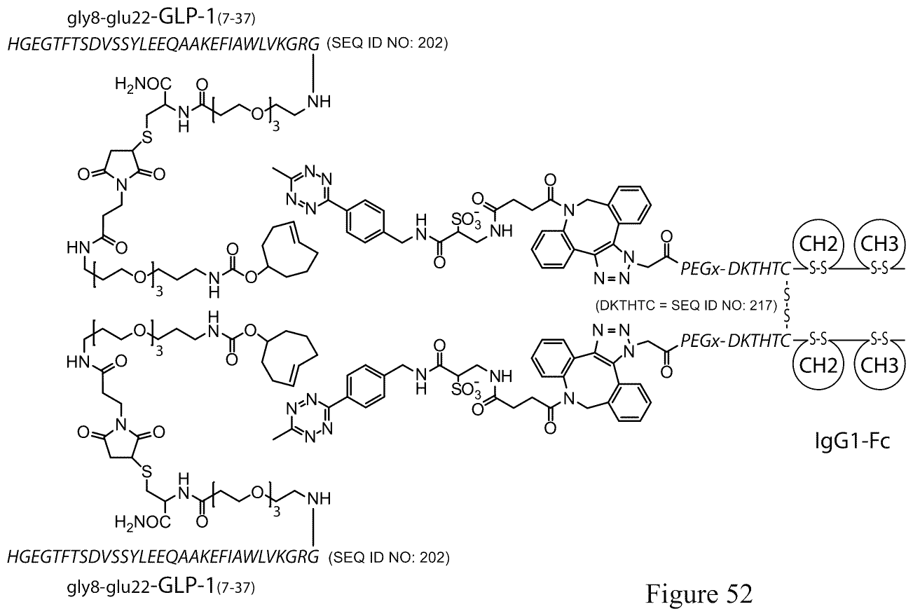

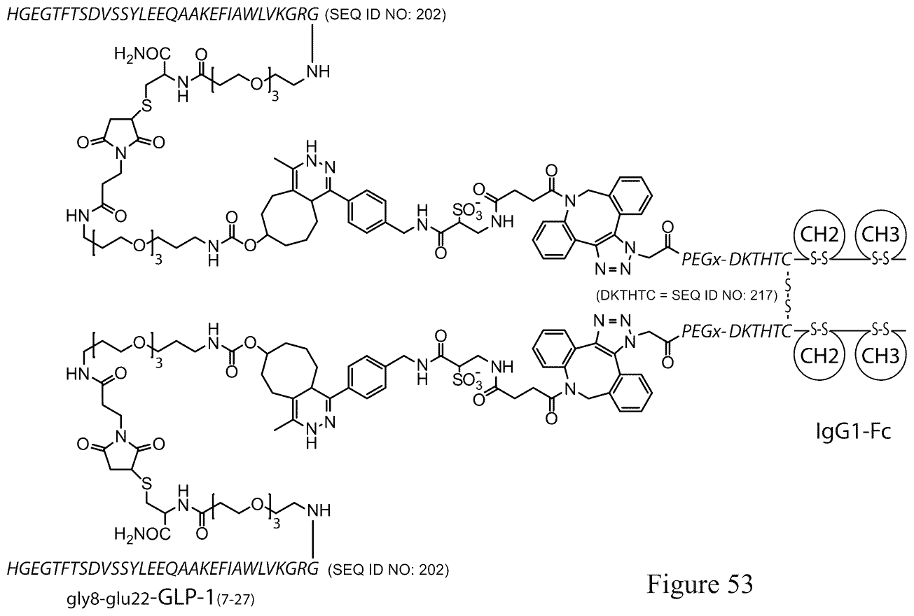

The GLP1-P6-Tco peptide was reacted individually with each of the Tet-Px-Fc proteins (FIG. 52), to generate the GLP1-P3-TT-Px-Fc series of hybrid immunoglobulins (FIG. 53). Reactions (0.99 ml) contained 0.1 M sodium phosphate pH 7.0, 0.145 mg of GLP1-P6-Tco peptide and 0.33 mg of each Tet-Px-Fc proteins. Reactions were carried out at room temperature for 30 minutes. The GLP1-P6-TT-Px-Fc hybrid immunoglobulins were then purified by chromatography on HiTrap Protein A HP.

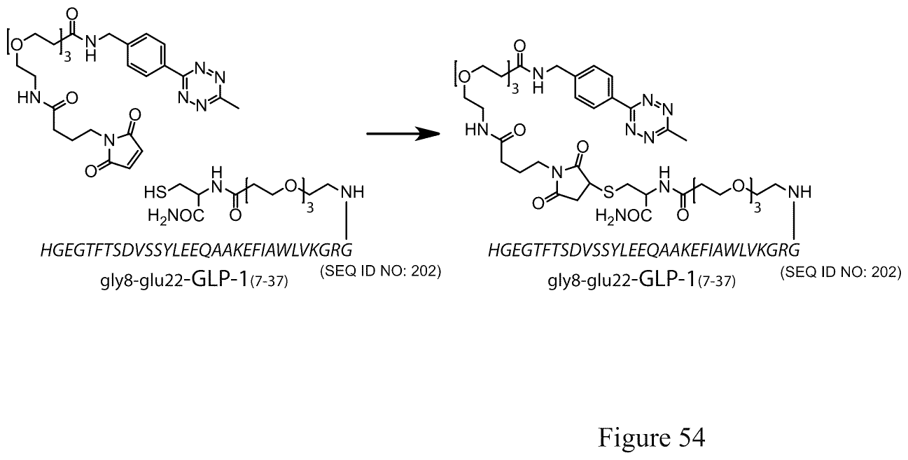

To prepare the tetrazine-modified GLP-1 analog, the gly8-glu22-GLP-1(7-37)-PEG3-cys-NH2 peptide is reacted with a heterobifunctional linker, Tetrazine-PEG4-Maleimide, which contains a maleimide group capable of reacting with the free thiol group on the C-terminal cysteine residue (FIG. 54).

Tetrazine-PEG4-Maleimide (C₂₉H₃₉N₇O, mol weight 613.66) was obtained from Click Chemistry Tools (Item No. A139). Prior to use, the linker is dissolved at a concentration of 25 mg/mL in DMSO. Reactions (0.42 ml) contain 50 mM MES pH 6.5, 5 mM EDTA, 0.45 mg of gly8-glu22-GLP-1(7-37)-PEG3-cys-NH2 peptide and 0.375 mg of the Tetrazine-PEG4-Maleimide linker. Reactions are carried out at room temperature for 60 minutes. Excess unreacted linker is removed by buffer-exchange into 0.02 M sodium phosphate pH 7.0 using a 5 mL HiTrap Desalting Column. FIG. 54 shows the structure of the tetrazine-modified GLP-1 analog (GLP1-P7-Tet).

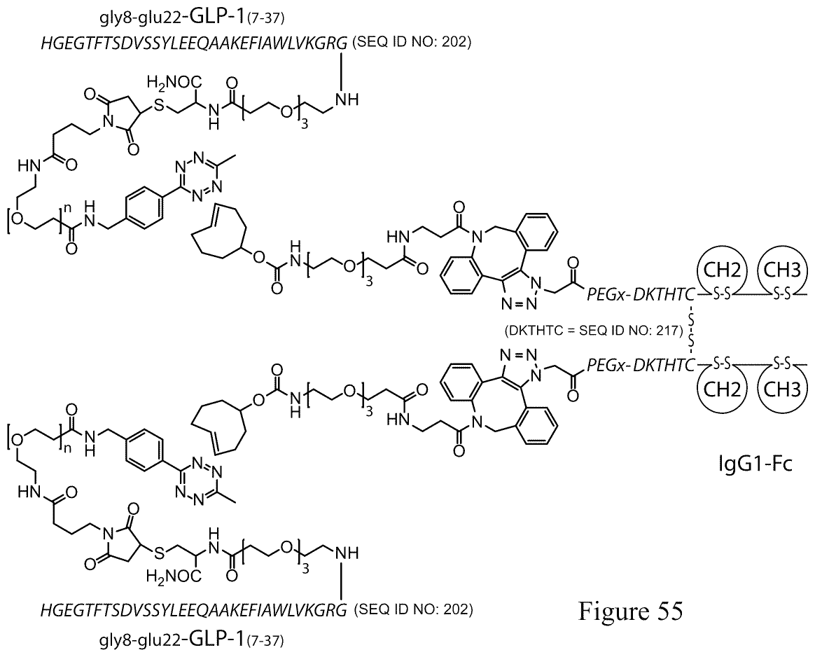

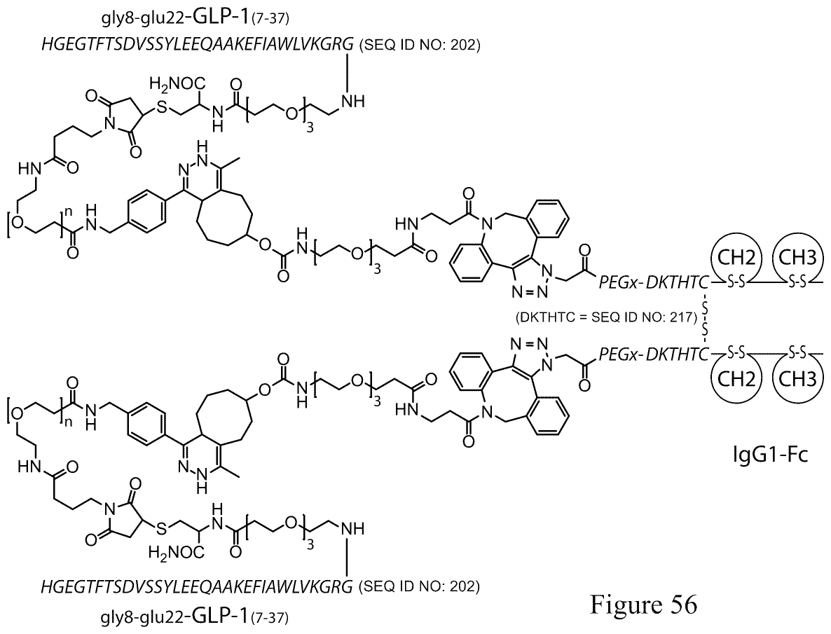

The GLP1-P7-Tet peptide is reacted individually with each of the Tco-Px-Fc proteins (FIG. 55), to generate the GLP1-P7-TetTco-Px-Fc series of hybrid immunoglobulins (FIG. 56). Reactions (0.99 ml) contain 0.1 M sodium phosphate pH 7.0, 0.145 mg of GLP1-P7-Tet peptide and 0.33 mg of each Tco-Px-Fc protein. Reactions are carried out at room temperature for 30 minutes. The GLP1-P7-Tet/Tco-Px-Fc hybrid immunoglobulins are then purified by chromatography on HiTrap Protein A HP.

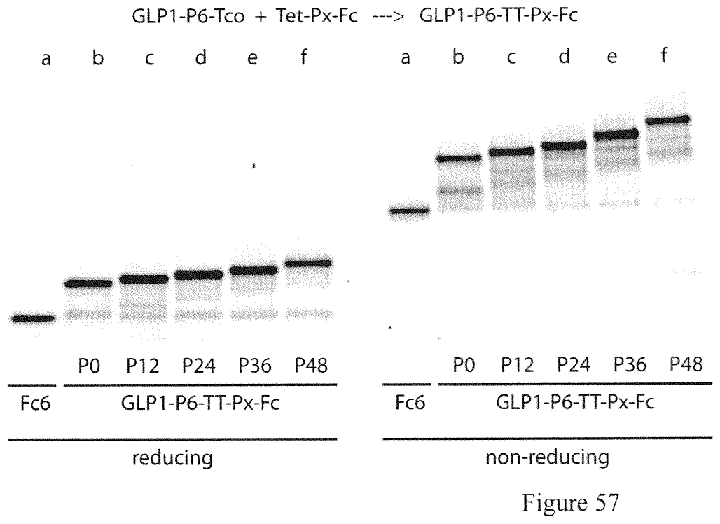

FIG. 57 shows the purified GLP1-dihydropyridizine-Fc hybrid immunoglobulins by SDS-PAGE under reducing conditions (left) and non-reducing conditions (right): Fc6 control (lanes a), GLP1-P6-TT-P0-Fc (lanes b), GLP1-P6-TT-P12-Fc (lanes c), GLP1-P6-TT-P24-Fc (lanes d), GLP1-P6-TT-P36-Fc (lanes e), and GLP1-P6-TT-P48-Fc (lanes f). The size of GLP1-dihydropyridizine-Fc hybrid immunoglobulins increased with PEG linker length comparable to the Tet-Px-Fc proteins.

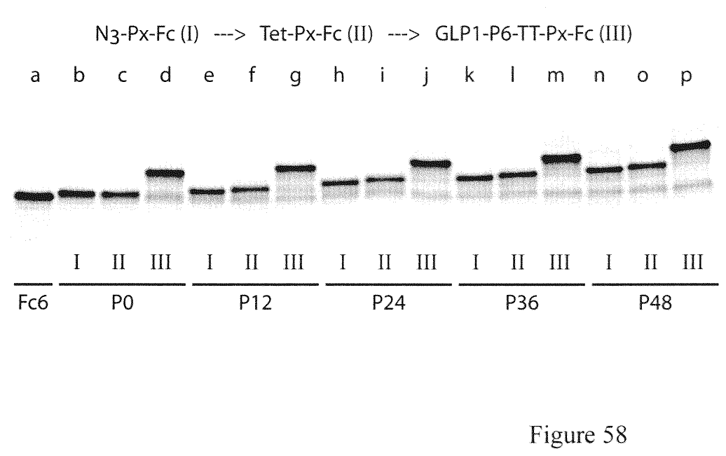

FIG. 58 directly compares the N3-Px-Fc (I) proteins, the Tet-Px-Fc (II) proteins, and the GLP1-dihydropyridizine-Fc (III) hybrid immunoglobulins by SDS-PAGE under reducing conditions: Fc6 control (lane a), N₃-P0-Fc (lane b), Tet-P0-Fc (lane c), GLP1-P6-TT-P0-Fc (lane d), N₃-P12-Fc (lane e), Tet-P12-Fc (lane f), GLP1-P6-TT-P12-Fc (lane g), N₃-P24-Fc (lane h), Tet-P24-Fc (lane i), GLP1-P6-TT-P24-Fc (lane j), N₃-P36-Fc (lane k), Tet-P36-Fc (lane l), GLP1-P6-TT-P36-Fc (lane m), N₃-P48-Fc (lane n), Tet-P48-Fc (lane o), GLP1-P6-TT-P48-Fc (lane p). The conversion of each Tet-Px-Fc protein to the corresponding GLP1-P6-TT-Px-Fc hybrid immunoglobulin was approximately 92%.

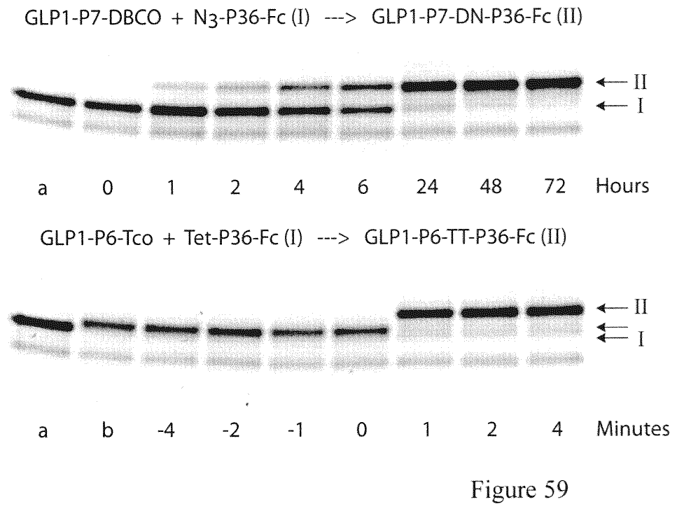

FIG. 59 shows a time course for the reaction of GLP1-P7-DBCO with N₃-P36-Fc and a time course for the reaction of GLP1-P6-Tco with Tet-P36-Fc. Reactions were carried out as described above for the various times indicated, except that each reaction was terminated by the addition of an excess of competitor. For the reaction of GLP1-P7-DBCO with N₃-P36-Fc, sodium azide was added to a final concentration of 0.1%; for the reaction of GLP1-P6-Tco with Tet-P36-Fc, TCO-PEG3-Maleimide was added to a final concentration of 3.5 mg/ml. Each reaction was analyzed SDS-PAGE under reducing conditions: (upper panel) N₃-P36-Fc alone (lane a), N₃-P36-Fc+GLP1-P7-DBCO for the following times, 0, 1, 2, 4, 6, 24, 48, 72 hours; (lower panel) Tet-P36-Fc alone (lane a), Tet-P36-Fc+TCO-PEG3-Maleimide alone (lane b), Tet-P36-Fc+GLP1-P6-Tco for the following times, −4, −2, −1, 0, 1, 2, 4 minutes. The reaction of GLP1-P6-Tco with Tet-P36-Fc (I) leading to the formation of GLP1-P7-DN-P36-Fc (II) is much faster, reaching completion within 1 minute, whereas the reaction of the GLP1-P7-DBCO with N₃-P36-Fc (I) leading to the formation of GLP1-P7-DN-P36-Fc (II) is only 50% complete after 6 hours.

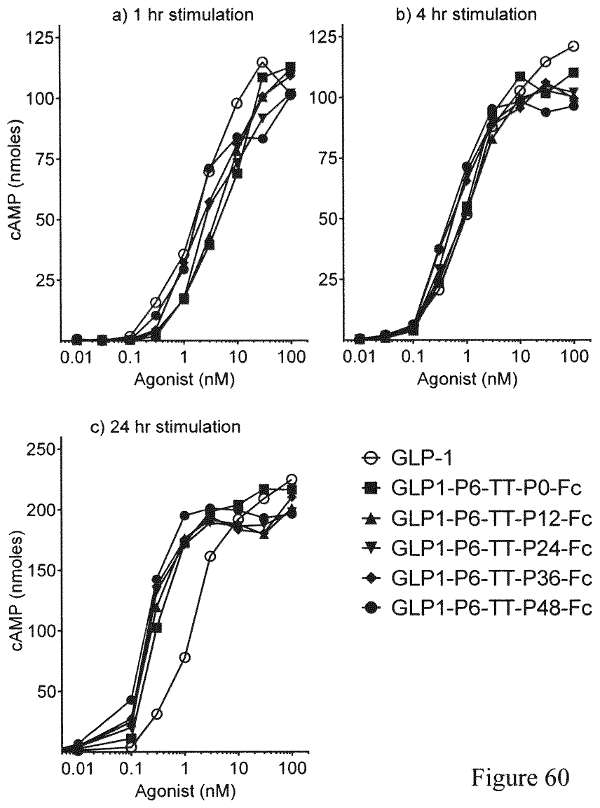

The biological activity of GLP1-P6-dihydropyridizine-Px-Fc hybrid immunoglobulins was evaluated in a cell-based assay as described in Example 6. FIG. 60 shows the results for GLP-1(7-37) peptide and the GLP1-P6-TT-Px-Fc proteins (x=0, 12, 24, 36, 48). All five GLP1-dihydropyridizine-Fc hybrid immunoglobulins induced cAMP levels comparable to GLP-1(7-37) peptide. Stimulation by GLP-1(7-37) was similar whether cells were exposed to agonist for 1, 4 or 24 hours, with an EC50 of ~2 nM at 24 hours, whereas stimulation by the GLP1-dihydropyridizine-Fc hybrid immunoglobulins increased dramatically as cells were exposed to agonist for longer times, with an EC50 of ~0.2 nM at 24 hours.

### Example 14: Adalimumab Fab-dihydropyridizine-Fc Hybrid Immunoglobulins

A series of Fab-dihydropyridizine-Fc hybrid immunoglobulins (Fab-P3-TT-Px-Fc) were prepared by reacting a transcycloctene-modified Fab fragment with the Tet-Px-Fc proteins of Example 11. Fab-dihydropyridizine-Fc hybrid immunoglobulins are also prepared by reacting a a tetrazine-modified Fab fragment with the Tco-Px-Fc proteins of Example 12.

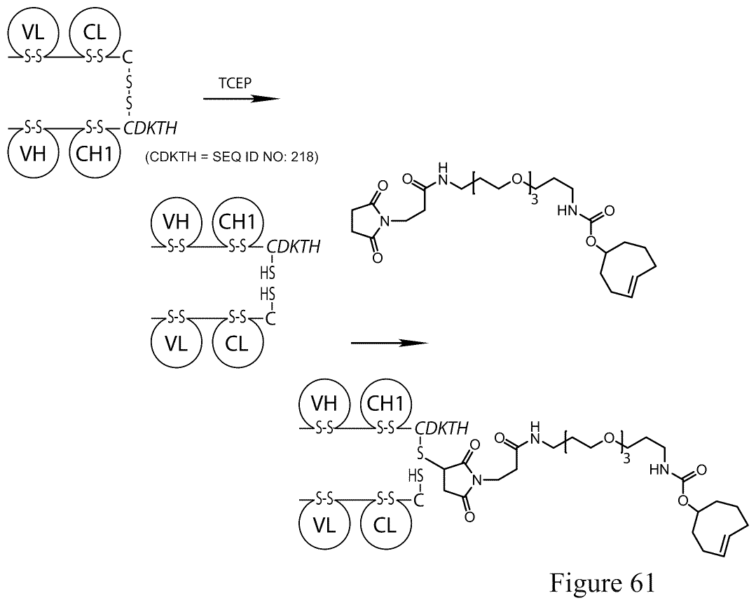

To prepare the transcyclooctene-modified Fab, TCEP-treated Fab was reacted with a heterobifunctional linker, TCO-PEG3-Maleimide, which contains a maleimide group capable of reacting with a free thiol group on the TCEP-treated Fab (FIG. 61). The Fab fragment was generated by papain digestion of 10 mg of adalimumab (Humira™) obtained from Abbott using a Pierce™ Fab Preparation Kit (Cat. No. 44985) according to the manufacturer's instructions. Following digestion, the Fab fragment was purified by chromatography on HiTrap Protein A HP to remove the Fc fragment and undigested antibody. The flow-through fractions, containing the Fab fragment, were buffer-exchanged into PBS, and concentrated to 5 mg/ml.

For the partial reduction of the Fab fragment with TCEP, reactions (0.26 ml) contained 0.1 M sodium phosphate pH 7.0, 0.5 mg of Fab, and 0.08 mg/ml TCEP. Following incubation for at room temperature for 60 minutes, the reaction was brought to 0.72 ml with the addition of 0.24 ml of 0.3 M sodium phosphate pH 7.0 and 0.22 ml of water. The TCO-PEG3-Maleimide linker was then added to the reaction (0.12 ml of at a concentration of 50 μg/ml in DMSO) and the reaction incubated for 20 minutes at room temperature. The transcyclooctene-modified Fab was buffered-exchanged on a PD-10 column into 0.02 M sodium phosphate pH 7.0 to remove excess linker, and the final product concentrated to 2.7 mg/ml. Under these conditions, greater than 90% of the Fab heavy chain and less than 10% of the Fab light chain was modified by the TCO-PEG3-Maleimide linker. FIG. 61 shows the structure of the transcyclooctene-modified Fab protein (Fab-P3-Tco).

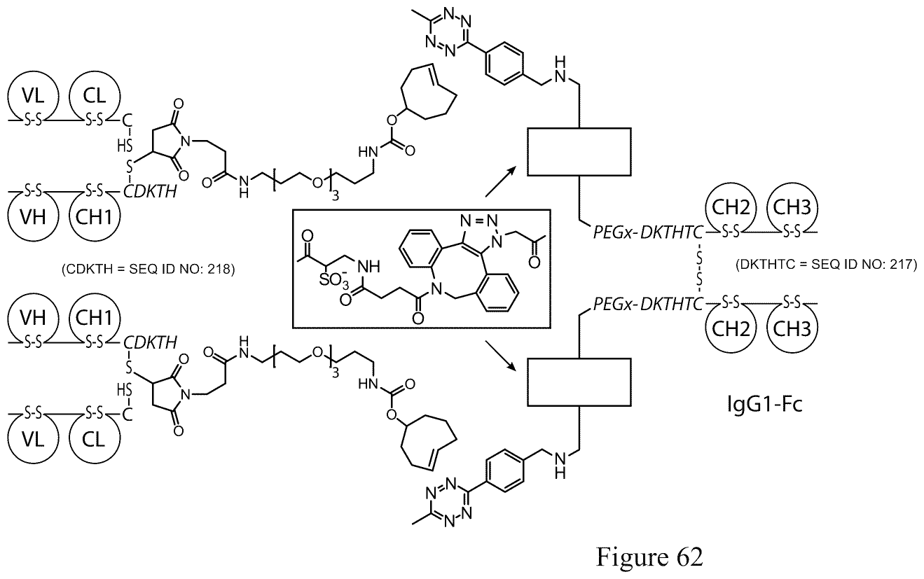

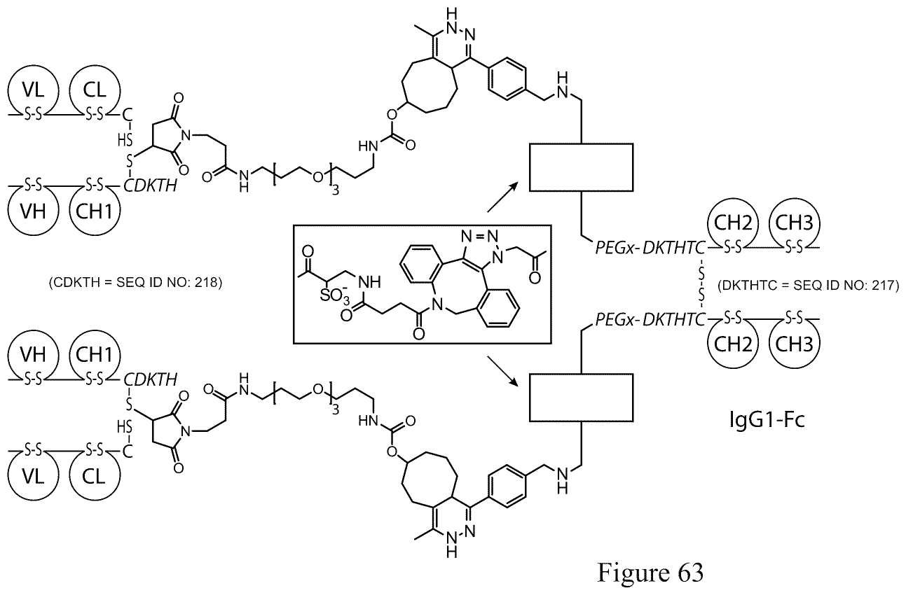

The Fab-P3-Tco protein was reacted individually with each of the Tet-Px-Fc proteins (FIG. 62), to generate the Fab-P3-TT-Px-Fc series of hybrid immunoglobulins (FIG. 63). Reactions (6 ul) contained 0.1 M sodium phosphate pH 7.0, 3.6 ug of the Fab-P3-Tco protein and 1 ug of each Tet-Px-Fc protein. Reactions were carried out at room temperature for 60 minutes.

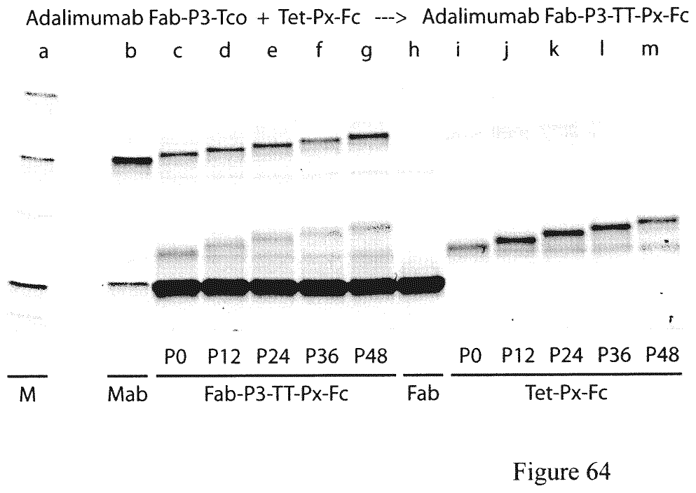

FIG. 64 shows the Fab-dihydropyridizine-Fc hybrid immunoglobulins by SDS-PAGE under reducing conditions: markers (lanes a), adalimumab (lane b), Fab-P3-TT-P0-Fc (lane c), Fab-P3-TT-P12-Fc (lane d), Fab-P3-TT-P24-Fc (lanes e), Fab-P3-TT-P36-Fc (lanes f), Fab-P3-TT-P48-Fc (lane g), Fab-P3-Tco (lane h), Tet-P0-Fc (lane i), Tet-P12-Fc (lane j), Tet-P24-Fc (lane k), Tet-P36-Fc (lane l)m Tet-P48-Fc (lane m). By comparison with adalimumab (lane b), the Fab-dihydropyridizine-Fc hybrid immunoglobulins had the expected size, showing an increase with PEG linker length comparable to the Tet-Px-Fc proteins. The conversion of each Tet-Px-Fc protein to the corresponding Fab-P3-TT-Px-Fc hybrid immunoglobulin was approximately 75%.

### Example 15: Olanzapine-dihydropyridizine-Fc Hybrid Immunoglobulins

In this example, hybrid immunoglobulins are prepared with an azide-derivative of a primary amine, secondary amine or alcohol compound. The azide-derivatized compound may be prepared as described in Pothukanuri, S. and Winssinger, N., Org Lett. 2007; 9(11):2223-5, hereby incorporated by reference. The primary amine, secondary amine or alcohol compound is first reacted with chloroalkyl chloroformate to obtain the chloroalkyl carbamate, followed by an azide displacement of the chloride, affording the azidoalkyl carbamate. All chemicals are obtained from Sigma-Aldrich.

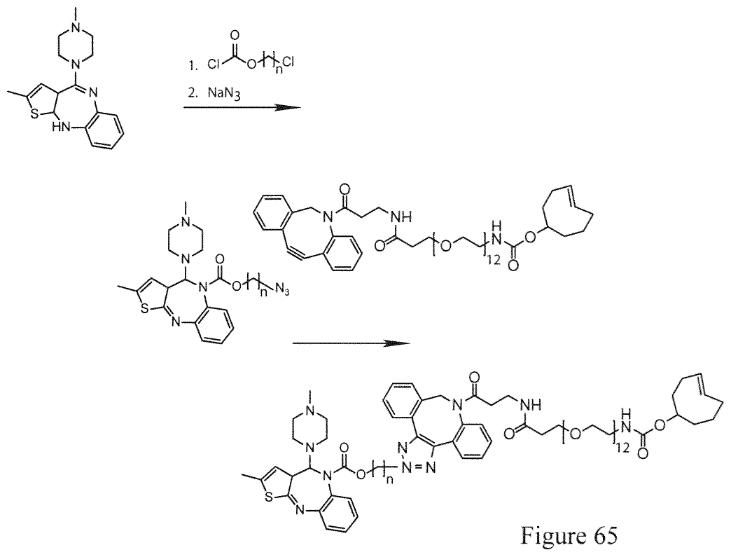

Olanzapine (Sigma Cat. No. 01141) is first reacted with chloromethyl chloroformate as described in U.S. patent application Ser. No. 13/801,344, published Oct. 10, 2013, Publication No. US20130267505 A1, hereby incorporated by reference. A solution of olazapine (60 mmoles) and triethylamine (120 mmoles) in anhydrous dichloromethane (250 ml) is warmed to 35° C. until a clear solution is formed, then cooled to 5° C. Chloromethyl chloroformate (90 mmoles) is then added over 20 minutes. Other suitable chloroalkyl chloroformates include 2-chloroethyl chloroformate, 3-chloropropyl chloroformate, and 4-chlorobutyl chloroformate. The reaction is stirred at room temperature for 30 min and allowed to warm to room temperature. After 15 min at room temperature the reaction mixture is diluted with dichloromethane (100 ml) then washed with aqueous saturated NaHCO₃ (75 ml) and water (350 ml). The organic phase is dried over MgSO4 and filtered. The organic phase is then concentrated under vacuum at 45° C. to a volume of 150 ml. The mixture is diluted with 30 ml ethyl acetate and is further evaporated (20-30 ml) under vacuum. The mixture is cooled to room temperature and the resulting solid precipitate is filtered and washed with ethyl acetate. After drying under vacuum at 35° C. for 90 min, chloromethyl 2-methyl-4-(4-methylpiperazin-1-yl)-5H-benzo[b]thieno[2,3-e][1,4]diazepine-5-carboxylate is obtained. This compound (1.5 eq) is then treated with NaN₃ (1.5 eq) in CH₃CN:H2O (1:1, 0.3 M) at room temperature for 8 to 36 hours. The reaction mixture is diluted with ethyl acetate and the organic phase is washed with water, brine then dried over Na₂SO₄ and concentrated in vacuo. Purification by HPLC affords the azide-olanzapine derivative, azidomethyl 2-methyl-4-(4-methylpiperazin-1-yl)-5H-benzo[b]thieno[2,3-e][1,4]diazepine-5-carboxylate (FIG. 65).

The azide-olanzapine derivative is then used to prepare series of olanzapine-dihydropyridizine-Fc hybrid immunoglobulins (Ola-P12-TT-Px-Fc) as follows. In a first step, the azide-olanzapine derivative is modified with a transcyclooctene functional group using a heterobifunctional linker. In a second step, the transcycloctene-modified olanzapine is reacted with the Tet-Px-Fc proteins of Example 11.

To prepare the transcyclooctene-modified olanzapine, the azide-olanzapine derivative is reacted with the heterobifunctional linker TCO-PEG12-DBCO which contains a cyclooctyne group capable of reacting with the azide group (FIG. 65). Reactions (1 ml) contain 0.5 mg of the azide-olanzapine derivative and 5 mg of the TCO-PEG12-DBCO linker in DMSO. Reactions are carried out at room temperature for 3 to 20 hours. The transcycloctene-modified olanzapine (Ola-P12-Tco) is purified by HPLC to remove excess unreacted TCO-PEG12-DBCO linker. Prior to use, Ola-P12-Tco is dissolved at a concentration of 1 mg/mL in DMSO.

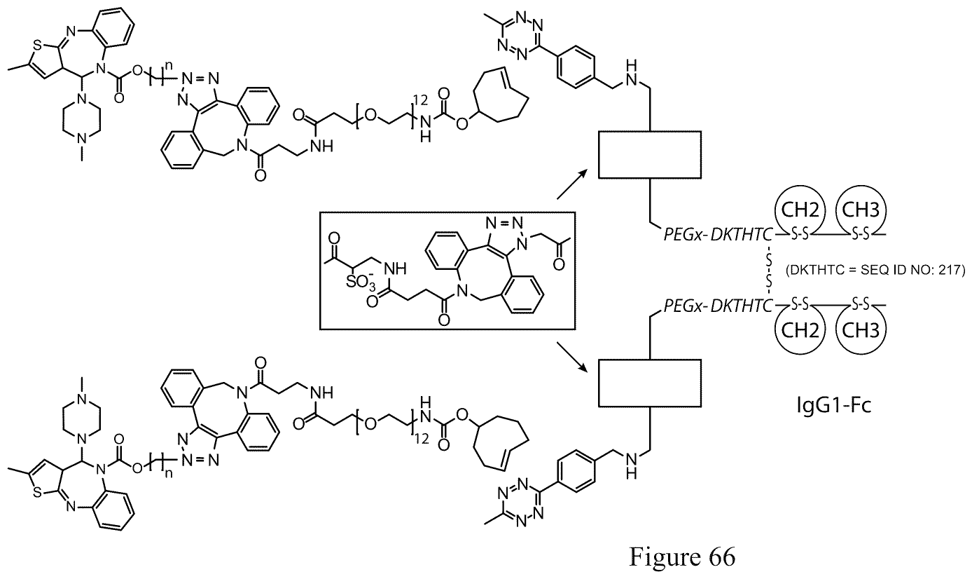

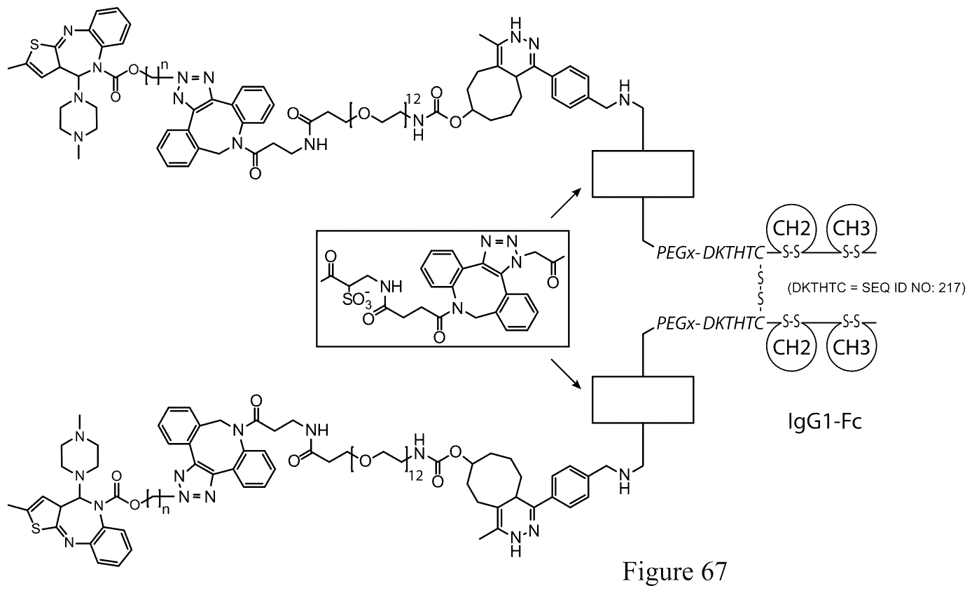

Ola-P12-Tco is reacted individually with each of the Tet-Px-Fc proteins (FIG. 66), to generate the Ola-P12-TT-Px-Fc series of hybrid immunoglobulins (FIG. 67). Reactions (1 ml) contain 0.1 mg of GLP1-P7-Tet peptide and 0.33 mg of each Tco-Px-Fc protein in DMSO. Reactions are carried out at room temperature for 60 minutes. The Ola-P12-TT-Px-Fc hybrid immunoglobulins are then purified by chromatography on HiTrap Protein A HP.

### DISCUSSION

Aspects of the present invention provide the chemical semisynthesis of antibodies with nonprotein hinges that incorporate large binding domains such as the Fab itself or receptor extracellular domains. The present invention relates to the identification of ligation reactions that are compatible with the native structure and function of the cognate proteins and proceed efficiently. Aspects of the present invention provide compounds having nonprotein chains that are both flexible and extendible. Antibody-like molecules provided in embodiments of the invention have enormous potential as therapeutic candidates with improved binding affinity for their disease targets.

1. A compound having the structure: 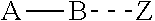
wherein A is a biologically active structure of the compound;
wherein Z is a protein component of the compound, which protein component comprises one or more polypeptides, wherein at least one of the one or more polypeptides comprises consecutive amino acids which (i) are identical to a stretch of consecutive amino acids present in a chain of an F_{c} domain of an antibody; (ii) bind to an F_{c} receptor; and (iii) have at their N-terminus a cysteine or a selenocysteine;
wherein B is (a) an organic acid residue or (b) a stretch of consecutive amino acid residues which is, or is present in any of the following sequences: EPKSCDKTHTCPPCP (SEQ ID NO: 213), ERKCCVECPPCP (SEQ ID NO: 214), ELKTPLGDTTHTCPRCP(EPKSCDTPPPCPRCP)3 (SEQ ID NO: 215), or ESKYGPPCPSC (SEQ ID NO: 216);
wherein the dashed line between B and Z represents a peptidyl linkage between the N-terminal cysteine or selenocysteine of Z and an amino acid residue or an organic acid residue of B; and
wherein the solid line between A and B represents a nonpeptidyl linkage comprising the structure: 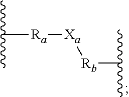
wherein Xₐ is a chemical structure containing a cyclooctane fused to a dihydropyridazine,
wherein Rₐ represents an organic structure which connects to one of A or B and R_{b} represents an organic structure which connects to the other of A or B.

2-6. (canceled)

7. The compound according to claim 6, wherein Xₐ has the structure: 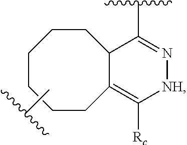
wherein R_{c} is H, alkyl or aryl;
or a tautomer thereof.

8. The compound according to claim 6, wherein Rₐ is connected to the cyclooctane and R_{b} is connected to the dihydropyridazine.

9. The compound according to claim 6, wherein the solid line between A and B represents a nonpeptidyl linkage comprising the structure: 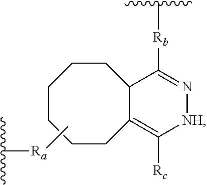
wherein R_{c} is H, alkyl or aryl;
or a tautomer thereof.

10. The compound according to claim 6, wherein Rₐ and R_{b} are independently a bond or an organic structure comprising or consisting of a chain of 1, 2, 3, 4, 5, 6, 7, 8, 9, 10 or more moieties, wherein each moiety is independently selected from the group consisting of [PEG(y)]z, polyalkylene glycol, polyoxyalkylated polyol, polyvinyl alcohol, polyvinyl alkyl ether, poly(lactic acid), poly(lactic-glycolic acid), polysaccharide, a branched residue, C₁-C₁₀ alkyl, C₃-C₁₀ cycloalkane, C₂-C₁₀ alkene, C₅-C₁₀ cycloalkene, amine, sulfur, oxygen, succinimide, maleimide, glycerol, triazole, isoxazolidine, C₂-C₅ acyl, C₂-C₅ acylamino, C₂-C₅ acyloxy, succinyl, malonyl, glutaryl, phthalyl, adipoyl, an amino acid, an aryl group, a heteroaryl group, a carbamate, a chemical structure containing a cyclooctane fused to a dihydropyridazine, a chemical structure containing a cyclooctene fused to a triazole, a chemical structure containing a cyclooctene fused to a isoxazolidine, a dibenzocyclooctene, a dibenzoazacyclooctene, 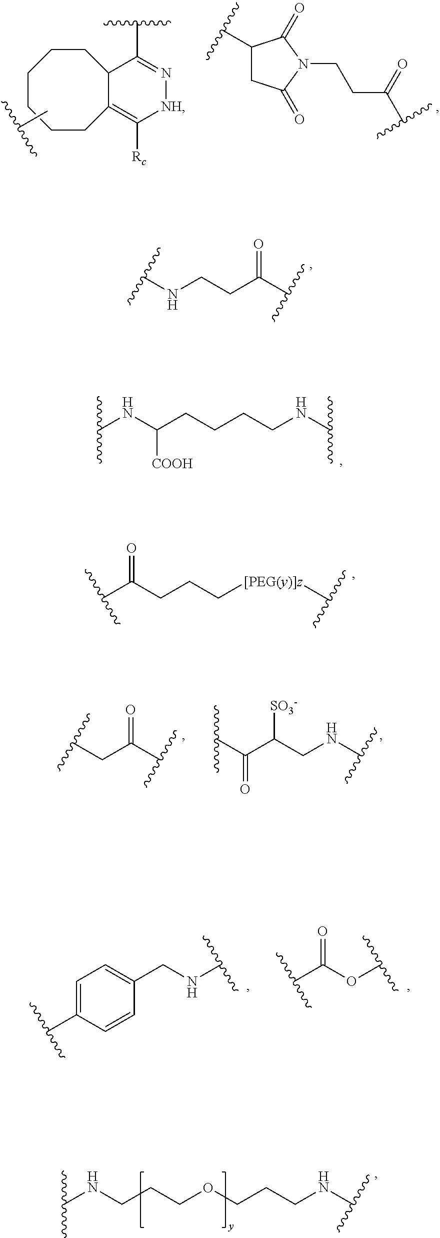 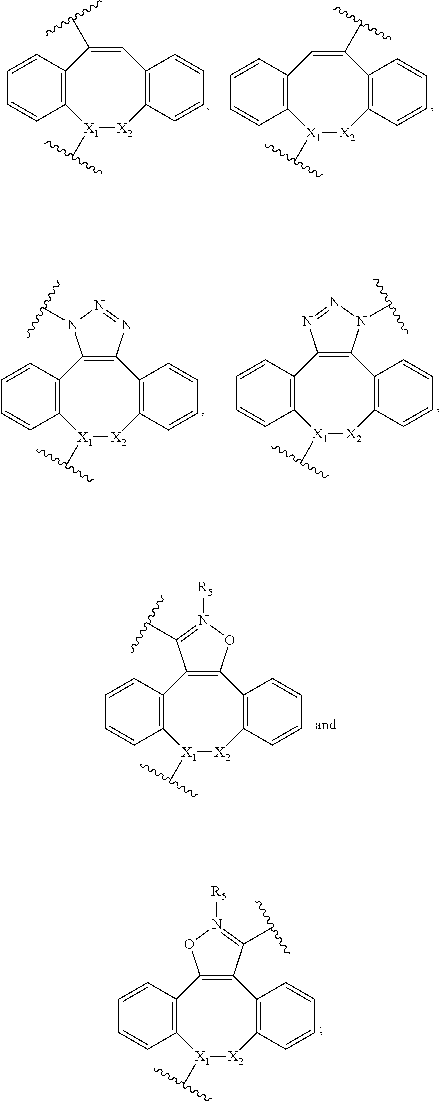
wherein X₁ is CH or N, X₂ is CH₂ or a carbonyl group, and R₅ is an aryl or alkyl group;
wherein [PEG(y)]z is: 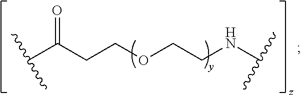
wherein y=1-100 and z=1-10.

11. The compound according to claim 6, wherein Ra or Rb
i) is attached to A via a carbon-nitrogen bond or a carbon-sulfur bond;
ii) is attached to A via a carbon-nitrogen bond;
iii) is attached to A via a carbon-nitrogen bond, wherein the carbon-nitrogen bond is an amide bond;
iv) is attached to A via a biologically labile bond;
v) is attached to A via an amide bond between the C-terminal amino acid of A and an amino group in B;
vi) is attached to A via an amide bond between the C-terminal amino acid of A and an amino group in B, wherein the terminal amino acid is cysteine;
vii) is attached to A via a carbon-sulfur bond;
viii) is attached to A via a carbon-sulfur bond formed between R₂ and a free thiol;
ix) is is attached to A via a succinimide-sulfur bond;
x) comprises a branched residue; or
xi) is attached to more than one A via the branched residue.

12. The compound according to claim 1, wherein A
i) comprises the structure of a compound that is a drug approved for treating a subject afflicted with a disease;
ii) comprises the structure of an organic compound having a molecular weight less than 1000 Daltons, a DNA aptamer, an RNA aptamer, an oligonucleotide, or a protein that is biologically active;
iii) comprises a primary or a secondary amine;
iv) is linked to B via the primary or secondary amine;
v) is aripiprazole or oseltamivir;
vi) comprises a secondary amine;
vii) is a respiratory drug, an antiasthmatic agent, an analgesic agent, an antidepressant, an antianginal agent, an antiarrhythmic agent, an antihypertensive agent, an antidiabetic agent, an antihistamine, an anti-infective agent, an antibiotic, an antiinflammatory agent, an antiparkinsonism drug, an antipsychotics, an antipyretic agent, an antiulcer agent, an attention deficit hyperactivity disorder (ADHD) drug, a central nervous system stimulant, a decongestant, or a psychostimulant;
viii) is alprenolol, acebutolol, amidephrine, amineptine, amosulalol, amoxapine, amphetaminil, atenolol, atomoxetine, balofloxacin, bamethan, befunolol, benazepril, benfluorex, benzoctamine, betahistine, betaxolol, bevantolol, bifemelane, bisoprolol, brinzolamide, bufeniode, butethamine, camylofine, carazolol, carticaine, carvedilol, cephaeline, ciprofloxacin, cloZapine, clobenZorex, clorprenaline, cyclopentamine, delapril, demexiptiline, denopamine, desipramine, desloratadine, diclofenac, dimetofrine, dioxadrol, dobutamine, dopexamine, doripenem, dorzolamide, droprenilamine, duloxetine, eltopraZine, enalapril, enoxacin, epinephrine, ertapenem, esapraZole, esmolol, etoxadrol, fasudil, fendiline, fenethylline, fenfluramine, fenoldopam, fenoterol, fenproporex, flecamide, fluoxetine, formoterol, frovatriptan, gaboxadol, garenoxacin, gatifloxacin, grepafloxacin, hexoprenaline, imidapril, indalpine, indecainide, indeloxazine hydrochloride, isoxsuprine, ispronicline, labetalol, landiolol, lapatinib, levophacetoperane, lisinopril, lomefloxacin, lotrafiban, maprotiline, mecamylamine, mefloquine, mepindolol, meropenem, metapramine, metaproterenol, methoxyphenamine, dextrorotary methylphenidate, methylphenidate, metipranolol, metoprolol, mitoxantrone, mivazerol, moexipril, moprolol, moxifloxacin, nebivolol, nifenalol, nipradilol, norfloxacin, nortriptyline, nylidrin, olanZapine, oxamniquine, oxprenolol, oxyfedrine, paroxetine, perhexyline, phenmetrazine, phenylephrine, phenylpropylmethylamine, pholedrine, picilorex, pimethylline, pindolol, pipemidic acid, piridocaine, practolol, pradofloxacin, pramipexole, pramiverin, prenalterol, prenylamine, prilocalne, procaterol, pronethalol, propafenone, propranolol, propylhexedrine, protokylol, protriptyline, pseudoephedrine, reboxetine, rasagiline, (r)-rasagiline, repinotan, reproterol, rimiterol, ritodrine, safinamide, salbutamol/albuterol, salmeterol, sarizotan, sertraline, silodosin, sotalol, soterenol, sparfloxacin, spirapril, sulfinalol, synephrine, tamsulosin, tebanicline, tianeptine, tirofiban, tretoquinol, trimetazidine, troxipide, varenicline, vildagliptin, viloxazine, viquidil or xamoterol;
ix) comprises a protein that is biologically active;
x) comprises a secreted protein;
xi) comprises an extracellular domain of a protein
xii) is biologically active such that it has target-binding activity;
xiii) is an independently-folding protein or a portion thereof;
xiv) is a glycosylated protein;
xv) comprises intra-chain disulfide bonds;
xvi) binds a cytokine;
xvii) binds to a cytokine, wherein the cytokine is TNFα;
xviii) comprises Atrial Natriuretic Peptide (ANP), Calcitonin, Corticotropin Releasing Hormone (CRH), Endothelin, Exenatide, Gastric Inhibitory Peptide (GIP), Glucagon-Like Peptide-1 (GLP-1), Glucagon-Like Peptide-2 (GLP-2), an analog of GLP-1 or GLP-2, Glucagon Vasoactive Intestinal Peptide (GVIP), Ghrelin, Peptide YY or Secretin, or a portion thereof;
ixx) comprises a stretch of consecutive amino acids in the sequence HGEGTFTSDVSSYLEEQAAKEFIAWLVKGRG;
xx) comprises at least one stretch of consecutive amino acids which are identical to a stretch of consecutive amino acids present in the heavy chain of a Fab or a Fab′ of an antibody;
xxi) comprises at least one at least one stretch of consecutive amino acids which are identical to a stretch of consecutive amino acids present in the light chain of a Fab or a Fab′ of an antibody;
xxii) comprises at least one Fab or Fab′ of an antibody, or a portion of at least one Fab or Fab′;
xxiii) comprises Fab-1 or Fab′1, or a portion thereof of an antibody;
xxiv) comprises Fab-2 or Fab′2, or a portion thereof of an antibody;
xxv) comprises two Fab or Fab′ hands of an antibody;
xxvi) comprises at least one stretch of consecutive amino acids which are identical to a stretch of consecutive amino acids present in a single chain antibody; or
xxvii) comprises at least one stretch of consecutive amino acids which are identical to a stretch of consecutive amino acids present in a TNFα receptor.

13. A compound having the structure: 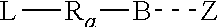
wherein Z is a protein component of the compound, which protein component comprises one or more polypeptides, wherein at least one of the one or more polypeptides comprises consecutive amino acids which (i) are identical to a stretch of consecutive amino acids present in a chain of an F_{c} domain of an antibody; (ii) bind to an F_{c} receptor; and (iii) have at their N-terminus a cysteine or a selenocysteine;
wherein B is a chemical structure linking Rₐ and C;
wherein the dashed line between B and Z represents a peptidyl linkage;
wherein L is selected from the group consisting of: a tetrazine and a trans-cyclooctene; and
wherein Rₐ is a bond or an organic structure comprising or consisting of a chain of 1, 2, 3, 4, 5, 6, 7, 8, 9, 10 or more moieties, wherein each moiety is independently selected from the group consisting of [PEG(y)]z, polyalkylene glycol, polyoxyalkylated polyol, polyvinyl alcohol, polyvinyl alkyl ether, poly(lactic acid), poly(lactic-glycolic acid), polysaccharide, a branched residue, C₁-C₁₀ alkyl, C₃-C₁₀ cycloalkane, C₂-C₁₀ alkene, C₅-C₁₀ cycloalkene, amine, sulfur, oxygen, succinimide, maleimide, glycerol, triazole, isoxazolidine, C₂-C₅ acyl, C₂-C₅ acylamino, C₂-C₅ acyloxy, succinyl, malonyl, glutaryl, phthalyl, adipoyl, an amino acid, an aryl group, a heteroaryl group, a carbamate, a chemical structure containing a cyclooctane fused to a dihydropyridazine, a chemical structure containing a cyclooctene fused to a triazole, a chemical structure containing a cyclooctene fused to a isoxazolidine, a dibenzocyclooctene, a dibenzoazacyclooctene, 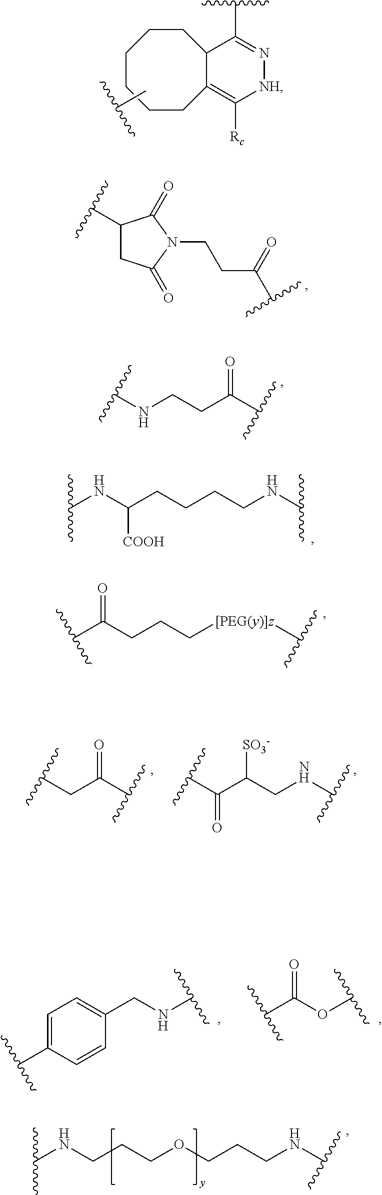 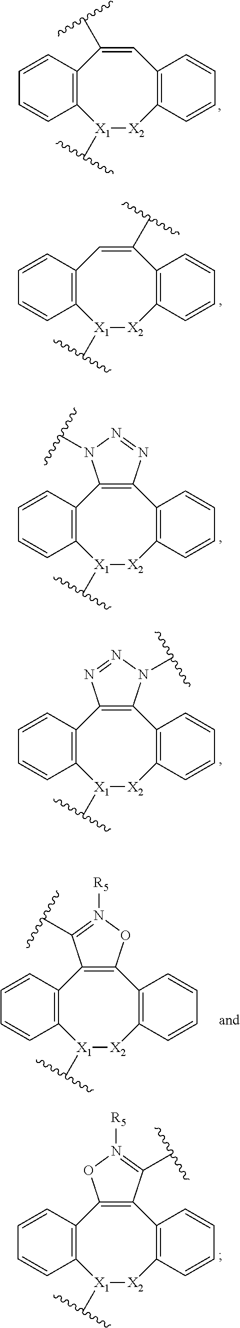
wherein X₁ is CH or N, X₂ is CH₂ or a carbonyl group, and R₅ is an aryl or alkyl group;
wherein [PEG(y)]z is: 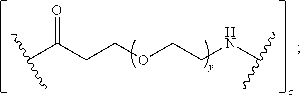
wherein y=1-100 and z=1-10.

14-15. (canceled)

16. The compound according to claim 13, wherein L is a tetrazine, and the tetrazine has the structure: 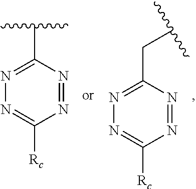
wherein Rc is H, alkyl or aryl.

17. The compound according to claim 13, wherein L is trans-cyclooctene, and the trans-cyclooctene has the structure:

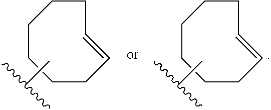

18. The compound according to claim 6, wherein Ra or Rb
i) is an organic structure comprising a [PEG(y)]z group;
ii) is an organic structure comprising a polyalkylene glycol, polyoxyalkylated polyol, polyvinyl alcohol, polyvinyl alkyl ether, poly(lactic acid), poly(lactic-glycolic acid), or polysaccharide group;
iii) is an organic structure comprising a C₁-C₄ alkyl group
iv) is an organic structure comprising a succinimide
v) is an organic structure comprising an amine;
vi) is an organic structure comprising a succinyl, malonyl, glutaryl, phthalyl or adipoyl;
vii) is an organic structure comprising a malonyl;
viii) is an organic structure comprising an amino acid;
ix) is an organic structure comprising a cysteine;
x) is an organic structure comprising a lysine;
xi) is an organic structure consisting of a chain of 3 moieties selected from the group consisting of [PEG(y)]z, polyalkylene glycol, polyoxyalkylated polyol, polyvinyl alcohol, polyvinyl alkyl ether, poly(lactic acid), poly(lactic-glycolic acid), polysaccharide, a branched residue, C₁-C₁₀ alkyl, C₃-C₁₀ cycloalkane, C₂-C₁₀ alkene, C₅-C₁₀ cycloalkene, amine, sulfur, oxygen, succinimide, maleimide, glycerol, triazole, isoxazolidine, C₂-C₅ acyl, C₂-C₅ acylamino, C₂-C₅ acyloxy, succinyl, malonyl, glutaryl, phthalyl, adipoyl, an amino acid, an aryl group, a heteroaryl group, a carbamate, a chemical structure containing a cyclooctane fused to a dihydropyridazine, a chemical structure containing a cyclooctene fused to a triazole, a chemical structure containing a cyclooctene fused to a isoxazolidine, a dibenzocyclooctene, a dibenzoazacyclooctene, 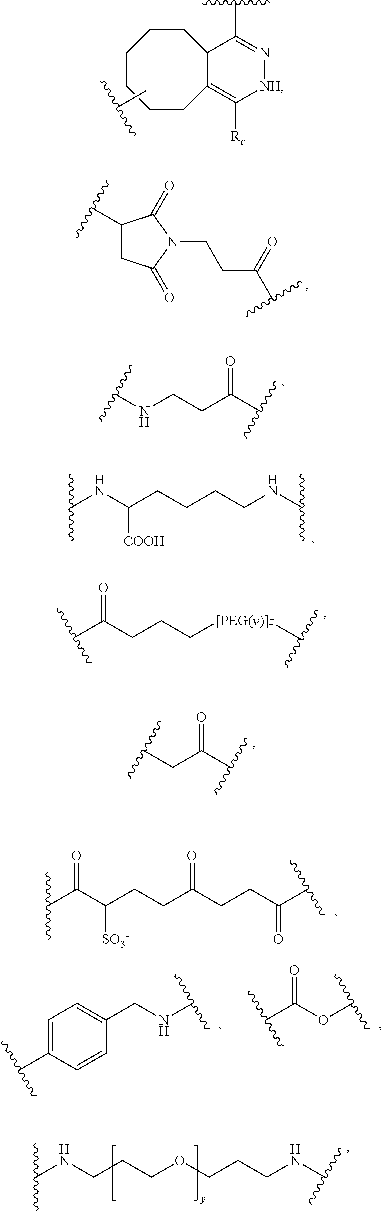 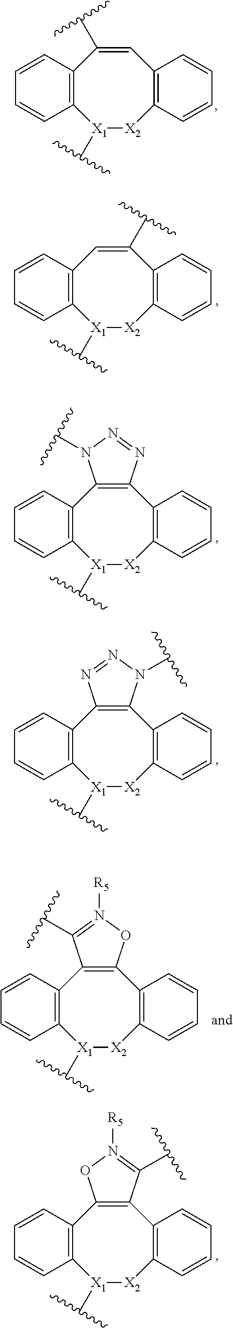
xii) is an organic structure consisting of a chain of four moieties selected from the group consisting of [PEG(y)]z, polyalkylene glycol, polyoxyalkylated polyol, polyvinyl alcohol, polyvinyl alkyl ether, poly(lactic acid), poly(lactic-glycolic acid), polysaccharide, a branched residue, C₁-C₁₀ alkyl, C₃-C₁₀ cycloalkane, C₂-C₁₀ alkene, C₅-C₁₀ cycloalkene, amine, sulfur, oxygen, succinimide, maleimide, glycerol, triazole, isoxazolidine, C₂-C₅ acyl, C₂-C₅ acylamino, C₂-C₅ acyloxy, succinyl, malonyl, glutaryl, phthalyl, adipoyl, an amino acid, an aryl group, a heteroaryl group, a carbamate, a chemical structure containing a cyclooctane fused to a dihydropyridazine, a chemical structure containing a cyclooctene fused to a triazole, a chemical structure containing a cyclooctene fused to a isoxazolidine, a dibenzocyclooctene, a dibenzoazacyclooctene, 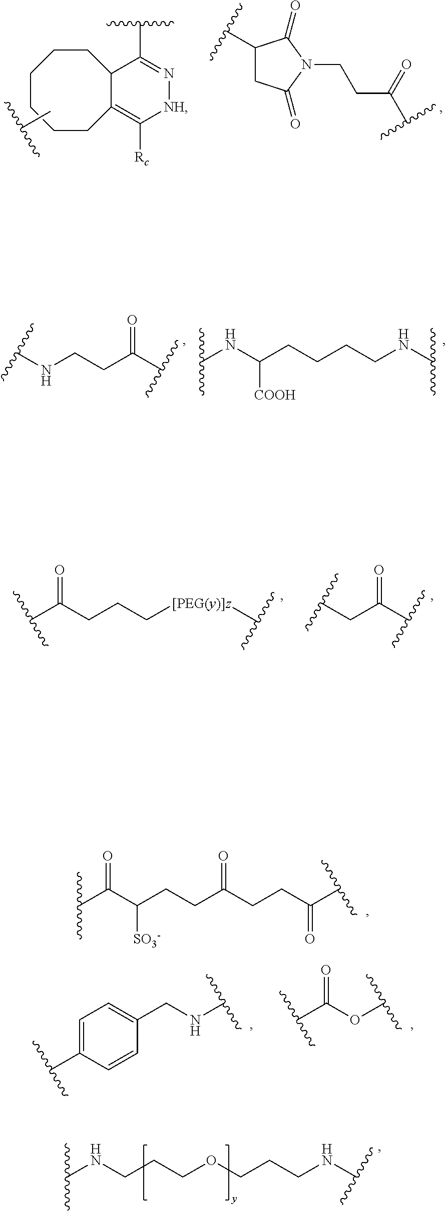 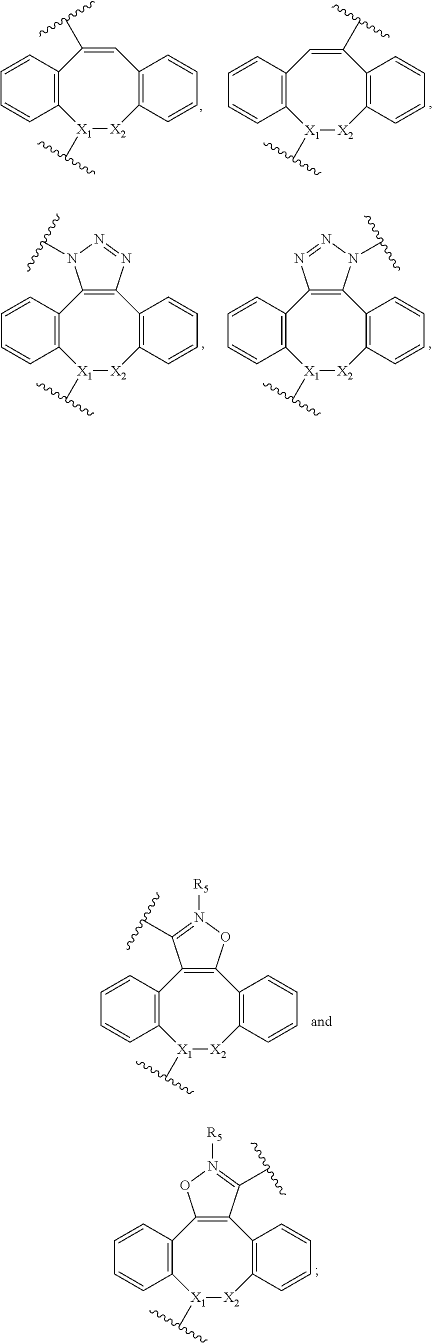
xiii) is an organic structure consisting of a chain of five moieties selected from the group consisting of [PEG(y)]z, polyalkylene glycol, polyoxyalkylated polyol, polyvinyl alcohol, polyvinyl alkyl ether, poly(lactic acid), poly(lactic-glycolic acid), polysaccharide, a branched residue, C₁-C₁₀ alkyl, C₃-C₁₀ cycloalkane, C₂-C₁₀ alkene, C₅-C₁₀ cycloalkene, amine, sulfur, oxygen, succinimide, maleimide, glycerol, triazole, isoxazolidine, C₂-C₅ acyl, C₂-C₅ acylamino, C₂-C₅ acyloxy, succinyl, malonyl, glutaryl, phthalyl, adipoyl, an amino acid, an aryl group, a heteroaryl group, a carbamate, a chemical structure containing a cyclooctane fused to a dihydropyridazine, a chemical structure containing a cyclooctene fused to a triazole, a chemical structure containing a cyclooctene fused to a isoxazolidine, a dibenzocyclooctene, a dibenzoazacyclooctene, 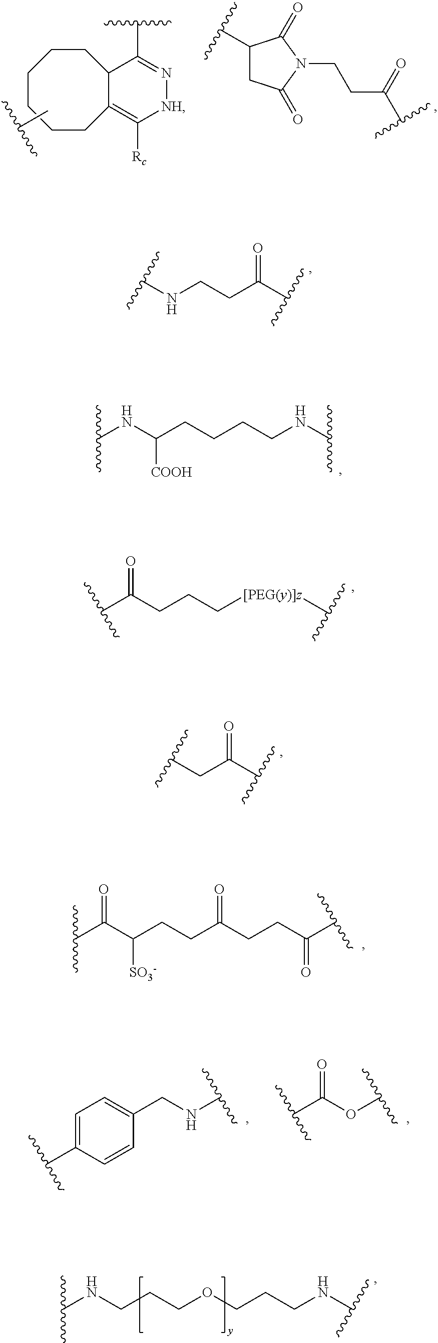 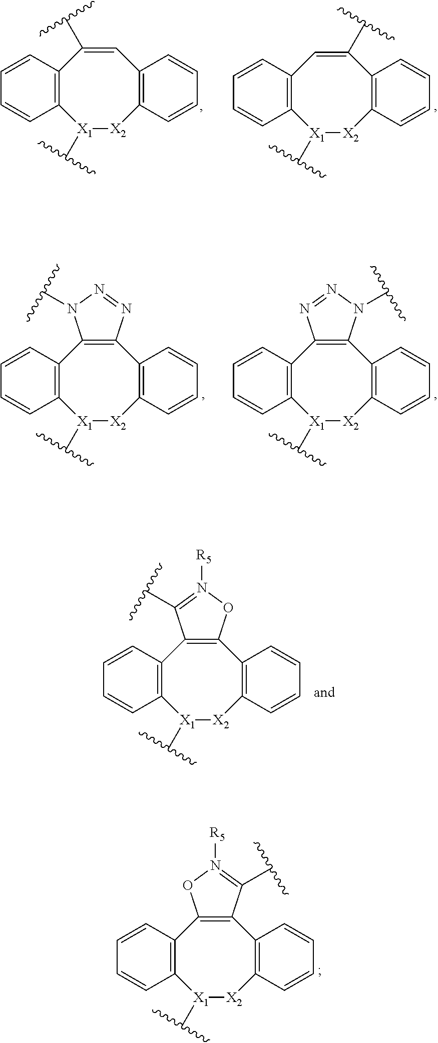
xiv) comprises a [PEG(y)]z group bonded to a lysine;
xv) comprises a C₁-C₄ acyl group bonded to a succinimide group;
xvi) comprises a lysine bonded to a C₁-C₄ acyl
xvii) comprises a [PEG(y)]z group, which is bonded to a glutaryl;
xviii) is an organic structure consisting of a chain of three, four or five moieties selected from the group consisting of [PEG(y)]z, C₂-C₅ acyl, succinyl, malonyl, glutaryl, an amino acid, a chemical structure containing a cyclooctane fused to a dihydropyridazine, a chemical structure containing a cyclooctene fused to a triazole, a chemical structure containing a cyclooctene fused to a isoxazolidine, a dibenzocyclooctene, a dibenzoazacyclooctene, 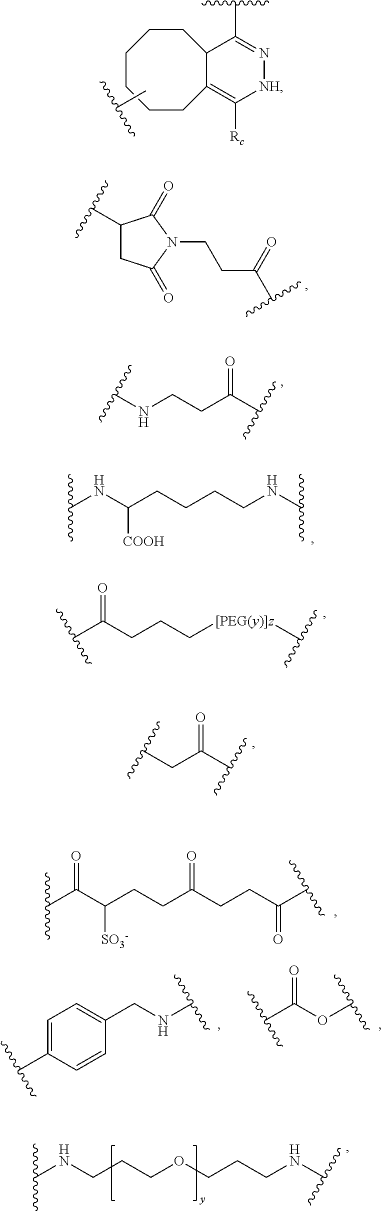 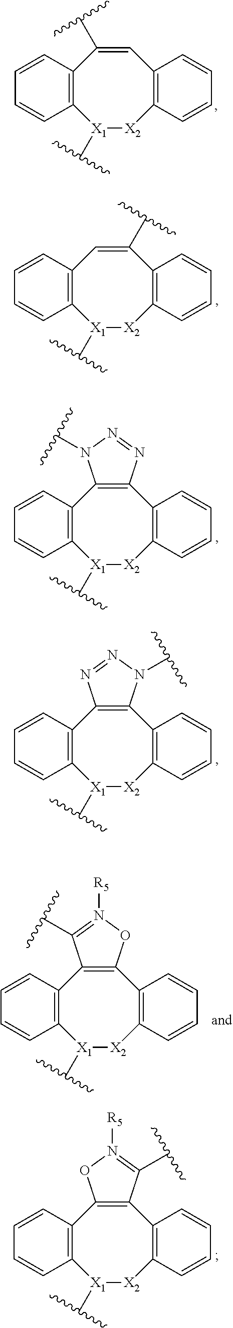
wherein X₁ is CH or N, X₂ is CH₂ or a carbonyl group, and R₅ is an aryl or alkyl group;
wherein [PEG(y)]z is: 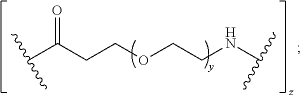
wherein y=1-100 and z=1-10;
ixx) is a bond;
xx) is a cysteine;
xxi) has a linear structure; or
xxii) has a branched structure;
xxiii) has the structure: 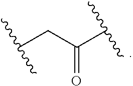 [SMILES: *CC(*)=O]
xxiv) is: 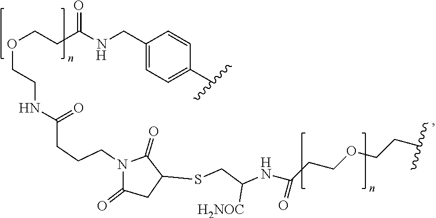
wherein n is 1, 2, 3, 4, 5, 6, 7, 8, 9, 10, 11, 12, 13, 14, 15, 16, 17, 18, 19, 20, 1-30, 1-40, or 1-50;
xxv) is: 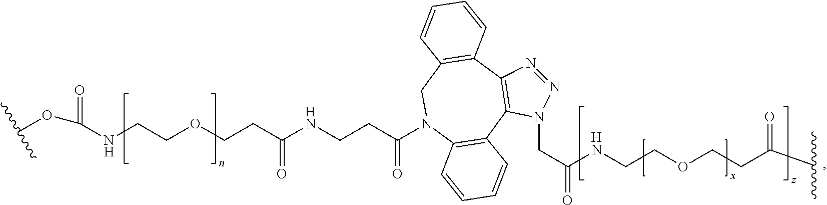
wherein n is 1, 2, 3, 4, 5, 6, 7, 8, 9, 10, 11, 12, 13, 14, 15, 16, 17, 18, 19, 20, 1-30, 1-40, or 1-50, x is 1, 2, 3, 4, 5, 6, 7, 8, 9, 10, 11, 12, 13, 14, 15, 16, 17, 18, 19, 20, 1-30, 1-40, or 1-50 and z is 1, 2, 3, 4, 5, 6, 7, 8, 9, 10, 11, 12, 13, 14, 15, 16, 17, 18, 19, 20, 1-30, 1-40, or 1-50.
xxvi) is: 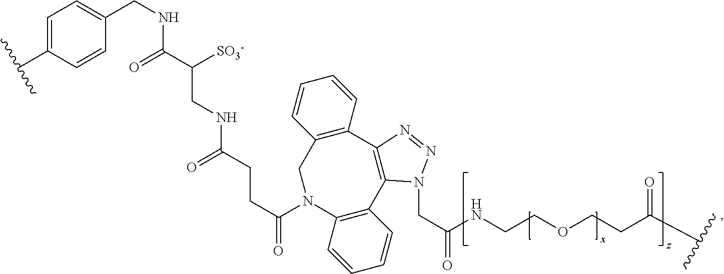
wherein x is 1, 2, 3, 4, 5, 6, 7, 8, 9, 10, 11, 12, 13, 14, 15, 16, 17, 18, 19, 20, 1-30, 1-40, or 1-50 and z is 1, 2, 3, 4, 5, 6, 7, 8, 9, 10, 11, 12, 13, 14, 15, 16, 17, 18, 19, 20, 1-30, 1-40, or 1-50; or
xxvii) is: 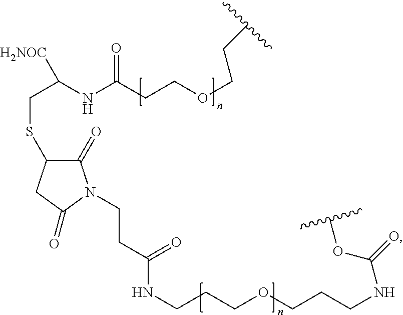
wherein n is 1, 2, 3, 4, 5, 6, 7, 8, 9, 10, 11, 12, 13, 14, 15, 16, 17, 18, 19, 20, 1-30, 1-40, or 1-50.

19. The compound according to any one of claims 1-18, wherein Z comprises one C, wherein C is a first polypeptide, which first polypeptide comprises consecutive amino acids which (i) are identical to a stretch of consecutive amino acids present in a chain of an F_{c} domain of an antibody; (ii) bind to an F_{c} receptor; and (iii) have at their N-terminus a sequence selected from the group consisting of a cysteine, selenocysteine, CP, CPXCP (where X=P, R, or S) (SEQ ID NOs: 206-208), CDKTHTCPPCP (SEQ ID NO: 209), CVECPPCP(SEQ ID NO: 210), CCVECPPCP (SEQ ID NO: 211) and CDTPPPCPRCP (SEQ ID NO: 212).

20. The compound according to claim 19, wherein C
i) comprises consecutive amino acids which (i) are identical to a stretch of consecutive amino acids present in a chain of an F_{c} domain of an antibody; (ii) bind to an F_{c} receptor; and (iii) have at their N-terminus a sequence comprising a naturally occurring cysteine selected from the group consisting of CP, CPXCP (where X=P, R, or S) (SEQ ID NOs: 206-208), CDKTHTCPPCP (SEQ ID NO: 209), CVECPPCP(SEQ ID NO: 210), CCVECPPCP (SEQ ID NO: 211) and CDTPPPCPRCP (SEQ ID NO: 212);
ii) is a polypeptide component of the compound, which polypeptide component comprises consecutive amino acids which (i) are identical to a stretch of consecutive amino acids present in a chain of an F_{c} domain of an antibody; (ii) bind to an F_{c} receptor; and (iii) have at their N-terminus a sequence comprising a non-naturally occurring cysteine or selenocysteine;
iii) comprises consecutive amino acids which are identical to a stretch of consecutive amino acids present in the chain of an Fc domain of an antibody selected from the group consisting of IgG, IgM, IgA, IgD, and IgE;
iv) comprises consecutive amino acids which are identical to a stretch of consecutive amino acids present in the chain of an Fc6 domain of an antibody
v) comprises consecutive amino acids which are identical to a stretch of consecutive amino acids present in a chain of an antibody other than a chain of a Fc domain of the antibody;
vi) consecutive amino acids which are identical to a stretch of consecutive amino acids present in a heavy chain of a Fab or a Fab′ of an antibody; or
vii) comprises consecutive amino acids which are identical to a stretch of consecutive amino acids present in the light chain of a Fab or a Fab′ of an antibody.

21. The compound according to claim 19, wherein Z further comprises a second polypeptide, which second polypeptide comprises consecutive amino acids which are identical to a stretch of consecutive amino acids present in a chain of an antibody other than a chain of a Fc domain of the antibody.

22. The compound according to claim 21, wherein the second polypeptide comprises
i) consecutive amino acids which are identical to a stretch of consecutive amino acids present in a heavy chain of a Fab or a Fab′ of an antibody; or
ii) consecutive amino acids which are identical to a stretch of consecutive amino acids present in the light chain of a Fab or a Fab′ of an antibody.

23. The compound according to claim 1, wherein Z
i) comprises an antibody or a portion thereof;
ii) comprises at least one Fab or Fab′ of an antibody, or a portion of the at least one Fab or Fab′
iii) comprises Fab-1 or Fab′1, or a portion thereof of an antibody;
iv) comprises Fab-2 or Fab′2, or a portion thereof of an antibody;
v) comprises two Fab or Fab′ hands of an antibody;
vi) comprises at least one stretch of consecutive amino acids which are identical to a stretch of consecutive amino acids present in a single chain antibody; or
vii) comprises a second polypeptide, and B is linked to Z via a peptidyl linkage between the N-terminal cysteine or selenocysteine of the second polypeptide of Z and an amino acid residue or an organic acid residue of B.24. The compound according to any one of claims 19-23, wherein the C-terminus of C
i) comprises a stretch of consecutive amino acids present in a chain of an F_{c} domain of an antibody that has been modified; or
ii) is a cysteine, selenocysteine, homocysteine, or homoselenosysteine, or a derivative of cysteine, selenocysteine, homocysteine, or homoselenosysteine.

25. (canceled)

26. A homodimer or a heterodimer comprising the compound of claim 1.

27. The homodimer or heterodimer of claim 26, wherein each compound of the homodimer or heterodimer
i) is capable of binding to the other by at least one disulfide bond;
ii) is capable of binding to the other by at least one disulfide bond between the Z or the second polypeptide of each compound;
iii) is bound to the other by at least one disulfide bond;
iv) is bound to the other by at least one disulfide bond between the Z or the second polypeptide of each compound.

28. (canceled)
